(19) United States
(12) Patent Application Publication
(10) Pub. No.: US 2026/0315452 A1
(43) Pub. Date: Oct. 15, 2026

(54) PROTEASE INHIBITORS AND USES THEREOF

(71) Applicant: WESTLAKE PHARMACEUTICALS (HANGZHOU) CO., LTD., Zhejiang (CN)

(72) Inventors: Hongtao YU, Zhejiang (CN); Qi HU, Zhejiang (CN); Jing HUANG, Zhejiang (CN); Tingliang WANG, Zhejiang (CN); Lijing ZHANG, Zhejiang (CN); Dongdong LIANG, Zhejiang (CN); Qi WU, Zhejiang (CN)

(21) Appl. No.: 19/474,099

(22) PCT Filed: Apr. 11, 2023
(86) PCT No.: PCT/CN2023/087543

§ 371 Date: Oct. 9, 2025

(51) Int. Cl. A61K 31/444; A61P 31/14; C07D 471/04

(52) U.S. Cl. CPC A61K 31/444; A61P 31/14; C07D 471/04

(57) ABSTRACT

The disclosure provides compounds of Formula I, compositions comprising the same, and methods of using the same, including use in treating a disease and/or a symptom of a disease caused by a coronavirus.

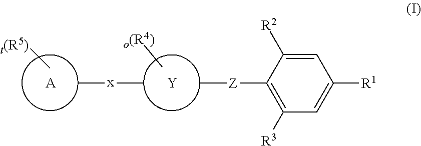

### FIELD OF THE DISCLOSURE

The present disclosure relates to compounds that inhibit proteases such as 3C-like protease (3CLpro), compositions comprising the compounds, methods of preparing the compounds, and methods of using the compounds to treat a disease or a symptom of a disease caused by a coronavirus, e.g., severe acute respiratory syndrome coronavirus 2 (SARS-CoV-2), MERS-CoV, or SARS-CoV.

### BACKGROUND OF THE DISCLOSURE

The COVID-19 pandemic is caused by a novel coronavirus, SARS-CoV-2. There is a great need for drugs that specifically treat diseases caused by SARS-CoV-2 or other coronaviruses. The positive-sense RNA virus SARS-CoV-2 has more than 70% of its genome encoding 16 non-structural proteins (nsps), named nsp1 to nsp16 (1, 2). The 16 nsps are translated as two polyproteins pp1a and pp1ab from which the individual nsps are generated by proteolytic cleavage. Specifically, pp1a is cleaved into nsp1 to nsp 11, while pp1ab is cleaved into nsp1 to nsp10 and nsp12 to nsp16 (1). The cleavage is carried out by two proteases included in the 16 nsps nsp3 and nsp5. Nsp3 is a large protein containing several domains among which the papain-like protease (PLpro) domain is responsible for the cleavage of the peptide bonds between nsp1 and 2, nsp2 and 3, and nsp3 and 4 (1, 3). Nsp5 is 3CLpro, a cysteine protease that cleaves peptide bonds to release nsp4 to nsp16 (1). 3CLpro has almost the same function in other coronaviruses. Examples of coronavirus 3CLpro protein sequences can be found in table 2 of WO2022150962.

After cleaving itself from pp1a and pp1ab, 3CLpro forms a homodimer with a significantly increased protease activity, facilitating it to cleave peptide bonds between other nsps. At the catalytic center of 3CLpro there is a catalytic dyad composed by His41 and Cys145. The working mechanism and the protein sequence of the catalytic domain of 3CLpro are conserved among different coronaviruses. The sequence identity between the 3CLpro of SARS-CoV-2 and that of SARS-CoV reaches 96%.

Inhibition of the protease activity of 3CLpro would block the release of nsp4 to nsp16 that are necessary for coronavirus replication. For example, nsp12 and nsp13, also known as the RNA-dependent RNA polymerase (RdRp) and helicase, respectively, are enzymes that catalyze the replication of the virus RNA genome. Therefore, 3CLpro is a promising target for the development of anti-coronavirus drugs. Several research teams have spent significant efforts in the development of inhibitors of SARS-CoV 3CLpro and SARS-CoV-2 3CLpro. The majority of these inhibitors are covalent inhibitors optimized from peptidic scaffolds (4). A large number of non-covalent inhibitors were also reported, however, most of them only showed moderate activity in cell-free or cell-based assays. Recently a noncovalent oral SARS-CoV-2 3CLpro inhibitor S-217622 was reported (5). Certain 3CLpro inhibitors are described in WO2022150962 and WO2023036140.

Accordingly, there is a need for compounds that are 3CLpro inhibitors with improved antiviral activity for the treatment a disease and/or a symptom of a disease caused by a coronavirus, for example, an infection caused by SARS-Cov-2, MERS-CoV, or SARS-CoV.

### SUMMARY OF THE DISCLOSURE

The present disclosure provides non-covalent inhibitors of proteases such as SARS-CoV-2 3CLpro, as well as pharmaceutical compositions comprising these inhibitors and uses of these inhibitors in the treatment of diseases and/or symptoms thereof caused by a coronavirus infection in a subject.

One aspect of this disclosure provides a compound selected from compounds of Formulae I, II-1, II-2, II-3, II-4, II-5, III, IV, V, VI, VII, VIII, and IX:

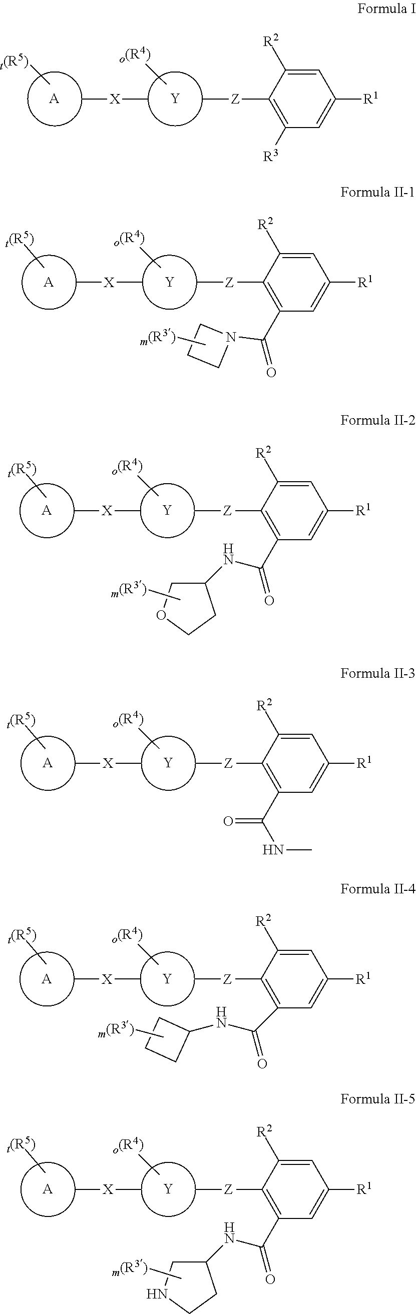

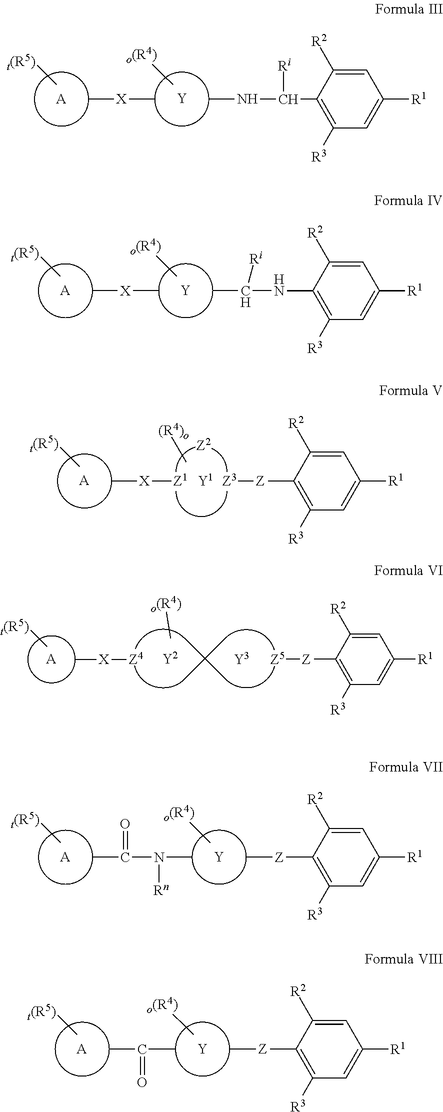

a solvate thereof, a tautomer thereof, a stereoisomer thereof, a deuterated derivative of the compound, or a pharmaceutically acceptable salt of the foregoing, wherein:
R¹ is hydrogen, halogen, or C₁-C₂ alkyl that is optionally substituted with 1 to 3 groups of halogen;
R² is —NO₂, —SO₃H, —SO₂CH₃, —CN or —CF₃;
R³ is 5- or 6-membered heteroaryl, —C(═O)R^{a}, —C(═O)OR^{a}, —C(═O)NR^{b}R^{c} or

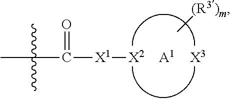

wherein:
   R^{a} is hydrogen, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{a} is optionally substituted with 1-3 groups selected from halogen and deuterium;
   R^{b} is hydrogen, C₁-C₄ alkyl, C₂-C₄ alkenyl, or C₂-C₄ alkynyl wherein the C₁-C₄ alkyl, C₂-C₄ alkenyl, or C₂-C₄ alkynyl of R¹ is optionally substituted with 1-3 groups selected from halogen and deuterium;
   R_{c} is hydrogen, NH₂, —OH, C₁-C₄ alkyl, C₂-C₄ alkenyl, or C₂-C₄ alkynyl wherein the C₁-C₄ alkyl, C₂-C₄ alkenyl, or C₂-C₄ alkynyl of R¹ is optionally substituted with 1-3 groups selected from halogen, deuterium, and —OH;
   Ring A¹ is 3-, 4-, 5- or 6-membered cycloalkyl, 3-, 4-, 5- or 6-heterocycloalkyl or 5- or 6-heteroaryl, wherein the heterocycloalkyl or heteroaryl of Ring A¹ contains one or more heteroatoms selected from N and O;
   X¹ is absent, —CH₂—, —NH— or —O—;
   X² is CH or N;
   X³ is —O—, —CH₂, or —NH—;
   R^{3′}, for each occurrence, is independently selected from halogen, —OR^{d}, —NR^{e}R^{f}, and C₁-C₄ alkyl that is optionally substituted with 1-3 groups of halogen, wherein:
      R^{d} is hydrogen, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{d} is optionally substituted with 1-3 groups of halogen;
      R^{e} is hydrogen, C₁-C₄ alkyl, C₂-C₄ alkenyl, or C₂-C₄ alkynyl wherein the C₁-C₄ alkyl, C₂-C₄ alkenyl, or C₂-C₄ alkynyl of R^{e} is optionally substituted with 1-3 groups of halogen;
      R^{f} is hydrogen, C₁-C₄ alkyl, C₂-C₄ alkenyl, or C₂-C₄ alkynyl wherein the C₁-C₄ alkyl, C₂-C₄ alkenyl, or C₂-C₄ alkynyl of R^{f} is optionally substituted with 1-3 groups of halogen;
   R^{3′}, for each occurrence, may be attached to any of the ring atoms of the Ring A¹, as long as valence permits; and
   m is an integer selected from 0, 1, 2 and 3;
Z is absent, —NR^{g}—, —C(R^{h}Rⁱ)—NR^{g}—, —NR^{g}—C(R^{h}Rⁱ)—, or —O—, wherein:
   R^{g} is hydrogen, C₁-C₆ alkyl, C₂-C₆ alkenyl, or C₂-C₆ alkynyl wherein the C₁-C₆ alkyl, C₂-C₆ alkenyl, or C₂-C₆ alkynyl of R^{g} is optionally substituted with 1-3 groups of halogen;
   R^{h} is hydrogen, C₁-C₆ alkyl, C₂-C₆ alkenyl, or C₂-C₆ alkynyl wherein the C₁-C₆ alkyl, C₂-C₆ alkenyl, or C₂-C₆ alkynyl of R^{h} is optionally substituted with 1-3 groups of halogen;
   Rⁱ is hydrogen, C₁-C₆ alkyl, C₂-C₆ alkenyl, or C₂-C₆ alkynyl wherein the C₁-C₆ alkyl, C₂-C₆ alkenyl, or C₂-C₆ alkynyl is optionally substituted with 1-3 groups of halogen;
Ring Y is phenyl or selected from the following rings:

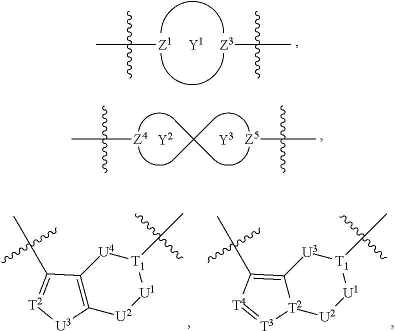

wherein:
   Ring Y¹ is 4-, 5-, 6-, 7-, 8-, or 9-membered cycloalkyl or 4-, 5-, 6-, 7-, 8-, or 9-membered heterocycloalkyl, wherein the heterocycloalkyl of Ring Y¹ contains one or more heteroatoms selected from N and O;
   Ring Y² is 4-, 5-, 6-, or 7-membered cycloalkyl or 4-, 5-, 6-, or 7-membered nitrogen heterocycloalkyl;
   Ring Y³ is 4-, 5-, 6-, or 7-membered cycloalkyl or 4-, 5-, 6-, or 7-membered nitrogen heterocycloalkyl;
   Z¹, Z³, Z⁴, and Z⁵, for each occurrence, are each independently CH or N;
   U¹, U², U³, and U⁴, for each occurrence, are each independently CH₂ or NH;
   T¹, T², T³, and T⁴, for each occurrence, are each independently C_{H} or N;
R⁴, for each occurrence, is independently halogen, —OH, —C(═O)R^{j}, C₁-C₄ alkyl, C₂-C₄ alkenyl, or C₂-C₄ alkynyl, wherein the C₁-C₄ alkyl, C₂-C₄ alkenyl, or C₂-C₄ alkynyl is optionally substituted with 1-3 groups of halogens, wherein:
   R^{j} is C₁-C₄ alkyl, C₂-C₄ alkenyl, or C₂-C₄ alkynyl wherein the C₁-C₄ alkyl, C₂-C₄ alkenyl, or C₂-C₄ alkynyl is optionally substituted with 1-3 groups of halogen;
R⁴, for each occurrence, may be attached to any of the ring atoms of Ring Y, as long as valence permits;
o is an integer selected from 0, 1 and 2;
X is absent, —CR^{k}R^{L}—, —C(═O)—, —C(═O)NRⁿ—, or —R^{m}—C(═O)NRⁿ—R^{o}—, wherein:
   R^{k}, R^{L}, and Rⁿ, for each occurrence, are each independently selected from hydrogen, halogen, and C₁-C₂ alkyl that is optionally substituted with 1-3 groups of halogen;
   R^{m} and R^{o}, for each occurrence, are each independently selected from hydrogen and C₁-C₂ alkyl that is optionally substituted with 1-3 groups of halogen;
Ring A is 5- or 6-membered nitrogen heteroalkyl or nitrogen heteroaryl, or 9- or 10-membered nitrogen heterocyclyl, wherein:
Ring A contains —N═, or ═N— in the meta position to the ring atom to which X is attached, or —NH— in the meta position or para position to the ring atom to which X is attached;
the —NH— in the meta position is attached to —C(═O)—in the ortho position, or the —NH— in the para position is attached to —C(═O)—in the meta position;
Ring A is substituted by t groups of R⁵, and R⁵ may be attached to any of the ring atoms of Ring A, as long as valence permits;
R⁵, for each occurrence, is independently halogen, —CN, —OR^{p}, —NHR^{p}, —CH₂NHBoc, 5- or 6-membered cycloalkyl, 5- or 6-membered heterocyclyl, 5- or 6-membered heteroaryl, C₁-C₄ alkyl, C₂-C₄ alkenyl, or C₂-C₄ alkynyl wherein the C₁-C₄ alkyl, C₂-C₄ alkenyl, or C₂-C₄ alkynyl of R⁵ is optionally substituted with 1-3 groups of halogen, wherein:
   R^{p}, for each occurrence, is independently selected from halogen and C₁-C₄ alkyl; and
t is 0, 1, 2, 3, 4, or 5.

Another aspect of the disclosure provides a pharmaceutical composition comprising a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, and a pharmaceutically acceptable carrier.

A further aspect of the disclosure provides a pharmaceutical composition comprising a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, and a pharmaceutically acceptable carrier in combination with an additional active pharmaceutical agent.

A further aspect of the disclosure provides methods of treating a disease and/or a symptom of a disease caused by a coronavirus (e.g., a respiratory tract infectious disease) in a subject in need thereof, comprising administering to the subject, a therapeutically effective amount of a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, or a pharmaceutical composition comprising any of the foregoing.

A further aspect of the disclosure provides methods of treating a disease and/or a symptom of a disease caused by a coronavirus (e.g., a respiratory tract infectious disease) in a subject in need thereof, comprising administering to the subject, a therapeutically effective amount of a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, or a pharmaceutical composition comprising any of the foregoing in combination with a therapeutically effective amount of an additional active pharmaceutical agent, either in the same pharmaceutical composition as the compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing or in a separate composition.

Also disclosed herein are methods of reducing or inhibiting the activity of a protease of a coronavirus in a subject in need thereof, comprising administering to the subject a therapeutically effective amount of a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, or a pharmaceutical composition comprising any of the foregoing.

A further aspect of the disclosure provides a method of reducing or inhibiting the activity of a protease of a coronavirus in a biological sample, comprising contacting said sample, coronavirus or protease with a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, or a pharmaceutical composition comprising any of the foregoing.

Further disclosed herein are methods of reducing or inhibiting the replication of a coronavirus in a subject in need thereof, comprising administering to the subject, a therapeutically effective amount of a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, or a pharmaceutical composition comprising any of the foregoing.

A further aspect of the disclosure provides a method of reducing or inhibiting the replication of a coronavirus in a biological sample, comprising contacting said sample, coronavirus or a protease of the coronavirus with a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, or a pharmaceutical composition comprising any of the foregoing.

The present disclosure also provides a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, or a pharmaceutical composition comprising any of the foregoing, for use as a medicament.

The present disclosure also provides a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, or a pharmaceutical composition comprising any of the foregoing, for use in treating disorders associated with or caused by a coronavirus.

The present disclosure also provides use of a compound of Formulae I, II-1, II-2, II-3, II-4, II-5, III, IV, V, VI, VII, or VIII, a solvate thereof, a tautomer thereof, a stereoisomer thereof, a deuterated derivative of the compound, or a pharmaceutically acceptable salt of the foregoing, or a pharmaceutical composition comprising any of the foregoing, for the manufacture of a medicament for treating disorders associated with or caused by a coronavirus.

The compounds of the disclosure as potent 3CLpro inhibitors have improved antiviral activity and suitable pharmaceutical profile and thus are useful for the treatment a disease and/or a symptom of a disease caused by a coronavirus.

### BRIEF DESCRIPTION OF THE DRAWINGS

FIG. 1 shows the chemical structures of Compounds 1, 14, 18, 19, 20, 26, 29, 30, 32, 37, 39, 41, 43, 49, 50, 75, 77, 86, 89, 96, 98, 106, 119, 138, 151, 167, 169 and 174.

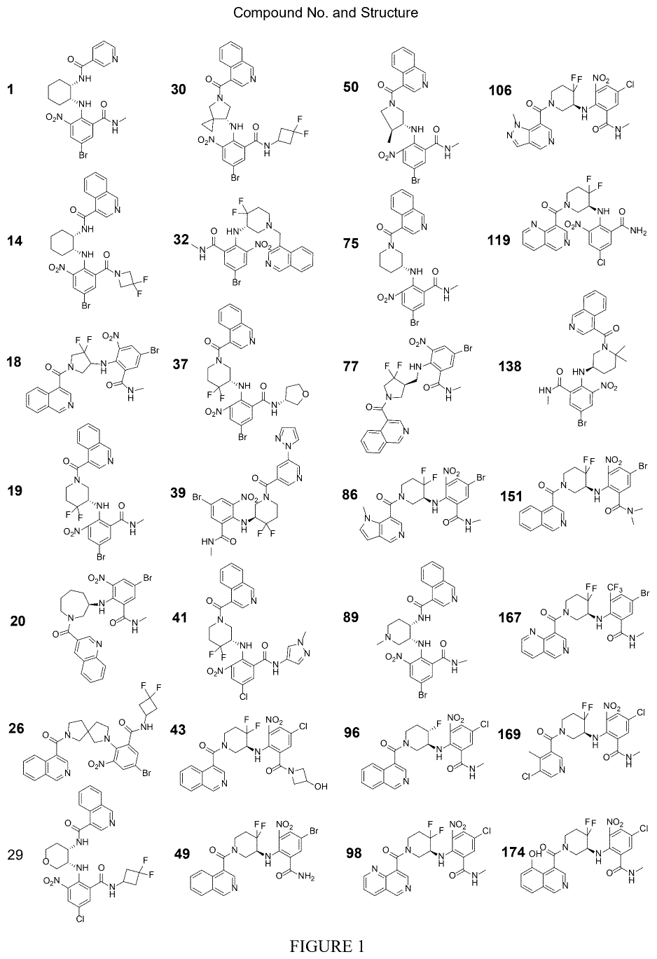

FIGS. 2A and 2B show the docking of Compound 1 to the structure of SARS-CoV-2 3CLpro. FIG. 2A shows that Compound 1 (sticks) docks well into the catalytic pocket of 3CLpro. The surface electrostatic potential of 3CLpro was calculated by PyMol. FIG. 2B shows interactions between Compound 1 and 3CLpro. The hydrogen bonds are indicated by dashed lines.

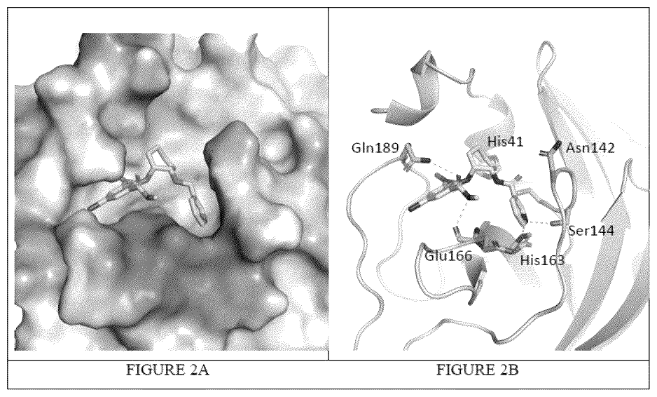

FIGS. 3A and 3B show crystal structure of Compound 19 in complex with SARS-CoV-2 3CLpro. FIG. 3A shows that Compound 19 (sticks) binds into the catalytic pocket of 3CLpro. The surface electrostatic potential of 3CLpro was calculated by PyMol. FIG. 3B shows interactions between Compound 19 and 3CLpro. The hydrogen bonds are indicated by dashed lines.

FIGS. 4A and 4B show crystal structure of Compound 98 in complex with SARS-CoV-2 3CLpro. FIG. 4A shows that Compound 98 (sticks) binds into the catalytic pocket of 3CLpro. The surface electrostatic potential of 3CLpro was calculated by PyMol. FIG. 4B shows interactions between Compound 98 and 3CLpro. The hydrogen bonds are indicated by dashed lines.

FIG. 5 shows the binding affinity of Compound Ref-2(compound P2), 19 and 30 with SARS-CoV-2 3CLpro determined by isothermal titration calorimetry (ITC).

FIG. 6 shows the anti-SARS-CoV-2 activity and the cytotoxicity of Compound 1 (FIG. 6A) and Compound 14 (FIG. 6B) in A549 cells.

FIG. 7 shows the anti-SARS-CoV-2 activity of Compounds Ref-2(compound P2), 18, 19, 26, 29, 30, 37, 41, 43, 75, 89 and 151 in A549 cells.

FIG. 8 shows the anti-Omicron SARS-CoV-2 activity of Compounds Ref-2 and 98 in Caco-2 cells.

FIG. 9 shows the CYP inhibition evaluation of Compounds Ref-2(compound P2), 97, 98 and 170 in human liver microsomes.

FIG. 10 shows the free plasma protein binding (PPB) ration of Compounds Ref-2(compound P2), 86, 97, 98 and 170 in human and mouse.

FIGS. 11A, 11B and 11C show the inhibitory activities of the Compounds Ref-2 and 98 against the 3CLpro(P132H) of SARS-CoV-2 (FIG. 11A), the 3CLpro of MERS-CoV (FIG. 11B), and the 3CLpro of SARS-CoV (FIG. 11C). Using an in vitro enzymatic assay, the half maximal inhibitory concentrations (IC50s) of Compounds Ref-2 and 98 were measured.

### DETAILED DESCRIPTION OF THE DISCLOSURE

### I. Definitions

The term “a” or “an” when referring to a noun as used herein encompasses the expression “at least one” and therefore encompasses both singular and plural units of the noun. For example, “an additional pharmaceutical agent” means a single or two or more additional pharmaceutical agents.

The term “coronavirus” as used herein refers to a group of related RNA viruses that cause diseases in mammals and birds, such as typically respiratory tract infectious diseases in humans and birds that can range from mild to lethal, diarrhea in cows and pigs, and hepatitis and encephalomyelitis in mice. Coronaviruses are enveloped viruses with a positive-sense single-stranded RNA genome and a nucleocapsid of helical symmetry. They have characteristic club-shaped spikes that project from their surface, which in electron micrographs create an image reminiscience of the solar corona, from which their names. The lethal varieties of coronavirus include those that can cause SARS (severe acute respiratory syndrome), MERS (Middle East respiratory syndrome), and COVID-19 (contagious disease caused by severe acute respiratory syndrome coronavirus 2 or SARS-CoV-2).

The term “inhibitor” as used herein refers to an organic chemistry small molecule compound (≤10 kDa) that has the ability to reduce or inhibit the expression of, and/or to reduce or inhibit the activity of any one or more proteases (e.g., by blocking the active site of the protease) of a coronavirus as defined above, including but not limited to the 3C-like protease (3CLU^{pro} or 3CLpro) or formally known as C30 endopeptidase that is the main protease of coronaviruses. The 3C-like protease cleaves the coronavirus polyprotein at multiple conserved sites having a glutamine-serine/glutamine-alanine/glutamine-glycine peptide bond, and is important in the processing of the coronavirus replicase polyprotein P0C6U8. Other examples of coronavirus proteases include the papain-like protease PLpro that is required for processing viral polyproteins to generate a functional replicase complex and enable viral spread.

The term “compound,” when referring to a compound of this disclosure, refers to a collection of molecules having an identical chemical structure unless otherwise indicated as a collection of stereoisomers (for example, a collection of racemates, a collection of cis/trans stereoisomers, or a collection of (E) and (Z) stereoisomers), except that there may be isotopic variation among the constituent atoms of the molecules. Thus, it will be clear to those of skill in the art that a compound represented by a particular chemical structure containing indicated deuterium atoms, will also contain lesser amounts of isotopologues having hydrogen atoms at one or more of the designated deuterium positions in that structure. The relative amount of such isotopologues in a compound of this disclosure will depend upon a number of factors, including, for example, the isotopic purity of reagents used to make the compound and the efficiency of incorporation of isotopes in the various synthesis steps used to prepare the compound. However, as set forth above the relative amount of such isotopologues in toto will be less than 49.9% of the compound. In other embodiments, the relative amount of such isotopologues in toto will be less than 47.5%, less than 40%, less than 32.5%, less than 25%, less than 17.5%, less than 10%, less than 5%, less than 3%, less than 1%, or less than 0.5% of the compound.

As used herein, the phrase “optionally substituted” is interchangeable with the phrase “substituted or unsubstituted.” In general, the term “substituted,” refers to the replacement of hydrogen radicals in a given structure with the radical of a specified substituent. Unless otherwise indicated, an “optionally substituted” group may have a substituent at each substitutable position of the group, and when more than one position in any given structure may be substituted with more than one substituent chosen from a specified group, the substituent may be either the same or different at every position.

Combinations of certain chemical components (e.g., substituents, ring structures, and or heteroatoms) envisioned by this disclosure are those that result in the formation of stable or chemically feasible compounds. For abbreviation or according to common practice, certain hydrogen atoms attached to a certain atom (e.g., a carbon atom C or a nitrogen atom N) are not specifically spelled out in a chemical structure, formula, or notation; hydrogen atoms are deemed to be present to the extent the valences of the certain atom (e.g., C or N) are completed.

The term “isotopologue” refers to a species in which the chemical structure differs from only in the isotopic composition thereof. Additionally, unless otherwise stated, structures depicted herein are also meant to include compounds that differ only in the presence of one or more isotopically enriched atoms. For example, compounds having the present structures except for the replacement of hydrogen by deuterium or tritium, or the replacement of a carbon by a ³C or ¹⁴C are also within the scope of this disclosure.

Unless otherwise indicated, structures depicted herein are also meant to include all isomeric forms of the structure, e.g., racemic mixtures, cis/trans isomers, geometric (or conformational) isomers, such as (Z) and (E) double bond isomers, and (Z) and (E) conformational isomers. Therefore, geometric and conformational mixtures of the present compounds are within the scope of the disclosure. Unless otherwise stated, all tautomeric forms of the compounds of the disclosure are within the scope of the disclosure.

The term “solvate” as used herein, refers to a molecular complex comprising the compound of the present disclosure and one or more pharmaceutically acceptable solvent molecules, for example, ethanol, DMSO or water. When the solvent is water, the term “hydrate” may optionally be used interchangeable with the term “solvate.”

The term “tautomer” as used herein, refers to one of two or more isomers of a compound that exist together in equilibrium, and are readily interchanged by migration of an atom, e.g., a hydrogen atom, or a group within the molecule.

The term “stereoisomer” as used herein refers to enantiomers and diastereomers.

As used herein, “deuterated derivative” refers to a compound having the same chemical structure as a reference compound, but with one or more hydrogen atoms replaced by a deuterium atom (“D” or “²H”). It will be recognized that some variation of natural isotopic abundance occurs in a synthesized compound depending on the origin of chemical materials used in the synthesis. The concentration of naturally abundant stable hydrogen isotopes, notwithstanding this variation is small and immaterial as compared to the degree of stable isotopic substitution of deuterated derivatives described herein. Thus, unless otherwise stated, when a reference is made to a “deuterated derivative” of a compound of the disclosure, at least one hydrogen is replaced with deuterium at a level that is well above its natural isotopic abundance, which is typically about 0.015%. In some embodiments, the deuterated derivatives disclosed herein have an isotopic enrichment factor for each deuterium atom, of at least 3500 (52.5% deuterium incorporation at each designated deuterium), at least 4500 (67.5% deuterium incorporation at each designated deuterium), at least 5000 (75% deuterium incorporation at each designated deuterium), at least 5500 (82.5% deuterium incorporation at each designated deuterium), at least 6000 (90% deuterium incorporation at each designated deuterium), at least 6333.3 (95% deuterium incorporation at each designated deuterium), at least 6466.7 (97% deuterium incorporation at each designated deuterium), or at least 6600 (99% deuterium incorporation at each designated deuterium).

The term “isotopic enrichment factor” as used herein means the ratio between the isotopic abundance and the natural abundance of a specified isotope.

The term “alkyl” as used herein, means a linear or branched, substituted or unsubstituted hydrocarbon chain that is completely saturated. Unless otherwise specified, an alkyl group contains 1 to 20 alkyl carbon atoms. In some embodiments, an alkyl group contains 1 to 10 aliphatic carbon atoms. In some embodiments, an alkyl group contains 1 to 8 aliphatic carbon atoms. In some embodiments, an alkyl group contains 1 to 6 alkyl carbon atoms. In some embodiments, an alkyl group contains 1 to 4 alkyl carbon atoms. In other embodiments, an alkyl group contains 1 to 3 alkyl carbon atoms. And in yet other embodiments, an alkyl group contains 1 to 2 alkyl carbon atoms. In some embodiments, alkyl groups are substituted. In some embodiments, alkyl groups are unsubstituted. In some embodiments, alkyl groups are linear or straight-chain or unbranched. In some embodiments, alkyl groups are branched. For example, alkyl groups include, but are not limited to, methyl, ethyl, n-propyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl and n-decyl.

The term “cycloalkyl” refers to a monocyclic C₃₋₈ hydrocarbon or a spirocyclic, fused, or bridged bicyclic or tricyclic C₈₋₁₄hydrocarbon that is completely saturated, wherein any individual ring in said bicyclic ring system has 3 to 7 members. In some embodiments, cycloalkyl groups are substituted. In some embodiments, cycloalkyl groups are unsubstituted. In some embodiments, the cycloalkyl is a C₃ to C₁₂ cycloalkyl. In some embodiments, the cycloalkyl is a C₃ to C₈ cycloalkyl. In some embodiments, the cycloalkyl is a C₃ to C₆ cycloalkyl. Non-limiting examples of monocyclic cycloalkyls include cyclopropyl, cyclobutyl, cyclopentanyl, and cyclohexyl.

The term “carbocyclyl” encompasses the term “cycloalkyl” and refers to a monocyclic C₃₋₈ hydrocarbon or a spirocyclic, fused, or bridged bicyclic or tricyclic C₈₋₁₄hydrocarbon that is completely saturated, or is partially saturated as it contains one or more units of unsaturation but is not aromatic, wherein any individual ring in said bicyclic ring system has 3 to 7 members.

Bicyclic carbocyclyls include combinations of a monocyclic carbocyclic ring fused to, for example, a phenyl. In some embodiments, carbocyclyl groups are substituted. In some embodiments, carbocyclyl groups are unsubstituted. In some embodiments, the carbocyclyl is a C₃ to C₁₂ carbocyclyl. In some embodiments, the carbocyclyl is a C₃ to C₁₀ carbocyclyl. In some embodiments, the carbocyclyl is a C₃ to C₈ carbocyclyl.

The term “alkenyl” as used herein, means a linear or branched, substituted or unsubstituted hydrocarbon chain that contains one or more double bonds. In some embodiments, alkenyl groups are substituted. In some embodiments, alkenyl groups are unsubstituted. In some embodiments, alkenyl groups are linear, straight-chain, or unbranched. In some embodiments, alkenyl groups are branched. In some embodiments, the term “alkenyl” refers to C₂-C₁₀ alkenyl, preferably C₂-C₆ alkenyl, more preferably C₂-C₄ alkenyl. Some examples of alkenyls include, but are not limited to, vinyl, allyl, butenyl, pentenyl, and hexenyl.

The term “alkynyl” refers to a straight or branched alkynyl group of 2 to 24 carbon atoms, such as acetylenyl, n-propynyl, etc., that contains at least one triple bond. Usually, but not necessarily, the alkynyl group in the disclosure contains 2 to 12 carbon atoms, or 2 to 6 carbon atoms, preferably 4 carbon atoms, more preferably 3 carbons, most preferably 2 carbons. The terms “substituted alkynyl” refer to an alkynyl group that is replaced by one or more substituting groups, and the terms “hetero-atomic alkynyl” and “hetero-alkynyl” refer to an alkynyl group in which at least one carbon atom is replaced by a heteroatom. If not indicated otherwise, then the term “alkynyl” includes alkynyl groups that are straight, branched, unsubstituted, substituted, and/or contain heteroatoms.

The term “heterocyclyl” as used herein means non-aromatic (i.e., completely saturated or partially saturated as in it contains one or more units of unsaturation but is not aromatic), monocyclic, or spirocyclic, fused, or bridged bicyclic or tricyclic ring systems in which one or more ring members is an independently chosen heteroatom. Bicyclic heterocyclyls include, for example, the following combinations of monocyclic rings: a monocyclic heteroaryl fused to a monocyclic heterocyclyl; a monocyclic heterocyclyl fused to another monocyclic heterocyclyl; a monocyclic heterocyclyl fused to phenyl; a monocyclic heterocyclyl fused to a monocyclic carbocyclyl/cycloalkyl; and a monocyclic heteroaryl fused to a monocyclic carbocyclyl/cycloalkyl. In some embodiments, the “heterocyclyl” group contains 3 to 14 ring members in which one or more ring members is a heteroatom independently chosen, for example, from oxygen, sulfur, nitrogen, and phosphorus. In some embodiments, each ring in a bicyclic or tricyclic ring system contains 3 to 7 ring members. In some embodiments, the heterocycle has at least one unsaturated carbon-carbon bond. In some embodiments, the heterocycle has at least one unsaturated carbon-nitrogen bond. In some embodiments, the heterocycle has one heteroatom independently chosen from oxygen, sulfur, nitrogen, and phosphorus. In some embodiments, the heterocycle has one heteroatom that is a nitrogen atom.

In some embodiments, the heterocycle has one heteroatom that is an oxygen atom. In some embodiments, the heterocycle has two heteroatoms that are each independently selected from nitrogen and oxygen. In some embodiments, the heterocycle has three heteroatoms that are each independently selected from nitrogen and oxygen. In some embodiments, heterocycles are substituted. In some embodiments, heterocycles are unsubstituted. In some embodiments, the heterocyclyl is a 3- to 12-memberedheterocyclyl. In some embodiments, the heterocyclyl is a 3- to 10-membered heterocyclyl. In some embodiments, the heterocyclyl is a 4- to 9-membered heterocyclyl, for example, a 4- to 9-membered heterocyclyl containing at least one N atom and optionally at least one O atom. In some embodiments, the heterocyclyl is a 5- to 10-membered heterocyclyl. In some embodiments, the heterocyclyl is a 5- to 8-membered heterocyclyl. In some embodiments, the heterocyclyl is a 5- or 6-membered heterocyclyl. In some embodiments, the heterocyclyl is a 6-membered heterocyclyl. Non-limiting examples of monocyclic heterocyclyls include piperidinyl, piperazinyl, tetrahydropyranyl, azetidinyl, tetrahydrothiophenyl 1,1-dioxide, etc.

Also used herein, the term “spiro-heterocyclyl” refers to a ring system in which a three-to seven-membered heterocyclyl has one or more additional ring, wherein the one or more additional ring is three- to seven-membered cycloalkyl or three- to seven-membered heterocyclyl, where a single atom of the one or more additional ring is also an atom of the three- to seven-membered heterocyclyl.

The term “hetero atom” means one or more of oxygen, sulfur, phosphorus, and nitrogen, including, any oxidized form of nitrogen or sulfur; the quatemized form of any basic nitrogen or a substitutable nitrogen of a heterocyclic ring, for example N (as in 3,4-dihydro-2H-pyrrolyl), NH (as in pyrrolidinyl) or NR⁺ (as in N-substituted pyrrolidinyl). The term “nitrogen heteroalkyl,” “nitrogen heteroaryl,” or “nitrogen heterocyclyl” means the ring atoms of the heteroalkyl, heteroaryl, or heterocyclyl ring contains one or more hetero atoms and the one or more hetero atoms are nitrogen. The term “oxygen heteroalkyl,” “oxygen heteroaryl,” or “oxygen heterocyclyl” means the ring atoms of the heteroalkyl, heteroaryl, or heterocyclyl ring contains one or more hetero atoms and the one or more hetero atoms are oxygen.

The term “unsaturated”, as used herein, means that a moiety has one or more units or degrees of unsaturation. Unsaturation is the state in which not all of the available valence bonds in a compound are satisfied by substituents and thus the compound contains one or more double or triple bonds.

The term “alkoxy” as used herein, refers to an alkyl group, as defined above, wherein one carbon of the alkyl group is replaced by an oxygen (“alkoxy”) atom, provided that the oxygen atom is linked between two carbon atoms.

The term “halogen” includes F, Cl, Br, and I, i.e., fluoro, chloro, bromo, and iodo, respectively.

As used herein, a “cyano” or “nitrile” group refers to —C≡N.

As used herein, an “aromatic ring” refers to a carbocyclic or heterocyclic ring that contains conjugated, planar ring systems with delocalized pi electron orbitals comprised of [4n+2] p orbital electrons, wherein n is an integer of 0 to 6. A “non-aromatic” ring refers to a carbocyclic or heterocyclic that does not meet the requirements set forth above for an aromatic ring, and can be either completely or partially saturated. Nonlimiting examples of aromatic rings include aryl and heteroaryl rings that are further defined as follows.

The term “aryl” used alone or as part of a larger moiety as in “arylalkyl,” “arylalkoxy,” or “aryloxyalkyl,” refers to monocyclic or spirocyclic, fused, or bridged bicyclic or tricyclic ring systems having a total of five to fourteen ring members, wherein every ring in the system is an aromatic ring containing only carbon atoms and wherein each ring in a bicyclic or tricyclic ring system contains 3 to 7 ring members. Nonlimiting examples of aryl groups include phenyl (C₆) and naphthyl (C₁₀) rings. In some embodiments, aryl groups are substituted. In some embodiments, aryl groups are unsubstituted.

The term “heteroaryl” refers to monocyclic or spirocyclic, fused, or bridged bicyclic or tricyclic ring systems having a total of five to fourteen ring members, wherein at least one ring in the system is aromatic, at least one ring in the system contains one or more heteroatoms, and wherein each ring in a bicyclic or tricyclic ring system contains 3 to 7 ring members. Bi-cyclic heteroaryls include, for example, the following combinations of monocyclic rings: a monocyclic heteroaryl fused to another monocyclic heteroaryl; and a monocyclic heteroaryl fused to a phenyl. Non-limiting examples of bi-cyclic heteroaryls are isoquinolinyl, quinolinyl, quinazolinyl, phthalazinyl, purinyl, and 1H-pyrrolo[2,3-c]pyridinyl. In some embodiments, heteroaryl groups are substituted. In some embodiments, heteroaryl groups have one or more heteroatoms chosen, for example, from nitrogen, oxygen, and sulfur. In some embodiments, heteroaryl groups have one heteroatom. In some embodiments, heteroaryl groups have two heteroatoms. In some embodiments, heteroaryl groups are monocyclic ring systems having five ring members. In some embodiments, heteroaryl groups are monocyclic ring systems having six ring members. In some embodiments, heteroaryl groups are unsubstituted. In some embodiments, the heteroaryl is a 3- to 12-membered heteroaryl. In some embodiments, the heteroaryl is a 3- to 10-memberedheteroaryl. In some embodiments, the heteroaryl is a 3- to 8-membered heteroaryl. In some embodiments, the heteroaryl is a 5- to 10-membered heteroaryl. In some embodiments, the heteroaryl is a 5- to 8-membered heteroaryl. In some embodiments, the heteroaryl is a 5- or 6-membered heteroaryl. In some embodiments, the heteroaryl is a 6-membered heteroaryl. Non-limiting examples of monocyclic heteroaryls are pyridinyl, pyrimidinyl, thiophenyl, thiazolyl, isoxazolyl, etc.

Non-limiting examples of suitable solvents that may be used in this disclosure include water, methanol (MeOH), ethanol (EtOH), dichloromethane or “methylene chloride” (CH₂Cl₂), toluene, acetonitrile (MeCN), dimethylformamide (DMF), dimethyl sulfoxide (DMSO), methyl acetate (MeOAc), ethyl acetate (EtOAc), heptanes, isopropyl acetate (IPAc), tert-butyl acetate (t-BuOAc), isopropyl alcohol (IPA), tetrahydrofuran (THF), 2-methyl tetrahydrofuran (2-Me THF), methyl ethyl ketone (MEK), tert-butanol, diethyl ether (Et₂O), methyl-tert-butyl ether (MTBE), 1,4-dioxane, and N-methyl pyrrolidone (NMP).

Non-limiting examples of suitable bases that may be used in this disclosure include 1,8-diazabicyclo[5.4.0]undec-7-ene (DBU), potassium tert-butoxide (KOtBu), potassium carbonate (K₂CO₃), N-methylmorpholine (NMM), triethylamine (Et₃N; TEA), diisopropyl-ethyl amine (i-Pr₂EtN; DIPEA), pyridine, potassium hydroxide (KOH), sodium hydroxide (NaOH), lithium hydroxide (LiOH) and sodium methoxide (NaOMe; NaOCH₃).

Disclosed herein are pharmaceutically acceptable salts of the disclosed compounds. A salt of a compound is formed between an acid and a basic group of the compound, such as an amino functional group, or a base and an acidic group of the compound, such as a carboxyl functional group.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this disclosure. Suitable pharmaceutically acceptable salts are, for example, those disclosed in S. M. Berge, et al. J. Pharmaceutical Sciences, 1977, 66, pp. 1 to 19.

Acids commonly employed to form pharmaceutically acceptable salts include inorganic acids such as hydrogen bisulfide, hydrochloric acid, hydrobromic acid, hydroiodic acid, carbonic acid, sulfuric acid, sulfurous acid, and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and those formed with organic acids such as maleic acid.

Pharmaceutically acceptable salts derived from appropriate bases include alkali metal, alkaline earth metal, ammonium, and N⁺(C₁₋₄alkyl)₄ salts. This disclosure also envisions the quaternization of any basic nitrogen-containing groups of the compounds disclosed herein. Suitable non-limiting examples of alkali and alkaline earth metal salts include sodium, lithium, potassium, calcium, and magnesium. Further non-limiting examples of pharmaceutically acceptable salts include ammonium, quaternary ammonium, and amine cations formed using counterions such as halide, hydroxide, carboxylate, sulfate, phosphate, nitrate, lower alkyl sulfonate and aryl sulfonate. Other suitable, non-limiting examples of pharmaceutically acceptable salts include besylate and glucosamine salts.

The term “subject” refers to an animal including a human.

The term “biological sample”, as used herein, includes, without limitation, cell cultures or extracts thereof; biopsied material obtained from a mammal or extracts thereof; and blood, saliva, urine, feces, semen, tears, or other body fluids or extracts thereof.

The term “therapeutically effective amount” refers to that amount of a compound that produces the desired effect for which it is administered (e.g., improvement in a disease and/or a symptom of a disease caused by a coronavirus, lessening the severity of a disease and/or a symptom of a disease caused by a coronavirus, and/or reducing progression of a disease and/or a symptom caused by a coronavirus). The exact amount of a therapeutically effective amount will depend on the purpose of the treatment and will be ascertainable by one skilled in the art using known techniques (see, e.g., Lloyd (1999), The Art, Science and Technology of Pharmaceutical Compounding).

As used herein, the term “treatment” and its cognates refer to slowing or stopping disease progression. “Treatment” and its cognates as used herein include, but are not limited to the following: complete or partial remission, curing a disease caused by a coronavirus, lower risk of a disease and/or a symptom of a disease caused by a coronavirus. Improvements in or lessening the severity of any of these symptoms can be assessed according to methods and techniques known in the art.

The terms “about” and “approximately,” when used in connection with a number such as a percentage include the number as specified, and a range of the number (e.g., a range of percentages) that is recognized by one of ordinary skill in the art.

### II. Compounds and Compositions

In a first embodiment, a compound of this disclosure is a compound of the following structural formula I:

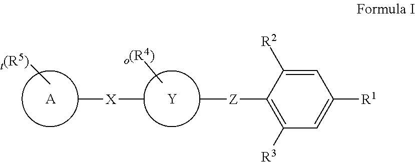

a solvate thereof, a tautomer thereof, a stereoisomer thereof, a deuterated derivative of the compound, or a pharmaceutically acceptable salt of the foregoing, wherein:
R¹ is hydrogen, halogen, or C₁-C₂ alkyl that is optionally substituted with 1 to 3 groups of halogen;
R² is —NO₂, —SO₃H, —SO₂CH₃, —CN or —CF₃;
R³ is 5- or 6-membered heteroaryl, —C(═O)R^{a}, —C(═O)OR^{a}, —C(═O)NR^{b}R^{c} or

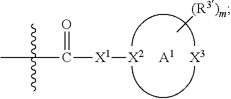

wherein:
R^{a} is hydrogen, C₁-C₄ alkyl, C₂-C₄ alkenyl, or C₂-C₄ alkynyl, wherein the C₁-C₄ alkyl, C₂-C₄ alkenyl, or C₂-C₄ of R^{a} is optionally substituted with 1-3 groups selected from halogen and deuterium;
R^{b} is hydrogen, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{b} is optionally substituted with 1-3 groups selected from halogen and deuterium;
R^{c} is hydrogen, NH₂, —OH, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{c} is optionally substituted with 1-3 groups selected from halogen, deuterium, and —OH;
Ring A¹ is 3-, 4-, 5- or 6-membered cycloalkyl, or 4-, 5- or 6-membered heterocycloalkyl or 5- or 6-membered heteroaryl, wherein the heterocycloalkyl or heteroaryl of Ring A¹ contains one or more heteroatoms selected from N and O;
X¹ is absent, —CH₂—, —NH— or —O—;
X² is CH or N;
X³ is —O—, —CH₂, or —NH—;
R^{3′}, for each occurrence, is independently selected from halogen, —OR^{d}, —NR^{e}R^{f}, and C₁-C₄ alkyl that is optionally substituted with 1-3 groups of halogen; wherein:
R^{d} is hydrogen, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{d} is optionally substituted with 1-3 groups of halogen;
R^{e} is hydrogen, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{f} is optionally substituted with 1-3 groups of halogen;
R^{f} is hydrogen, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{f} is optionally substituted with 1-3 groups of halogen;
R^{3′}, for each occurrence, may be attached to any of the ring atoms of the Ring A¹, as long as valence permits; and
m is an integer selected from 0, 1, 2 and 3;
Z is absent, —NR^{g}—, —C(R^{h}Rⁱ)—NR^{g}—, —NR^{g}—C(R^{h}Rⁱ)—, or —O—, wherein:
R^{g} is hydrogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl of R^{g} is optionally substituted with 1-3 groups of halogen;
R^{h} is hydrogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl of R^{b} is optionally substituted with 1-3 groups of halogen;
Rⁱ is hydrogen, C₁-C₆ alkyl, C₂-C₆ alkenyl, or C₂-C₆ alkynyl, wherein the C₁-C₆ alkyl, C₂-C₆ alkenyl, or C₂-C₆ alkynyl of Rⁱ is optionally substituted with 1-3 groups of halogen;
Ring Y is phenyl or selected from the following rings:

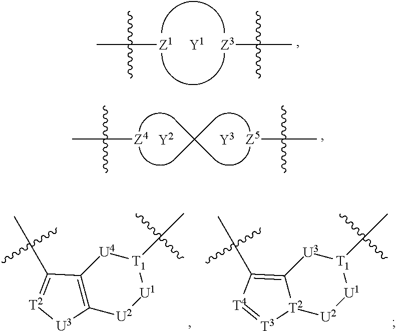

wherein:
Ring Y¹ is 4-, 5-, 6-, 7-, 8-, or 9-membered cycloalkyl or 4-, 5-, 6-, 7-, 8-, or 9-membered heterocycloalkyl, wherein the heterocycloalkyl of Ring Y¹ contains one or more heteroatoms selected from N and O;
Ring Y² is 4-, 5-, 6-, or 7-membered cycloalkyl or 4-, 5-, 6-, or 7-membered nitrogen heterocycloalkyl;
Ring Y³ is 4-, 5-, 6-, or 7-membered cycloalkyl or 4-, 5-, 6-, or 7-membered nitrogen heterocycloalkyl;
Z¹, Z³, Z⁴, and Z⁵, for each occurrence, are each independently CH or N;
U¹, U², U³, and U⁴, for each occurrence, are each independently CH₂ or NH;
T¹, T², T³, and T⁴, for each occurrence, are each independently CH or N;
R⁴, for each occurrence, is independently halogen, —OH, or —C(═O)RJ, C₁-C₄ alkyl, C₂-C₄ alkenyl, or C₂-C₄ alkynyl wherein the C₁-C₄ alkyl, C₂-C₄ alkenyl, or C₂-C₄ alkynyl of R⁴ is optionally substituted with 1-3 groups of halogens; wherein:
R^{j} is C₁-C₄ alkyl, C₂-C₄ alkenyl, or C₂-C₄ alkynyl wherein the C₁-C₄ alkyl, C₂-C₄ alkenyl, or C₂-C₄ alkynyl of R^{j} is optionally substituted with 1-3 groups of halogens;
R⁴, for each occurrence, may be attached to any of the ring atoms of Ring Y, as long as valence permits;
is an integer selected from 0, 1 or 2;
X is absent, —CR^{k}R^{L}—, —C(═O)—, —C(═O)NRⁿ—, or —R^{m}—C(═O)NRⁿ—R^{o}—; wherein:
R^{k}, R^{L}, and Rⁿ, for each occurrence, are each independently selected from hydrogen, halogen, and C₁-C₂ alkyl that is optionally substituted with 1-3 groups of halogen;
R^{m} and R^{o}, for each occurrence, are each independently selected from hydrogen and C₁-C₂ alkyl that is optionally substituted with 1-3 groups of halogen;
Ring A is 5- or 6-membered nitrogen heteroalkyl or nitrogen heteroaryl, or 9- or 10-membered nitrogen heterocyclyl; wherein:
Ring A contains —N═, or ═N— in the meta position to the ring atom to which X is attached, or —NH— in the meta position or para position to the ring atom to which X is attached; the —NH— in the meta position is attached to —C(═O)— in the ortho position, or the —NH— in the para position is attached to —C(═O)— in the meta position;
Ring A is substituted by t groups of R⁵, and R⁵ may be attached to any of the ring atoms of the Ring A, as long as valence permits;
R⁵, for each occurrence, is independently halogen, —CN, —OR^{p}, —NHR^{p}, —CH₂NHBoc, 5- or 6-membered cycloalkyl, 5- or 6-membered heterocyclyl, 5- or 6-membered heteroaryl, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R⁵ is optionally substituted with 1-3 groups of halogen; R^{p}, for each occurrence, is independently selected from halogen and C₁-C₄ alkyl; and
t is 0, 1, 2, 3, 4, or 5;
   provided that the compound is not any one of Compounds P 1 through P23:

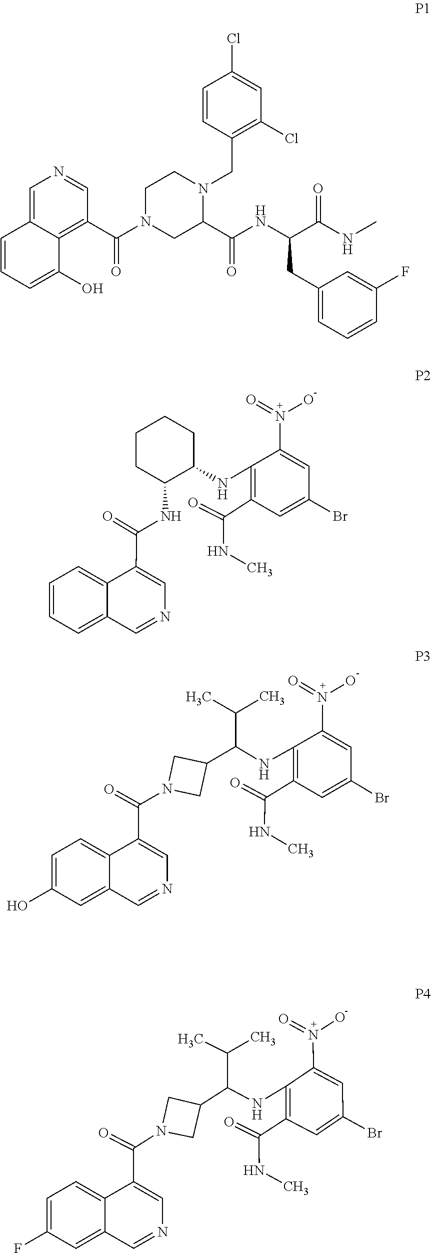

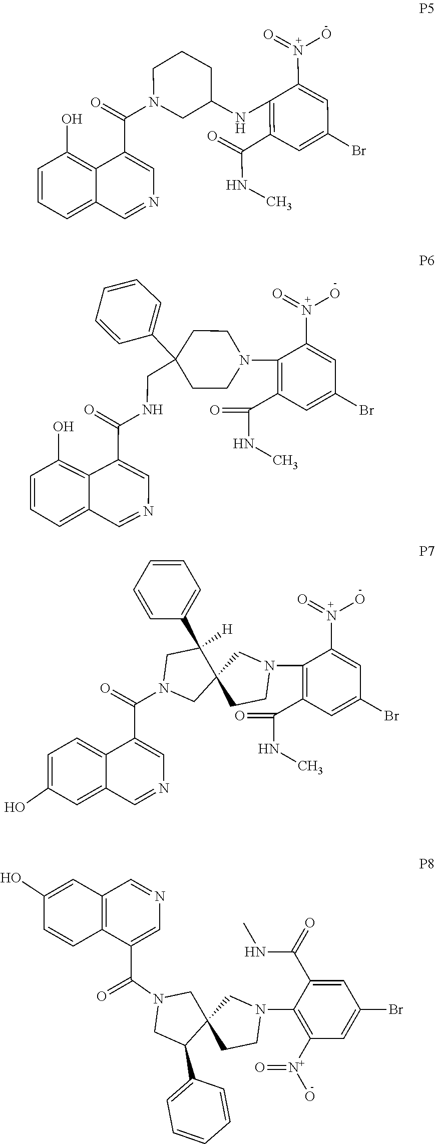

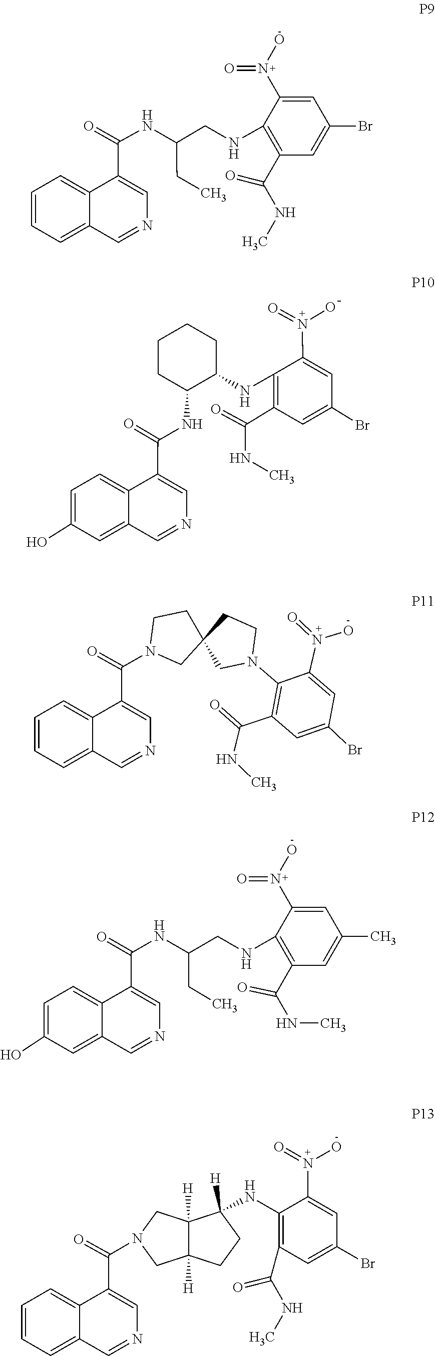

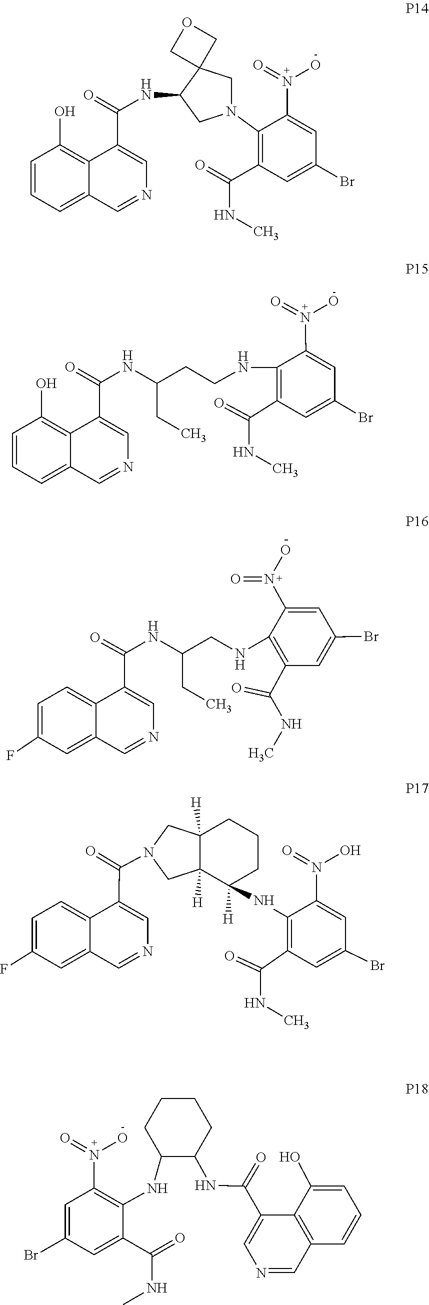

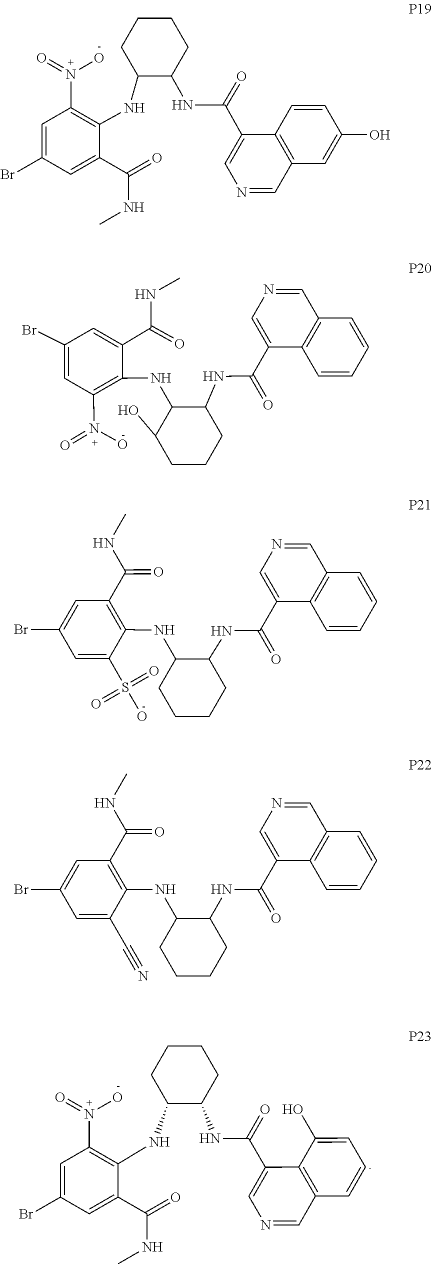

In one embodiment of formula I, wherein R³ is

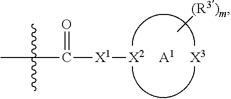

wherein Ring A¹, X¹, X², X³, R^{3′} and m are as defined in the first embodiment.

In one embodiment of formula I, wherein R³ is

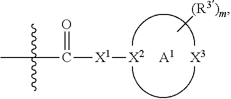

wherein X¹ is —CH₂—, —NH— or —O—, and Ring A¹, X², X³, R^{3′} and m are as defined in the first embodiment.

In one embodiment of formula I, wherein R³ is

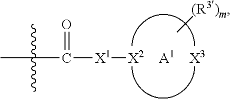

wherein X¹ is —NH—, and Ring A¹, X², X³, R^{3′} and m are as defined in the first embodiment.

In one embodiment of formula I, wherein R³ is

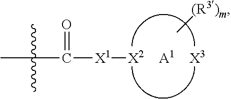

wherein X¹ is —NH—, and Ring A¹ is 3-, 4-, 5- or 6-membered cycloalkyl, and X², X³R^{3′} and m are as defined in the first embodiment.

In one embodiment of formula I, wherein R³ is

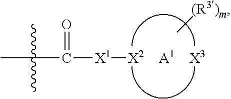

wherein X¹ is —NH—, and Ring A¹ is 4-, 5- or 6-membered heterocycloalkyl, wherein the heterocycloalkyl of Ring A¹ contains one or more heteroatoms selected from N and O, and X², X³, R^{3′} and m are as defined in the first embodiment.

In one embodiment of formula I, R¹ is Br, F or Cl; and all other variables not specifically defined herein are as defined in the first embodiment.

In one embodiment of formula I, R³ is 5- to 7-membered nitrogen and/or oxygen heterocyclyl; and all other variables not specifically defined herein are as defined in the first embodiment.

In one embodiment of formula I, R³ is oxazolyl or triazolyl; and all other variables not specifically defined herein are as defined in the first embodiment.

In one embodiment of formula I, R³ is

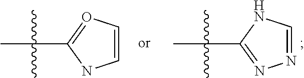

and all other variables not specifically defined herein are as defined in the first embodiment.

In some embodiments, R^{3′} is —NHCH₃, —NHCD₃, —NHCHD₂, or —NHCH₂D, and all other variables not specifically defined herein are as defined in the first embodiment.

In some embodiments, when R^{b} is H, R^{c} is H, NH₂, —OH, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{b} is optionally substituted with 1-3 groups selected from halogen, deuterium, and —OH, and all other variables not specifically defined herein are as defined in the first embodiment.

In some embodiments, a compound of the disclosure is of the following structural Formula II-1, II-2, II-3, II-4, or II-5:

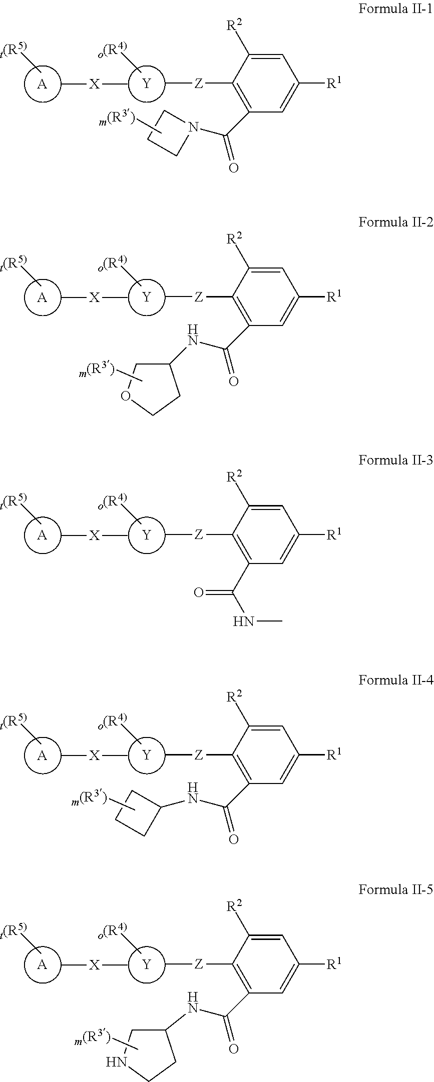

In some embodiments, R³ is 4- or 5-membered cycloalkyl, 4- or 5-membered heterocyclyl, or 4- or 5-membered heteroaryl, wherein the heterocyclyl and heteroaryl of R³ contains one or more hetero atoms selected from N and O, and all other variables not specifically defined herein are as defined in the first embodiment.

In some embodiments, R³ is —C(═O)NHCH₃, —C(═O)NH₂, —C(═O)N(CH₃)₂, —C(═O)OCH₃, —C(═O)NHCH₃, —C(═O)NHCH₂CH═CH₂, —COOH, —C(═O)NHNH₂, —C(═O)NHCH₂CF₃, —C(═O) NHCH(CH₃)CF₃, —C(═O)NHCH₂CHF₂, —C(═O)NHCH₃, —C(═O)NHCD₃, —C(═O) NHCH(CH₃)CF₃, —C(═O)NHC(CH₃)₃, —C(═O)NHCH₂(CH₃)₂CH₂OH,

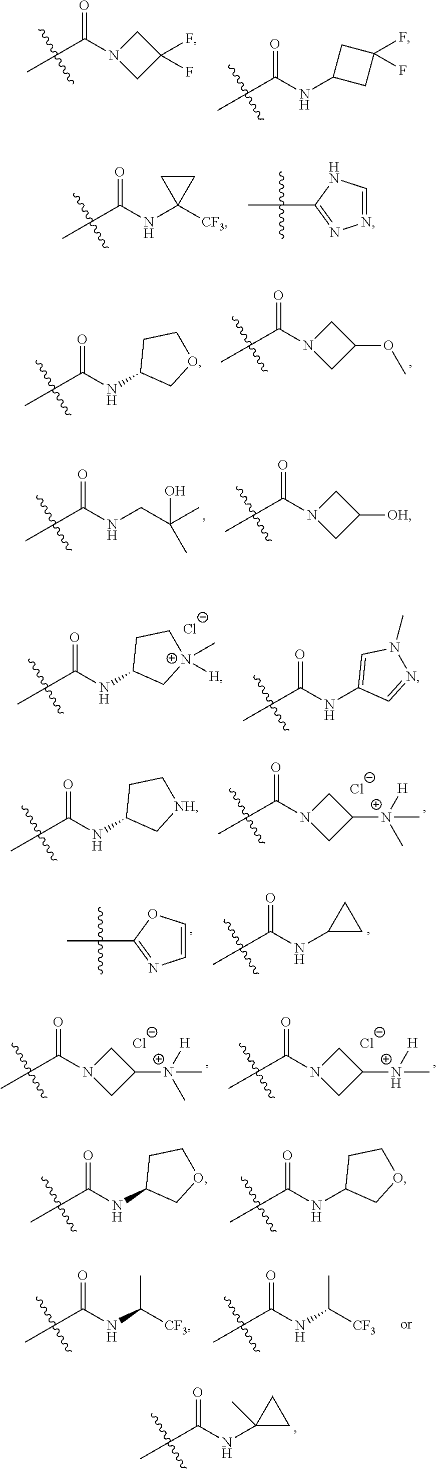

and all other variables not specifically defined herein are as defined in the first embodiment.

In some embodiments, R³ is —C(═O)OCH₃, —C(═O)NHCH₂CH═CH₂, —COOH, —C(═O) NHNH₂, —C(═O)NHCH(CH₃)CF₃, —C(═O)NHCH₂CHF₂, —C(═O)NHCH₃, —C(═O)NHCD₃, —C(═O)NHCH(CH₃)CF₃, —C(═O)NHCH₂(CH₃)₂CH₂OH, ,

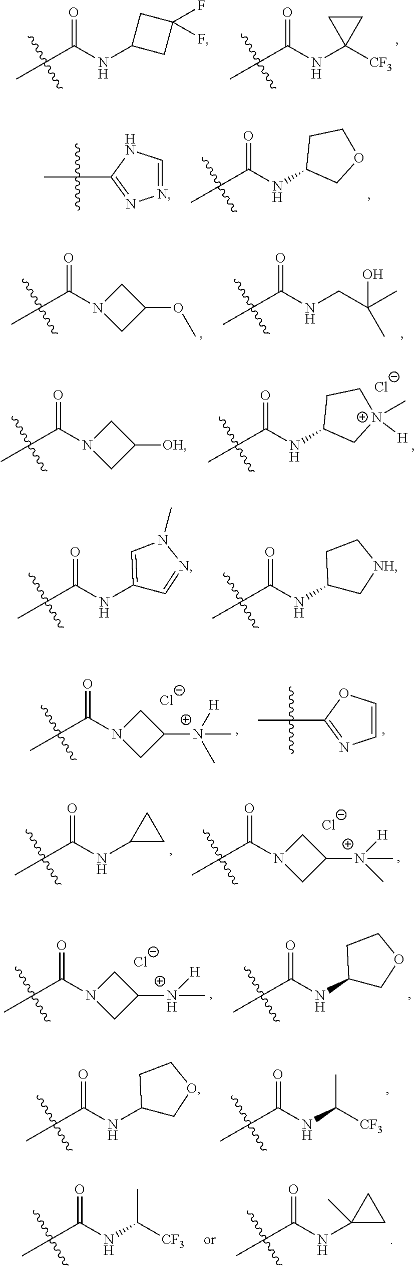

In some embodiments, R³ is 4- or 5-membered heterocyclyl, wherein heterocyclyl of R³ contains one or more hetero atoms selected from N and O, and all other variables not specifically defined herein are as defined in the first embodiment.

In some embodiments, R³ is 5-membered nitrogen heteroaryl, and all other variables not specifically defined herein are as defined in the first embodiment.

In some embodiments, R³ is

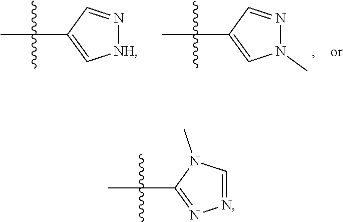

and all other variables not specifically defined herein are as defined in the first embodiment.

In some embodiments, when X¹ is —NH—, X² is CH; when X is —CH₂—, X² is N, and all other variables not specifically defined herein are as defined in the first embodiment.

In some embodiments, R^{d} is H, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{d} is optionally substituted with 1-3 groups of halogen, and all other variables not specifically defined herein are as defined in the first embodiment.

In some embodiments, R^{e} is H, R^{f} is H, NH₂, —OH, C₁-C₄ alkyl, C₂-C₄ alkenyl, or C₂-C₄ alkynyl, wherein the C₁-C₄ alkyl, C₂-C₄ alkenyl, or C₂-C₄ alkynyl of R^{f} is optionally substituted with 1-3 groups of halogen, and all other variables not specifically defined herein are as defined in the first embodiment.

In some embodiments, R^{g} is H, and all other variables not specifically defined herein are as defined in the first embodiment.

In some embodiments, a compound of the disclosure is of the following structural Formula III:

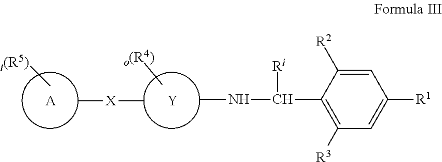

and all other variables not specifically defined herein are as defined in the first embodiment.

In some embodiments, a compound of the disclosure is of the following structural Formula IV:

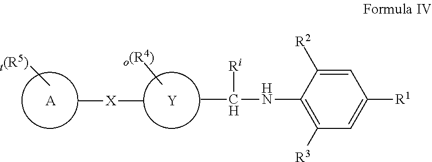

and all other variables not specifically defined herein are as defined in the first embodiment.

In some embodiments, R¹ is C₁-C₆ branched alkyl that is optionally substituted with 1-3 groups of halogen, or C₄-C₈ alkyl with C₃-C₆ carbocyclyl side chain and optionally substituted with 1-3 groups of halogen, and all other variables not specifically defined herein are as defined in the first embodiment.

In some embodiments, Z¹ is in ortho position or meta position to Z³, and all other variables not specifically defined herein are as defined in the first embodiment.

In some embodiments, a compound of the disclosure is of the following structural Formula V:

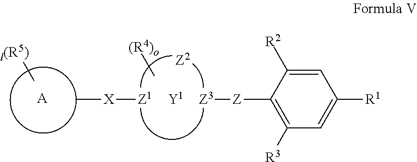

Ring Y¹ is 4-, 5-, 6- or 7-membered nitrogen heterocyclyl;
when Z¹ is CH, X is —C(═O)NRⁿ— or —R^{m}—C(═O)NRⁿR^{o}—;
when Z¹ is N, X is absent, —CR^{k}R^{L}—, or —C(═O)—; and
when Z³ is CH, Z is —NR^{g}—, —C(R^{h}Rⁱ)—NR^{g}—, —NR^{g}—C(R^{h}Rⁱ)— or —O—;
when Z³ is N, Z is absent,
and all other variables not specifically defined herein are as defined in the first embodiment.

In one embodiment of formula V, wherein R³ is

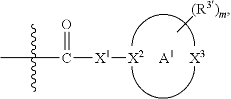

wherein Ring A¹, X¹, X², X³, R^{3′} and m are as defined in the first embodiment.

In one embodiment of formula V, wherein R³ is

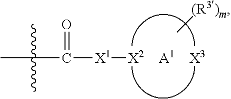

wherein X¹ is —CH₂—, —NH— or —O—, and Ring A¹, X², X³, R^{3′} and m are as defined in the first embodiment.

In one embodiment of formula V, wherein R³ is

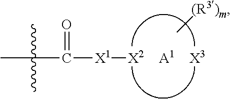

wherein X¹ is —NH—, and Ring A¹, X², X³, R^{3′} and m are as defined in the first embodiment.

In one embodiment of formula V, wherein R³ is

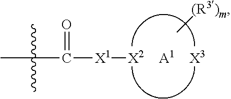

wherein X¹ is —NH—, and Ring A¹ is 3-, 4-, 5- or 6-membered cycloalkyl, and X², X³, R and m are as defined in the first embodiment.

In one embodiment of formula V, wherein R³ is

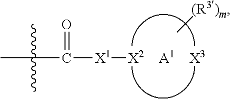

wherein X¹ is —NH—, and Ring A¹ is 4-, 5- or 6-membered heterocycloalkyl, wherein the heterocycloalkyl of Ring A¹ contains one or more heteroatoms selected from N and O, and X², X³, R^{3′} and m are as defined in the first embodiment.

In one embodiment of formula V, wherein Ring A is 10-membered nitrogen heteroaryl, and R³ is

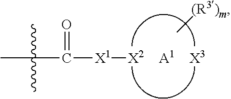

wherein Ring A¹, X¹, X², X³, R^{3′} and m are as defined in the first embodiment.

In one embodiment of formula V, wherein Ring A is 10-membered nitrogen heteroaryl, and R³ is

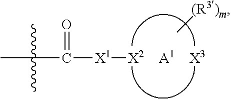

wherein X¹ is —CH₂—, —NH— or —O—, and Ring A¹, X², X³, R^{3′} and m are as defined in the first embodiment.

In one embodiment of formula V, wherein Ring A is 10-membered nitrogen

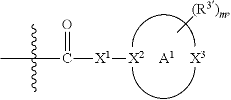

heteroaryl, and R³ is, wherein X¹ is —NH—, and Ring A¹, X², X³, R^{3′} and m are as defined in the first embodiment.

In one embodiment of formula V, R³ is

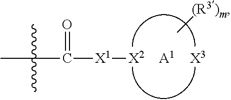

wherein X¹ is —NH—, and Ring A¹ is 3-, 4-, 5- or 6-membered cycloalkyl, and X², X³, R³ and m are as defined in the first embodiment.

In one embodiment of formula V, wherein Ring A is 10-membered nitrogen heteroaryl, and R³ is

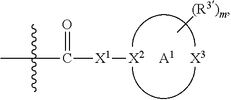

wherein X¹ is —NH—, and Ring A¹ is 4-, 5- or 6-membered heterocycloalkyl, wherein the heterocycloalkyl of Ring A¹ contains one or more heteroatoms selected from N and O, and X², X³, R^{3′} and m are as defined in the first embodiment.

In some embodiments, a compound of the disclosure is of the following structural Formula VI:

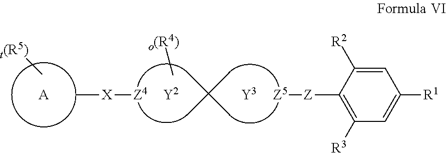

Ring Y² is 4- or 5-membered nitrogen heterocyclyl;
Ring Y³ is 4-, 5- or 6-membered nitrogen heterocyclyl;
R⁴ substitution occurs on any of the ring atoms of Ring Y² and/or Ring Y³, as long as valence permits;
when Z⁴ is CH, X is —C(═O)NRⁿ— or —R^{m}—C(═O)NRⁿ—R^{o}—;
when Z⁴ is N, X is absent, —CR^{k}R^{L}—, or —C(═O)—; and
when Z⁵ is CH, Z is —NR^{g}—, —C(R^{h}Rⁱ)—NR^{g}, —NR^{g}—C(R^{h}Rⁱ)- [symbol] —O—;
when Z⁵ is N, Z is absent, and all other variables not specifically defined herein are as defined in the first embodiment. In some embodiments, Z⁴ and Z⁵ are N, and all other variables not specifically defined herein are as defined in the first embodiment.

In one embodiment of formula VI, wherein R³ is

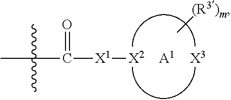

wherein Ring A¹, X¹, X², X³, R^{3′} and m are as defined in the first embodiment.

In one embodiment of formula VI, wherein R³ is

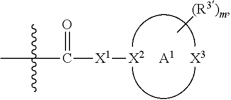

wherein X¹ is —CH₂—, —NH— or —O—, and Ring A¹, X², X³, R^{3′} and m are as defined in the first embodiment.

In one embodiment of formula VI, wherein R³ is

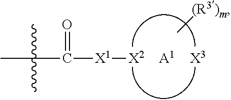

wherein X¹ is —NH—, and Ring A¹, X², X³, R^{3′} and m are as defined in the first embodiment.

In one embodiment of formula VI, wherein R³ is

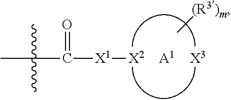

wherein X¹ is —NH—, and Ring A¹ is 3-, 4-, 5- or 6-membered cycloalkyl, and X², X³, R^{3′} and m are as defined in the first embodiment.

In one embodiment of formula VI, wherein R³ is

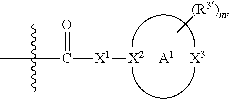

wherein X¹ is —NH—, and Ring A¹ is 4-, 5- or 6-membered heterocycloalkyl, wherein the heterocycloalkyl of Ring A¹ contains one or more heteroatoms selected from N and O, and X², X³, R^{3′} and m are as defined in the first embodiment.

In one embodiment of formula VI, wherein Ring A is 10-membered nitrogen heteroaryl, and R³ is

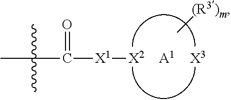

wherein Ring A¹, X¹, X², X³, R^{3′} and m are as defined in the first embodiment.

In one embodiment of formula VI, wherein Ring A is 10-membered nitrogen heteroaryl, and R³ is

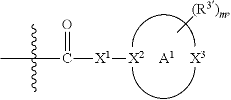

wherein X¹ is —CH₂—, —NH— or —O—, and Ring A¹, X², X³, R^{3′} and m are as defined in the first embodiment.

In one embodiment of formula VI, wherein Ring A is 10-membered nitrogen heteroaryl, and R³ is

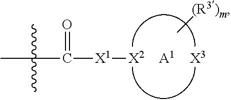

wherein X¹ is —NH—, and Ring A¹, X², X³, R^{3′} and m are as defined in the first embodiment.

In one embodiment of formula VI, R³ is

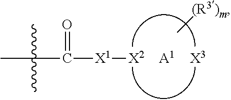

wherein X¹ is —NH—, and Ring A¹ is 3-, 4-, 5- or 6-membered cycloalkyl, and X², X³, R^{3′} and m are as defined in the first embodiment.

In one embodiment of formula VI, wherein Ring A is 10-membered nitrogen heteroaryl, and R³ is

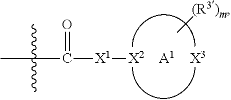

wherein X¹ is —NH—, and Ring A¹ is 4-, 5- or 6-membered heterocycloalkyl, wherein the heterocycloalkyl of Ring A¹ contains one or more heteroatoms selected from N and O, and X², X³, R^{3′} and m are as defined in the first embodiment.

In some embodiments, Z⁴ is in the meta position to the spiro atom, and all other variables not specifically defined herein are as defined in the first embodiment.

In some embodiments, Z⁵ is in the meta position to the spiro atom, and all other variables not specifically defined herein are as defined in the first embodiment.

In some embodiments, Y is

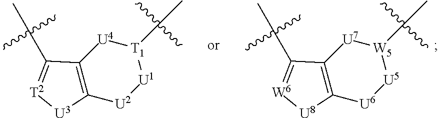

U¹, U², and U⁴ are CH₂; U³ is NH; T¹ and T² are N, and all other variables not specifically defined herein are as defined in the first embodiment.

In some embodiments, Y is

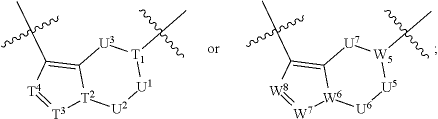

T¹, T² and T⁴ are N; T³ is CH; U¹, U² and U³ are CH₂, and all other variables not specifically defined herein are as defined in the first embodiment.

In some embodiments, Ring Y is

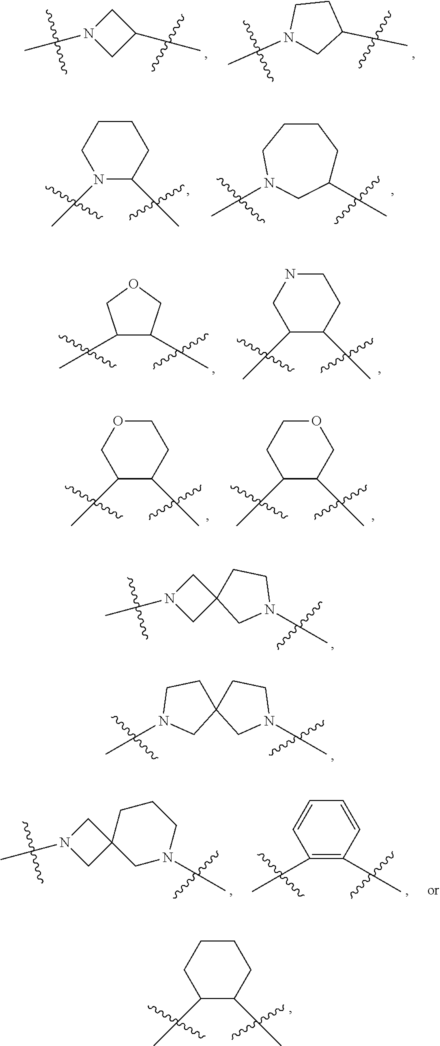

and all other variables not specifically defined herein are as defined in the first embodiment.

In some embodiments, Ring Y is

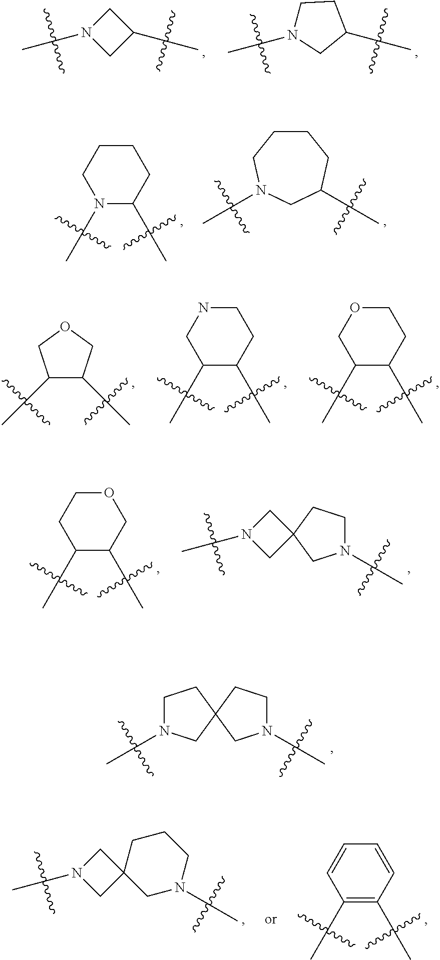

and all other variables not specifically defined herein are as defined in the first embodiment.

In some embodiments, the moiety

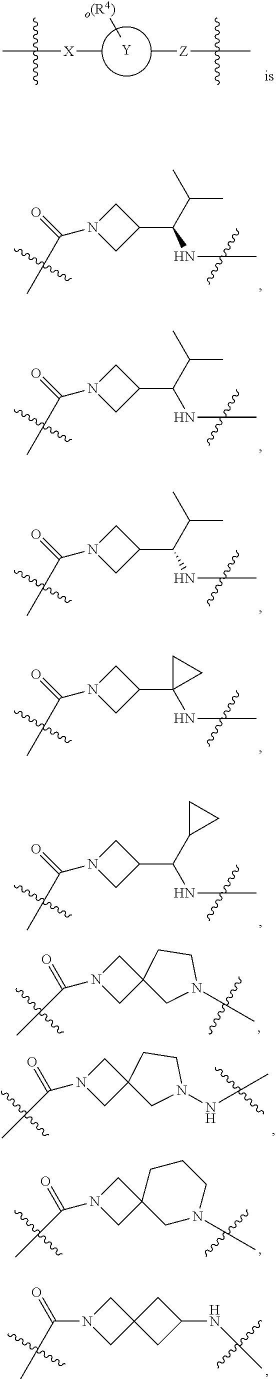

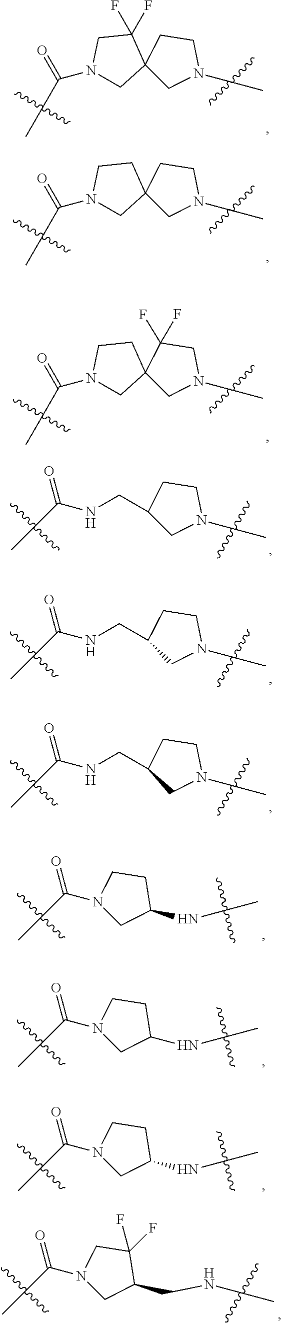

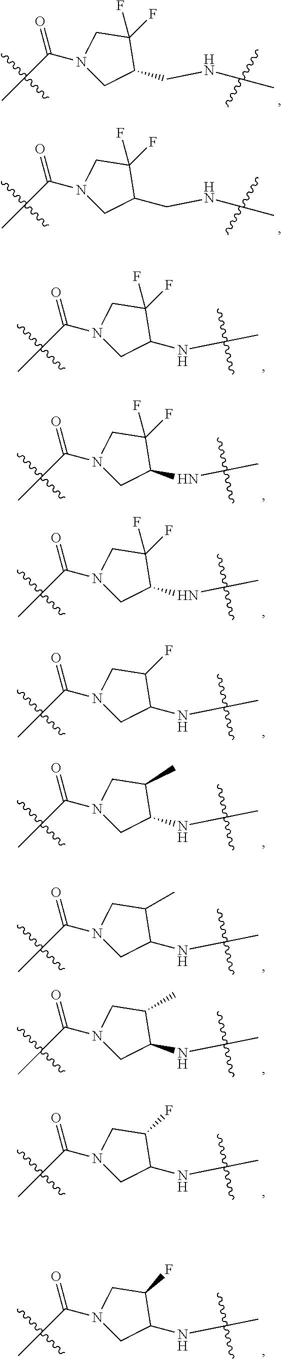

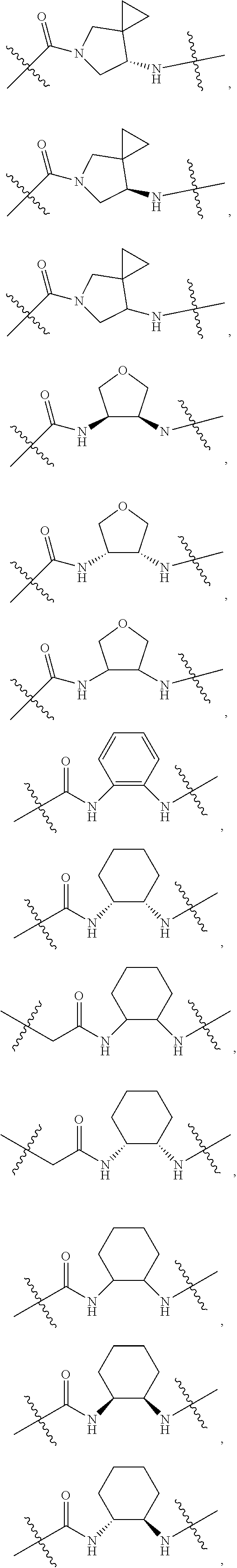

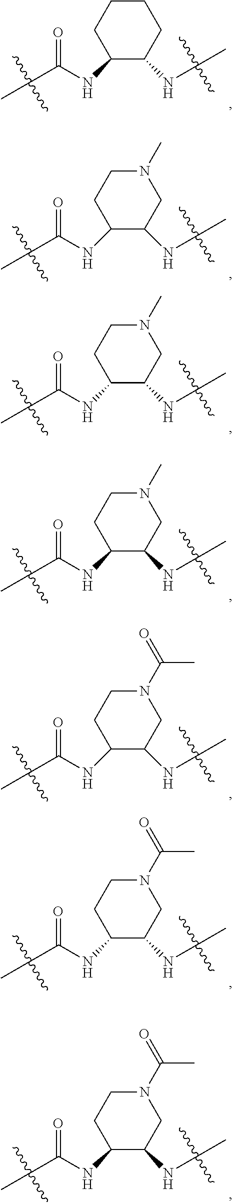

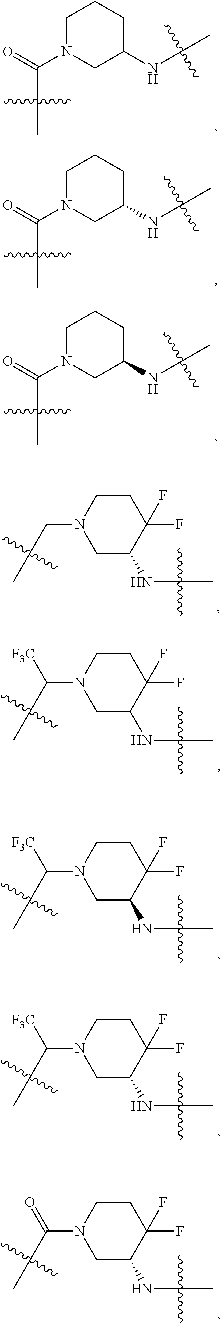

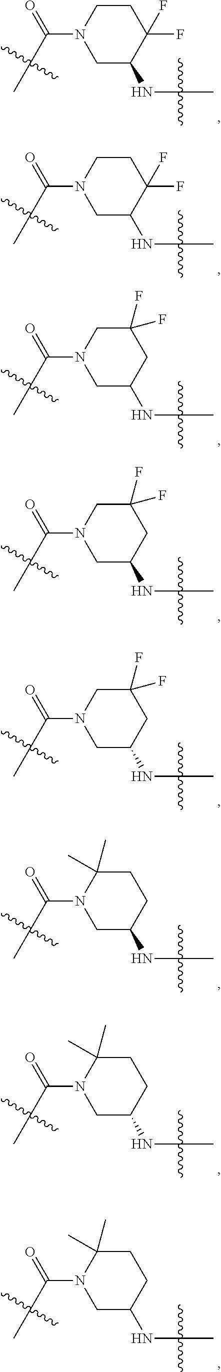

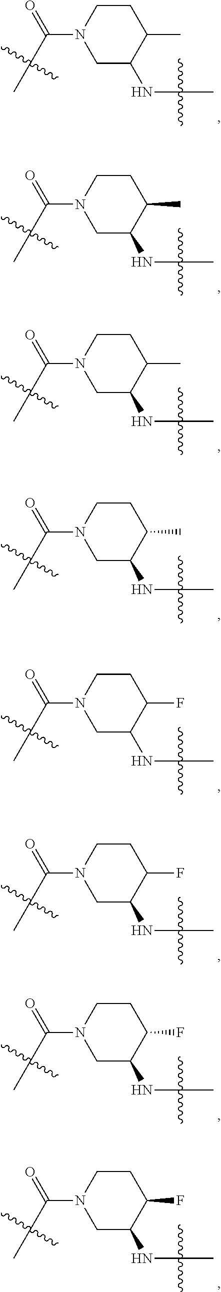

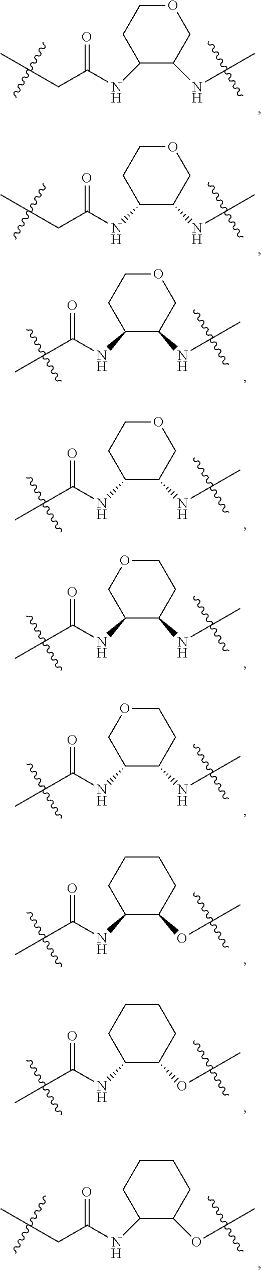

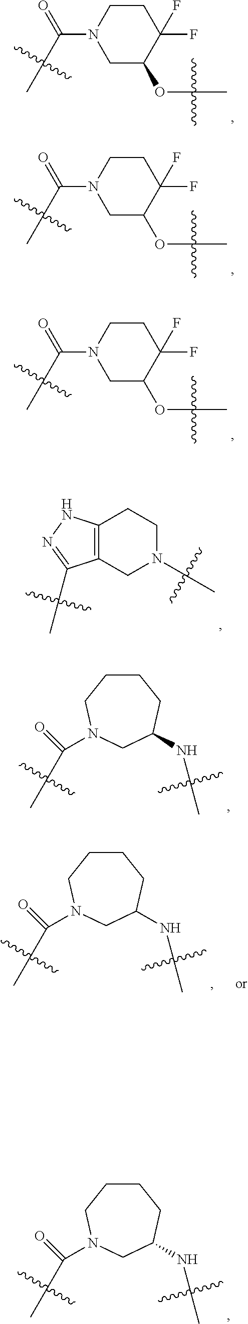

and all other variables not specifically defined herein are as defined in the first embodiment.

In some embodiments, the moiety

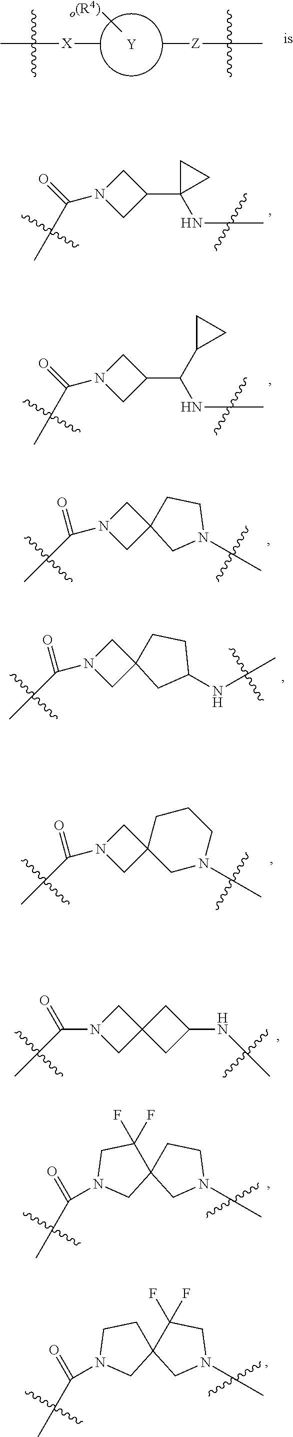

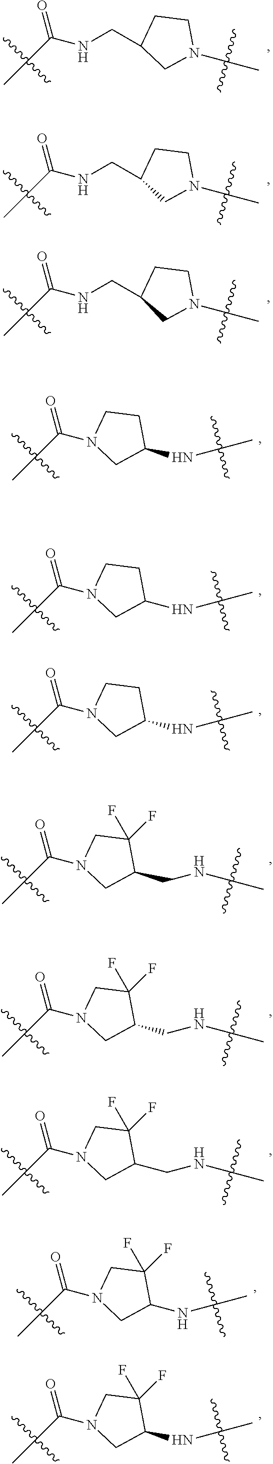

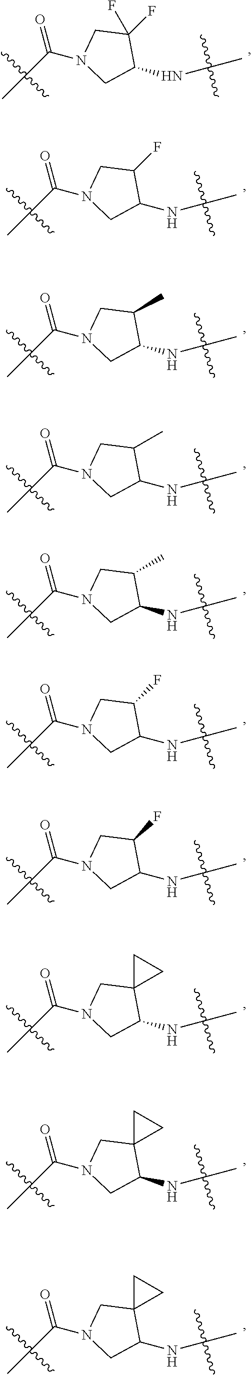

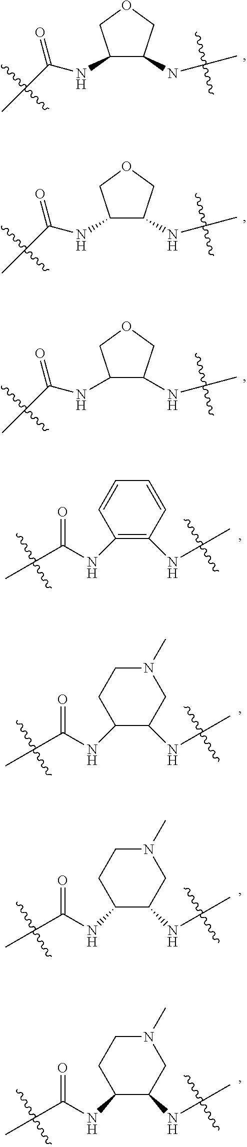

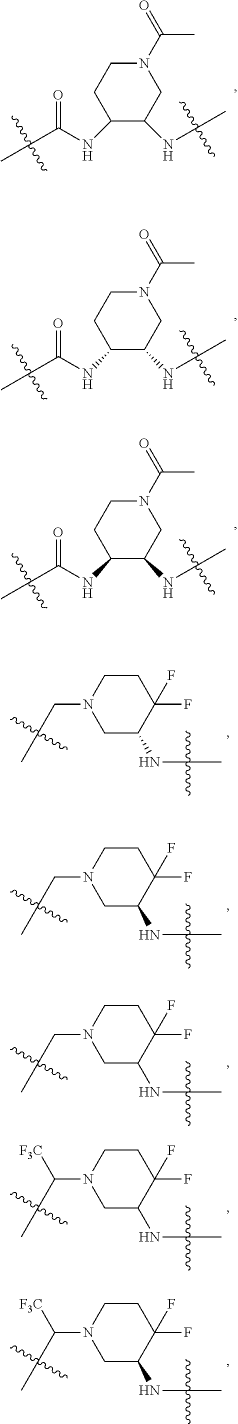

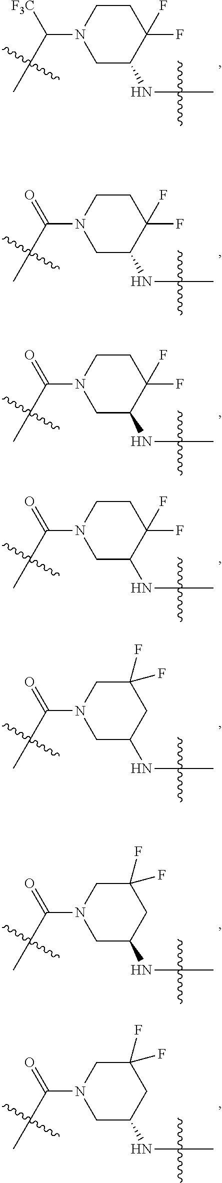

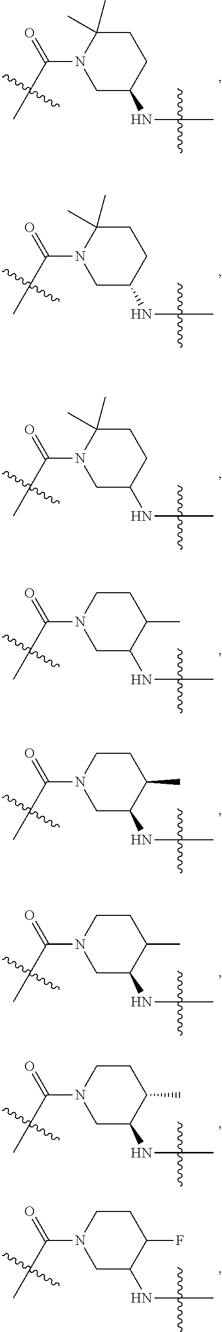

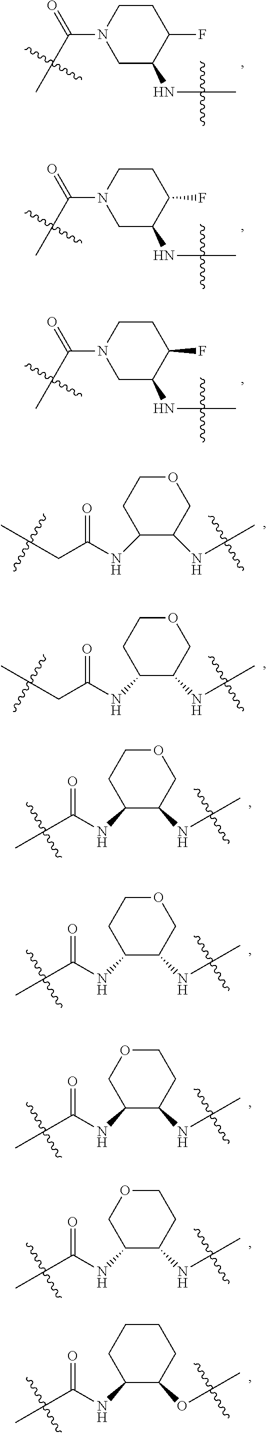

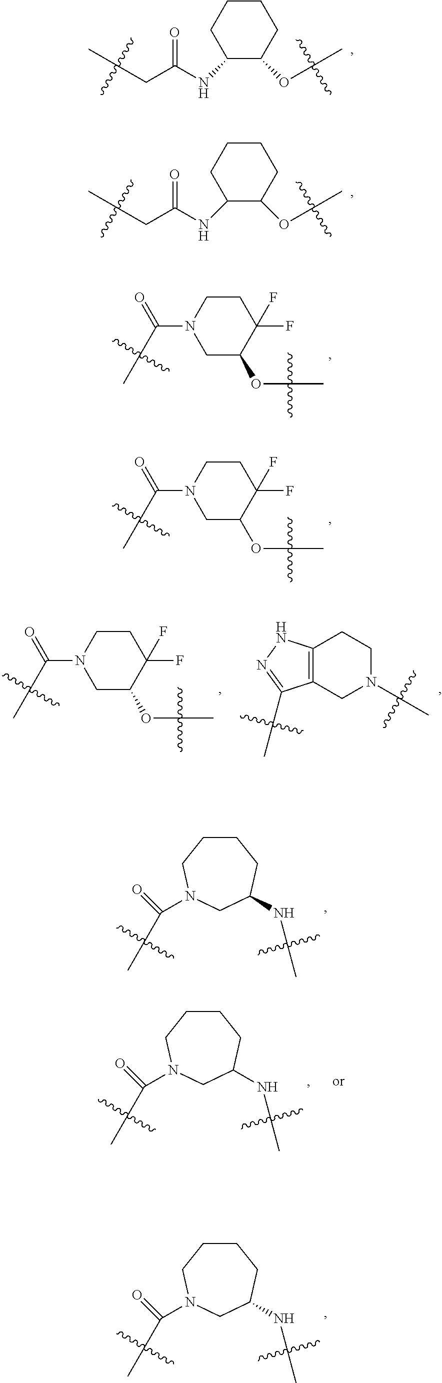

and all other variables not specifically defined herein are as defined in the first embodiment.

In some embodiments, the moiety

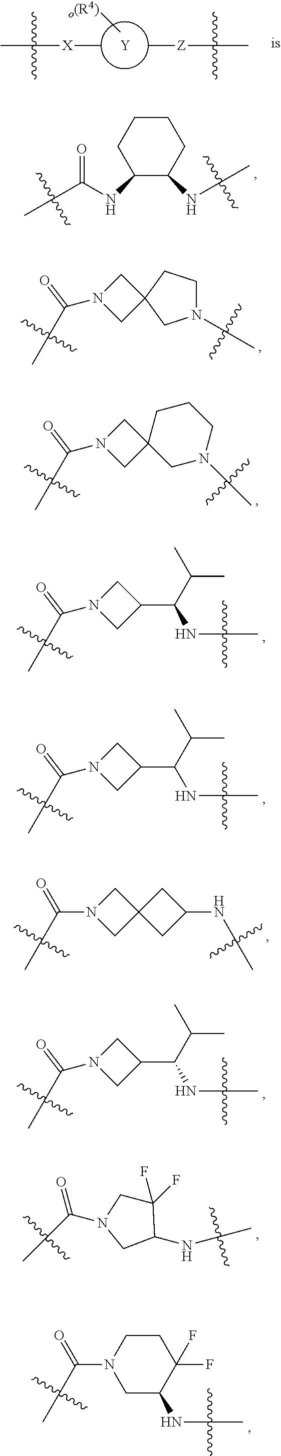

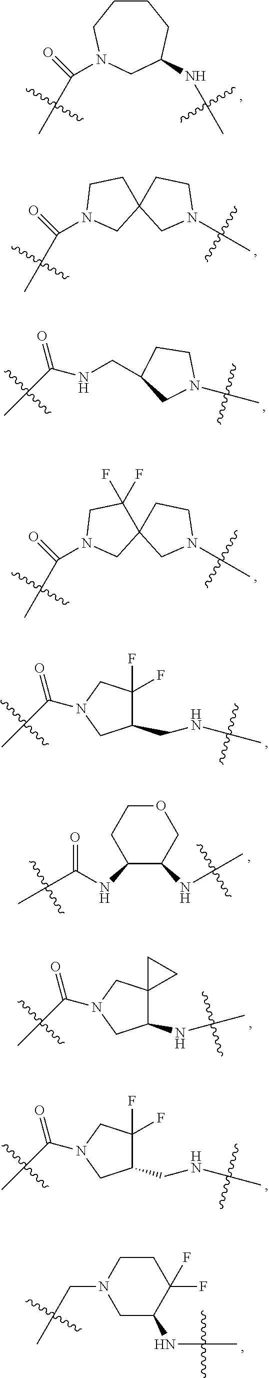

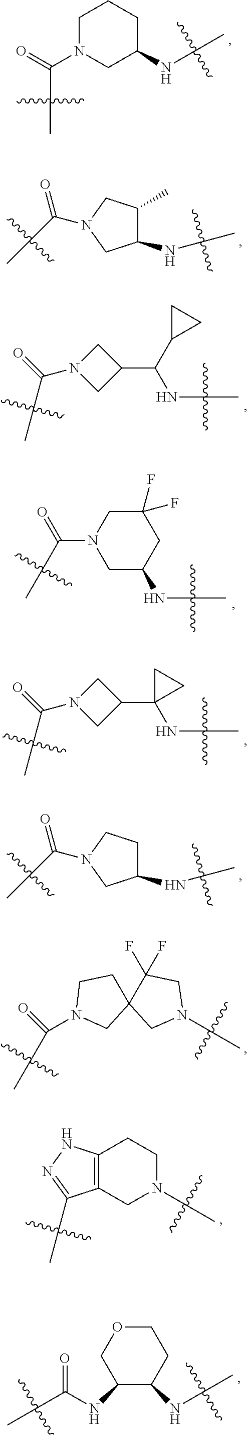

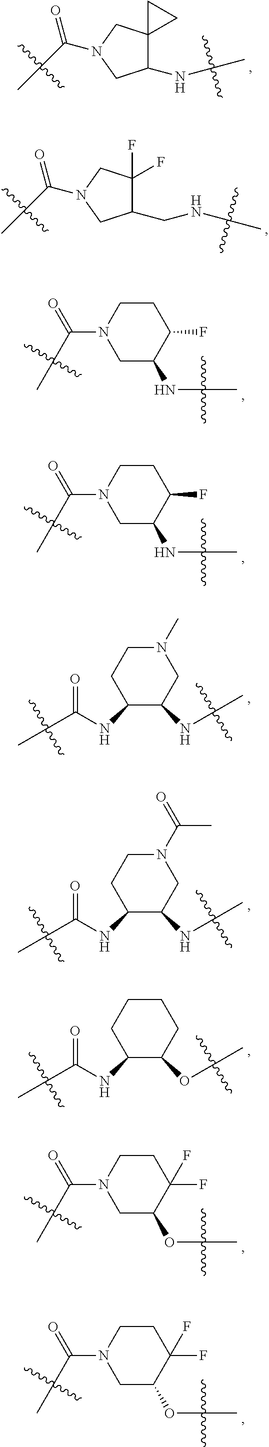

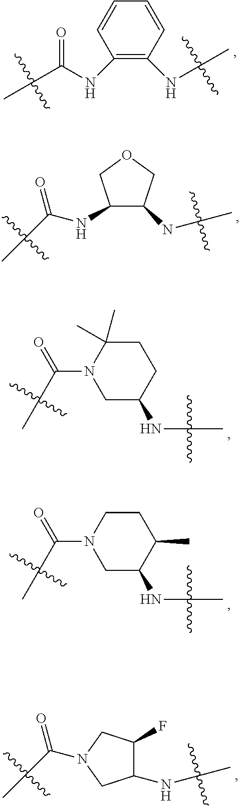 [SMILES: *Nc1ccccc1NC(*)=O ; *N[C@@H]1COC[C@@H]1NC(*)=O ; *N[C@@H]1CCC(C)(C)N(C(*)=O)C1 ; *N[C@H]1CN(C(*)=O)CC[C@H]1C ; *NC1CN(C(*)=O)C[C@H]1F]

and all other variables not specifically defined herein are as defined in the first embodiment.

In some embodiments, the moiety

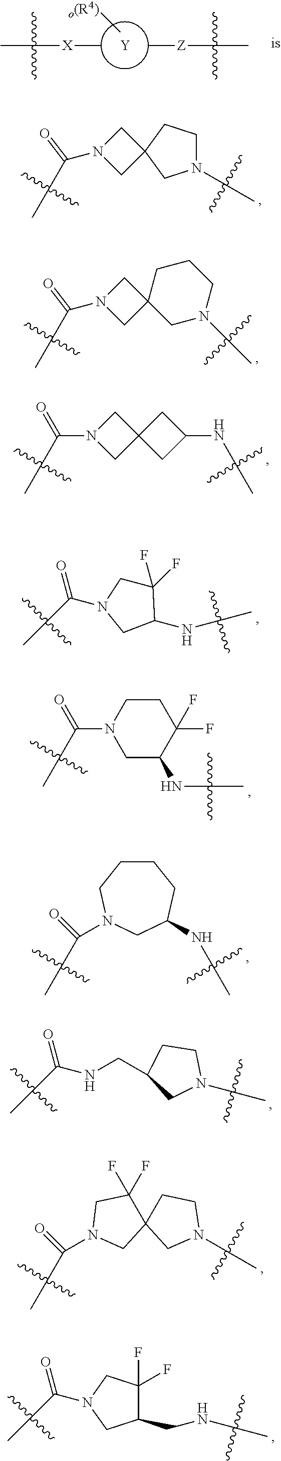

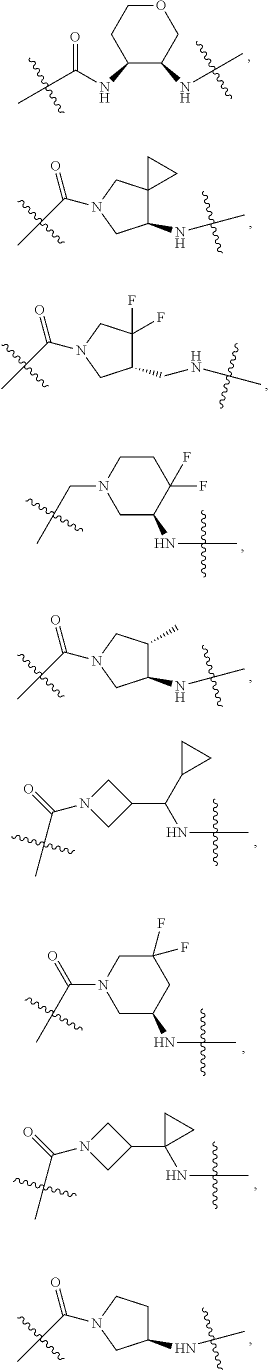

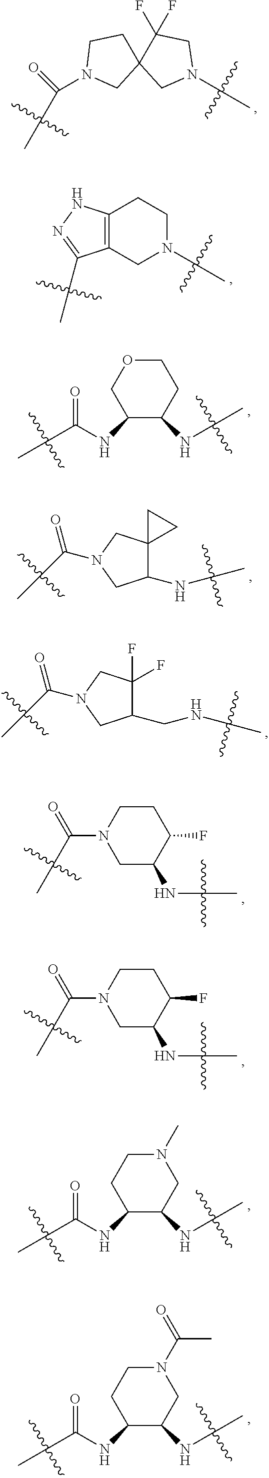

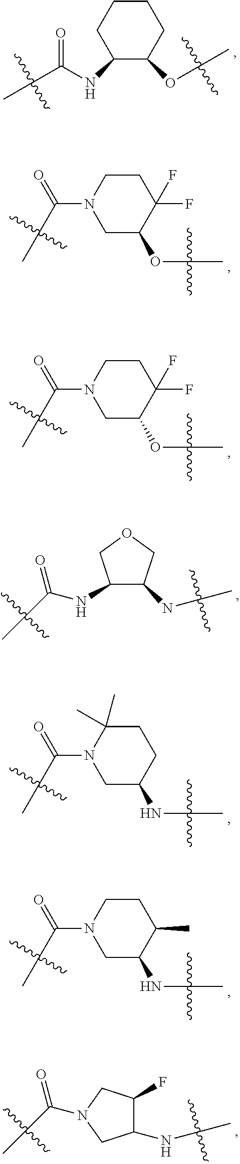

and all other variables not specifically defined herein are as defined in the first embodiment.

In some embodiments, R^{k} is H, R^{L} is CF₃, and all other variables not specifically defined herein are as defined in the first embodiment.

In some embodiments, R^{m} is —CH₂—, R^{c} is absent, and all other variables not specifically defined herein are as defined in the first embodiment.

In some embodiments, R^{o} is —CH₂—, R^{m} is absent, and all other variables not specifically defined herein are as defined in the first embodiment.

In some embodiments, the compound is of the following structural Formula VII:

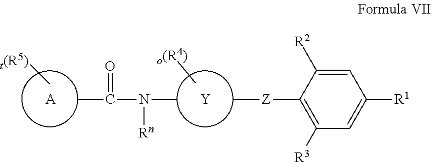

Y is

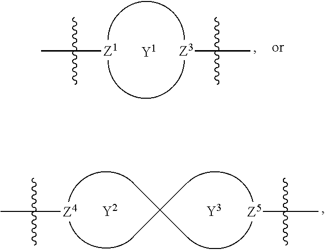

wherein: Z¹ is CH, or Z⁴ is CH, and all other variables not specifically defined herein are as defined in the first embodiment.

In some embodiments, Rⁿ is H, and all other variables not specifically defined herein are as defined in the first embodiment.

In some embodiments, the compound is of the following structural Formula VIII:

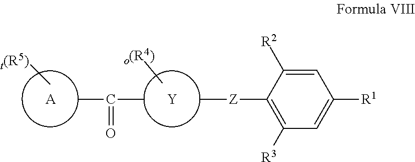

Z¹ or Z⁴ is N, Y is

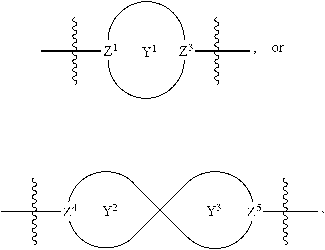

wherein: and all other variables not specifically defined herein are as defined in the first embodiment.

In some embodiments, Ring A is

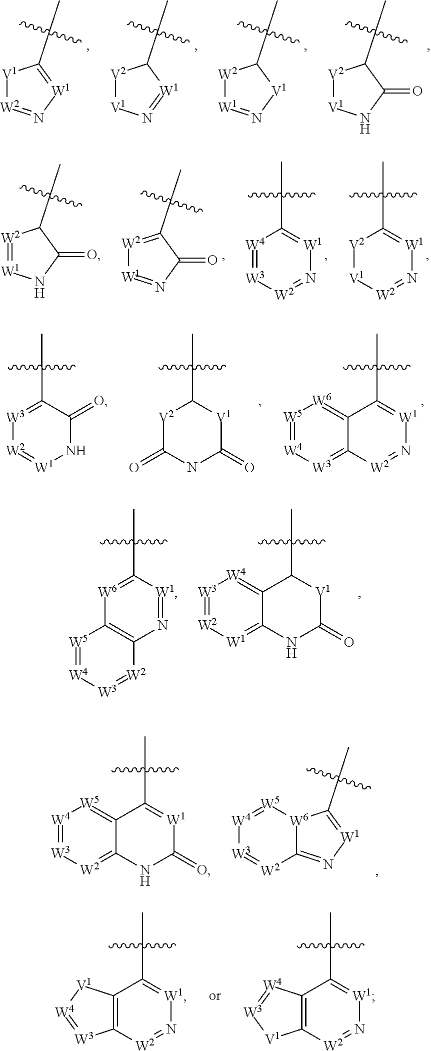

for each Ring A group, W¹, W², W³, W⁴, W⁵ and W⁶ are each independently N or CH; for each Ring A group, V¹ and V² are each independently NH or CH₂, and all other variables not specifically defined herein are as defined in the first embodiment.

In some embodiments, Ring A is

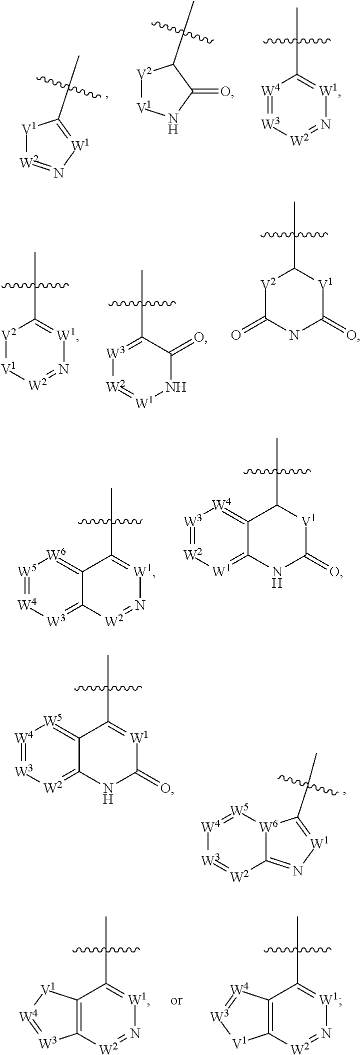

for each Ring A group, W¹, W², W³, W⁴, W⁵ and W⁶ are each independently N or CH; for each Ring A group, V¹ and V² are each independently NH or CH₂, and all other variables not specifically defined herein are as defined in the first embodiment.

In some embodiments, Ring A is pyridinyl, pyrimidinyl, pentanolactam, butyrolactam, glutarimide, triazolyl, tetrazolyl, isoquinolinyl, quinolinyl, pyridin-2(1H)-one, quinolin-2(1H)-one, 3,4-dihydroquinolin-2(1H)-one, naphthyridinyl, pyrrolopyridinyl, or imidazopyridinyl, wherein the pyridinyl, pyrimidinyl, pentanolactam, butyrolactam, glutarimide, triazolyl, tetrazolyl, isoquinolinyl, quinolinyl, pyridin-2(1H)-one, quinolin-2(1H)-one, 3,4-dihydroquinolin-2(1H)-one, naphthyridinyl, pyrrolopyridinyl, or imidazopyridinyl substituted by t groups of R⁵, and all other variables not specifically defined herein are as defined in the first embodiment.

In some embodiments, Ring A is

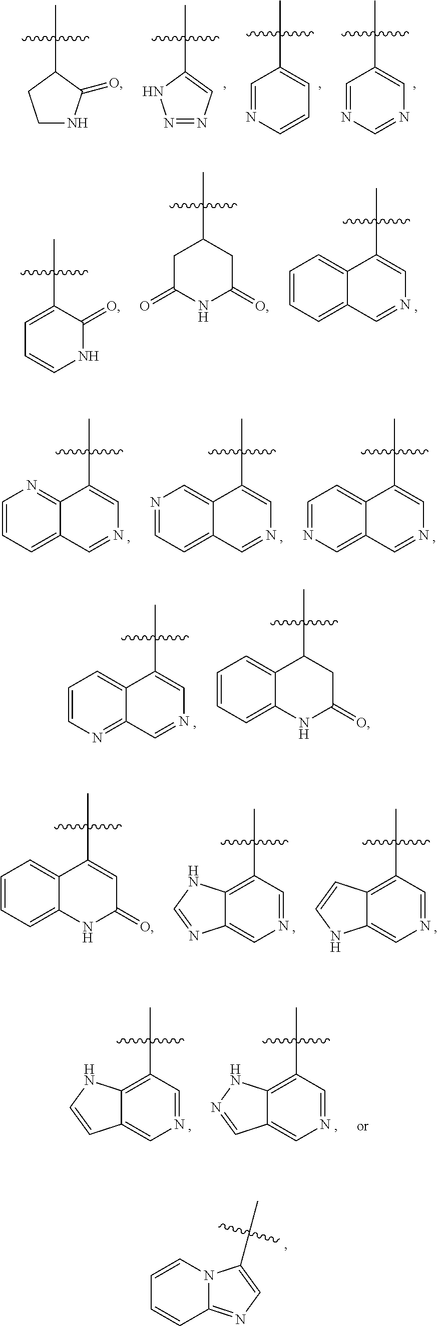

wherein Ring A is substituted by t groups of R⁵, the substitution occurs on any of atoms of the Ring A, as long as valence permits, and all other variables not specifically defined herein are as defined in the first embodiment.

In some embodiments, the moeity

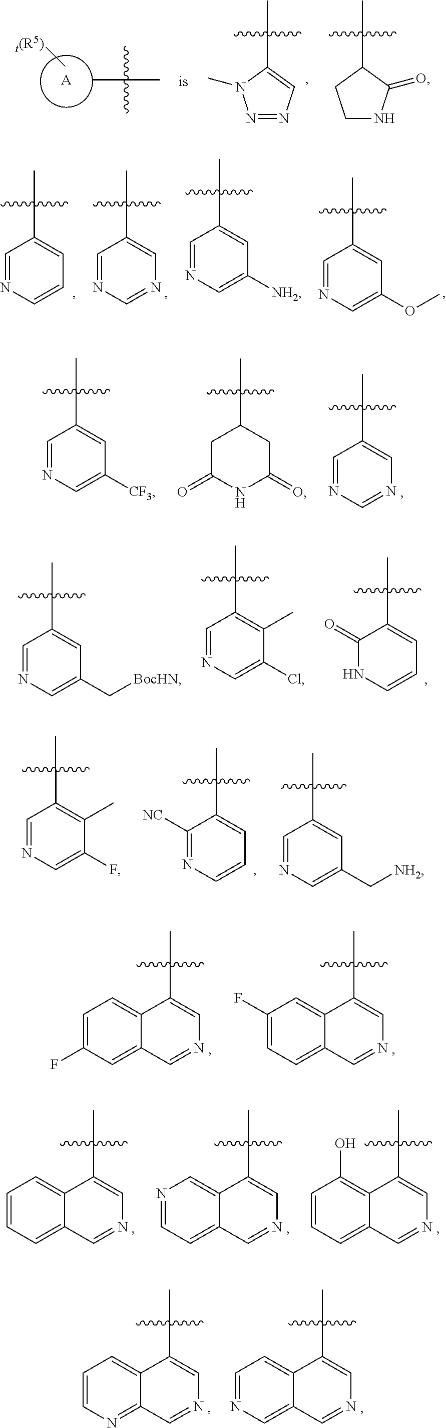

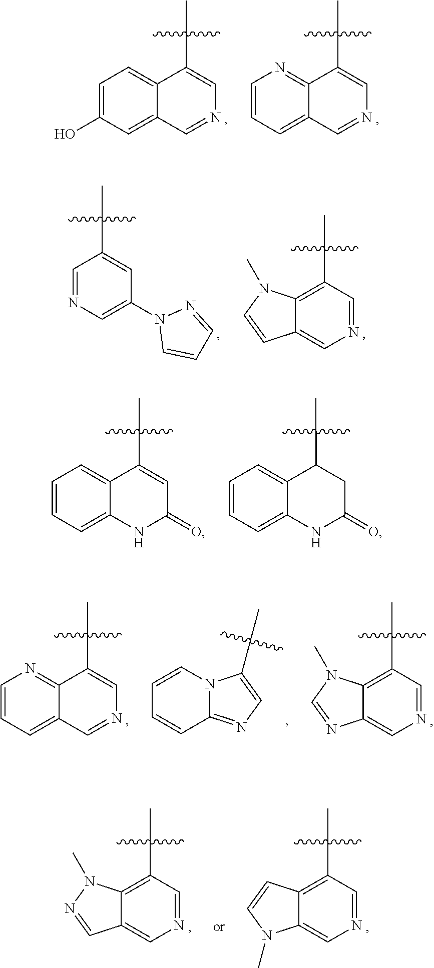

and all other variables not specifically defined herein are as defined in the first embodiment

In some embodiments, the moiety

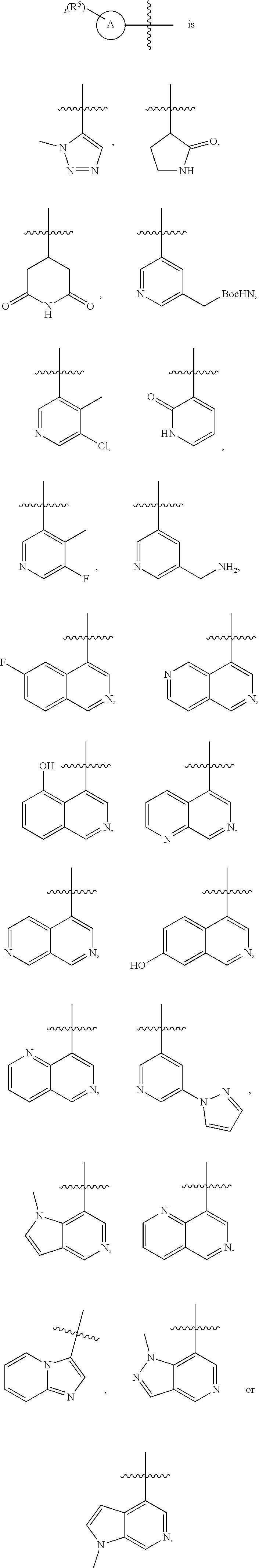

and all other variables not specifically defined herein are as defined in the first embodiment.

In some embodiments, the 5- or 6-memberedheteroaryl of R⁵ is pyrroyl, pyrazolyl, or imidazolyl, and all other variables not specifically defined herein are as defined in the first embodiment.

In some embodiments, the 5- or 6-membered heteroaryl of R⁵ is

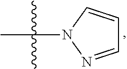 [SMILES: *n1cccn1]

and all other variables not specifically defined herein are as defined in the first embodiment.

In some embodiments, when R³ is —C(═O)NHCH₃, Ring A isn't isoquinolinyl; or when Ring A is isoquinolinyl, R³ isn't —C(═O)NHCH₃, and all other variables not specifically defined herein are as defined in the first embodiment.

Ring Y isn't cyclohexyl, 6-membered nitrogen heterocyclyl, 5-membered nitrogen heterocyclyl or diazaspiro[4.4], and all other variables not specifically defined herein are as defined in the first embodiment.

In some embodiments, When X is absent, Ring Y is

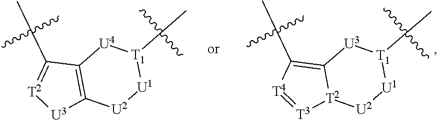

and all other variables not specifically defined herein are as defined in the first embodiment.

In certain embodiments, the at least one compound of the disclosure is selected from Compounds 1 to 183 depicted in Table 1, a solvate thereof, a tautomer thereof, a stereoisomer thereof, a deuterated derivative of the compound, or a pharmaceutically acceptable salt of the foregoing.

TABLE 1
Compounds 1 to 183 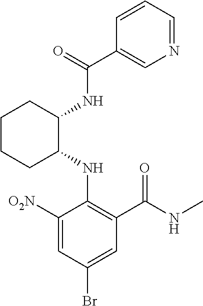 [SMILES: CNC(=O)c1cc(Br)cc([N+](=O)[O-])c1N[C@@H]1CCCC[C@@H]1NC(=O)c1cccnc1]
1 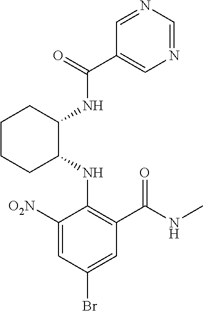 [SMILES: CNC(=O)c1cc(Br)cc([N+](=O)[O-])c1N[C@@H]1CCCC[C@@H]1NC(=O)c1cncnc1]
2 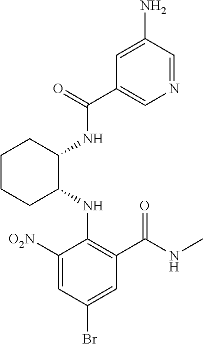 [SMILES: CNC(=O)c1cc(Br)cc([N+](=O)[O-])c1N[C@@H]1CCCC[C@@H]1NC(=O)c1cncc(N)c1]
3 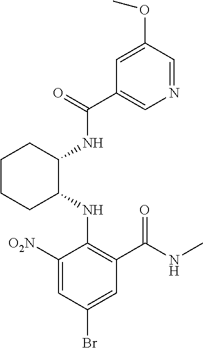 [SMILES: CNC(=O)c1cc(Br)cc([N+](=O)[O-])c1N[C@@H]1CCCC[C@@H]1NC(=O)c1cncc(OC)c1]
4 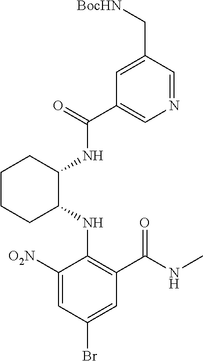 [SMILES: CNC(=O)c1cc(Br)cc([N+](=O)[O-])c1N[C@@H]1CCCC[C@@H]1NC(=O)c1cncc(CNC(=O)OC(C)(C)C)c1]
5 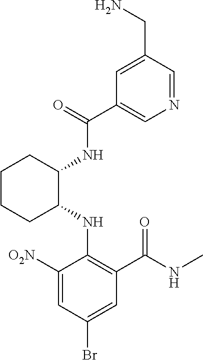 [SMILES: CNC(=O)c1cc(Br)cc([N+](=O)[O-])c1N[C@@H]1CCCC[C@@H]1NC(=O)c1cncc(CN)c1]
6 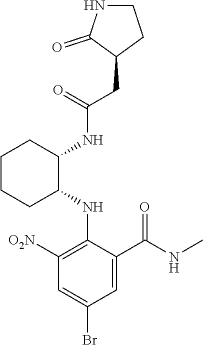 [SMILES: CNC(=O)c1cc(Br)cc([N+](=O)[O-])c1N[C@@H]1CCCC[C@@H]1NC(=O)C[C@@H]1CCNC1=O]
7 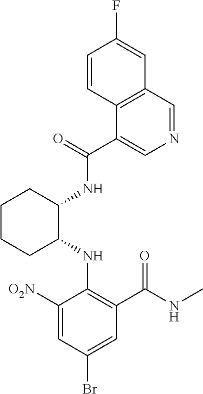 [SMILES: CNC(=O)c1cc(Br)cc([N+](=O)[O-])c1N[C@@H]1CCCC[C@@H]1NC(=O)c1cncc2cc(F)ccc12]
8 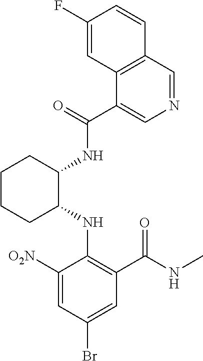 [SMILES: CNC(=O)c1cc(Br)cc([N+](=O)[O-])c1N[C@@H]1CCCC[C@@H]1NC(=O)c1cncc2ccc(F)cc12]
9 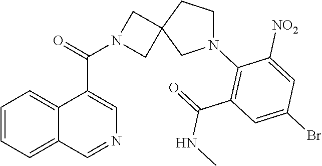 [SMILES: CNC(=O)c1cc(Br)cc([N+](=O)[O-])c1N1CCC2(CN(C(=O)c3cncc4ccccc34)C2)C1]
10 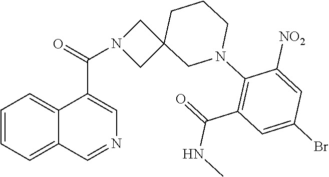 [SMILES: CNC(=O)c1cc(Br)cc([N+](=O)[O-])c1N1CCCC2(CN(C(=O)c3cncc4ccccc34)C2)C1]
11 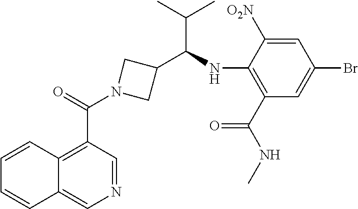 [SMILES: CNC(=O)c1cc(Br)cc([N+](=O)[O-])c1N[C@H](C(C)C)C1CN(C(=O)c2cncc3ccccc23)C1]
12 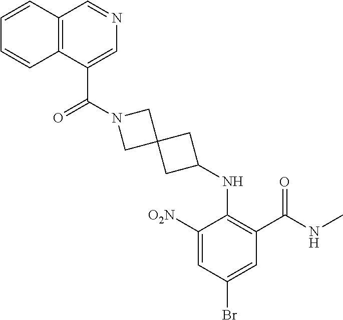 [SMILES: CNC(=O)c1cc(Br)cc([N+](=O)[O-])c1NC1CC2(C1)CN(C(=O)c1cncc3ccccc13)C2]
13 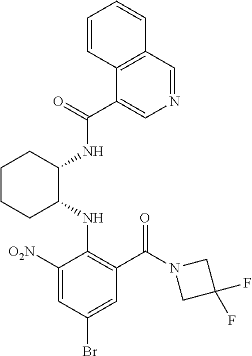 [SMILES: O=C(N[C@H]1CCCC[C@H]1Nc1c(C(=O)N2CC(F)(F)C2)cc(Br)cc1[N+](=O)[O-])c1cncc2ccccc12]
14 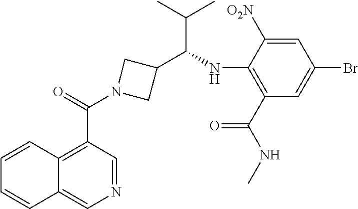 [SMILES: CNC(=O)c1cc(Br)cc([N+](=O)[O-])c1N[C@@H](C(C)C)C1CN(C(=O)c2cncc3ccccc23)C1]
15 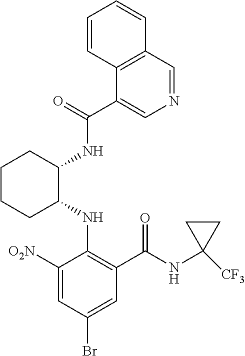 [SMILES: O=C(NC1(C(F)(F)F)CC1)c1cc(Br)cc([N+](=O)[O-])c1N[C@@H]1CCCC[C@@H]1NC(=O)c1cncc2ccccc12]
16 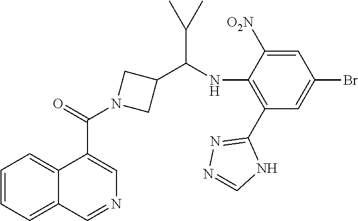 [SMILES: CC(C)C(Nc1c(-c2nnc[nH]2)cc(Br)cc1[N+](=O)[O-])C1CN(C(=O)c2cncc3ccccc23)C1]
17 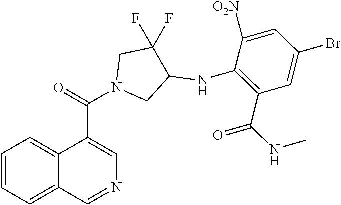 [SMILES: CNC(=O)c1cc(Br)cc([N+](=O)[O-])c1NC1CN(C(=O)c2cncc3ccccc23)CC1(F)F]
18 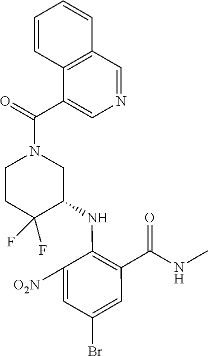 [SMILES: CNC(=O)c1cc(Br)cc([N+](=O)[O-])c1N[C@H]1CN(C(=O)c2cncc3ccccc23)CCC1(F)F]
19 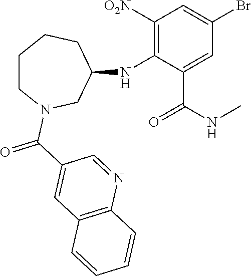 [SMILES: CNC(=O)c1cc(Br)cc([N+](=O)[O-])c1N[C@@H]1CCCCN(C(=O)c2cnc3ccccc3c2)C1]
20 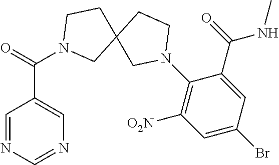 [SMILES: CNC(=O)c1cc(Br)cc([N+](=O)[O-])c1N1CCC2(CCN(C(=O)c3cncnc3)C2)C1]
21 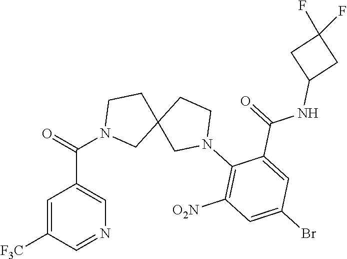 [SMILES: O=C(NC1CC(F)(F)C1)c1cc(Br)cc([N+](=O)[O-])c1N1CCC2(CCN(C(=O)c3cncc(C(F)(F)F)c3)C2)C1]
22 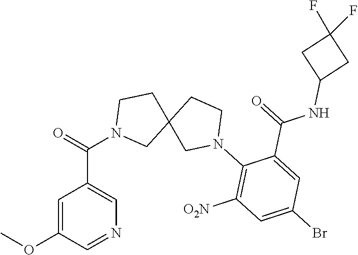 [SMILES: COc1cncc(C(=O)N2CCC3(CCN(c4c(C(=O)NC5CC(F)(F)C5)cc(Br)cc4[N+](=O)[O-])C3)C2)c1]
23 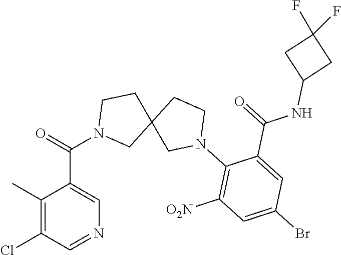 [SMILES: Cc1c(Cl)cncc1C(=O)N1CCC2(CCN(c3c(C(=O)NC4CC(F)(F)C4)cc(Br)cc3[N+](=O)[O-])C2)C1]
24 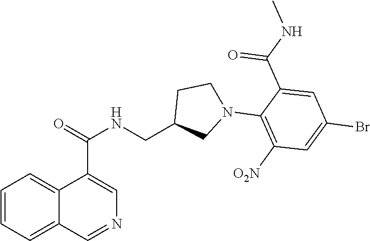 [SMILES: CNC(=O)c1cc(Br)cc([N+](=O)[O-])c1N1CC[C@H](CNC(=O)c2cncc3ccccc23)C1]
25 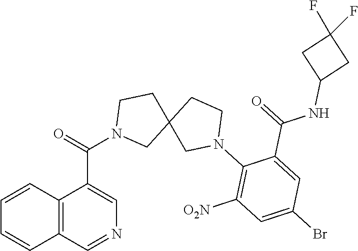 [SMILES: O=C(NC1CC(F)(F)C1)c1cc(Br)cc([N+](=O)[O-])c1N1CCC2(CCN(C(=O)c3cncc4ccccc34)C2)C1]
26 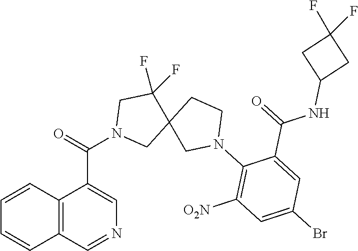 [SMILES: O=C(NC1CC(F)(F)C1)c1cc(Br)cc([N+](=O)[O-])c1N1CCC2(CN(C(=O)c3cncc4ccccc34)CC2(F)F)C1]
27 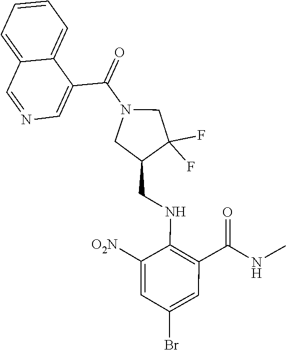 [SMILES: CNC(=O)c1cc(Br)cc([N+](=O)[O-])c1NC[C@H]1CN(C(=O)c2cncc3ccccc23)CC1(F)F]
28 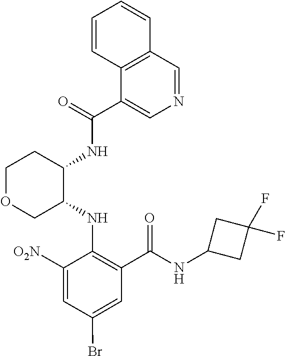 [SMILES: O=C(NC1CC(F)(F)C1)c1cc(Br)cc([N+](=O)[O-])c1N[C@@H]1COCC[C@@H]1NC(=O)c1cncc2ccccc12]
29 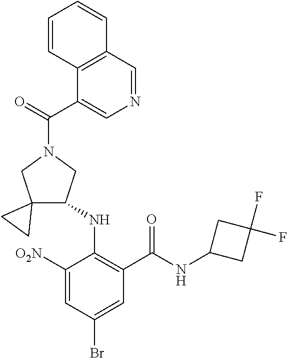 [SMILES: O=C(NC1CC(F)(F)C1)c1cc(Br)cc([N+](=O)[O-])c1N[C@H]1CN(C(=O)c2cncc3ccccc23)CC12CC2]
30 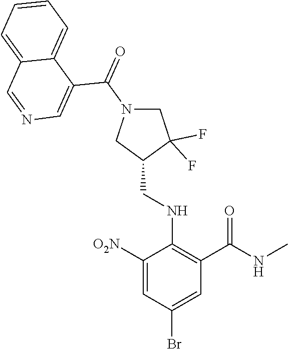 [SMILES: CNC(=O)c1cc(Br)cc([N+](=O)[O-])c1NC[C@@H]1CN(C(=O)c2cncc3ccccc23)CC1(F)F]
31 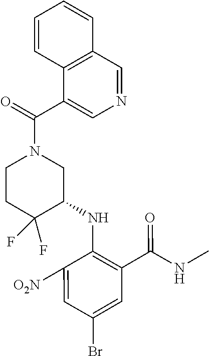 [SMILES: CNC(=O)c1cc(Br)cc([N+](=O)[O-])c1N[C@H]1CN(C(=O)c2cncc3ccccc23)CCC1(F)F]
32 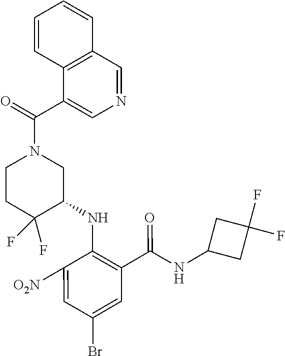 [SMILES: O=C(NC1CC(F)(F)C1)c1cc(Br)cc([N+](=O)[O-])c1N[C@H]1CN(C(=O)c2cncc3ccccc23)CCC1(F)F]
33 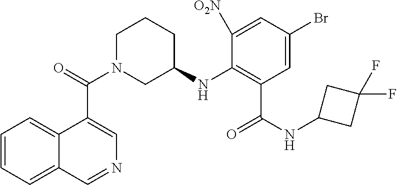 [SMILES: O=C(NC1CC(F)(F)C1)c1cc(Br)cc([N+](=O)[O-])c1N[C@@H]1CCCN(C(=O)c2cncc3ccccc23)C1]
34 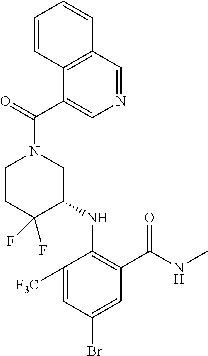 [SMILES: CNC(=O)c1cc(Br)cc(C(F)(F)F)c1N[C@H]1CN(C(=O)c2cncc3ccccc23)CCC1(F)F]
35 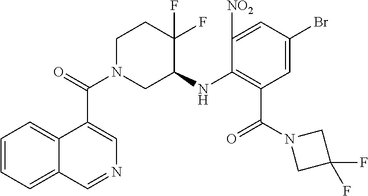 [SMILES: O=C(c1cc(Br)cc([N+](=O)[O-])c1N[C@H]1CN(C(=O)c2cncc3ccccc23)CCC1(F)F)N1CC(F)(F)C1]
36 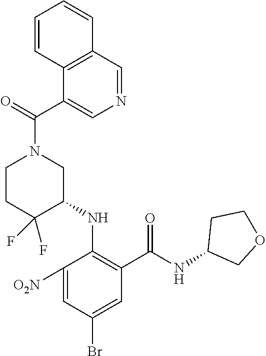 [SMILES: O=C(N[C@@H]1CCOC1)c1cc(Br)cc([N+](=O)[O-])c1N[C@H]1CN(C(=O)c2cncc3ccccc23)CCC1(F)F]
37 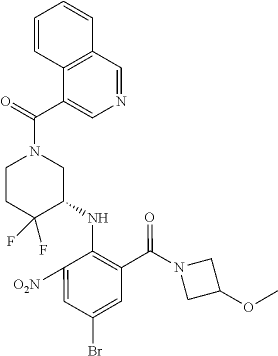 [SMILES: COC1CN(C(=O)c2cc(Br)cc([N+](=O)[O-])c2N[C@H]2CN(C(=O)c3cncc4ccccc34)CCC2(F)F)C1]
38 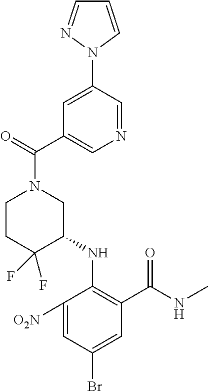 [SMILES: CNC(=O)c1cc(Br)cc([N+](=O)[O-])c1N[C@H]1CN(C(=O)c2cncc(-n3cccn3)c2)CCC1(F)F]
39 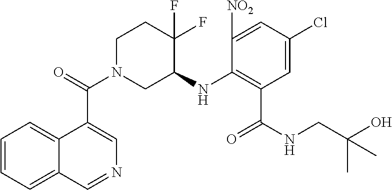 [SMILES: CC(C)(O)CNC(=O)c1cc(Cl)cc([N+](=O)[O-])c1N[C@H]1CN(C(=O)c2cncc3ccccc23)CCC1(F)F]
40 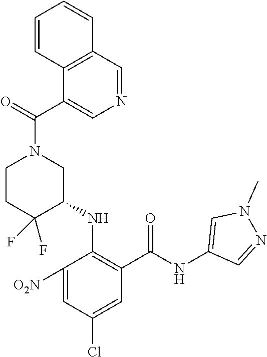 [SMILES: Cn1cc(NC(=O)c2cc(Cl)cc([N+](=O)[O-])c2N[C@H]2CN(C(=O)c3cncc4ccccc34)CCC2(F)F)cn1]
41 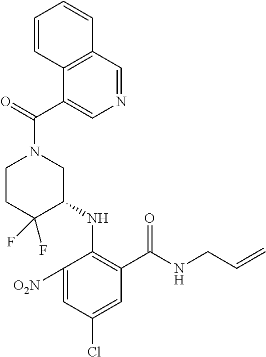 [SMILES: C=CCNC(=O)c1cc(Cl)cc([N+](=O)[O-])c1N[C@H]1CN(C(=O)c2cncc3ccccc23)CCC1(F)F]
42 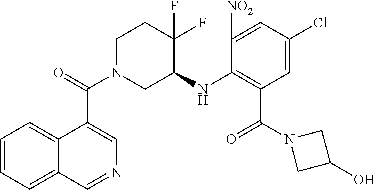 [SMILES: O=C(c1cc(Cl)cc([N+](=O)[O-])c1N[C@H]1CN(C(=O)c2cncc3ccccc23)CCC1(F)F)N1CC(O)C1]
43 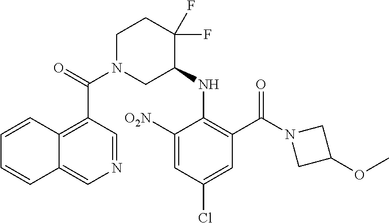 [SMILES: COC1CN(C(=O)c2cc(Cl)cc([N+](=O)[O-])c2N[C@H]2CN(C(=O)c3cncc4ccccc34)CCC2(F)F)C1]
44 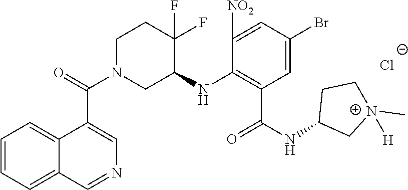 [SMILES: C[NH+]1CC[C@@H](NC(=O)c2cc(Br)cc([N+](=O)[O-])c2N[C@H]2CN(C(=O)c3cncc4ccccc34)CCC2(F)F)C1 ; [Cl-]]
45 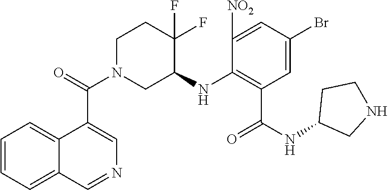 [SMILES: O=C(N[C@@H]1CCNC1)c1cc(Br)cc([N+](=O)[O-])c1N[C@H]1CN(C(=O)c2cncc3ccccc23)CCC1(F)F]
46 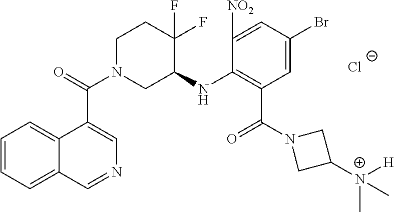 [SMILES: C[NH+](C)C1CN(C(=O)c2cc(Br)cc([N+](=O)[O-])c2N[C@H]2CN(C(=O)c3cncc4ccccc34)CCC2(F)F)C1 ; [Cl-]]
47 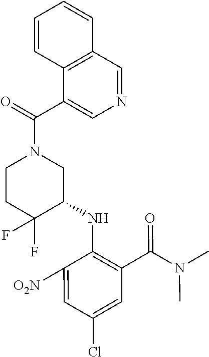 [SMILES: CN(C)C(=O)c1cc(Cl)cc([N+](=O)[O-])c1N[C@H]1CN(C(=O)c2cncc3ccccc23)CCC1(F)F]
48 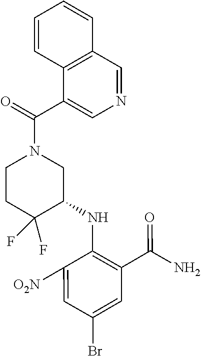 [SMILES: NC(=O)c1cc(Br)cc([N+](=O)[O-])c1N[C@H]1CN(C(=O)c2cncc3ccccc23)CCC1(F)F]
49 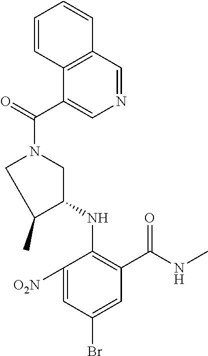 [SMILES: CNC(=O)c1cc(Br)cc([N+](=O)[O-])c1N[C@H]1CN(C(=O)c2cncc3ccccc23)C[C@@H]1C]
50 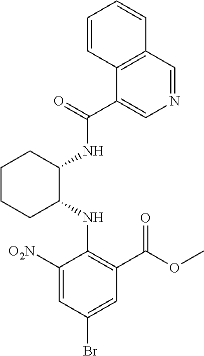 [SMILES: COC(=O)c1cc(Br)cc([N+](=O)[O-])c1N[C@@H]1CCCC[C@@H]1NC(=O)c1cncc2ccccc12]
51 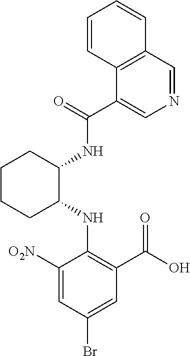 [SMILES: O=C(O)c1cc(Br)cc([N+](=O)[O-])c1N[C@@H]1CCCC[C@@H]1NC(=O)c1cncc2ccccc12]
52 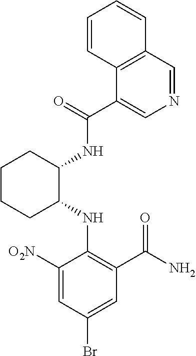 [SMILES: NC(=O)c1cc(Br)cc([N+](=O)[O-])c1N[C@@H]1CCCC[C@@H]1NC(=O)c1cncc2ccccc12]
53 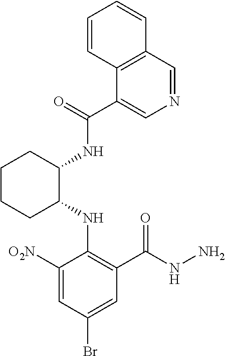 [SMILES: NNC(=O)c1cc(Br)cc([N+](=O)[O-])c1N[C@@H]1CCCC[C@@H]1NC(=O)c1cncc2ccccc12]
54 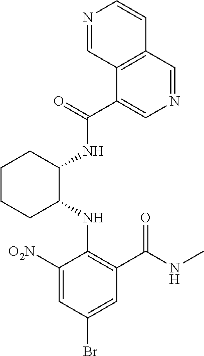 [SMILES: CNC(=O)c1cc(Br)cc([N+](=O)[O-])c1N[C@@H]1CCCC[C@@H]1NC(=O)c1cncc2ccncc12]
55 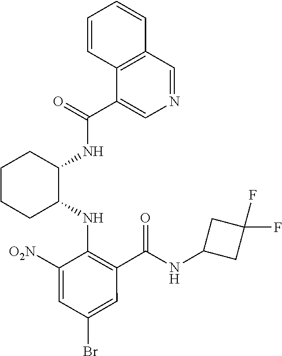 [SMILES: O=C(NC1CC(F)(F)C1)c1cc(Br)cc([N+](=O)[O-])c1N[C@@H]1CCCC[C@@H]1NC(=O)c1cncc2ccccc12]
56 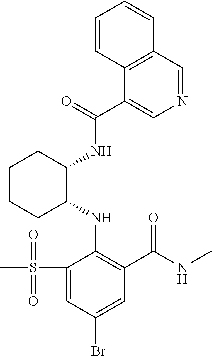
57 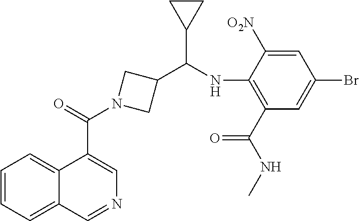 [SMILES: CNC(=O)c1cc(Br)cc([N+](=O)[O-])c1NC(C1CC1)C1CN(C(=O)c2cncc3ccccc23)C1]
58 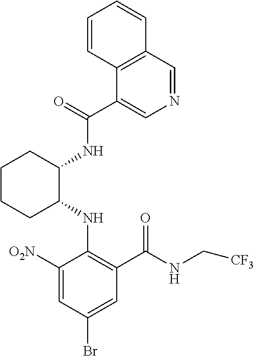 [SMILES: O=C(NCC(F)(F)F)c1cc(Br)cc([N+](=O)[O-])c1N[C@@H]1CCCC[C@@H]1NC(=O)c1cncc2ccccc12]
59 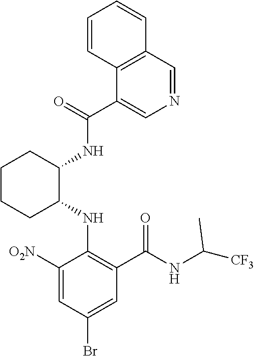 [SMILES: CC(NC(=O)c1cc(Br)cc([N+](=O)[O-])c1N[C@@H]1CCCC[C@@H]1NC(=O)c1cncc2ccccc12)C(F)(F)F]
60 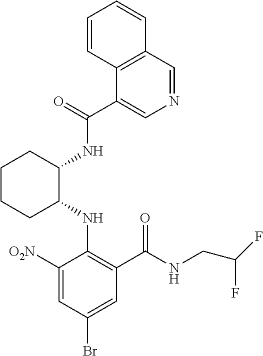 [SMILES: O=C(NCC(F)F)c1cc(Br)cc([N+](=O)[O-])c1N[C@@H]1CCCC[C@@H]1NC(=O)c1cncc2ccccc12]
61 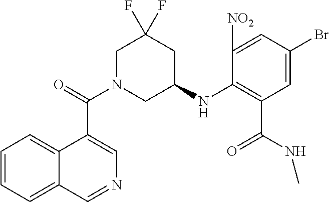 [SMILES: CNC(=O)c1cc(Br)cc([N+](=O)[O-])c1N[C@H]1CN(C(=O)c2cncc3ccccc23)CC(F)(F)C1]
62 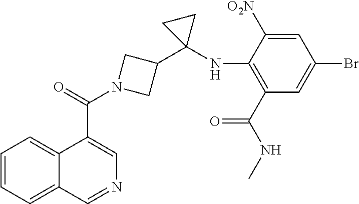 [SMILES: CNC(=O)c1cc(Br)cc([N+](=O)[O-])c1NC1(C2CN(C(=O)c3cncc4ccccc34)C2)CC1]
63 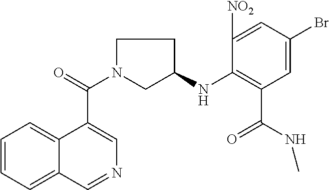 [SMILES: CNC(=O)c1cc(Br)cc([N+](=O)[O-])c1N[C@@H]1CCN(C(=O)c2cncc3ccccc23)C1]
64 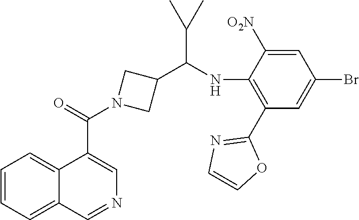 [SMILES: CC(C)C(Nc1c(-c2ncco2)cc(Br)cc1[N+](=O)[O-])C1CN(C(=O)c2cncc3ccccc23)C1]
65 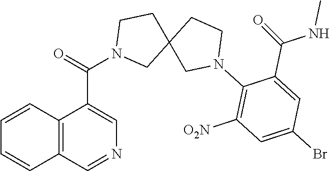 [SMILES: CNC(=O)c1cc(Br)cc([N+](=O)[O-])c1N1CCC2(CCN(C(=O)c3cncc4ccccc34)C2)C1]
66 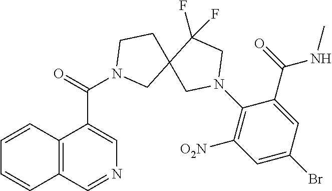 [SMILES: CNC(=O)c1cc(Br)cc([N+](=O)[O-])c1N1CC(F)(F)C2(CCN(C(=O)c3cncc4ccccc34)C2)C1]
67 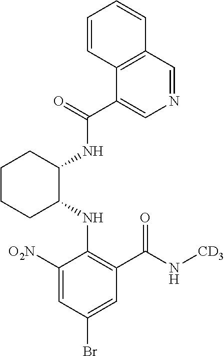 [SMILES: [2H]C([2H])([2H])NC(=O)c1cc(Br)cc([N+](=O)[O-])c1N[C@@H]1CCCC[C@@H]1NC(=O)c1cncc2ccccc12]
68 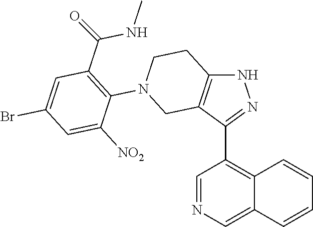 [SMILES: CNC(=O)c1cc(Br)cc([N+](=O)[O-])c1N1CCc2[nH]nc(-c3cncc4ccccc34)c2C1]
69 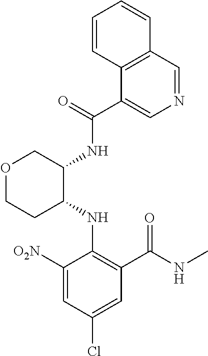 [SMILES: CNC(=O)c1cc(Cl)cc([N+](=O)[O-])c1N[C@@H]1CCOC[C@@H]1NC(=O)c1cncc2ccccc12]
70 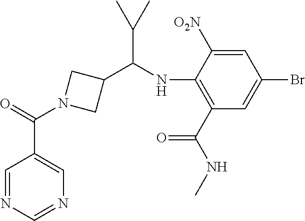 [SMILES: CNC(=O)c1cc(Br)cc([N+](=O)[O-])c1NC(C(C)C)C1CN(C(=O)c2cncnc2)C1]
71 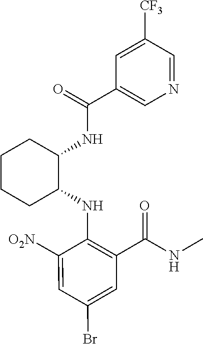 [SMILES: CNC(=O)c1cc(Br)cc([N+](=O)[O-])c1N[C@@H]1CCCC[C@@H]1NC(=O)c1cncc(C(F)(F)F)c1]
72 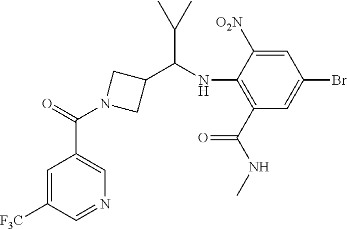 [SMILES: CNC(=O)c1cc(Br)cc([N+](=O)[O-])c1NC(C(C)C)C1CN(C(=O)c2cncc(C(F)(F)F)c2)C1]
73 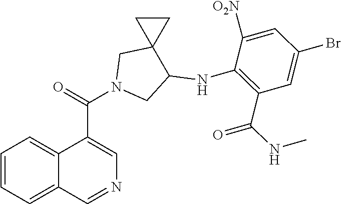 [SMILES: CNC(=O)c1cc(Br)cc([N+](=O)[O-])c1NC1CN(C(=O)c2cncc3ccccc23)CC12CC2]
74 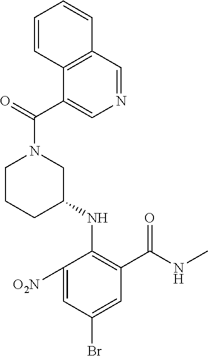 [SMILES: CNC(=O)c1cc(Br)cc([N+](=O)[O-])c1N[C@@H]1CCCN(C(=O)c2cncc3ccccc23)C1]
75 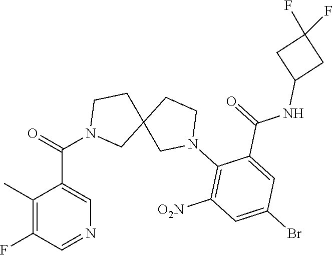 [SMILES: Cc1c(F)cncc1C(=O)N1CCC2(CCN(c3c(C(=O)NC4CC(F)(F)C4)cc(Br)cc3[N+](=O)[O-])C2)C1]
76 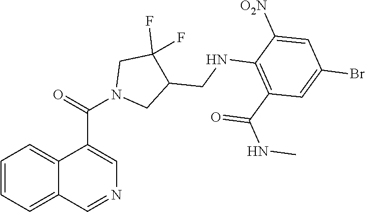 [SMILES: CNC(=O)c1cc(Br)cc([N+](=O)[O-])c1NCC1CN(C(=O)c2cncc3ccccc23)CC1(F)F]
77 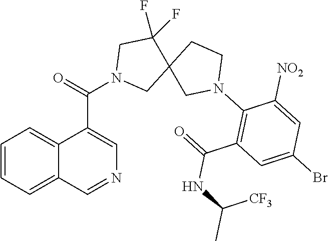 [SMILES: C[C@@H](NC(=O)c1cc(Br)cc([N+](=O)[O-])c1N1CCC2(CN(C(=O)c3cncc4ccccc34)CC2(F)F)C1)C(F)(F)F]
78 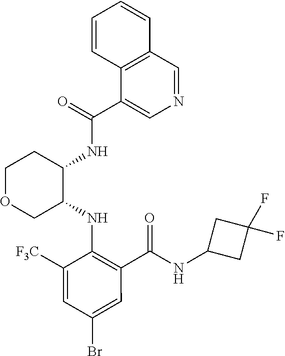 [SMILES: O=C(NC1CC(F)(F)C1)c1cc(Br)cc(C(F)(F)F)c1N[C@@H]1COCC[C@@H]1NC(=O)c1cncc2ccccc12]
79 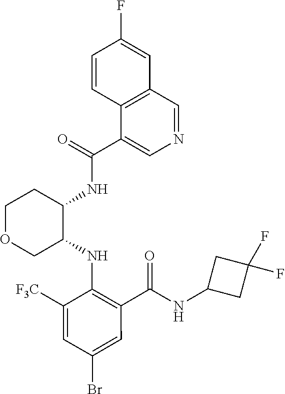 [SMILES: O=C(NC1CC(F)(F)C1)c1cc(Br)cc(C(F)(F)F)c1N[C@@H]1COCC[C@@H]1NC(=O)c1cncc2cc(F)ccc12]
80 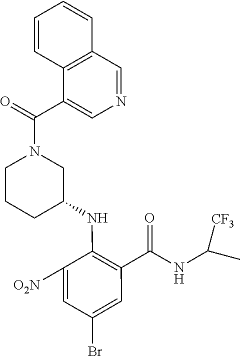 [SMILES: CC(NC(=O)c1cc(Br)cc([N+](=O)[O-])c1N[C@@H]1CCCN(C(=O)c2cncc3ccccc23)C1)C(F)(F)F]
81 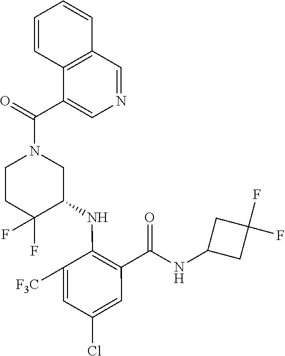 [SMILES: O=C(NC1CC(F)(F)C1)c1cc(Cl)cc(C(F)(F)F)c1N[C@H]1CN(C(=O)c2cncc3ccccc23)CCC1(F)F]
82 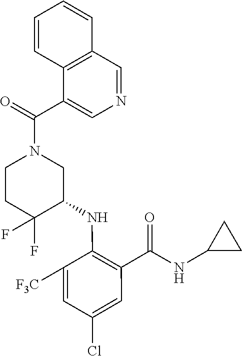 [SMILES: O=C(NC1CC1)c1cc(Cl)cc(C(F)(F)F)c1N[C@H]1CN(C(=O)c2cncc3ccccc23)CCC1(F)F]
83 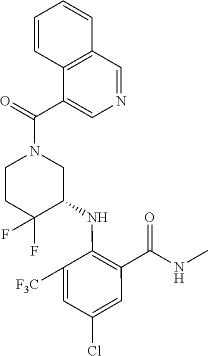 [SMILES: CNC(=O)c1cc(Cl)cc(C(F)(F)F)c1N[C@H]1CN(C(=O)c2cncc3ccccc23)CCC1(F)F]
84 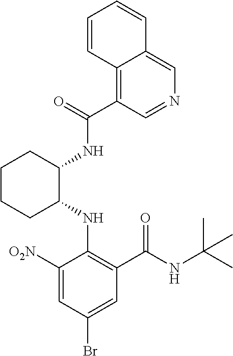 [SMILES: CC(C)(C)NC(=O)c1cc(Br)cc([N+](=O)[O-])c1N[C@@H]1CCCC[C@@H]1NC(=O)c1cncc2ccccc12]
85 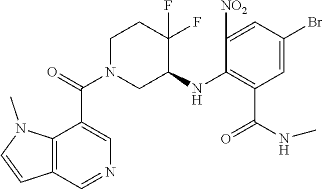 [SMILES: CNC(=O)c1cc(Br)cc([N+](=O)[O-])c1N[C@H]1CN(C(=O)c2cncc3ccn(C)c23)CCC1(F)F]
86 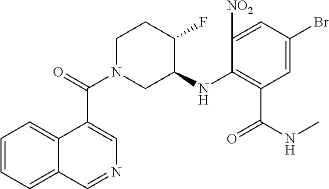 [SMILES: CNC(=O)c1cc(Br)cc([N+](=O)[O-])c1N[C@H]1CN(C(=O)c2cncc3ccccc23)CC[C@@H]1F]
87 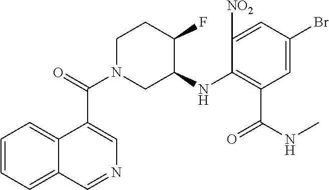 [SMILES: CNC(=O)c1cc(Br)cc([N+](=O)[O-])c1N[C@H]1CN(C(=O)c2cncc3ccccc23)CC[C@H]1F]
88 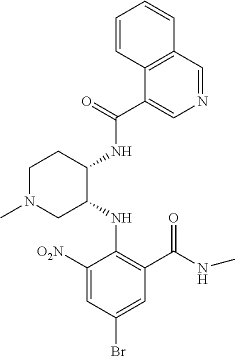
89 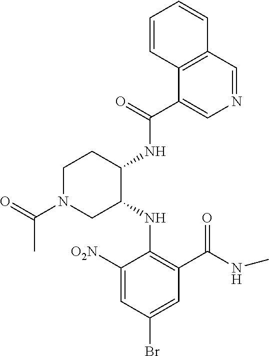 [SMILES: CNC(=O)c1cc(Br)cc([N+](=O)[O-])c1N[C@@H]1CN(C(C)=O)CC[C@@H]1NC(=O)c1cncc2ccccc12]
90 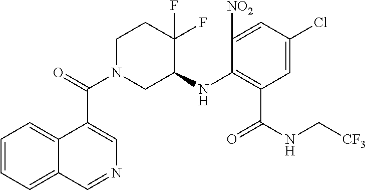 [SMILES: O=C(NCC(F)(F)F)c1cc(Cl)cc([N+](=O)[O-])c1N[C@H]1CN(C(=O)c2cncc3ccccc23)CCC1(F)F]
91 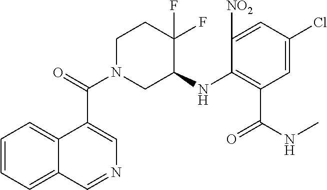 [SMILES: CNC(=O)c1cc(Cl)cc([N+](=O)[O-])c1N[C@H]1CN(C(=O)c2cncc3ccccc23)CCC1(F)F]
92 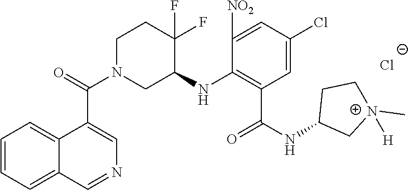 [SMILES: C[NH+]1CC[C@@H](NC(=O)c2cc(Cl)cc([N+](=O)[O-])c2N[C@H]2CN(C(=O)c3cncc4ccccc34)CCC2(F)F)C1 ; [Cl-]]
93 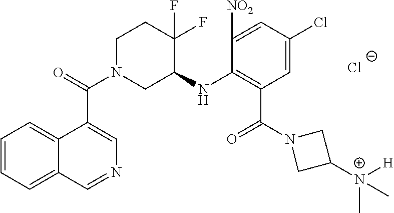 [SMILES: C[NH+](C)C1CN(C(=O)c2cc(Cl)cc([N+](=O)[O-])c2N[C@H]2CN(C(=O)c3cncc4ccccc34)CCC2(F)F)C1 ; [Cl-]]
94 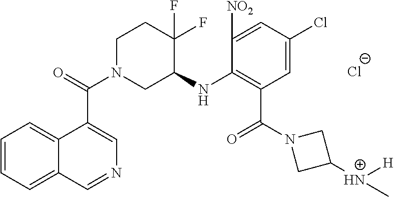 [SMILES: C[NH2+]C1CN(C(=O)c2cc(Cl)cc([N+](=O)[O-])c2N[C@H]2CN(C(=O)c3cncc4ccccc34)CCC2(F)F)C1 ; [Cl-]]
95 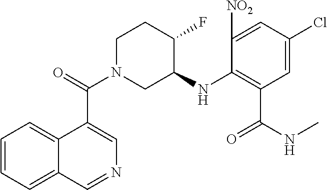 [SMILES: CNC(=O)c1cc(Cl)cc([N+](=O)[O-])c1N[C@H]1CN(C(=O)c2cncc3ccccc23)CC[C@@H]1F]
96 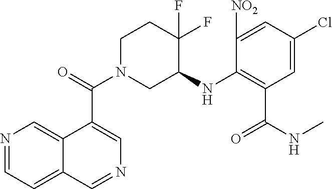 [SMILES: CNC(=O)c1cc(Cl)cc([N+](=O)[O-])c1N[C@H]1CN(C(=O)c2cncc3ccncc23)CCC1(F)F]
97 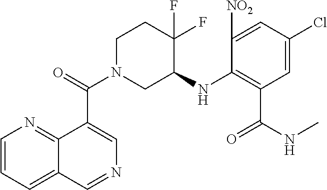 [SMILES: CNC(=O)c1cc(Cl)cc([N+](=O)[O-])c1N[C@H]1CN(C(=O)c2cncc3cccnc23)CCC1(F)F]
98 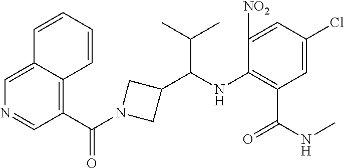 [SMILES: CNC(=O)c1cc(Cl)cc([N+](=O)[O-])c1NC(C(C)C)C1CN(C(=O)c2cncc3ccccc23)C1]
99 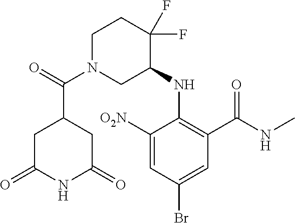 [SMILES: CNC(=O)c1cc(Br)cc([N+](=O)[O-])c1N[C@H]1CN(C(=O)C2CC(=O)NC(=O)C2)CCC1(F)F]
100 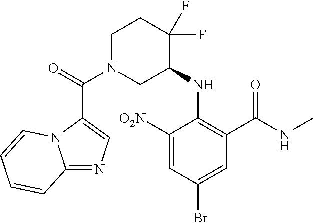 [SMILES: CNC(=O)c1cc(Br)cc([N+](=O)[O-])c1N[C@H]1CN(C(=O)c2cnc3ccccn23)CCC1(F)F]
101 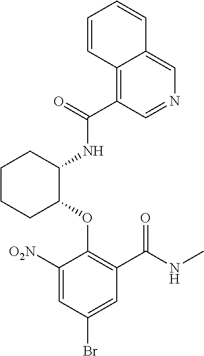 [SMILES: CNC(=O)c1cc(Br)cc([N+](=O)[O-])c1O[C@@H]1CCCC[C@@H]1NC(=O)c1cncc2ccccc12]
102 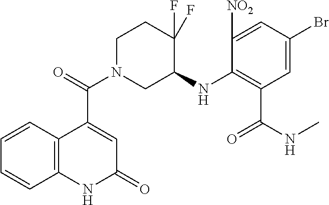 [SMILES: CNC(=O)c1cc(Br)cc([N+](=O)[O-])c1N[C@H]1CN(C(=O)c2cc(=O)[nH]c3ccccc23)CCC1(F)F]
103 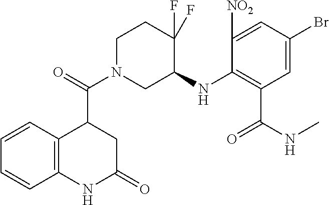 [SMILES: CNC(=O)c1cc(Br)cc([N+](=O)[O-])c1N[C@H]1CN(C(=O)C2CC(=O)Nc3ccccc32)CCC1(F)F]
104 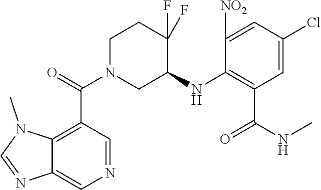 [SMILES: CNC(=O)c1cc(Cl)cc([N+](=O)[O-])c1N[C@H]1CN(C(=O)c2cncc3ncn(C)c23)CCC1(F)F]
105 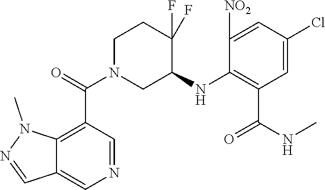 [SMILES: CNC(=O)c1cc(Cl)cc([N+](=O)[O-])c1N[C@H]1CN(C(=O)c2cncc3cnn(C)c23)CCC1(F)F]
106 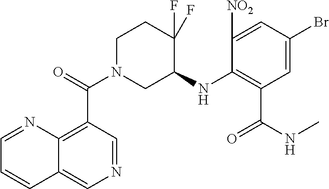 [SMILES: CNC(=O)c1cc(Br)cc([N+](=O)[O-])c1N[C@H]1CN(C(=O)c2cncc3cccnc23)CCC1(F)F]
107 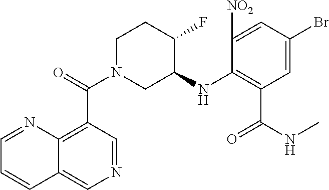 [SMILES: CNC(=O)c1cc(Br)cc([N+](=O)[O-])c1N[C@H]1CN(C(=O)c2cncc3cccnc23)CC[C@@H]1F]
108 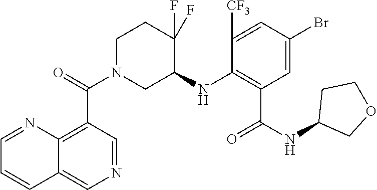 [SMILES: O=C(N[C@H]1CCOC1)c1cc(Br)cc(C(F)(F)F)c1N[C@H]1CN(C(=O)c2cncc3cccnc23)CCC1(F)F]
109 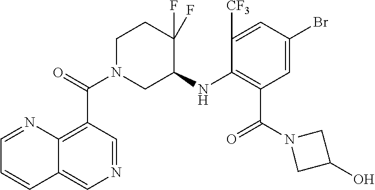 [SMILES: O=C(c1cc(Br)cc(C(F)(F)F)c1N[C@H]1CN(C(=O)c2cncc3cccnc23)CCC1(F)F)N1CC(O)C1]
110 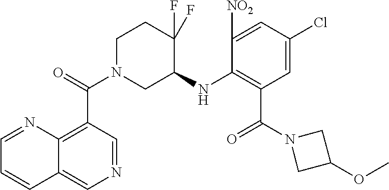 [SMILES: COC1CN(C(=O)c2cc(Cl)cc([N+](=O)[O-])c2N[C@H]2CN(C(=O)c3cncc4cccnc34)CCC2(F)F)C1]
111 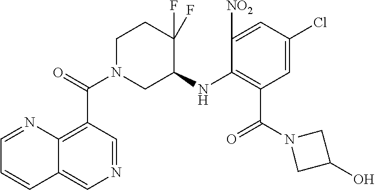 [SMILES: O=C(c1cc(Cl)cc([N+](=O)[O-])c1N[C@H]1CN(C(=O)c2cncc3cccnc23)CCC1(F)F)N1CC(O)C1]
112 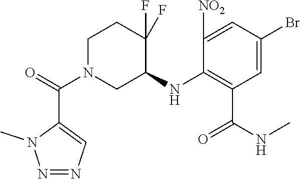
113 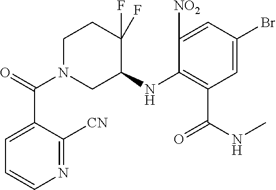 [SMILES: CNC(=O)c1cc(Br)cc([N+](=O)[O-])c1N[C@H]1CN(C(=O)c2cccnc2C#N)CCC1(F)F]
114 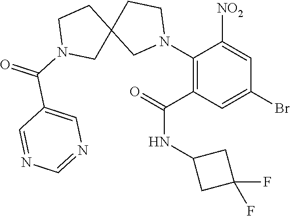 [SMILES: O=C(NC1CC(F)(F)C1)c1cc(Br)cc([N+](=O)[O-])c1N1CCC2(CCN(C(=O)c3cncnc3)C2)C1]
115 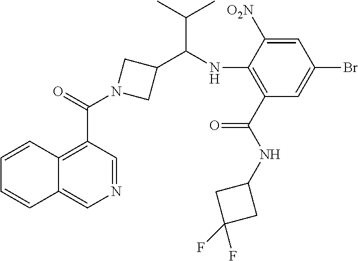 [SMILES: CC(C)C(Nc1c(C(=O)NC2CC(F)(F)C2)cc(Br)cc1[N+](=O)[O-])C1CN(C(=O)c2cncc3ccccc23)C1]
116 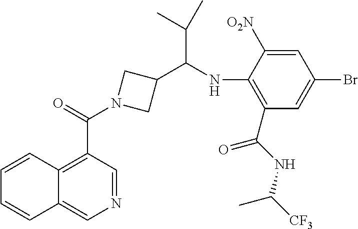
117 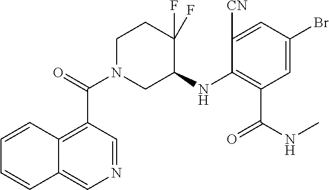 [SMILES: CNC(=O)c1cc(Br)cc(C#N)c1N[C@H]1CN(C(=O)c2cncc3ccccc23)CCC1(F)F]
118 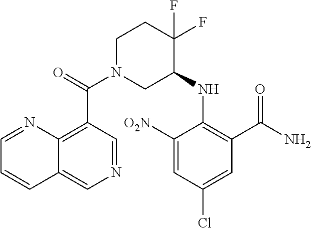 [SMILES: NC(=O)c1cc(Cl)cc([N+](=O)[O-])c1N[C@H]1CN(C(=O)c2cncc3cccnc23)CCC1(F)F]
119 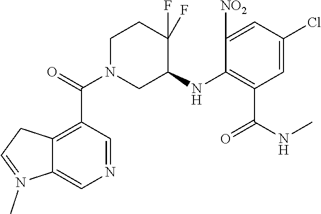
120 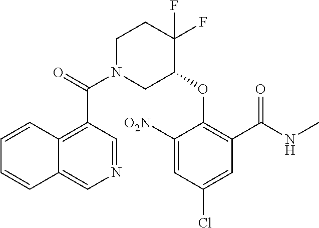 [SMILES: CNC(=O)c1cc(Cl)cc([N+](=O)[O-])c1O[C@@H]1CN(C(=O)c2cncc3ccccc23)CCC1(F)F]
121 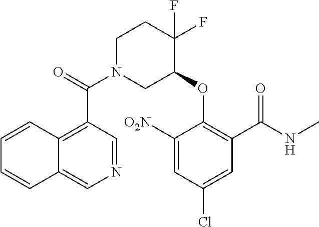 [SMILES: CNC(=O)c1cc(Cl)cc([N+](=O)[O-])c1O[C@H]1CN(C(=O)c2cncc3ccccc23)CCC1(F)F]
122 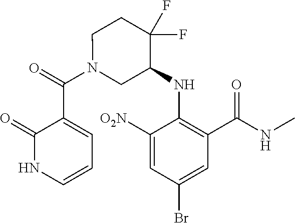 [SMILES: CNC(=O)c1cc(Br)cc([N+](=O)[O-])c1N[C@H]1CN(C(=O)c2ccc[nH]c2=O)CCC1(F)F]
123 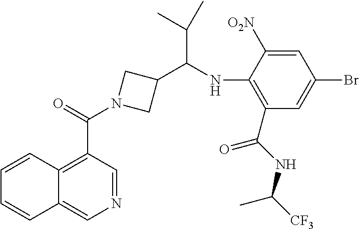 [SMILES: CC(C)C(Nc1c(C(=O)N[C@H](C)C(F)(F)F)cc(Br)cc1[N+](=O)[O-])C1CN(C(=O)c2cncc3ccccc23)C1]
124 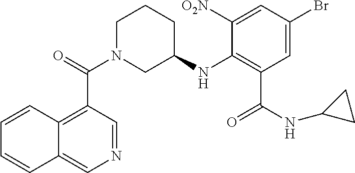 [SMILES: O=C(NC1CC1)c1cc(Br)cc([N+](=O)[O-])c1N[C@@H]1CCCN(C(=O)c2cncc3ccccc23)C1]
125 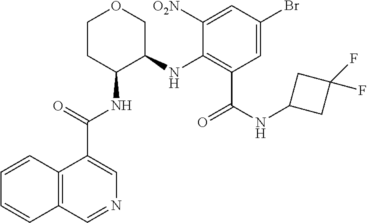 [SMILES: O=C(NC1CC(F)(F)C1)c1cc(Br)cc([N+](=O)[O-])c1N[C@@H]1COCC[C@@H]1NC(=O)c1cncc2ccccc12]
126 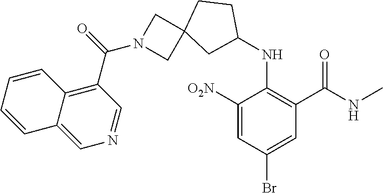 [SMILES: CNC(=O)c1cc(Br)cc([N+](=O)[O-])c1NC1CCC2(C1)CN(C(=O)c1cncc3ccccc13)C2]
127 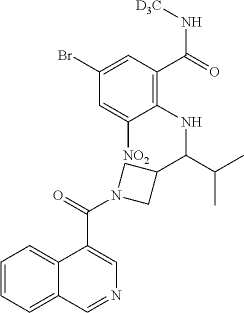 [SMILES: [2H]C([2H])([2H])NC(=O)c1cc(Br)cc([N+](=O)[O-])c1NC(C(C)C)C1CN(C(=O)c2cncc3ccccc23)C1]
128 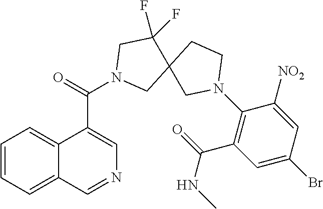 [SMILES: CNC(=O)c1cc(Br)cc([N+](=O)[O-])c1N1CCC2(CN(C(=O)c3cncc4ccccc34)CC2(F)F)C1]
129 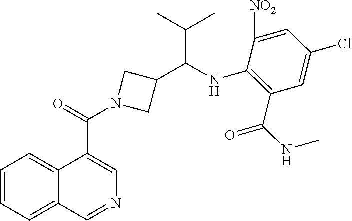 [SMILES: CNC(=O)c1cc(Cl)cc([N+](=O)[O-])c1NC(C(C)C)C1CN(C(=O)c2cncc3ccccc23)C1]
130 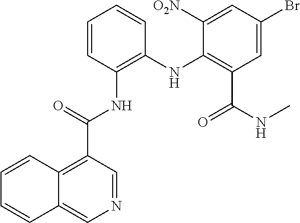 [SMILES: CNC(=O)c1cc(Br)cc([N+](=O)[O-])c1Nc1ccccc1NC(=O)c1cncc2ccccc12]
131 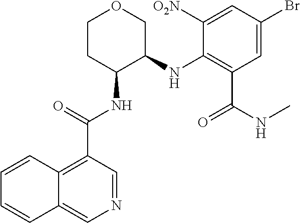 [SMILES: CNC(=O)c1cc(Br)cc([N+](=O)[O-])c1N[C@@H]1COCC[C@@H]1NC(=O)c1cncc2ccccc12]
132 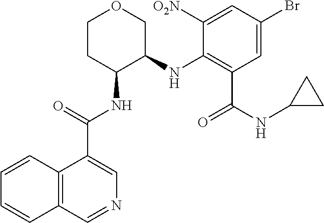 [SMILES: O=C(NC1CC1)c1cc(Br)cc([N+](=O)[O-])c1N[C@@H]1COCC[C@@H]1NC(=O)c1cncc2ccccc12]
133 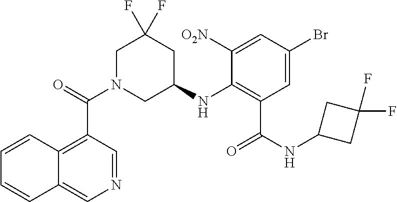 [SMILES: O=C(NC1CC(F)(F)C1)c1cc(Br)cc([N+](=O)[O-])c1N[C@H]1CN(C(=O)c2cncc3ccccc23)CC(F)(F)C1]
134 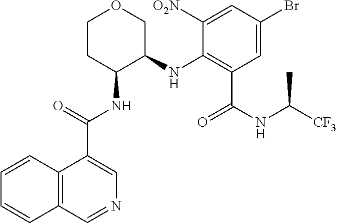 [SMILES: C[C@H](NC(=O)c1cc(Br)cc([N+](=O)[O-])c1N[C@@H]1COCC[C@@H]1NC(=O)c1cncc2ccccc12)C(F)(F)F]
135 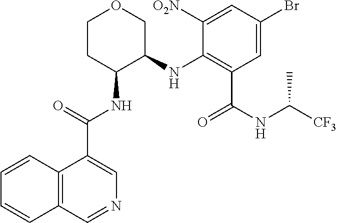 [SMILES: C[C@@H](NC(=O)c1cc(Br)cc([N+](=O)[O-])c1N[C@@H]1COCC[C@@H]1NC(=O)c1cncc2ccccc12)C(F)(F)F]
136 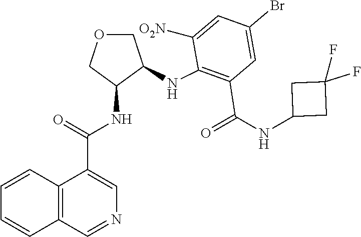 [SMILES: O=C(NC1CC(F)(F)C1)c1cc(Br)cc([N+](=O)[O-])c1N[C@@H]1COC[C@@H]1NC(=O)c1cncc2ccccc12]
137 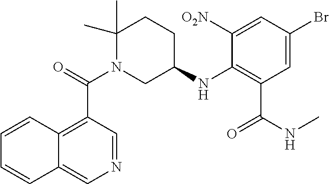 [SMILES: CNC(=O)c1cc(Br)cc([N+](=O)[O-])c1N[C@@H]1CCC(C)(C)N(C(=O)c2cncc3ccccc23)C1]
138 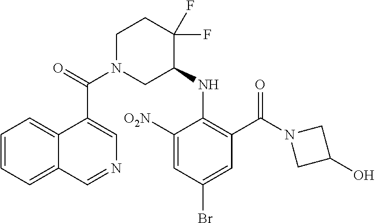 [SMILES: O=C(c1cc(Br)cc([N+](=O)[O-])c1N[C@H]1CN(C(=O)c2cncc3ccccc23)CCC1(F)F)N1CC(O)C1]
139 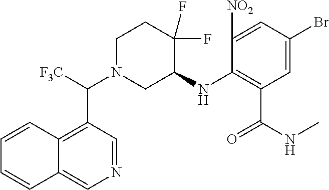 [SMILES: CNC(=O)c1cc(Br)cc([N+](=O)[O-])c1N[C@H]1CN(C(c2cncc3ccccc23)C(F)(F)F)CCC1(F)F]
140 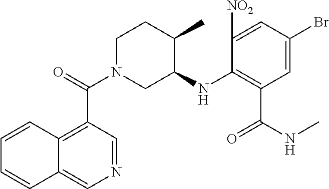 [SMILES: CNC(=O)c1cc(Br)cc([N+](=O)[O-])c1N[C@H]1CN(C(=O)c2cncc3ccccc23)CC[C@H]1C]
141 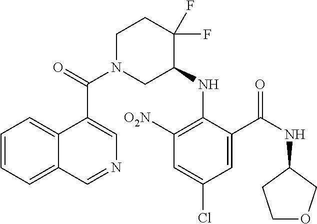 [SMILES: O=C(N[C@@H]1CCOC1)c1cc(Cl)cc([N+](=O)[O-])c1N[C@H]1CN(C(=O)c2cncc3ccccc23)CCC1(F)F]
142 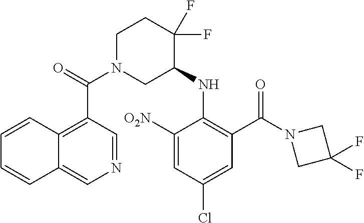 [SMILES: O=C(c1cc(Cl)cc([N+](=O)[O-])c1N[C@H]1CN(C(=O)c2cncc3ccccc23)CCC1(F)F)N1CC(F)(F)C1]
143 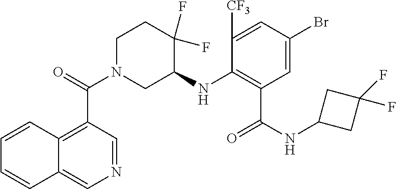 [SMILES: O=C(NC1CC(F)(F)C1)c1cc(Br)cc(C(F)(F)F)c1N[C@H]1CN(C(=O)c2cncc3ccccc23)CCC1(F)F]
144 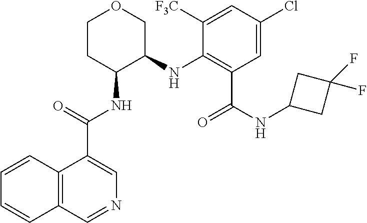 [SMILES: O=C(NC1CC(F)(F)C1)c1cc(Cl)cc(C(F)(F)F)c1N[C@@H]1COCC[C@@H]1NC(=O)c1cncc2ccccc12]
145 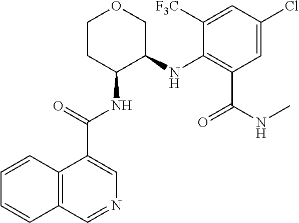 [SMILES: CNC(=O)c1cc(Cl)cc(C(F)(F)F)c1N[C@@H]1COCC[C@@H]1NC(=O)c1cncc2ccccc12]
146 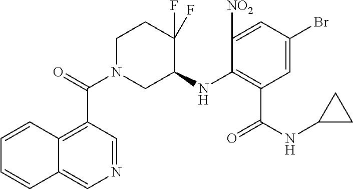 [SMILES: O=C(NC1CC1)c1cc(Br)cc([N+](=O)[O-])c1N[C@H]1CN(C(=O)c2cncc3ccccc23)CCC1(F)F]
147 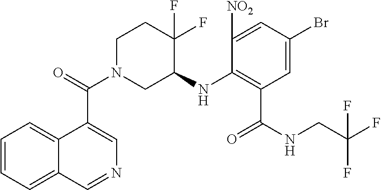 [SMILES: O=C(NCC(F)(F)F)c1cc(Br)cc([N+](=O)[O-])c1N[C@H]1CN(C(=O)c2cncc3ccccc23)CCC1(F)F]
148 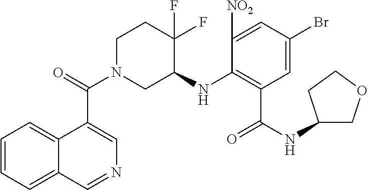 [SMILES: O=C(N[C@H]1CCOC1)c1cc(Br)cc([N+](=O)[O-])c1N[C@H]1CN(C(=O)c2cncc3ccccc23)CCC1(F)F]
149 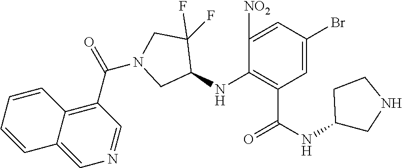 [SMILES: O=C(N[C@@H]1CCNC1)c1cc(Br)cc([N+](=O)[O-])c1N[C@H]1CN(C(=O)c2cncc3ccccc23)CC1(F)F]
150 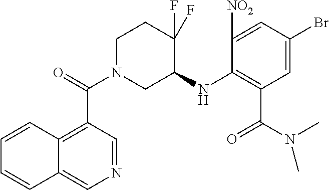 [SMILES: CN(C)C(=O)c1cc(Br)cc([N+](=O)[O-])c1N[C@H]1CN(C(=O)c2cncc3ccccc23)CCC1(F)F]
151 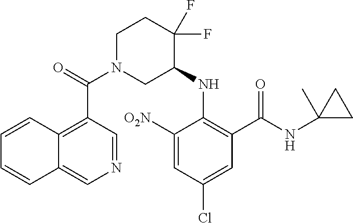 [SMILES: CC1(NC(=O)c2cc(Cl)cc([N+](=O)[O-])c2N[C@H]2CN(C(=O)c3cncc4ccccc34)CCC2(F)F)CC1]
152 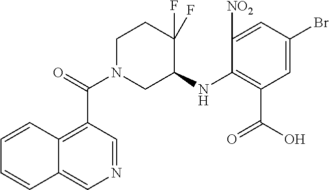 [SMILES: O=C(O)c1cc(Br)cc([N+](=O)[O-])c1N[C@H]1CN(C(=O)c2cncc3ccccc23)CCC1(F)F]
153 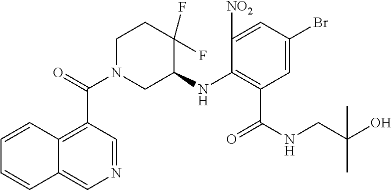 [SMILES: CC(C)(O)CNC(=O)c1cc(Br)cc([N+](=O)[O-])c1N[C@H]1CN(C(=O)c2cncc3ccccc23)CCC1(F)F]
154 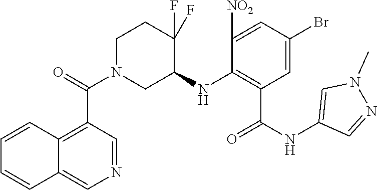 [SMILES: Cn1cc(NC(=O)c2cc(Br)cc([N+](=O)[O-])c2N[C@H]2CN(C(=O)c3cncc4ccccc34)CCC2(F)F)cn1]
155 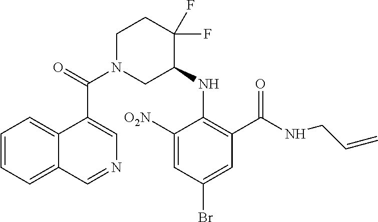 [SMILES: C=CCNC(=O)c1cc(Br)cc([N+](=O)[O-])c1N[C@H]1CN(C(=O)c2cncc3ccccc23)CCC1(F)F]
156 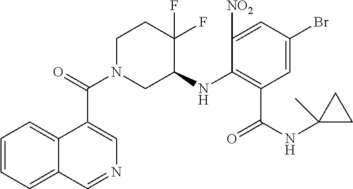 [SMILES: CC1(NC(=O)c2cc(Br)cc([N+](=O)[O-])c2N[C@H]2CN(C(=O)c3cncc4ccccc34)CCC2(F)F)CC1]
157 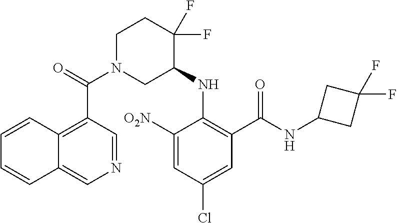 [SMILES: O=C(NC1CC(F)(F)C1)c1cc(Cl)cc([N+](=O)[O-])c1N[C@H]1CN(C(=O)c2cncc3ccccc23)CCC1(F)F]
158 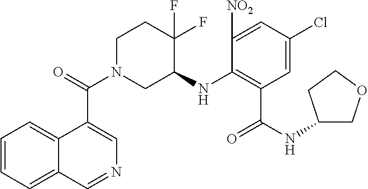 [SMILES: O=C(N[C@@H]1CCOC1)c1cc(Cl)cc([N+](=O)[O-])c1N[C@H]1CN(C(=O)c2cncc3ccccc23)CCC1(F)F]
159 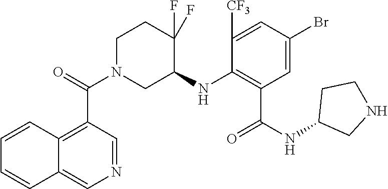 [SMILES: O=C(N[C@@H]1CCNC1)c1cc(Br)cc(C(F)(F)F)c1N[C@H]1CN(C(=O)c2cncc3ccccc23)CCC1(F)F]
160 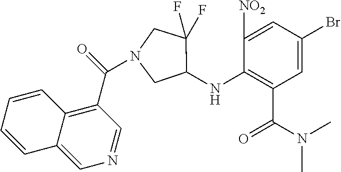 [SMILES: CN(C)C(=O)c1cc(Br)cc([N+](=O)[O-])c1NC1CN(C(=O)c2cncc3ccccc23)CC1(F)F]
161 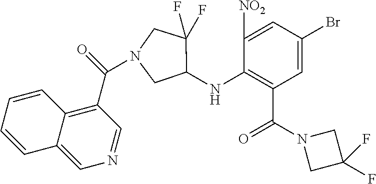 [SMILES: O=C(c1cc(Br)cc([N+](=O)[O-])c1NC1CN(C(=O)c2cncc3ccccc23)CC1(F)F)N1CC(F)(F)C1]
162 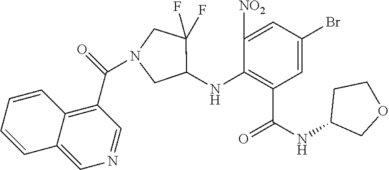 [SMILES: O=C(N[C@@H]1CCOC1)c1cc(Br)cc([N+](=O)[O-])c1NC1CN(C(=O)c2cncc3ccccc23)CC1(F)F]
163 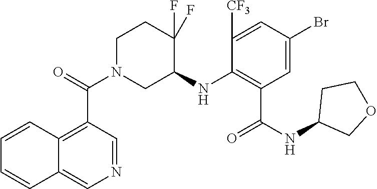 [SMILES: O=C(N[C@H]1CCOC1)c1cc(Br)cc(C(F)(F)F)c1N[C@H]1CN(C(=O)c2cncc3ccccc23)CCC1(F)F]
164 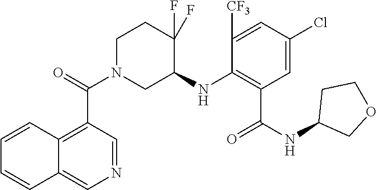 [SMILES: O=C(N[C@H]1CCOC1)c1cc(Cl)cc(C(F)(F)F)c1N[C@H]1CN(C(=O)c2cncc3ccccc23)CCC1(F)F]
165 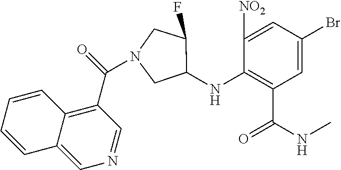 [SMILES: CNC(=O)c1cc(Br)cc([N+](=O)[O-])c1NC1CN(C(=O)c2cncc3ccccc23)C[C@H]1F]
166 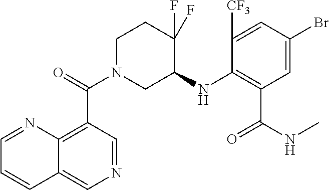 [SMILES: CNC(=O)c1cc(Br)cc(C(F)(F)F)c1N[C@H]1CN(C(=O)c2cncc3cccnc23)CCC1(F)F]
167 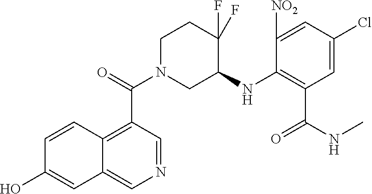 [SMILES: CNC(=O)c1cc(Cl)cc([N+](=O)[O-])c1N[C@H]1CN(C(=O)c2cncc3cc(O)ccc23)CCC1(F)F]
168 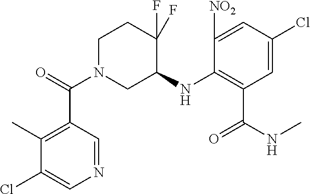
169 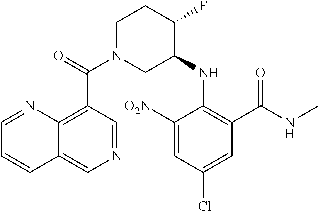 [SMILES: CNC(=O)c1cc(Cl)cc([N+](=O)[O-])c1N[C@H]1CN(C(=O)c2cncc3cccnc23)CC[C@@H]1F]
170 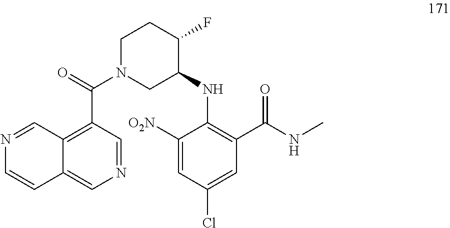 [SMILES: CNC(=O)c1cc(Cl)cc([N+](=O)[O-])c1N[C@H]1CN(C(=O)c2cncc3ccncc23)CC[C@@H]1F]
171 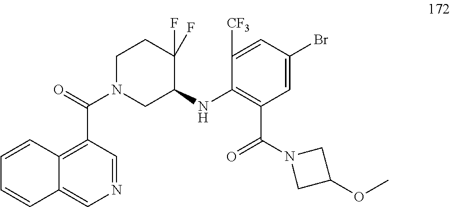 [SMILES: COC1CN(C(=O)c2cc(Br)cc(C(F)(F)F)c2N[C@H]2CN(C(=O)c3cncc4ccccc34)CCC2(F)F)C1]
172 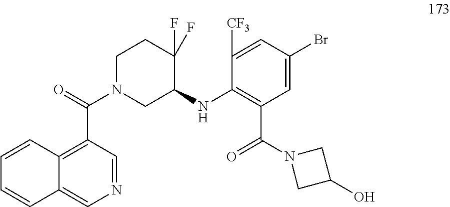 [SMILES: O=C(c1cc(Br)cc(C(F)(F)F)c1N[C@H]1CN(C(=O)c2cncc3ccccc23)CCC1(F)F)N1CC(O)C1]
173 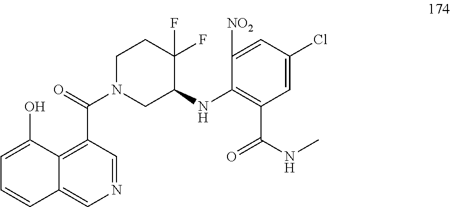 [SMILES: CNC(=O)c1cc(Cl)cc([N+](=O)[O-])c1N[C@H]1CN(C(=O)c2cncc3cccc(O)c23)CCC1(F)F]
174 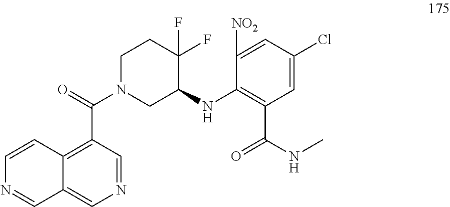 [SMILES: CNC(=O)c1cc(Cl)cc([N+](=O)[O-])c1N[C@H]1CN(C(=O)c2cncc3cnccc23)CCC1(F)F]
175 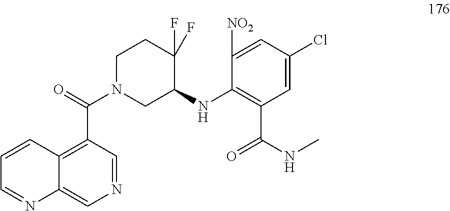 [SMILES: CNC(=O)c1cc(Cl)cc([N+](=O)[O-])c1N[C@H]1CN(C(=O)c2cncc3ncccc23)CCC1(F)F]
176 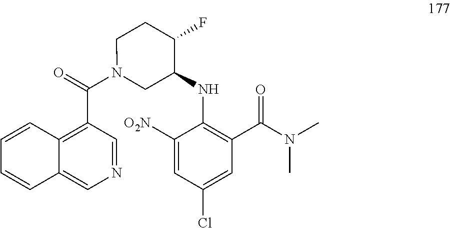 [SMILES: CN(C)C(=O)c1cc(Cl)cc([N+](=O)[O-])c1N[C@H]1CN(C(=O)c2cncc3ccccc23)CC[C@@H]1F]
177 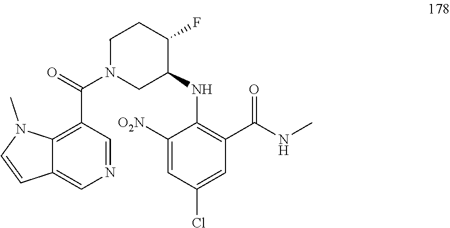 [SMILES: CNC(=O)c1cc(Cl)cc([N+](=O)[O-])c1N[C@H]1CN(C(=O)c2cncc3ccn(C)c23)CC[C@@H]1F]
178 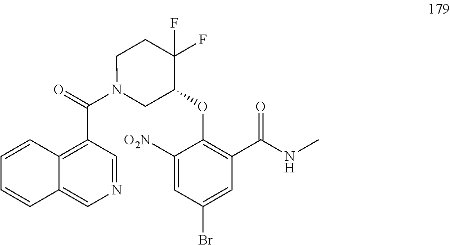 [SMILES: CNC(=O)c1cc(Br)cc([N+](=O)[O-])c1O[C@@H]1CN(C(=O)c2cncc3ccccc23)CCC1(F)F]
179 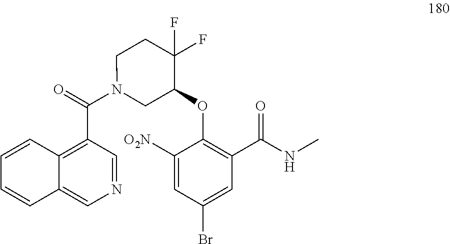 [SMILES: CNC(=O)c1cc(Br)cc([N+](=O)[O-])c1O[C@H]1CN(C(=O)c2cncc3ccccc23)CCC1(F)F]
180 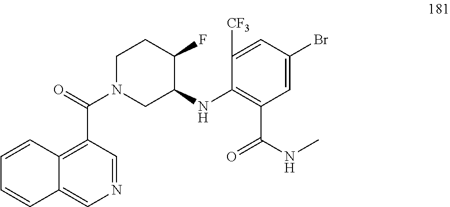 [SMILES: CNC(=O)c1cc(Br)cc(C(F)(F)F)c1N[C@H]1CN(C(=O)c2cncc3ccccc23)CC[C@H]1F]
181 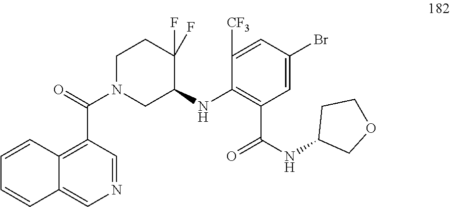 [SMILES: O=C(N[C@@H]1CCOC1)c1cc(Br)cc(C(F)(F)F)c1N[C@H]1CN(C(=O)c2cncc3ccccc23)CCC1(F)F]
182 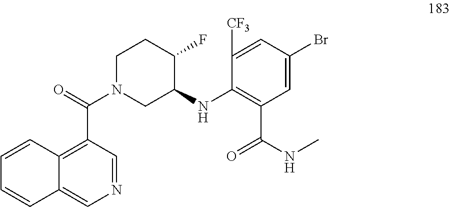 [SMILES: CNC(=O)c1cc(Br)cc(C(F)(F)F)c1N[C@H]1CN(C(=O)c2cncc3ccccc23)CC[C@@H]1F]
183

TABLE 2
Activities of Compounds 1-183 (inhibition of SARS-CoV-2 3CLpro
(IC₅₀) and inhibition of SARS-CoV-2 replication (EC₅₀) in mammalian cells)

| Com- | Inhibitory | Antiviral | Com- | Inhibitory | Antiviral | Com- | Inhibitory | Antiviral | Com- | Inhibitory | Antiviral |
|---|---|---|---|---|---|---|---|---|---|---|---|
| pound | Activity | Activity | pound | Activity | Activity | pound | Activity | Activity | pound | Activity | Activity |
| Number | (IC₅₀) | (EC₅₀) | Number | (IC₅₀) | (EC₅₀) | Number | (IC₅₀) | (EC₅₀) | Number | (IC₅₀) | (EC₅₀) |
| 1 | *** | ++ | 47 | **** | ++++ | 93 | **** | ++++ | 139 | **** | ++++ |
| 2 | * | | 48 | **** | ++++ | 94 | **** | ++++ | 140 | * | |
| 3 | ** | | 49 | **** | | 95 | **** | ++++ | 141 | *** | |
| 4 | ** | | 50 | **** | | 96 | **** | ++++ | 142 | **** | ++++ |
| 5 | * | | 51 | *** | | 97 | **** | | 143 | **** | ++++ |
| 6 | * | | 52 | ** | | 98 | **** | ++++ | 144 | **** | ++++ |
| 7 | ** | + | 53 | *** | ++ | 99 | *** | +++ | 145 | *** | +++ |
| 8 | **** | +++ | 54 | ** | | 100 | *** | | 146 | *** | +++ |
| 9 | **** | +++ | 55 | *** | +++ | 101 | **** | | 147 | **** | ++++ |
| 10 | * | | 56 | **** | | 102 | **** | | 148 | **** | |
| 11 | * | | 57 | * | | 103 | **** | | 149 | **** | ++++ |
| 12 | **** | ++++ | 58 | *** | | 104 | **** | | 150 | **** | |
| 13 | * | + | 59 | *** | +++ | 105 | **** | | 151 | **** | ++++ |
| 14 | *** | +++ | 60 | *** | +++ | 106 | **** | | 152 | **** | |
| 15 | * | | 61 | **** | ++++ | 107 | **** | | 153 | *** | |
| 16 | *** | ++ | 62 | *** | +++ | 108 | **** | | 154 | **** | ++++ |
| 17 | *** | | 63 | ** | | 109 | **** | | 155 | **** | ++++ |
| 18 | *** | +++ | 64 | *** | +++ | 110 | **** | | 156 | **** | ++++ |
| 19 | **** | ++++ | 65 | ** | | 111 | **** | | 157 | **** | ++++ |
| 20 | *** | | 66 | *** | +++ | 112 | **** | | 158 | **** | ++++ |
| 21 | * | | 67 | *** | +++ | 113 | * | | 159 | **** | ++++ |
| 22 | ** | | 68 | **** | ++++ | 114 | ** | | 160 | **** | |
| 23 | ** | | 69 | ** | | 115 | * | | 161 | **** | |
| 24 | ** | | 70 | *** | +++ | 116 | *** | +++ | 162 | **** | |
| 25 | *** | | 71 | ** | | 117 | | | 163 | **** | |
| 26 | **** | +++ | 72 | ** | + | 184 | **** | | 164 | **** | |
| 27 | **** | +++ | 73 | ** | | 185 | **** | | 165 | **** | |
| 28 | | | 74 | *** | ++++ | 186 | **** | | 166 | **** | |
| 29 | **** | ++++ | 75 | **** | ++++ | 187 | * | | 167 | **** | |
| 30 | **** | ++++ | 76 | ** | | 188 | *** | | 168 | **** | |
| 31 | | | 77 | ** | | 189 | * | | 169 | **** | |
| 32 | *** | | 78 | **** | | 124 | **** | ++++ | 170 | **** | |
| 33 | **** | ++++ | 79 | **** | +++ | 125 | **** | ++++ | 171 | **** | |
| 34 | **** | ++++ | 80 | *** | +++ | 126 | **** | ++++ | 172 | **** | |
| 35 | **** | ++++ | 81 | **** | ++++ | 127 | ** | + | 173 | **** | |
| 36 | **** | ++++ | 82 | **** | ++++ | 128 | *** | +++ | 174 | **** | |
| 37 | **** | ++++ | 83 | **** | ++++ | 129 | *** | +++ | 175 | **** | |
| 38 | **** | | 84 | **** | ++++ | 130 | *** | | 176 | **** | |
| 39 | *** | | 85 | *** | +++ | 131 | * | | 177 | **** | |
| 40 | **** | ++++ | 86 | **** | | 132 | **** | ++++ | 178 | **** | |
| 41 | **** | ++++ | 87 | **** | | 133 | **** | ++++ | 179 | *** | |
| 42 | **** | ++++ | 88 | **** | ++++ | 134 | *** | | 180 | * | |
| 43 | **** | | 89 | ** | ++ | 135 | **** | ++++ | 181 | **** | |
| 44 | **** | ++++ | 90 | ** | | 136 | *** | | 182 | **** | |
| 45 | **** | ++++ | 91 | **** | ++++ | 137 | ** | | 183 | **** | |
| 46 | **** | ++++ | 92 | **** | ++++ | 138 | *** | | | | |

****: IC₅₀ < 100 nM
***: 100 nM ≤ IC₅₀ < 1 μM
**: 1 μM ≤ IC₅₀ < 10 μM
*: IC₅₀ ≥ 10 μM
++++: EC₅₀ < 100 nM
+++: 100 nM ≤ EC₅₀ < 1 μM
++: 1 μM ≤ EC₅₀ < 10 μM
+: EC₅₀ ≥ 10 μM

Another aspect of the disclosure provides a pharmaceutical composition comprising at least one compound selected from a compound of Formulae I, II-1, II-2, II-3, II-4, II-5, III, IV, V, VI, VII, VIII, Compounds 1 to 183, a solvate thereof, a tautomer thereof, a stereoisomer thereof, a deuterated derivative of the compound, or a pharmaceutically acceptable salt of the foregoing, and at least one pharmaceutically acceptable carrier.

In some embodiments, the pharmaceutically acceptable carrier is selected from pharmaceutically acceptable vehicles and pharmaceutically acceptable adjuvants. In some embodiments, the pharmaceutically acceptable carrier is chosen from pharmaceutically acceptable fillers, disintegrants, surfactants, binders, and lubricants.

It will also be appreciated that a pharmaceutical composition of this disclosure can be employed in combination therapies; that is, the pharmaceutical compositions described herein can further include an additional active pharmaceutical agent. Alternatively, a pharmaceutical composition comprising a compound selected from a compound of Formulae I, II-1, II-2, II-3, II-4, II-5, III, IV, V, VI, VII, VIII, Compounds 1 to 183, a solvate thereof, a tautomer thereof, a stereoisomer thereof, a deuterated derivative of the compound, or a pharmaceutically acceptable salt of the foregoing can be administered as a separate composition concurrently with, prior to, or subsequent to, a composition comprising an additional active pharmaceutical agent. For example, the additional active pharmaceutical agents that can be used in combination with a compound of Formulae I, II-1, II-2, II-3, II-4, II-5, III, IV, V, VI, VII, VIII, Compounds 1 to 183, a solvate thereof, a tautomer thereof, a stereoisomer thereof, a deuterated derivative of the compound, or a pharmaceutically acceptable salt according to the present invention include but are not limited to one or more of the following: an interferon or a pegylated form thereof, remdesivir, galidesivir, favilavir, favipiravir, tenofivir, boceprevir, Ribavirin, β-Thymidine, Chlorhexidine, clevudine, dalcetrapib, dexamethasone, hydrocortisone, monoclonal antibodies such as regdanvimab, ravulizumab, bamlanivimab, mavrilimab, leronlimab lenzilumab, infliximab, adalimumab, lanadelumab, canakinumab, gimsilumab, otilimab, tocilizumab, and sarilumab, abivertinib, bemeentinib, acalabrutinib, baricitinib, tofacitinib, chloroquine, hydroxychloroquine, and azithromycin.

As described above, the pharmaceutical compositions disclosed herein comprise a pharmaceutically acceptable carrier. The pharmaceutically acceptable carrier may be chosen from adjuvants and vehicles. The pharmaceutically acceptable carrier, as used herein, can be chosen, for example, from any and all solvents, diluents, other liquid vehicles, dispersion aids, suspension aids, surface active agents, isotonic agents, thickening agents, emulsifying agents, preservatives, solid binders, and lubricants, which are suited to the particular dosage form desired. Remington: The Science and Practice of Pharmacy, 21st edition, 2005, ed. D. B. Troy, Lippincott Williams & Wilkins, Philadelphia, and Encyclopedia ofPharmaceutical Technology, eds. J. Swarbrick and J. C. Boylan, 1988 to 1999, Marcel Dekker, New York discloses various carriers used in formulating pharmaceutical compositions and known techniques for the preparation thereof. Except insofar as any conventional carrier is incompatible with the compounds of this disclosure, such as by producing any undesirable biological effect or otherwise interacting in a deleterious manner with any other component(s) of the pharmaceutical composition, its use is contemplated to be within the scope of this disclosure. Non-limiting examples of suitable pharmaceutically acceptable carriers include ion exchangers, alumina, aluminum stearate, lecithin, serum proteins (such as human serum albumin), buffer substances (such as phosphates, glycine, sorbic acid, and potassium sorbate), partial glyceride mixtures of saturated vegetable fatty acids, water, salts, and electrolytes (such as protamine sulfate, disodium hydrogen phosphate, potassium hydrogen phosphate, sodium chloride, and zinc salts), colloidal silica, magnesium trisilicate, polyvinyl pyrrolidone, polyacrylates, waxes, polyethylene-polyoxypropylene-block polymers, wool fat, sugars (such as lactose, glucose and sucrose), starches (such as corn starch and potato starch), cellulose and its derivatives (such as sodium carboxymethyl cellulose, ethyl cellulose and cellulose acetate), powdered tragacanth, malt, gelatin, talc, excipients (such as cocoa butter and suppository waxes), oils (such as peanut oil, cottonseed oil, safflower oil, sesame oil, olive oil, corn oil and soybean oil), glycols (such as propylene glycol and polyethylene glycol), esters (such as ethyl oleate and ethyl laurate), agar, buffering agents (such as magnesium hydroxide and aluminum hydroxide), alginic acid, pyrogen-free water, isotonic saline, Ringer's solution, ethyl alcohol, phosphate buffer solutions, non-toxic compatible lubricants (such as sodium lauryl sulfate and magnesium stearate), coloring agents, releasing agents, coating agents, sweetening agents, flavoring agents, perfuming agents, preservatives, and antioxidants.

The pharmaceutical compositions of the present disclosure may be formulated in the form of tablets, hard or soft gelatin capsules, powders, granules, a solutions or suspension in an aqueous or non-aqueous liquid, sterile injectable preparation, nasal or eye drops, or an oil-in-water liquid emulsion or a water-in-oil liquid emulsion. The desired dosage forms may be formulated according to the known art using the suitable excipients commonly used in the pharmacy. The formulations may conveniently be presented in unit dosage form and may be prepared by any of the methods well known in the art of pharmacy.

### III. Methods of Treatment and Uses

In another aspect of this disclosure, provided is a compound, solvate, tautomer, stereoisomer, deuterated derivative, or pharmaceutically acceptable salt as described herein, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof, for use in treating a disease and/or a symptom of a disease caused by a coronavirus. In another aspect, disclosed herein is use of a compound, solvate, tautomer, stereoisomer, deuterated derivative, or pharmaceutically acceptable salt as described herein, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof, for the manufacture of a medicament for treating a disease and/or a symptom or a disease caused by a coronavirus. In yet another aspect, disclosed herein is a method of treating a disease and/or a symptom of a disease in a subject, comprising administering to the subject a therapeutically effective amount of a compound, solvate, tautomer, stereoisomer, deuterated derivative, or pharmaceutically acceptable salt as described herein, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof. The compounds of the present invention can be used in the methods of the invention in combination with an additional active pharmaceutical agent. For example, the additional active pharmaceutical agents that can be used in combination with the compounds of the present invention include but are not limited to one or more of the following: an interferon or a pegylated form thereof, remdesivir, galidesivir, favilavir, favipiravir, tenofivir, boceprevir, Ribavirin, β-Thymidine, Chlorhexidine, clevudine, dalcetrapib, dexamethasone, hydrocortisone, monoclonal antibodies such as regdanvimab, ravulizumab, bamlanivimab, mavrilimab, leronlimab lenzilumab, infliximab, adalimumab, lanadelumab, canakinumab, gimsilumab, otilimab, tocilizumab, and sarilumab, abivertinib, bemcentinib, acalabrutinib, baricitinib, tofacitinib, chloroquine, hydroxychloroquine, and azithromycin.

In some embodiments, the disease caused by a coronavirus is a respiratory tract infection. In some embodiments, the disease caused by a coronavirus is a severe acute respiratory syndrome. In one embodiment, the disease caused by a coronavirus is COVID-19. In one embodiment, the disease caused by a coronavirus that is severe acute respiratory syndrome coronavirus 2 (SARS-CoV-2). In some embodiments, the symptom of a disease caused by a coronavirus is selected from fever or chills, cough, shortness of breath or difficulty in breathing, fatigue, muscle or body aches, headache, new loss of taste or smell, sore throat, congestion or runny nose, nausea or vomiting, diarrhea, persistent pain or pressure in the chest, new confusion, inability to wake or stay awake, bluish lips or face, and a combination thereof.

In another aspect of this disclosure, a compound, solvate, tautomer, stereoisomer, deuterated derivative, or pharmaceutically acceptable salt as described herein, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof, is for use in reducing or inhibiting the activity of a protease of a coronavirus. In another aspect, disclosed herein is use of a compound, solvate, tautomer, stereoisomer, deuterated derivative, or pharmaceutically acceptable salt as described herein, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof, for the manufacture of a medicament for reducing or inhibiting the activity of a protease of a coronavirus. In yet another aspect, disclosed herein is a method of reducing or inhibiting the activity of a protease of a coronavirus, comprising administering a therapeutically effective amount of a compound, solvate, tautomer, stereoisomer, deuterated derivative, or pharmaceutically acceptable salt as described herein to a subject, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof. In yet another aspect, disclosed herein is a method of reducing or inhibiting the activity of a protease of a coronavirus, comprising contacting said coronavirus or said protease with a compound, solvate, tautomer, stereoisomer, deuterated derivative, or pharmaceutically acceptable salt as described herein to a subject, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof. In one embodiment, the protease of a coronavirus is a 3C-like protease. In one embodiment, the coronavirus is SARS-CoV-2.

In another aspect of this disclosure, a compound, solvate, tautomer, stereoisomer, deuterated derivative, or pharmaceutically acceptable salt as described herein, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof, is for use in reducing or inhibiting the replication of a coronavirus. In another aspect, disclosed herein is use of a compound, solvate, tautomer, stereoisomer, deuterated derivative, or pharmaceutically acceptable salt as described herein, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof, for the manufacture of a medicament for reducing or inhibiting the replication of a coronavirus. In yet another aspect, disclosed herein is a method of reducing or inhibiting the replication of a coronavirus, comprising administering a therapeutically effective amount of a compound, solvate, tautomer, stereoisomer, deuterated derivative, or pharmaceutically acceptable salt as described herein to a subject, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof. In yet another aspect, disclosed herein is a method of reducing or inhibiting the replication of a coronavirus, comprising contacting said coronavirus or a protease of the coronavirus with a compound, solvate, tautomer, stereoisomer, deuterated derivative, or pharmaceutically acceptable salt as described herein to a subject, including a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof. In one embodiment, the protease of a coronavirus is a 3C-like protease. In one embodiment, the coronavirus is SARS-CoV-2.

A compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof may be administered once daily, twice daily, or three times daily, for example, for the treatment of a disease and/or a symptom of a disease caused by a coronavirus, for example, SARS-CoV-2.

In some embodiments, 2 mg to 1500 mg or 5 mg to 1000 mg of a compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof are administered once daily, twice daily, or three times daily.

A compound of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof may be administered, for example, by oral, parenteral, sublingual, topical, rectal, nasal, buccal, vaginal, transdermal, patch, pump administration or via an implanted reservoir, and a pharmaceutical compositions would be formulated accordingly. Parenteral administration includes intravenous, intraperitoneal, subcutaneous, intramuscular, transepithelial, nasal, intrapulmonary, intrathecal, rectal and topical modes of administration. Parenteral administration can be by continuous infusion over a selected period of time. Other forms of administration contemplated in this disclosure are as described in International Patent Application Nos. WO 2013/075083, WO 2013/075084, WO 2013/078320, WO 2013/120104, WO 2014/124418, WO 2014/151142, and WO 2015/023915.

One or more of the compounds of Formulae I, II-1, II-2, II-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing, or a pharmaceutical composition thereof may be administrated in combination with one or more additional pharmaceutical agents, for example, additional anti-SARS-CoV-2 agents or additional inhibitors of proteases of Coronaviridae viruses.

### EXAMPLES

In order that the disclosure described herein may be more fully understood, the following examples are disclosed herein. It should be understood that these examples are for illustrative purposes only and are not to be construed as limiting this disclosure in any way.

### Example 1. Design of 3CLpro Inhibitors

A series of non-covalent inhibitors of SARS-CoV-2 3CLpro (Table 1) were designed based on the compounds identified in a previous study (6).

### Example 2. Synthesis of Exemplary Compounds

The compounds of the disclosure may be made according to standard chemical practices or as described herein, including the following synthetic schemes and in the descriptions for preparing a compound selected from compounds of Formulae I, II-1, I1-2, I1-3, II-4, II-5, III, IV, V, VI, VII, VIII, or Compounds 1 to 183, a solvate thereof, a tautomer thereof, a stereoisomer thereof, a deuterated derivative of the compound, or a pharmaceutically acceptable salt of the foregoing.

### Synthesis of Compounds 1 to 5, 7 to 9, 55 and 72

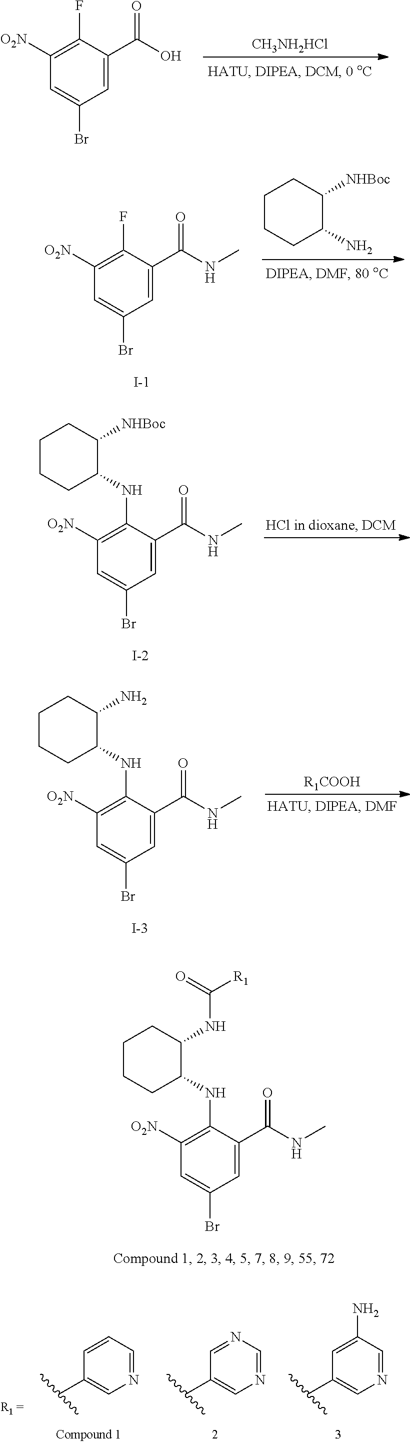

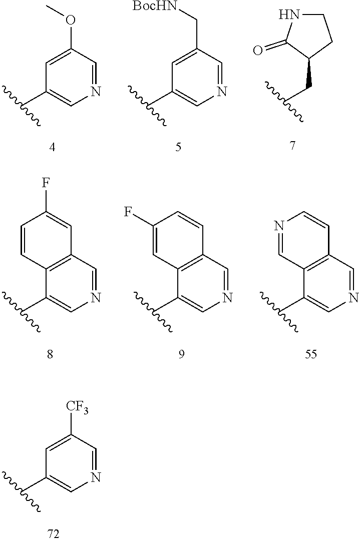

### Intermediate I-1: 5-bromo-2-fluoro-N-methyl-3-nitrobenzamide

To a solution of 5-bromo-2-fluoro-3-nitrobenzoic acid (2.64 g, 10 mmol) in DCM (20.0 mL), DIPEA (5.0 mL, 30 mmol), HATU (5.7 g, 15 mmol) and methylamine hydrochloride (0.81 g, 12 mmol) were added and the reaction was stirred at 0° C. for 2 hr. The reaction mixture was poured into H₂O (50.0 mL) and then extracted with DCM (50.0 mL*3). The combined organic layer was washed with brine (50.0 mL*2), dried over Na₂SO₄ and concentrated to give crude product which was purified by Flash Chromatography eluting with ethyl acetate in petroleum ether to afford the title compound (2.6 g, 94% yield) as a yellow solid. LC-MS (ESI) (M+H)⁺=277.1.

### Intermediate I-2: tert-butyl ((1S,2R)-2-((4-bromo-2-(methylcarbamoyl)-6-nitrophenyl)amino)cyclohexyl) carbamate

To a solution of 5-bromo-2-fluoro-N-methyl-3-nitrobenzamide (2.6 g, 9.4 mmol) in DMF (30 mL), tert-butyl ((1S,2R)-2-aminocyclohexyl)carbamate (2.22 g, 10.4 mmol) and DIPEA (4.66 mL, 28.2 mmol) were added and the reaction was stirred at 80° C. for 14 hr. The reaction mixture was poured into water (50 mL) and extracted with EtOAc (50 mL*3). The combined organic layer was washed with brine (50 mL*2), dried over Na₂SO₄ and concentrated under reduced pressure to obtain crude product (yellow solid). The residue was used directly without further purification.

### Intermediate I-3: 2-(((1R,2S)-2-aminocyclohexyl) amino)-5-bromo-N-methyl-3-nitrobenzamide

To a solution of tert-butyl ((1S,2R)-2-((4-bromo-2-(methylcarbamoyl)-6-nitrophenyl) amino) cyclohexyl) carbamate (400 mg, 0.84 mmol) in DCM (4.0 mL), 4 N HCl in dioxane (2.6 mL) was added and the reaction was stirred at 25° C. for 2 hr. The reaction mixture was dried in vacuo to afford the 2-(((1R,2S)-2-aminocyclohexyl) amino)-5-bromo-N-methyl-3-nitrobenzamide (290 mg, 0.78 mmol, 73% yield) as a yellow solid. LC-MS (ESI) (M+H)⁺=371.1 Compounds 1-5, 7-9, 55 and 72:

To a solution of 2-(((1R,2S)-2-aminocyclohexyl) amino)-5-bromo-N-methyl-3-nitrobenzamide (1.0 equiv) in DMF (5 mL), DIPEA (5.0 equiv), corresponding acid R₁COOH (1.0 equiv) and HATU (2.0 equiv) were added and the reaction was stirred at 25° C. for 12 hr. The reaction mixture was poured into water (30 mL) and extracted with EtOAc (30 mL*3). The combined organic layer was washed with saturated salt water (3*20 mL). The organic layers dried over Na₂SO₄ was filtered and concentrated under reduced pressure to dryness. The residue was purified by Prep-HPLC to give the target compound as a yellow solid.

| | |
|---|---|
| 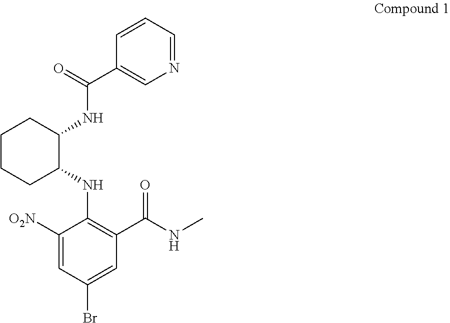 [SMILES: CNC(=O)c1cc(Br)cc([N+](=O)[O-])c1N[C@@H]1CCCC[C@@H]1NC(=O)c1cccnc1] Compound 1 | Compound 1: N-((1S, 2R)-2-((4- bromo-2-(methylcarbamoyl)-6- nitrophenyl)amino)cyclohexyl)nicotinamide ¹H NMR (500 MHz, CDCl₃) δ 8.90 (s, 1H), 8.67 (s, 1H), 8.15 (s, 1H), 8.03 (d, J = 7.4 Hz, 1H), 7.62 (d, J = 17.0 Hz, 2H), 7.37 (s, 1H), 6.79 (s, 2H), 4.25 (s, 1H), 3.82 (s, 1H), 3.02-2.91 (m, 3H), 1.67-1.52 (m, 8H). LC-MS (ESI) (M + H)⁺ = 476.31. |
| 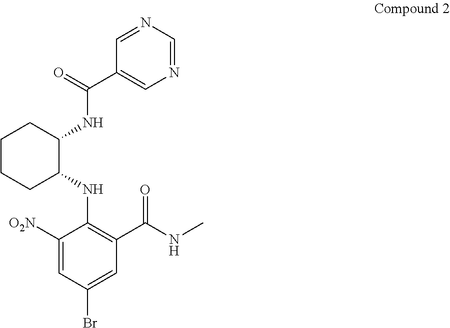 [SMILES: CNC(=O)c1cc(Br)cc([N+](=O)[O-])c1N[C@@H]1CCCC[C@@H]1NC(=O)c1cncnc1] Compound 2 | Compound 2: N-((1S, 2R)-2-((4- bromo-2-(methylcarbamoyl)-6- nitrophenyl)amino)cyclohexyl) pyrimidine-5-carboxamide ¹H NMR (500 MHz, Methanol-d₄) δ 9.24 (s, 1H), 9.01 (s, 2H), 8.25 (d, J = 2.4 Hz, 1H), 7.63 (d, J = 2.4 Hz, 1H), 4.31-4.21 (m, 1H), 3.86 (d, J = 3.3 Hz, 1H), 2.90 (s, 3H), 1.85-1.61 (m, 6H), 1.57-1.43 (m, 2H). LC-MS (ESI) (M + H)⁺ = 477.23. |
| 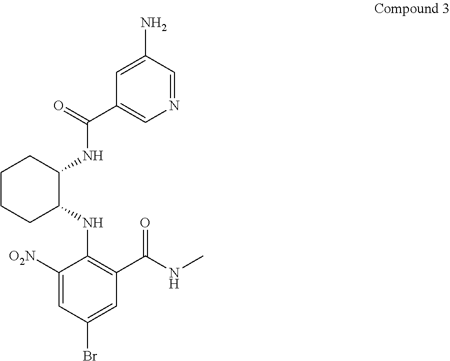 [SMILES: CNC(=O)c1cc(Br)cc([N+](=O)[O-])c1N[C@@H]1CCCC[C@@H]1NC(=O)c1cncc(N)c1] Compound 3 | Compound 3: 5-amino-N-((1S, 2R)-2- ((4-bromo-2-(methylcarbamoyl)-6- nitrophenyl)amino)cyclohexyl)nicotinamide ¹H NMR (500 MHz, CDCl₃) δ 8.36 (s, 1H), 8.16 (d, J = 2.3 Hz, 1H), 7.78 (d, J = 8.9 Hz, 1H), 7.65 (d, J = 2.1 Hz, 1H), 7.59 (d, J = 10.4 Hz, 1H), 6.74 (s, 1H), 6.52 (d, J = 8.7 Hz, 1H), 6.33 (d, J = 6.8 Hz, 1H), 5.36-5.26 (m, 2H), 4.22 (s, 1H), 3.82 (s, 1H), 2.99 (d, J = 4.8 Hz, 3H), 1.82-1.52 (m, 8H). LC-MS (ESI) (M + H)⁺ = 491.25. |
| 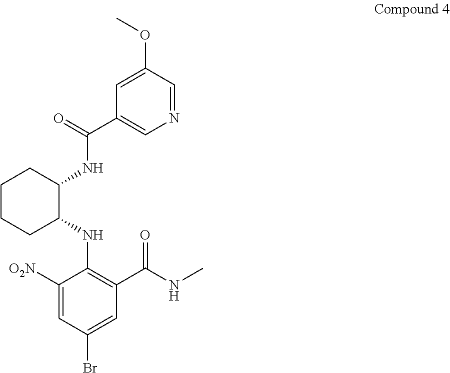 [SMILES: CNC(=O)c1cc(Br)cc([N+](=O)[O-])c1N[C@@H]1CCCC[C@@H]1NC(=O)c1cncc(OC)c1] Compound 4 | Compound 4: N-((1S, 2R)-2-((4- bromo-2-(methylcarbamoyl)-6- nitrophenyl)amino)cyclohexyl)- 5-methoxynicotinamide ¹H NMR (500 MHz, DMSO-d₆) δ 8.64 (d, J = 4.5 Hz, 1H), 8.42 (d, J = 1.4 Hz, 1H), 8.39 (d, J = 2.8 Hz, 1H), 8.30 (d, J = 8.5 Hz, 1H), 8.12 (d, J = 2.3 Hz, 1H), 7.66 (d, J = 2.4 Hz, 1H), 7.54-7.47 (m, 1H), 4.17 (s, 1H), 3.87 (s, 3H), 3.68 (s, 1H), 2.73 (d, J = 4.5 Hz, 3H), 1.82-1.28 (m, 8H). LC-MS (ESI) (M + H)⁺ = 506.32. |
| 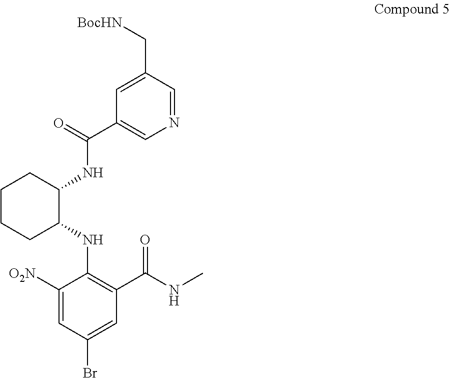 [SMILES: CNC(=O)c1cc(Br)cc([N+](=O)[O-])c1N[C@@H]1CCCC[C@@H]1NC(=O)c1cncc(CNC(=O)OC(C)(C)C)c1] Compound 5 | Compound 5: tert-butyl ((5-(((1S, 2R)-2- ((4-bromo-2-(methylcarbamoyl)-6- nitrophenyl)amino)cyclohexyl)carbamoyl) pyridin-3-yl)methyl)carbamate ¹H NMR (500 MHz, CDCl₃) δ 8.79 (s, 1H), 8.61 (s, 1H), 8.14 (d, J = 1.9 Hz, 1H), 7.93 (s, 1H), 7.65 (t, J = 8.3 Hz, 2H), 6.79 (s, 1H), 6.74 (s, 1H), 5.17 (s, 1H), 4.34 (s, 2H), 4.23 (s, 1H), 3.83 (d, J = 5.7 Hz, 1H), 2.96 (d, J = 4.7 Hz, 3H), 2.12-1.48 (m, 8H), 1.44 (s, 9H). LC-MS (ESI) (M + H)⁺ = 605.45. |
| 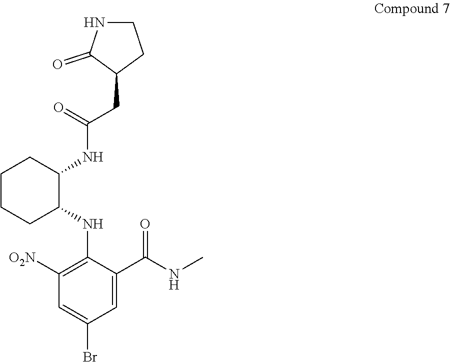 [SMILES: CNC(=O)c1cc(Br)cc([N+](=O)[O-])c1N[C@@H]1CCCC[C@@H]1NC(=O)C[C@@H]1CCNC1=O] Compound 7 | Compound 7: 5-bromo-N-methyl-3-nitro-2- (((1R, 2S)-2-(2-((S)-2-oxopyrrolidin- 3-yl)acetamido)cyclohexyl)amino)benzamide ¹H NMR (400 MHz, DMSO-d₆) δ 8.67 (m, 1H), 8.16 (d, J = 2.8 Hz, 1H), 8.01 (d, J = 10.0 Hz, 1H), 7.92 (d, J = 8.8 Hz, 1H), 7.65 (m, 2H), 4.00 (br s, 1H), 3.49 (br s, 3H), 3.12 (m, 2H), 2.75 (d, J = 4.8 Hz, 3H), 2.42 (m, 1H), 2.07-2.00 (m, 2H), 1.61-1.52 (m, 6H), 1.38 (m, 2H), 1.26 (m, 2H). LC-MS (ESI) (M + H)⁺ = 496.1. |
| 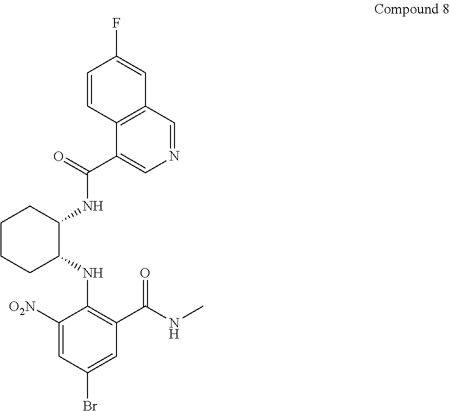 [SMILES: CNC(=O)c1cc(Br)cc([N+](=O)[O-])c1N[C@@H]1CCCC[C@@H]1NC(=O)c1cncc2cc(F)ccc12] Compound 8 | Compound 8: N-((1S, 2R)-2-((4-bromo-2- (methylcarbamoyl)-6-nitrophenyl) amino)cyclohexyl)-7-fluoroisoquinoline- 4-carboxamide ¹H NMR (400 MHz, Methanol-d₄) δ 9.28 (s, 1H), 8.44 (s, 1H), 8.26 (t, J = 4.0 Hz, 1H), 8.21 (dd, J = 8.0, 4.0 Hz, 1H), 7.84 (dt, J = 8.0, 4.0 Hz, 1H), 7.71-7.62 (m, J = 4.0 Hz, 2H), 4.39 (d, J = 4.0 Hz, 1H), 3.97 (s, 1H), 2.92 (d, J = 2.0 Hz, 3H), 1.91-1.49 (m, 8H). LC-MS (ESI) (M + H)⁺ = 544.1. |
| 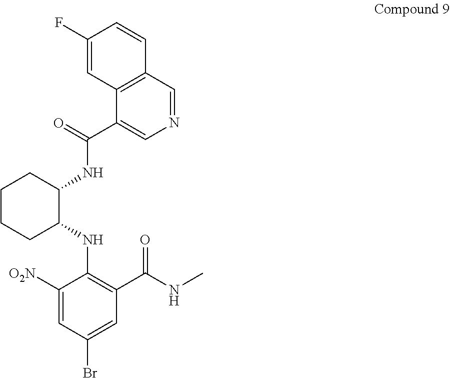 [SMILES: CNC(=O)c1cc(Br)cc([N+](=O)[O-])c1N[C@@H]1CCCC[C@@H]1NC(=O)c1cncc2ccc(F)cc12] Compound 9 | Compound 9: N-((1S, 2R)-2-((4- bromo-2-(methylcarbamoyl)-6- nitrophenyl)amino)cyclohexyl)-6- fluoroisoquinoline-4-carboxamide ¹H NMR (400 MHz, Methanol-d₄) δ 9.29 (s, 1H), 8.48 (s, 1H), 8.28-8.20 (m, 2H), 7.90-7.84 (m, 1H), 7.65 (t, J = 2.0 Hz, 1H), 7.55 (td, J = 8.0, 2.0 Hz, 1H), 4.39 (s, 1H), 3.97 (s, 1H), 2.92 (d, J = 2.0 Hz, 3H), 1.67 (d, J = 100 Hz, 8H). LC-MS (ESI) (M + H)⁺ = 544.1. |
| 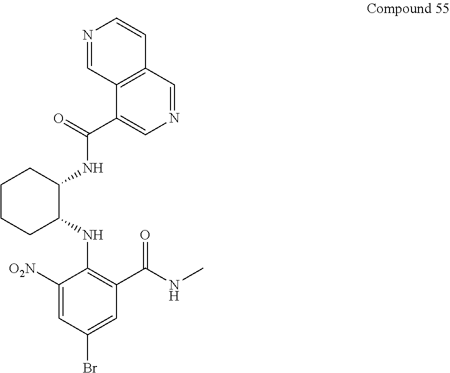 [SMILES: CNC(=O)c1cc(Br)cc([N+](=O)[O-])c1N[C@@H]1CCCC[C@@H]1NC(=O)c1cncc2ccncc12] Compound 55 | Compound 55: N-((1S, 2R)-2-((4- bromo-2-(methylcarbamoyl)-6- nitrophenyl)amino)cyclohexyl)-2,6- naphthyridine-4-carboxamide ¹H NMR (400 MHz, Methanol-d₄) δ 9.66 (s, 1H), 9.47 (s, 1H), 8.74 (d, J = 8.0 Hz, 1H), 8.70 (s, 1H), 8.25 (s, 1H), 8.07 (d, J = 4.0 Hz, 1H), 7.66 (s, 1H), 4.43 (s, 1H), 3.98 (s, 1H), 2.92 (s, 3H), 1.88 (s, 1H), 1.83- 1.72 (m, 4H), 1.67-1.49 (m, 3H). LC- MS (ESI) (M + H)⁺ = 527.1. |
| 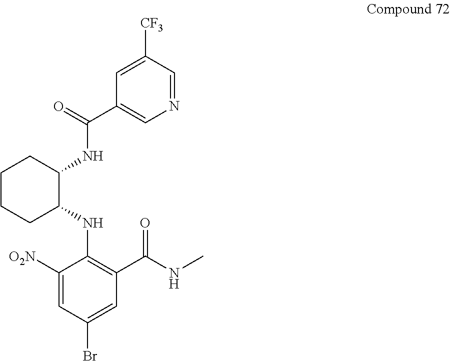 [SMILES: CNC(=O)c1cc(Br)cc([N+](=O)[O-])c1N[C@@H]1CCCC[C@@H]1NC(=O)c1cncc(C(F)(F)F)c1] Compound 72 | Compound 72: N-((1S, 2R)-2 ((4- bromo-2-(methylcarbamoyl)-6- nitrophenyl)amino)cyclohexyl)-5- (trifluoromethyl)nicotinamide ¹H NMR (400 MHz, Methanol-d₄) δ 9.07 (s, 1H), 9.01 (s, 1H), 8.40 (s, 1H), 8.24 (d, J = 2.0 Hz, 1H), 7.63 (d, J = 2.0 Hz, 1H), 4.27 (s, 1H), 3.91 (s, 1H), 2.91 (s, 3H), 1.81-1.66 (m, 6H), 1.52 (s, 2H). LC-MS (ESI) (M + H)⁺ = 544.1. |

### Synthesis of Compounds 108, 121, 122, 179 and 180

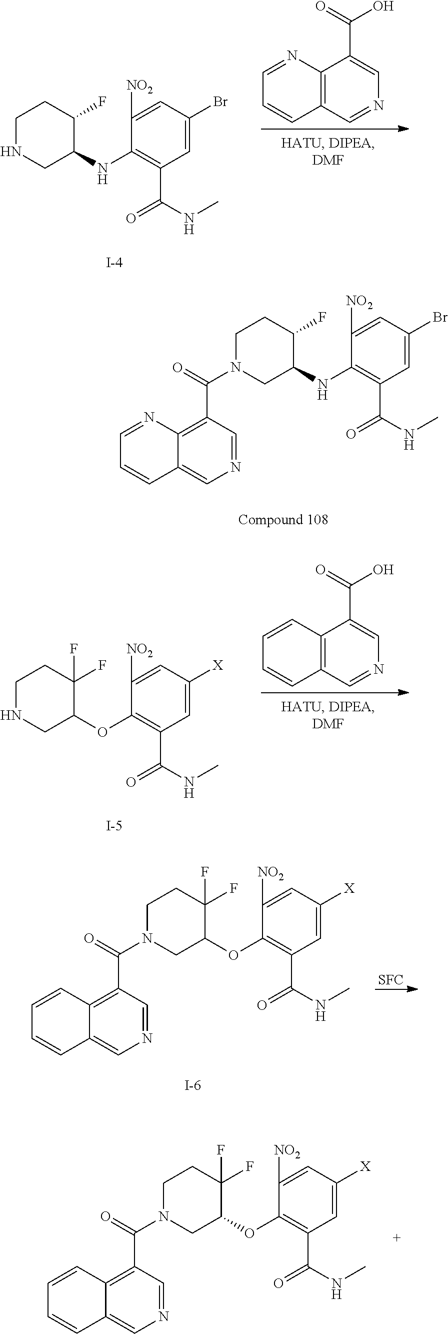

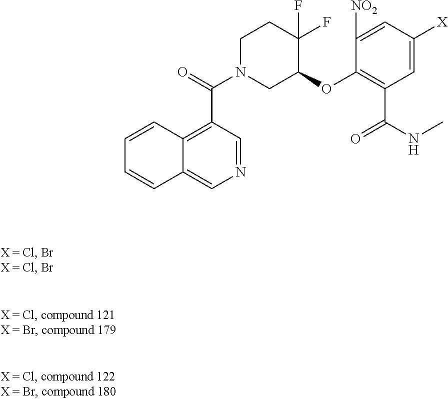

### Intermediates I-4 and I-5

Intermediates I-4 and I-5 were synthesized using the same route of intermediate I-3.

### Compound 108 and Intermediate I-6

The procedure for making compounds 108 and intermediate I-6 is similar to the synthetic procedure of compound 1.

### Compounds 121, 122, 179 and 180

Intermediate I-6 was further separated by SFC (column: DAICEL CHIRALCEL OX (250 mm*30 mm, 10 um)) to give compounds 121, 122, 179 and 180.

| | |
|---|---|
| 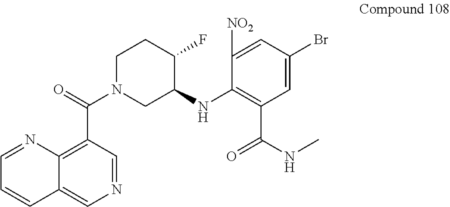 [SMILES: CNC(=O)c1cc(Br)cc([N+](=O)[O-])c1N[C@H]1CN(C(=O)c2cncc3cccnc23)CC[C@@H]1F] Compound 108 | Compound 108: 5-bromo-2-(((3S, 4S)-4- fluoro-1-(1,6-naphthyridine-8-carbonyl) piperidin-3-yl)amino)-N-methyl-3-nitrobenzamide ¹H NMR (400 MHz, DMSO-d₆) δ 9.52-9.40 (m, 1H), 9.24-9.11 (m, 1H), 8.90-8.52 (m, 3H), 8.29-7.59 (m, 4H), 4.91-3.60 (m, 5H), 3.26-2.95 (m, 3H), 2.64 (s, 1H), 1.92 (m, 2H). LC-MS (ESI) (M + H)⁺ = 533.2. |
| 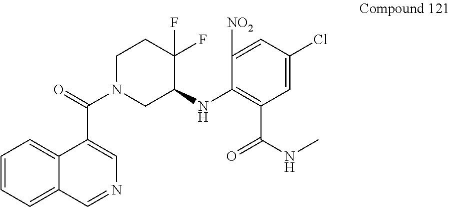 [SMILES: CNC(=O)c1cc(Cl)cc([N+](=O)[O-])c1N[C@H]1CN(C(=O)c2cncc3ccccc23)CCC1(F)F] Compound 121 | Compound 121: (R)-5-chloro-2- ((4,4-difluoro-1-(isoquinoline-4- carbonyl)piperidin-3-yl)oxy)- N-methyl-3-nitrobenzamide ¹H NMR (400 MHz, DMSO-d₆) δ 9.40 (m, 1H), 8.89 (m, 0.5H), 8.41-8.56 (m, 1H), 7.82-8.30 (m, 4.5H), 7.32-7.82 (m, 2H), 4.17- 4.71 (m, 2H), 3.37-3.95 (m, 2H), 3.14- 3.25 (m, 1H), 2.87 (m, 2H), 2.13-2.31 (m, 2H), 1.90-2.06 (m, 1H). LC-MS (ESI) (M + H)⁺ = 505.3. |
| 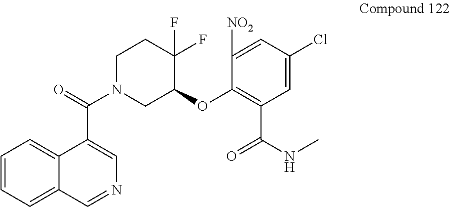 [SMILES: CNC(=O)c1cc(Cl)cc([N+](=O)[O-])c1O[C@H]1CN(C(=O)c2cncc3ccccc23)CCC1(F)F] Compound 122 | Compound 122: (S)-5-chloro-2-((4,4- difluoro-1-(isoquinoline-4- carbonyl)piperidin-3-yl)oxy)- N-methyl-3-nitrobenzamide ¹H NMR (400 MHz, DMSO-d₆) δ 9.33-9.48 (m, 1H), 8.89 (m, 0.5H), 8.41-8.54 (m, 1H), 8.18-8.32 (m, 1.5H), 7.48-8.18 (m, 5H), 4.20-4.74 (m, 2H), 3.60-3.93 (m, 2H), 3.11-3.28 (m, 1H), 2.87 (m, 2H), 2.16-2.40 (m, 2H), 1.86-2.07 (m, 1H). LC-MS (ESI) (M + H)⁺ = 505.3. |
| 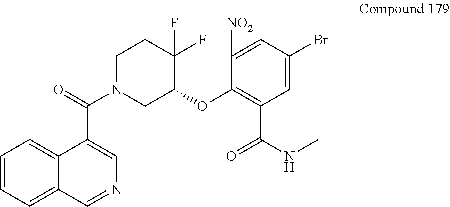 [SMILES: CNC(=O)c1cc(Br)cc([N+](=O)[O-])c1O[C@@H]1CN(C(=O)c2cncc3ccccc23)CCC1(F)F] Compound 179 | Compound 179: (R)-5-bromo-2- ((4,4-difluoro-1-(isoquinoline-4- carbonyl)piperidin-3-yl)oxy)- N-methyl-3-nitrobenzamide ¹H NMR (400 MHz, DMSO-d₆) δ 9.39 (m, 1H), 8.87 (m, 0.5H), 8.49 (s, 0.5H), 8.39- 8.47 (m, 0.5H), 8.29-8.33 (m, 0.5H), 8.05- 8.27 (m, 2H), 7.63-7.99 (m, 4H), 4.12- 4.74 (m, 2H), 3.40-3.94 (m, 2H), 2.87 (m, 2H), 2.23-2.40 (m, 2H), 1.72-2.15 (m, 2H). LC-MS (ESI) (M + H)⁺ = 551.3. |
| 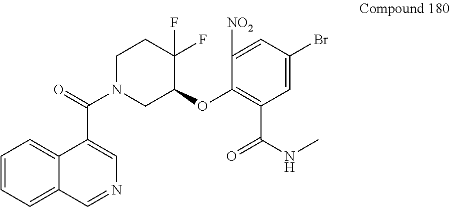 [SMILES: CNC(=O)c1cc(Br)cc([N+](=O)[O-])c1O[C@H]1CN(C(=O)c2cncc3ccccc23)CCC1(F)F] Compound 180 | Compound 180: (S)-5-bromo-2- ((4,4-difluoro-1-(isoquinoline-4- carbonyl)piperidin-3-yl)oxy)-N- methyl-3-nitrobenzamide ¹H NMR (400 MHz, DMSO-d₆) δ9.39 (m, 1H), 8.87 (m, 0.5H), 8.43-8.55 (m, 1H), 8.31 (m, 0.5H), 8.02-8.28 (m, 2H), 7.57- 8.00 (m, 4H), 4.20-4.73 (m, 2H), 3.43-3.89 (m, 2H), 3.22 (m, 1H), 2.87 (m, 2H), 2.17- 2.36 (m, 2H), 1.84-2.03 (m, 1H). LC-MS (ESI) (M + H)⁺ = 551.3. |

### Synthesis of Compound 57

The synthesis route of compound 57 is similar to that of compound 1 using 5-bromo-2-fluoro-3-(methylsulfonyl)benzoic acid as the start material.

| | |
|---|---|
| 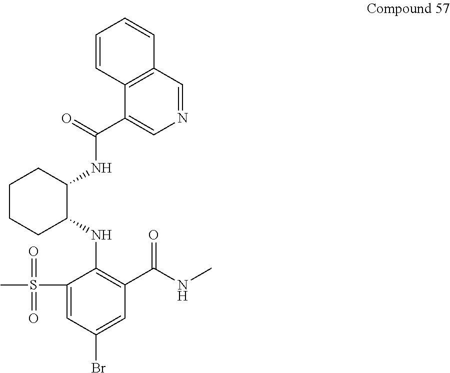 Compound 57 | Compound 57: N-((1S, 2R)-2-((4- bromo-2-(methylcarbamoyl)-6- (methylsulfonyl)phenyl)amino)cyclohexyl) isoquinoline-4-carboxamide ¹H NMR (400 MHz, Methanol-d₄) δ 9.32 (s, 1H), 8.69 (s, 1H), 8.17 (t, J = 7.2 Hz, 2H), 7.94 (d, J = 4.0 Hz, 1H), 7.86 (d, J = 8.0 Hz, 1H), 7.77 (d, J = 8.0 Hz, 1H), 7.61 (d, J = 2.4 Hz, 1H), 4.62 (s, 1H), 3.91 (s, 1H), 3.15 (s, 3H), 2.96 (s, 3H), 1.94- 1.30 (m, 8H). LC-MS (ESI) (M + H)⁺ = 559.1. |

### Synthesis of Compound 6

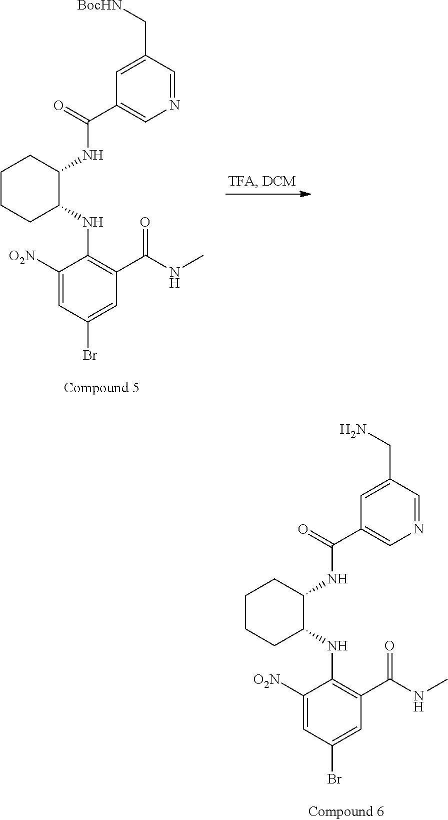 [SMILES: CNC(=O)c1cc(Br)cc([N+](=O)[O-])c1N[C@@H]1CCCC[C@@H]1NC(=O)c1cncc(CNC(=O)OC(C)(C)C)c1 ; CNC(=O)c1cc(Br)cc([N+](=O)[O-])c1N[C@@H]1CCCC[C@@H]1NC(=O)c1cncc(CN)c1]

### Compound 6: 5-(aminomethyl)-N-((1S,2R)-2-((4-bromo-2-(methylcarbamoyl)-6-nitrophenyl) amino) cyclohexyl) nicotinamide

Compound 5 (45 mg, 0.07 mmol) was dissolved in 5 mL of DCM, and 2 ml of TFA was added. The reaction mixture was stirred at room temperature for 3 h. The solvent was removed and the residue was neutralized with aqueous NaHCO₃ and extracted with ethyl acetate, followed by washing with saturated brine solution. The organic layer was dried on anhydrous Na₂SO₄. The solution was concentrated and purified by flash silica chromatography to give the target product (35 mg, 93% yield) as a yellow solid.

| | |
|---|---|
| 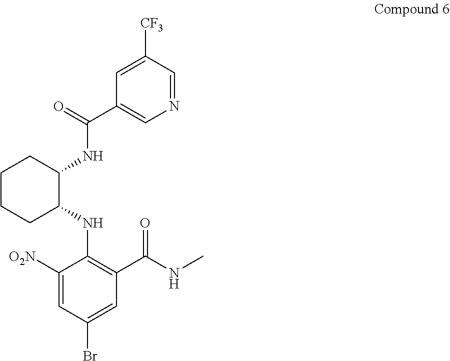 Compound 6 | Compound 6: 5-(aminomethyl)-N-((1S, 2R)-2-((4-bromo-2-(methylcarbamoyl)-6- nitrophenyl) amino) cyclohexyl) nicotinamide ¹H NMR (500 MHz, CDCl3) δ 8.77 (s, 1H), 8.64 (s, 1H), 8.14 (s, 1H), 7.95 (s, 1H), 7.64 (s, 2H), 6.77 (s, 1H), 6.61 (s, 1H), 4.24 (s, 1H), 3.98 (s, 2H), 3.84 (s, 1H), 2.98 (d, J = 4.7 Hz, 3H), 1.90- 1.44 (m, 8H). LC-MS (ESI) (M + H)⁺ = 505.34. |

### Synthesis of Compounds 10, 13, 18, 20, 58, 62, 63, 64, 66, 70, 75, 77, 87, 88, 99, 127, 129, 131, 132 and 141

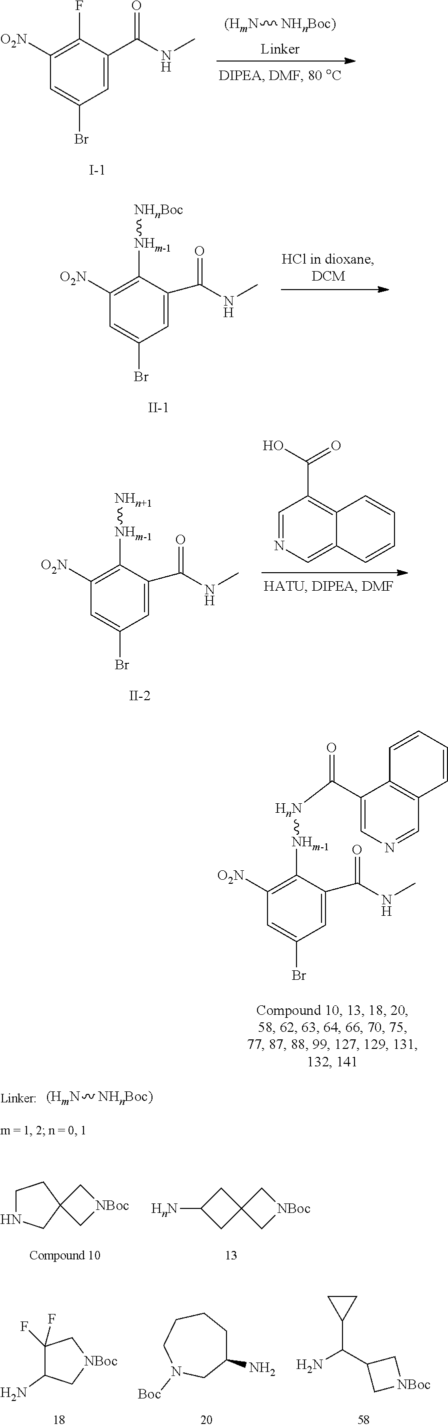

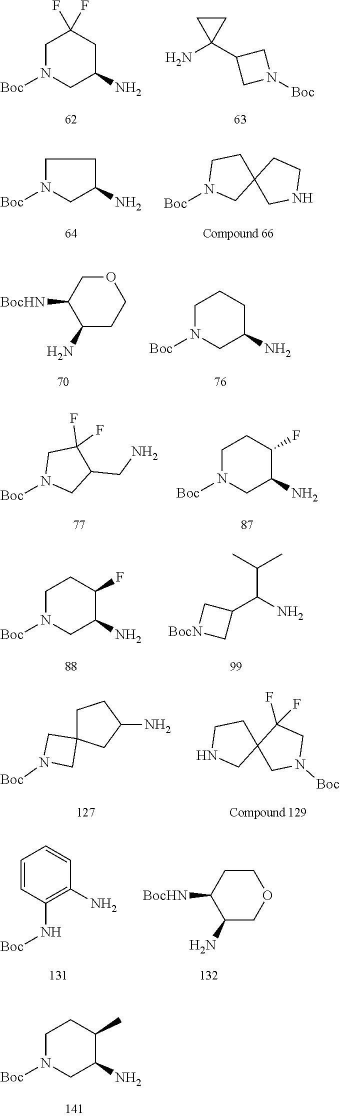

### Intermediate II-1

DIPEA (3.0 equiv) was added to a solution of 5-bromo-2-fluoro-N-methyl-3-nitrobenzamide (1.0 equiv) and a corresponding linker (1.2 equiv) in DMF (1 mL), and the reaction was stirred at 80° C. for 12 h. The reaction was diluted with water (5 mL) and extracted with EtOAc (3*10 mL). The organic layer was separated, washed with water and further saturated NaCl solution (3*10 mL), and dried over Na₂SO₄, then concentrated in vacuo. The residue was purified using silica gel column chromatography eluting with ethyl acetate in petroleum ether to afford intermediate II-1 as a yellow solid.

### Intermediate II-2

To a solution of intermediate II-1 (0.2-0.5 mmol) in DCM (1 mL) was added TFA (1 mL), and the reaction was stirred at room temperature for 2 hr. The reaction was concentrated under reduced pressure to give intermediate h-2 as a yellow solid.

### Compounds 10, 13, 18, 20, 58, 62, 63, 64, 66, 70, 75, 77, 87, 88, 99, 127, 129, 131, 132 and 141

To a solution of intermediate m-2 (1.0 equiv) in DMF, isoquinoline-4-carboxylic acid (1.2 equiv), DIPEA (3.0 equiv) and HATU (2.0 equiv) were added and the reaction was stirred at room temperature for 12 hr. The reaction was diluted with water and extracted with EtOAc three times. The organic layer was separated, washed with water and further saturated NaCl solution, and dried over Na₂SO₄, then concentrated in vacuo. The residue was purified using silica gel column chromatography eluting with ethyl acetate in petroleum ether to afford the target compounds as yellow solids.

| | |
|---|---|
| 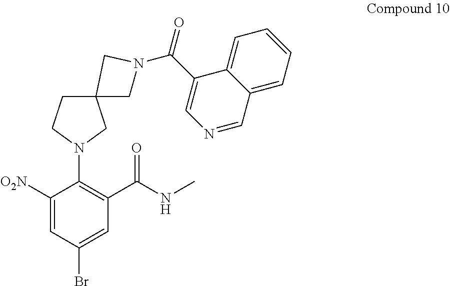 [SMILES: CNC(=O)c1cc(Br)cc([N+](=O)[O-])c1N1CCC2(CN(C(=O)c3cncc4ccccc34)C2)C1] Compound 10 | Compound 10: 5-bromo-2-(2-(isoquinoline- 4-carbonyl)-2,6-diazaspiro[3, 4]octan-6-yl)-N-methyl-3-nitrobenzamide ¹H NMR (400 MHz, Methanol-d₄) δ 9.33 (s, 1H), 8.55 (s, 1H), 8.20 (d, J = 8.0 Hz, 1H), 8.13 (d, J = 8.0 Hz, 1H), 7.94- 7.87 (m, 1H), 7.87-7.85 (m, 1H), 7.80-7.75 (m, 1H), 7.57 (d, J = 4.0 Hz, 1H), 4.27-4.20 (m, 2H), 4.04-3.94 (m, 2H), 3.43 (d, J = 12.0 Hz, 1H), 3.35 (d, J = 8.0 Hz, 1H), 3.28-3.23 (m, 2H), 2.83 (s, 3H), 2.25-2.14 (m, 2H). LC-MS (ESI) (M + H)⁺ = 524.1. |
| 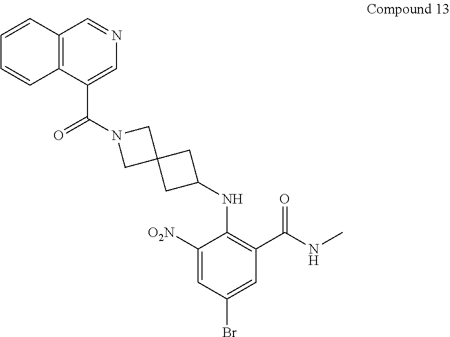 [SMILES: CNC(=O)c1cc(Br)cc([N+](=O)[O-])c1NC1CC2(C1)CN(C(=O)c1cncc3ccccc13)C2] Compound 13 | Compound 13: 5-bromo-2-((2-(isoquinoline- 4-carbonyl)-2-azaspiro[3.3] heptan-6-yl)amino)-N-methyl-3-nitrobenzamide ¹H NMR (400 MHz, DMSO-d₆) δ 9.66 (s, 1H), 8.67 (d, J = 4.0 Hz, 1H), 8.43 (d, J = 8.0 Hz, 1H), 8.28 (d, J = 8.0 Hz, 1H), 8.18 (d, J = 16.0 Hz, 2H), 7.98 (t, J = 8.0 Hz, 1H), 7.65 (d, J = 8.0 Hz, 1H), 4.33 (s, 1H), 4.26 (s, 1H), 4.09 (s, 1H), 4.01 (s, 1H), 3.81 (dt, J = 52.0, 8.0 Hz, 1H), 2.92 (s, 2H), 2.82 (s, 1H), 2.75-2.69 (m, 1H), 2.67-2.61 (m, 1H), 2.31-2.24 (m, 1H), 2.21-2.14 (m, 1H). LC-MS (ESI) (M + H)⁺ = 524.0. |
| 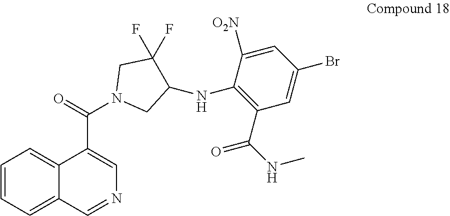 [SMILES: CNC(=O)c1cc(Br)cc([N+](=O)[O-])c1NC1CN(C(=O)c2cncc3ccccc23)CC1(F)F] Compound 18 | Compound 18: 5-bromo-2-((4,4- difluoro-1-(isoquinoline-4- carbonyl)pyrrolidin-3-yl)amino)- N-methyl-3-nitrobenzamide ¹H NMR (400 MHz, DMSO-d₆) δ 9.43 (d, J = 7.3 Hz, 1H), 8.95-8.67 (m, 1H), 8.58 (s, 1H), 8.29-8.07 (m, 2H), 8.04-7.45 (m, 5H), 4.44-3.55 (m, 5H), 2.71 (dd, J = 94.1, 4.5 Hz, 3H). LC-MS (ESI) (M + H)⁺ = 534.1. |
| 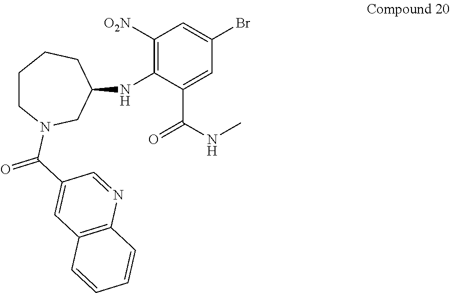 [SMILES: CNC(=O)c1cc(Br)cc([N+](=O)[O-])c1N[C@@H]1CCCCN(C(=O)c2cnc3ccccc3c2)C1] Compound 20 | Compound 20: (R)-5-bromo-N- methyl-3-nitro-2-((1-(quinoline-3- carbonyl)azepan-3-yl)amino)benzamide ¹H NMR (400 MHz, Methanol-d₄) δ 9.24-9.11 (m, 1H), 8.51-7.94 (m, 3H), 7.74 (dt, J = 48.2, 7.6 Hz, 3H), 7.52 (dd, J = 82.2, 2.5 Hz, 1H), 4.18-3.52 (m, 3H), 3.27 (s, 1H), 3.11 (dd, J = 14.0, 7.3 Hz, 1H), 2.87 (s, 3H), 2.09-1.44 (m, 6H). LC-MS (ESI) (M + H)⁺ = 526.1. |
| 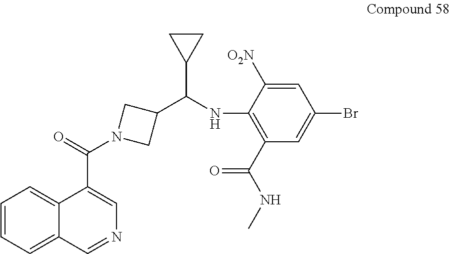 [SMILES: CNC(=O)c1cc(Br)cc([N+](=O)[O-])c1NC(C1CC1)C1CN(C(=O)c2cncc3ccccc23)C1] Compound 58 | Compound 58: 5-bromo-2- ((cyclopropyl(1-(isoquinoline-4- carbonyl)azetidin-3-yl)methyl)amino)- N-methyl-3-nitrobenzamide ¹H NMR (400 MHz, DMSO-d₆) δ 9.39 (d, J = 4.0 Hz, 1H), 8.77 (dd, J = 8.0, 4.0 Hz, 1H), 8.63-8.48 (m, 1H), 8.31- 8.13 (m, 2H), 8.12-8.02 (m, 1H), 7.95- 7.81 (m, 1H), 7.80-7.73 (m, 1H), 7.71 (t, J = 2.0 Hz, 1H), 4.79-4.44 (m, 1H), 4.36-4.14 (m, 1H), 4.06 (dd, J = 16.0, 8.0 Hz, 1H), 4.01-3.92 (m, 1H), 3.88- 3.69 (m, 1H), 3.09-2.93 (m, 2H), 2.72 (dd, J = 16.0, 4.0 Hz, 3H), 0.84 (dd, J = 8.0, 4.0 Hz, 1H), 0.51-0.04 (m, 4H). LC-MS (ESI) (M + H)⁺ = 538.1. |
| 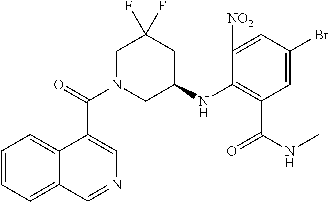 [SMILES: CNC(=O)c1cc(Br)cc([N+](=O)[O-])c1N[C@H]1CN(C(=O)c2cncc3ccccc23)CC(F)(F)C1] Compound 62 | Compound 62: (R)-5-bromo-2- ((5,5-difluoro-1-(isoquinoline-4- carbonyl)piperidin-3-yl)amino)- N-methyl-3-nitrobenzamide ¹H NMR (400 MHz, Methanol-d₄) δ 10.2 (d, J = 28.0 Hz, 1H), 9.74-8.95 (m, 3H), 8.85-8.50 (m, 4H), 5.05-4.24 (m, 5H), 3.72-3.33 (m, 3H), 3.32-3.03 (m, 2H). LC-MS (ESI) (M + H)⁺ = 548.0. |
| 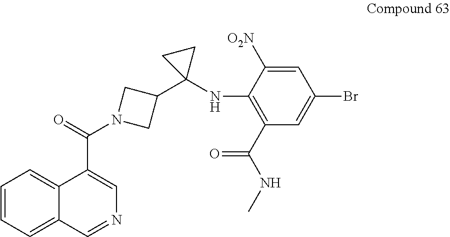 [SMILES: CNC(=O)c1cc(Br)cc([N+](=O)[O-])c1NC1(C2CN(C(=O)c3cncc4ccccc34)C2)CC1] Compound 63 | Compound 63: 5-bromo-2-((1-(1- (isoquinoline-4-carbonyl)azetidin-3- yl)cyclopropyl)amino)-N- methyl-3-nitrobenzamide ¹H NMR (400 MHz, Methanol-d₄) δ 9.35 (s, 1H), 8.53 (s, 1H), 8.23 (d, J = 8.0 Hz, 1H), 8.17-8.05 (m, 2H), 7.96-7.90 (m, 1H), 7.83-7.78 (m, 1H), 7.71 (d, J = 4.0 Hz, 1H), 4.33 (t, J = 20.0, 12.0 Hz, 1H), 4.12-3.93 (m, 2H), 3.84-3.69 (m, 1H), 3.41-3.35 (m, 1H), 2.85 (s, 3H), 0.96-0.83 (m, 4H). LC-MS (ESI) (M + H)⁺ = 524.1. |
| 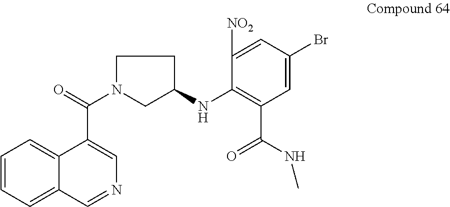 [SMILES: CNC(=O)c1cc(Br)cc([N+](=O)[O-])c1N[C@@H]1CCN(C(=O)c2cncc3ccccc23)C1] Compound 64 | Compound 64: (R)-5-bromo-2-((1- (isoquinoline-4-carbonyl)pyrrolidin-3- yl)amino)-N-methyl-3-nitrobenzamide ¹H NMR (400 MHz, Methanol-d₄) δ 9.32 (d, J = 8.0 Hz, 1H), 8.47 (d, J = 24.0 Hz, 1H), 8.31-8.15 (m, 2H), 7.93-7.78 (m, 3H), 7.77-7.58 (m, 1H), 4.35-4.03 (m, 1H), 3.92-3.81 (m, 2H), 3.53-3.36 (m, 1H), 3.29-3.07 (m, 1H), 2.95-2.75 (m, 3H), 2.38-1.97 (m, 2H). LC-MS (ESI) (M + H)⁺ = 498.1. |
| 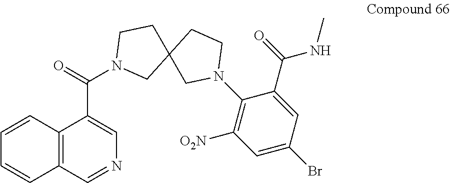 [SMILES: CNC(=O)c1cc(Br)cc([N+](=O)[O-])c1N1CCC2(CCN(C(=O)c3cncc4ccccc34)C2)C1] Compound 66 | Compound 66: 5-bromo-2-(7- (isoquinoline-4-carbonyl)-2,7- diazaspiro[4.4]nonan-2-yl)- N-methyl-3-nitrobenzamide ¹H NMR (400 MHz, Methanol-d₄) δ 9.32 (s, 1H), 8.47 (d, J = 8.0 Hz, 1H), 8.20 (t, J = 16.0, 8.0 Hz, 1H), 7.93-7.78 (m, 4H), 7.63-7.46 (m, 1H), 3.87-3.68 (m, 2H), 3.47-3.36 (m, 1H), 3.27-3.25 (m, 5H), 2.93-2.63 (m, 3H), 2.14-1.84 (m, 4H). LC-MS (ESI) (M + H)⁺ = 538.1. |
| 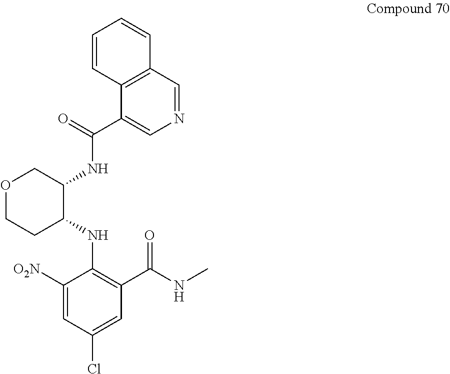 Compound 70 | Compound 70: N-((3R, 4R)-4- ((4-bromo-2-(methylcarbamoyl)-6- nitrophenyl)amino)tetrahydro-2H- pyran-3-yl)isoquinoline-4-carboxamide ¹H NMR (400 MHz, DMSO-d₆) δ 9.38 (s, 1H), 8.85 (d, J = 8.0 Hz, 1H), 8.76 (d, J = 4.0 Hz, 1H), 8.62 (s, 1H), 8.21- 8.13 (m, 3H), 8.04 (d, J = 8.0 Hz, 1H), 7.79 (t, J = 8.0 Hz, 1H), 7.75-7.69 (m, 2H), 4.34 (d, J = 8.0 Hz, 1H), 3.86 (t, J = 12.0 Hz, 2H), 3.77 (d, J = 12.0 Hz, 1H), 3.49 (d, J = 12.0 Hz, 1H), 3.37 (d, J = 12.0 Hz, 1H), 2.76 (d, J = 4.0 Hz, 3H), 2.01 (s, 1H), 1.70 (d, J = 12.0 Hz, 1H). LC-MS (ESI) (M + H)⁺ = 528.1. |
| 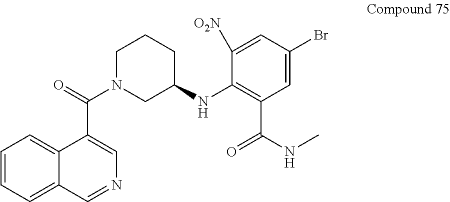 [SMILES: CNC(=O)c1cc(Br)cc([N+](=O)[O-])c1N[C@@H]1CCCN(C(=O)c2cncc3ccccc23)C1] Compound 75 | Compound 75: (R)-5-bromo-2-((1- (isoquinoline-4-carbonyl)piperidin-3- yl)amino)-N-methyl-3-nitrobenzamide ¹H NMR (400 MHz, DMSO-d₆) δ 9.35 (d, J = 27.4 Hz, 1H), 8.91-7.41 (m, 9H), 4.18-3.36 (m, 3H), 3.26-2.96 (m, 2H), 2.88-2.59 (m, 3H), 1.92-1.26 (m, 4H). LC-MS (ESI) (M + H)⁺ = 512.1. |
| 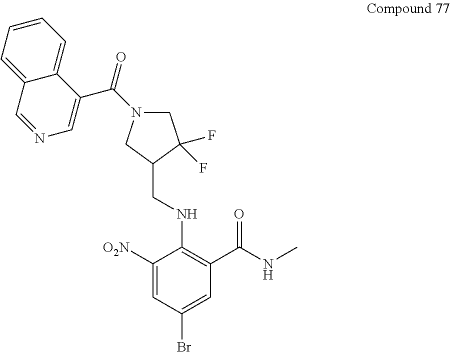 [SMILES: CNC(=O)c1cc(Br)cc([N+](=O)[O-])c1NCC1CN(C(=O)c2cncc3ccccc23)CC1(F)F] Compound 77 | Compound 77: 5-bromo-2-(((4,4- difluoro-1-(isoquinoline-4- carbonyl)pyrrolidin-3-yl)methyl)amino)- N-methyl-3-nitrobenzamide ¹H NMR (400 MHz, DMSO-d₆) δ 9.41 (s, 1H), 8.69 (dd, J = 64.0, 4.0 Hz, 1H), 8.55 (d, J = 2.0 Hz, 1H), 8.27-7.92 (m, 3H), 7.90-7.82 (m, 2H), 7.81-7.67 (m, 2H), 4.16-3.42 (m, 4H), 3.32-3.00 (m, 3H), 2.70 (m, 3H). LC-MS (ESI) (M + H)⁺ = 548.1. |
| 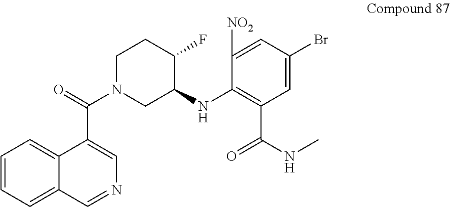 [SMILES: CNC(=O)c1cc(Br)cc([N+](=O)[O-])c1N[C@H]1CN(C(=O)c2cncc3ccccc23)CC[C@@H]1F] Compound 87 | Compound 87: 5-bromo-2-(((3S, 4S)-4-fluoro-1-(isoquinoline-4- carbonyl)piperidin-3-yl)amino)- N-methyl-3-nitrobenzamide ¹H NMR (400 MHz, DMSO-d₆) δ 9.54 (s, 1H), 8.98-8.26 (m, 4H), 8.20-7.64 (m, 5H), 4.98-4.17 (m, 3H), 3.21 (s, 2H), 2.98-2.70 (m, 3H), 2.45-1.37 (m, 3H). LC-MS (ESI) (M + H)⁺ = 530.1. |
| 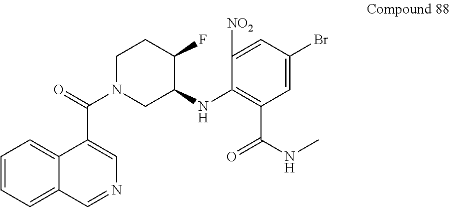 [SMILES: CNC(=O)c1cc(Br)cc([N+](=O)[O-])c1N[C@H]1CN(C(=O)c2cncc3ccccc23)CC[C@H]1F] Compound 88 | Compound 88: 5-bromo-2-(((3S, 4R)- 4-fluoro-1-(isoquinoline-4- carbonyl)piperidin-3-yl)amino)- N-methyl-3-nitrobenzamide ¹H NMR (400 MHz, DMSO-d₆) δ 9.38 (d, J = 20.0 Hz, 1H), 8.98-8.41 (m, 2H), 8.32-8.15 (m, 2H), 8.13-7.63 (m, 5H), 4.97 (dd, J = 50.0, 28.0 Hz, 1H), 4.22 (s, 1H), 3.84 (d, J = 54.0 Hz, 3H), 2.99 (d, J = 112.0 Hz, 3H), 2.73 (s, 1H), 1.94 (d, J = 78.0 Hz, 2H). LC-MS (ESI) (M + H)⁺ = 530.4. |
| 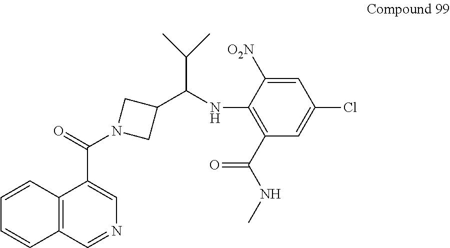 Compound 99 | Compound 99: 5-bromo-2-((1-(1-(isoquinoline- 4-carbonyl)azetidin-3-yl)-2- methylpropyl)amino)-N- methyl-3-nitrobenzamide ¹H NMR (400 MHz, DMSO-d₆) δ 9.36 (d, J = 4.0 Hz, 1H), 8.81 (s, 1H), 8.51 (d, J = 48.0 Hz, 1H), 8.21-8.12 (m, 2H), 8.05 (dd, J = 24.0, 8.0 Hz, 2H), 7.87- 7.78 (m, 1H), 7.74 (s, 1H), 7.68 (s, 1H), 4.25-3.62 (m, 4H), 3.43 (s, 1H), 2.98- 2.91 (m, 1H), 2.74 (s, 3H), 1.76-1.65 (m, 1H), 0.71 (dt, J = 20.0, 8.0 Hz, 6H). LC-MS (ESI) (M + H)⁺ = 540.1. |
| 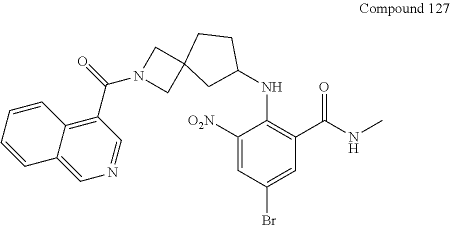 [SMILES: CNC(=O)c1cc(Br)cc([N+](=O)[O-])c1NC1CCC2(C1)CN(C(=O)c1cncc3ccccc13)C2] Compound 127 | Compound 127: 5-bromo-2-((2- (isoquinoline-4-carbonyl)-2-azaspiro[3, 4]octan-6-yl)amino)-N- methyl-3-nitrobenzamide ¹H NMR (400 MHz, Methanol-d₄) δ 9.29 (s, 1H), 8.40 (s, 1H), 8.31 (d, J = 2.0 Hz, 1H), 8.15 (d, J = 8.0 Hz, 1H), 8.10 (d, J = 8.0 Hz, 1H), 7.84 (ddd, J = 8.0, 8.0, 2.0 Hz, 1H), 7.77-7.70 (m, 2H), 4.47-4.26 (m, 2H), 3.99 (s, 1H), 3.00 (q, J = 8.0, 8.0 Hz, 2H), 2.78-2.65 (m, 3H), 1.89-1.47 (m, 6H). LC-MS (ESI) (M + H)⁺ = 538.1. |
| 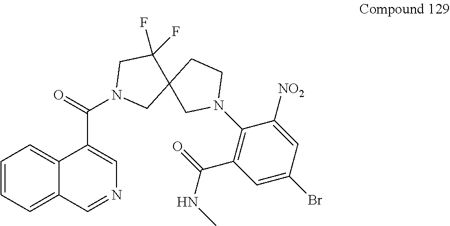 Compound 129 | Compound 129: 5-bromo-2-(9,9-difluoro- 7-(isoquinoline-4-carbonyl)-2,7- diazaspiro[4, 4]nonan-2-yl)- N-methyl-3-nitrobenzamide ¹H NMR (400 MHz, DMSO-d₆) δ 9.36 (d, J = 4.0 Hz, 1H), 8.81 (s, 1H), 8.51 (d, J = 48.0 Hz, 1H), 8.21-8.12 (m, 2H), 8.05 (dd, J = 24.0, 8.0 Hz, 2H), 7.87- 7.78 (m, 1H), 7.74 (s, 1H), 7.68 (s, 1H), 4.25-3.62 (m, 4H), 3.43 (s, 1H), 2.98- 2.91 (m, 1H), 2.74 (s, 3H), 1.76-1.65 (m, 1H), 0.71 (dt, J = 20.0, 8.0 Hz, 6H). LC-MS (ESI) (M + H)⁺ = 540.1. |
| 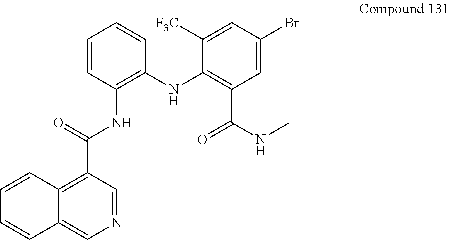 Compound 131 | Compound 131: N-(2-((4-bromo- 2-(methylcarbamoyl)-6- nitrophenyl)amino)phenyl) isoquinoline-4-carboxamide ¹H NMR (400 MHz, DMSO-d₆) δ 10.68 (s, 1H), 9.48 (s, 1H), 9.27 (s, 1H), 8.89 (s, 1H), 8.53 (d, J = 8.0 Hz, 1H), 8.43 (d, J = 4.0 Hz, 1H), 8.30-8.20 (m, 2H), 7.92-7.75 (m, 3H), 7.51 (dd, J = 8.0, 1.8 Hz, 1H), 7.20-7.07 (m, 2H), 6.95 (dd, J = 8.0, 1.7 Hz, 1H), 2.43 (d, J = 4.0 Hz, 3H). LC-MS (ESI) (M + H)⁺ = 520.0. |
| 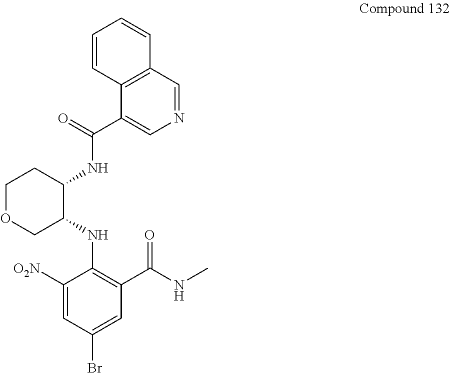 [SMILES: CNC(=O)c1cc(Br)cc([N+](=O)[O-])c1N[C@@H]1COCC[C@@H]1NC(=O)c1cncc2ccccc12] Compound 132 | Compound 132: N-((3S, 4S)-3-((4- bromo-2-(methylcarbamoyl)-6- nitrophenyl)amino)tetrahydro-2H-pyran- 4-yl)isoquinoline-4-carboxamide ¹H NMR (400 MHz, DMSO-d₆) δ 9.34 (d, J = 4.0 Hz, 1H), 8.75-8.44 (m, 3H), 8.27-8.04 (m, 4H), 7.83-7.67 (m, 3H), 4.40-4.28 (m, 1H), 4.02-3.82 (m, 3H), 3.71-3.47 (m, 2H), 2.74 (d, J = 4.0 Hz, 3H), 1.91 (s, 1H), 1.71 (dd, J = 12.0, 4.0 Hz, 1H). LC-MS (ESI) (M + H)⁺ = 528.1. |
| 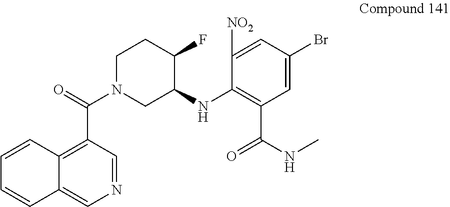 Compound 141 | Compound 141: 5-bromo-2-(((3R, 4R)-1-(isoquinoline-4-carbonyl)-4- methylpiperidin-3-yl)amino)- N-methyl-3-nitrobenzamide ¹H NMR (400 MHz, DMSO-d₆) δ 9.50 (s, 1H), 8.94 (s, 1H), 8.55 (s, 2H), 8.34-8.25 (m, 2H), 7.92 (d, J = 18.0 Hz, 4H), 4.68 (dd, J = 66.0, 14.0 Hz, 2H), 3.11 (d, J = 14.0 Hz, 3H), 2.90 (d, J = 6.0 Hz, 3H), 2.77 (d, J = 18.0 Hz, 4H), 2.13 (s, 2H). LC- MS (ESI) (M + H)⁺ = 526.2. |

### Synthesis of Compounds 12, 15, 28 and 31

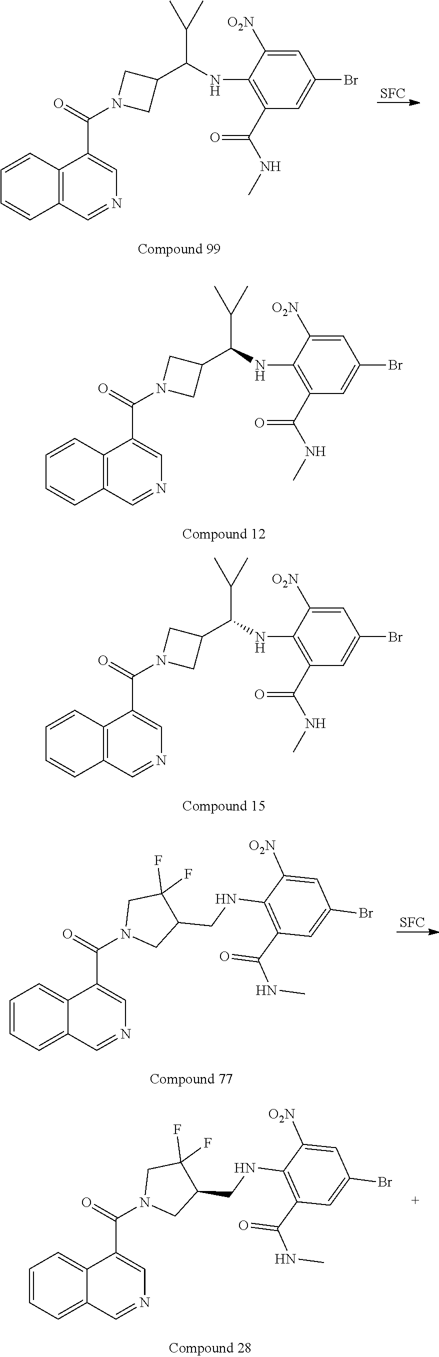

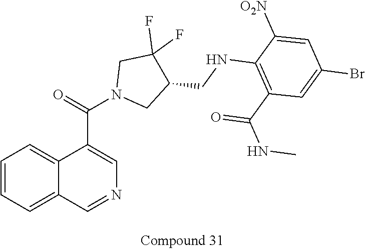 [SMILES: CNC(=O)c1cc(Br)cc([N+](=O)[O-])c1NC[C@@H]1CN(C(=O)c2cncc3ccccc23)CC1(F)F]

Compound 99 or 77 was separated by chiral SFC (column: ChiralPak AD (250 mm*30 mm, 10 μm); mobile phase: [CO2-EtOH]; B %: 15%-15% 1.556; 20 min) to give compound 12 and compound 15 or compound 28 and compound 31 as yellow solids.

| | |
|---|---|
| 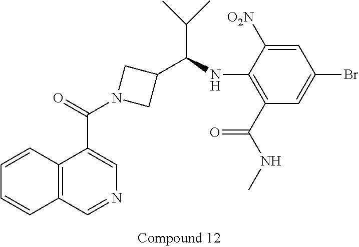 [SMILES: CNC(=O)c1cc(Br)cc([N+](=O)[O-])c1N[C@H](C(C)C)C1CN(C(=O)c2cncc3ccccc23)C1] | Compound 15: (S)-5-bromo-2-((1-(1-(isoquinoline-4-carbonyl)azetidin-3-yl)- 2-methylpropyl)amino)-N-methyl-3-nitrobenzamide ¹H NMR (400 MHz, DMSO-d₆) δ 9.37 (d, J = 5.4 Hz, 1H), 8.81 (d, J = 5.2 Hz, 1H), 8.52 (d, J = 47.9 Hz, 1H), 8.22-7.99 (m, 4H), 7.88-7.72 (m, 2H), 7.69 (s, 1H), 4.38-3.96 (m, 3H), 3.95-3.64 (m, 1H), 3.58-3.35 (m, 1H), 2.96 (s, 1H), 2.75 (t, J = 4.4 Hz, 3H), 1.70 (s, 1H), 0.72 (dt, J = 20.1, 6.6 Hz, 6H). LC-MS (ESI) (M + H)⁺ = 540.1. |
| 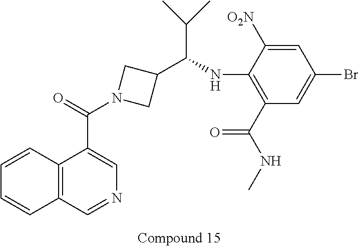 [SMILES: CNC(=O)c1cc(Br)cc([N+](=O)[O-])c1N[C@@H](C(C)C)C1CN(C(=O)c2cncc3ccccc23)C1] | Compound 15: (R)-5-bromo-2-((1-(1-(isoquinoline-4-carbonyl)azetidin-3-yl)- 2-methylpropyl)amino)-N-methyl-3-nitrobenzamide ¹H NMR (400 MHz, DMSO-d₆) δ 9.38 (d, J = 5.4 Hz, 1H), 8.81 (d, J = 5.2 Hz, 1H), 8.52 (d, J = 47.6 Hz, 1H), 8.22-7.99 (m, 4H), 7.88-7.72 (m, 2H), 7.69 (s, 1H), 4.25-3.95 (m, 3H), 3.89-3.44 (m, 2H), 2.97 (d, J = 8.7 Hz, 1H), 2.74 (d, J = 4.4 Hz, 3H), 1.71 (s, 1H), 0.72 (dt, J = 20.2, 6.4 Hz, 6H). LC-MS (ESI) (M + H)⁺ = 540.1. |
| 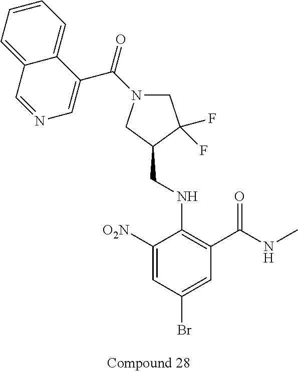 [SMILES: CNC(=O)c1cc(Br)cc([N+](=O)[O-])c1NC[C@H]1CN(C(=O)c2cncc3ccccc23)CC1(F)F] | Compound 28: (S)-5-bromo-2-(((4,4-difluoro-1-(isoquinoline-4- carbonyl)pyrrolidin-3-yl)methyl)amino)-N-methyl-3-nitrobenzamide ¹H NMR (400 MHz, DMSO-d₆) δ 9.41 (s, 1H), 8.69 (dd, J = 64.0, 6.0 Hz, 1H), 8.55 (d, J = 4.0 Hz, 1H), 8.27-7.92 (m, 3H), 7.90-7.82 (m, 2H), 7.81-7.67 (m, 2H), 4.16-3.42 (m, 4H), 3.32-3.00 (m, 3H), 2.70 (dd, J = 72.0, 6.0 Hz, 3H). LC-MS (ESI) (M + H)⁺ = 548.1. |
| 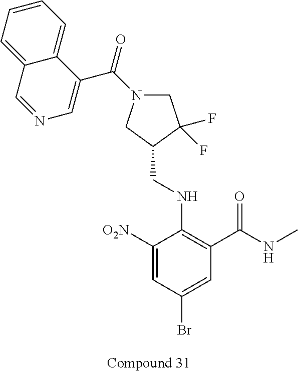 [SMILES: CNC(=O)c1cc(Br)cc([N+](=O)[O-])c1NC[C@@H]1CN(C(=O)c2cncc3ccccc23)CC1(F)F] | Compound 31: (R)-5-bromo-2-(((4,4-difluoro-1-(isoquinoline-4- carbonyl)pyrrolidin-3-yl)methyl)amino)-N-methyl-3-nitrobenzamide ¹H NMR (400 MHz, DMSO-d₆) δ 9.41 (s, 1H), 8.69 (dd, J = 64.0, 4.4 Hz, 1H), 8.55 (d, J = 2.4 Hz, 1H), 8.25-7.93 (m, 3H), 7.89-7.82 (m, 2H), 7.80-7.69 (m, 2H), 4.16-3.42 (m, 4H), 3.32-3.03 (m, 3H), 2.70 (dd, J = 72.0, 4.4 Hz, 3H). LC-MS (ESI) (M + H)⁺ = 548.2. |

### Synthesis of Compound 21

The synthesis route of compound 21 is similar to that of compound 10.

| | |
|---|---|
| 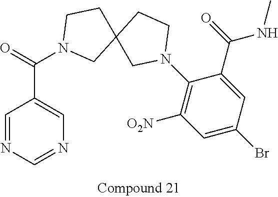 [SMILES: CNC(=O)c1cc(Br)cc([N+](=O)[O-])c1N1CCC2(CCN(C(=O)c3cncnc3)C2)C1] | Compound 21: 5-bromo-N-methyl-3-nitro-2-(7-(pyrimidine-5-carbonyl)-2,7- diazaspiro[4. 4]nonan-2-yl)benzamide ¹H NMR (400 MHz, DMSO-d₆) δ 9.27 (d, J = 2.0 Hz, 1H), 8.98 (d, J = 8.0 Hz, 2H), 8.62-8.43 (m, 1H), 7.99 (dd, J = 16.0, 2.0 Hz, 1H), 7.54 (dd, J = 12.0, 2.0 Hz, 1H), 3.63-3.43 (m, 4H), 3.32-3.15 (m, 2H), 3.07 (m, 2H), 2.71 (m, 3H), 2.05-1.83 (m, 4H). LC-MS (ESI) (M + H)⁺ = 489.1. |

### Synthesis of Compounds 11, 25, 67, 74 and 138

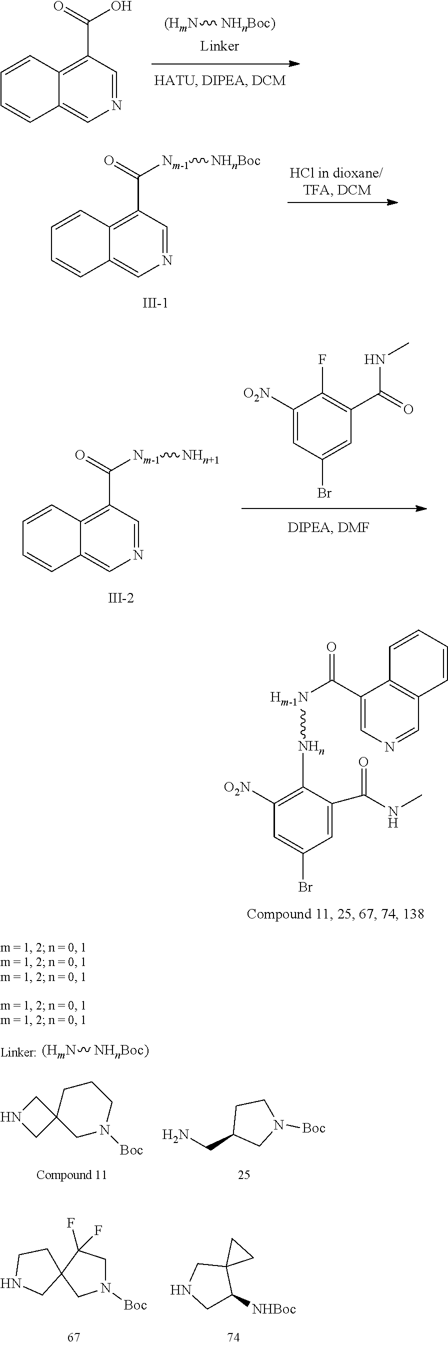

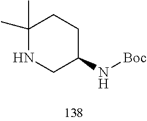 [SMILES: CC1(C)CC[C@@H](NC(=O)OC(C)(C)C)CN1]

### Intermediate III-1

To a solution of corresponding linker (1.0 equiv), isoquinoline-4-carboxylic acid (1.2 equiv) and DIPEA (3.0 equiv) in DMF (1 mL), HATU (1.1 equiv) was added and the reaction was stirred at room temperature for 12 hr. The reaction was diluted with water (5 mL) and extracted with EtOAc (3*10 mL). The organic layer was separated, washed with water and further saturated NaCl solution (3*10 mL), and dried over Na₂SO₄, and then concentrated in vacuo. The residue was purified using silica gel column chromatography eluting with ethyl acetate in petroleum ether to afford intermediate III-1.

### Intermediate 111-2

To a solution of intermediate III-1 in DCM (2 mL), HCl in dioxane or TFA was added and the reaction was stirred at room temperature for 2 hr. The reaction was concentrated under reduced pressure to give intermediate III-2 without further purification.

### Compounds 11, 25, 67, 74 and 138

To a solution of intermediates III-2 (1.0 equiv) and 5-bromo-2-fluoro-N-methyl-3-nitrobenzamide (1.2 equiv) in DMF (2 mL), DIPEA (3.0 equiv) was added and the reaction as stirred at 80 (C for 12 hr. The reaction was diluted with water (5 mL) and extracted with EtOAc (3*10 mL). The organic layer was separated, washed with water and further saturated NaCl solution (3*10 mL), and dried over Na₂SO₄, and then concentrated in vacuo. The residue was purified using silica gel column chromatography eluting with ethyl acetate in petroleum ether to afford the target compounds as yellow solids.

| | |
|---|---|
| 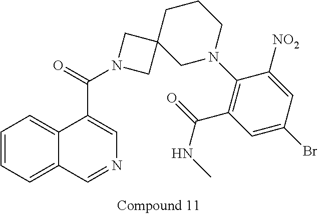 [SMILES: CNC(=O)c1cc(Br)cc([N+](=O)[O-])c1N1CCCC2(CN(C(=O)c3cncc4ccccc34)C2)C1] | Compound 11: (R)-5-bromo-2-((1-(isoquinoline-4-carbonyl)piperidin-3- yl)amino)-N-methyl-3-nitrobenzamide ¹H NMR (400 MHz, Methanol-d₄) δ 9.33 (s, 1H), 8.52 (s, 1H), 8.19 (d, J = 8.0 Hz, 1H), 8.08 (d, J = 8.0 Hz, 1H), 7.97-7.84 (m, 2H), 7.77 (t, J = 8.0 Hz, 1H), 7.67 (d, J = 4.0 Hz, 1H), 3.97-3.90 (m, 2H), 3.74-3.64 (m, 2H), 3.11-2.96 (m, 4H), 2.79 (s, 3H), 1.77 (t, J = 8.0 Hz, 2H), 1.64-1.46 (m, 2H). LC-MS (ESI) (M + H)⁺ = 538.1. |
| 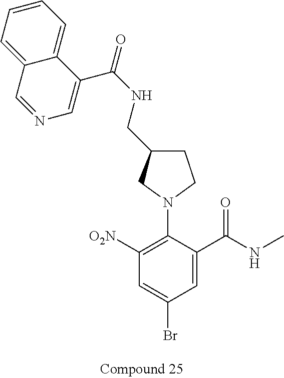 [SMILES: CNC(=O)c1cc(Br)cc([N+](=O)[O-])c1N1CC[C@H](CNC(=O)c2cncc3ccccc23)C1] | Compound 25: (R)-N-((1-(4-bromo-2-(methylcarbamoyl)-6- nitrophenyl)pyrrolidin-3-yl)methyl)isoquinoline-4-carboxamide ¹H NMR (400 MHz, DMSO-d₆) δ 9.40 (s, 1H), 8.86 (t, J = 6.0 Hz, 1H), 8.56 (d, J = 12.0 Hz, 2H), 8.20 (t, J = 8.0 Hz, 2H), 7.98 (d, J = 2.0 Hz, 1H), 7.80 (m, 2H), 7.52 (d, J = 2.0 Hz, 1H), 3.38 (dd, J = 12.0, 6.0 Hz, 2H), 3.29 (m, 4H), 3.04 (dd, J = 10.0, 8.0 Hz, 1H), 2.74 (d, J = 4.0 Hz, 3H), 2.11-1.98 (m, 1H), 1.74 (m, 1H). LC-MS (ESI) (M + H)⁺ = 512.1. |
| 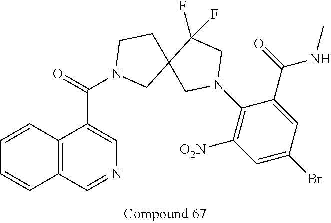 [SMILES: CNC(=O)c1cc(Br)cc([N+](=O)[O-])c1N1CC(F)(F)C2(CCN(C(=O)c3cncc4ccccc34)C2)C1] | Compound 67: 5-bromo-2-(4,4-difluoro-7-(isoquinoline-4-carbonyl)-2,7- diazaspiro[4.4]nonan-2-yl)-N-methyl-3-nitrobenzamide ¹H NMR (400 MHz, DMSO-d₆) δ 9.39 (d, J = 4.0 Hz, 1H), 8.69-8.37 (m, 2H), 8.22 (d, J = 8.0 Hz, 1H), 8.20 (dd, J = 36.0, 8.0 Hz, 1H), 7.89-7.80 (m, 2H), 7.79-7.67 (m, 2H), 3.96-3.34 (m, 6H), 3.29-3.14 (m, 2H), 2.80 (d, J = 4.0 Hz, 1H), 2.42 (d, J = 4.0 Hz, 2H), 2.31-1.90 (m, 2H). LC-MS (ESI) (M + H)⁺= 574.0. |
| 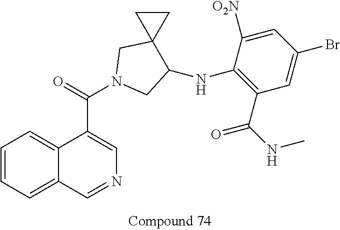 [SMILES: CNC(=O)c1cc(Br)cc([N+](=O)[O-])c1NC1CN(C(=O)c2cncc3ccccc23)CC12CC2] | Compound 74: (R)-5-bromo-2-((5-(isoquinoline-4-carbonyl)-5-azaspiro[2,4] heptan-7-yl)amino)-N-methyl-3-nitrobenzamide ¹H NMR (400 MHz, DMSO-d₆) δ 9.37 (d, J = 8.0 Hz, 1H), 8.79 (dd, J = 84.0, 4.0 Hz, 1H), 8.52-8.35 (m, 2H), 8.27-7.81 (m, 3H), 7.79-7.52 (m, 3H), 3.92 (dd, J = 40.0, 12.0 Hz, 1H), 3.56-3.42 (m, 2H), 3.27 (dd, J = 8.0, 4.0 Hz, 1H), 2.97 (dd, J = 12.0, 8.0 Hz, 1H), 2.73 (dd, J = 40.0, 4.0 Hz, 3H), 0.84-0.54 (m, 4H). LC-MS (ESI) (M + H)⁺ = 524.1. |
| 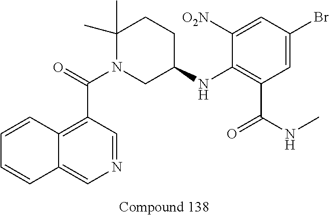 [SMILES: CNC(=O)c1cc(Br)cc([N+](=O)[O-])c1N[C@@H]1CCC(C)(C)N(C(=O)c2cncc3ccccc23)C1] | Compound 138: (R)-5-bromo-2-((1-(isoquinoline-4-carbonyl)-6,6- dimethylpiperidin-3-yl)amino)-N-methyl-3-nitrobenzamide ¹H NMR (400 MHz, DMSO-d₆) δ 9.27 (s, 1H), 8.67 (s, 1H), 8.31 (s, 1H), 8.16 (s, 1H), 8.05-7.42 (m, 6H), 3.27-3.08 (m, 2H), 2.79-2.69 (m, 3H), 1.94 (s, 1H), 1.61 (d, J = 34.0 Hz, 10H). LC-MS (ESI) (M + H)⁺ = 540.1. |

### Synthesis of Compounds 14, 16, 51, 52, 53, 54, 56, 59, 60, 61, 68 and 85

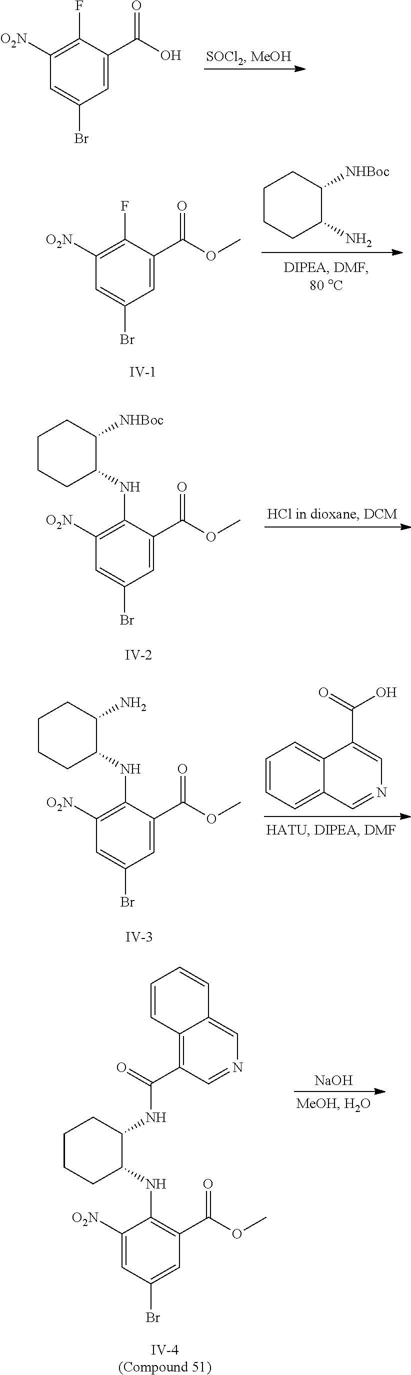

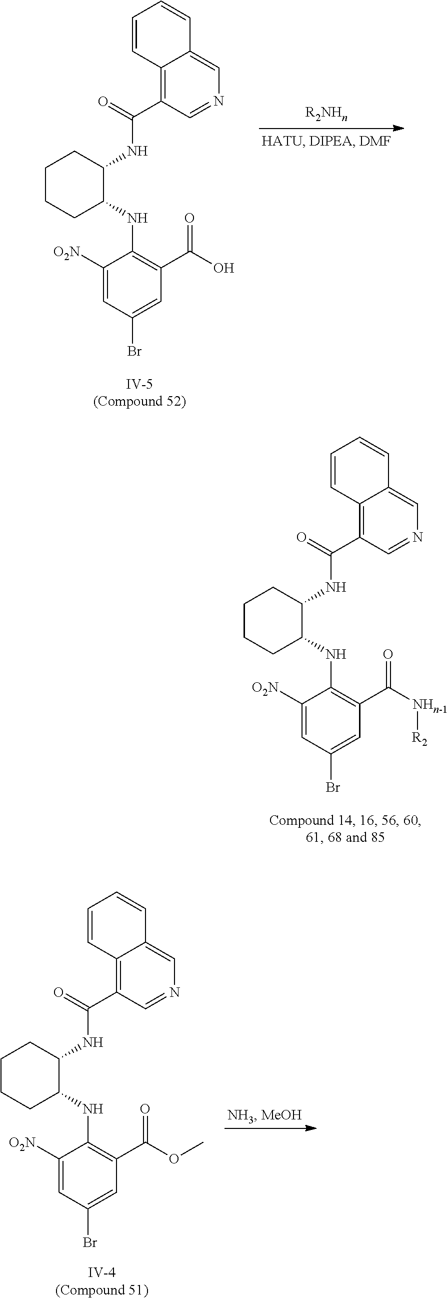

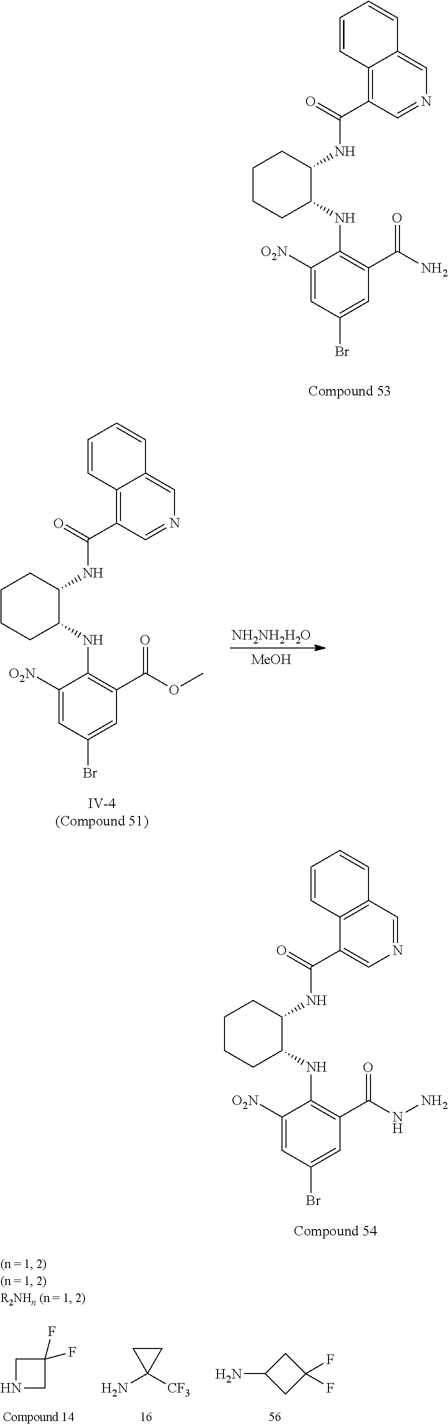

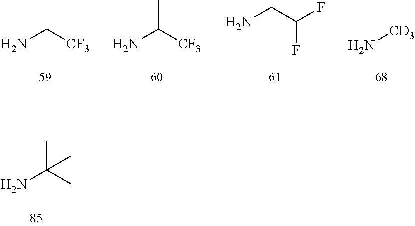

### Intermediate IV-1: methyl 5-bromo-2-fluoro-3-nitrobenzoate

Thionyl chloride (0.4 mL, 5.68 mmol) was added to a solution of 5-bromo-2-fluoro-3-nitrobenzoic acid (500 mg, 1.89 mmol) in MeOH (5.0 mL), and the reaction was stirred at room temperature for 16 hr. The reaction was concentrated in vacuo to get a solid, which was triturated with PE to afford the title compound (450 mg, 85% yield) as a yellow solid.

LC-MS (ESI) (M+H)⁺=278.0.

### Intermediate IV-2: methyl 5-bromo-2-(((1R,2S)-2-((tert-butoxycarbonyl)amino)cyclohexyl)amino)-3-nitrobenzoate

To a solution of methyl 5-bromo-2-fluoro-3-nitrobenzoate (50 mg, 0.18 mmol) in DMF (2.0 mL), tert-butyl ((1S,2R)-2-aminocyclohexyl)carbamate (58 mg, 0.27 mmol) and DIPEA (0.1 mL, 0.54 mmol) were added and the reaction was stirred at 80° C. for 16 hr. The reaction was diluted with water (50.0 mL) and extracted with EtOAc (3*50.0 mL). The organic layer was separated, washed with water and further saturated NaCl solution (3*30.0 mL), and dried over Na₂SO₄, and then concentrated in vacuo. The residue was purified using silica gel column chromatography eluting with ethyl acetate in petroleum ether (EtOAc:PE=1:2) to afford the title compound (75 mg, 88% yield) as a yellow solid.

LC-MS (ESI) (M+H)⁺=472.1.

### Intermediate IV-3: methyl 2-(((1R,2S)-2-aminocyclohexyl)amino)-5-bromo-3-nitrobenzoate

To a solution of methyl 5-bromo-2-(((1R,2S)-2-((tert-butoxycarbonyl)amino)cyclohexyl)amino)-3-nitrobenzoate (75 mg, 0.16 mmol) in DCM (2.0 mL), 4N HCl in dioxane (4.0 mL, 4.0 mol/L) was added and the reaction was stirred at room temperature for 2 hr. The reaction was dried in vacuo to afford the title compound (80 mg, crude) as a yellow solid.

LC-MS (ESI) (M+H)⁺=372.1.

### Intermediate IV-4 (compound 51): methyl 5-bromo-2-(((1R,2S)-2-(isoquinoline-4-carboxamido)cyclohexyl)amino)-3-nitrobenzoate

To a solution of methyl 2-(((1R,2S)-2-aminocyclohexyl)amino)-5-bromo-3-nitrobenzoate (80 mg, Crude) in DMF (2.0 mL), isoquinoline-4-carboxylic acid (118 mg, 0.68 mmol), DIPEA (0.3 mL, 1.71 mmol) and HATU (194 mg, 0.51 mmol) were added and the reaction was stirred at room temperature for 16 hr. The reaction was diluted with water (50.0 mL) and extracted with EtOAc (3*20.0 mL). The combined organic layer was washed with NH₄Cl solution (20.0 mL), NaHCO₃ solution (20.0 mL), water (20.0 mL) and further saturated NaCl solution (20.0 mL), and it was dried over Na₂SO₄, and then concentrated in vacuo. The residue was purified using silica gel column chromatography eluting with ethyl acetate in petroleum ether (EtOAc:PE=1:2) to afford the title compound (100 mg, 88% yield, 2 steps) as a yellow solid.

### Intermediate IV-5 (compounds 52): 5-bromo-2-(((1R,2S)-2-(isoquinoline-4-carboxamido)cyclohexyl)amino)-3-nitrobenzoic acid

To a solution of methyl 5-bromo-2-(((1R,2S)-2-(isoquinoline-4-carboxamido)cyclohexyl)amino)-3-nitrobenzoate (100 mg, 0.19 mmol) in MeOH/H₂O (v/v=5/1, 6 mL), NaOH (82 mg, 2.05 mmol) was added and the reaction was stirred at 55° C. for 16 hr. The reaction mixture was quenched with 1N HCl to pH 3 and then extracted with EtOAc (50 mL). The EtOAc layer was dried over Na₂SO₄and dried in vacuo to afford the title compound (71 mg, 73% yield) as a yellow solid.

### Compounds 14, 16, 56, 59, 60, 61, 68 and 85

To a solution of 5-bromo-2-(((1R,2S)-2-(isoquinoline-4-carboxamido)cyclohexyl)amino)-3-nitrobenzoic acid (1.0 equiv) in DMF (2.5 mL), corresponding amine (1.2 equiv), HATU (1.5 equiv) and DIPEA (3.0 equiv) were added and the reaction was stirred at room temperature overnight. The reaction was diluted with water (50.0 mL) and extracted with EtOAc (3*20.0 mL). The organic layer was separated, washed with water, NH₄Cl solution (20 mL), NaHCO₃ solution (20 mL) and further saturated NaCl solution (20 mL), and it was dried over Na₂SO₄ and concentrated in vacuo. The residue was purified using prep-TLC with ethyl acetate in petroleum ether and Prep-HPLC to give the target compounds as yellow solids.

### Compound 53

Compound 51 (53 mg, 0.1 mmol) was added to 7 N NH₃ in method (5.0 mL) and the reaction was stirred at 55° C. overnight under Ar atmosphere. The reaction was concentrated in vacuo and then purified using silica gel column chromatography eluting with methanol in DCM to give compound 53 (17 mg, 33.3% yield) as a yellow solid.

### Compound 54

Hydrazine monohydrate (2.0 mL) was added to a solution of compound 51 (100 mg, 0.19 mmol) in methanol (10.0 mL). The solution was stirred at room temperature for 4 hr. The solvent was removed under vacuum. The residue was resuspended with EtOAc and washed with NaHCO₃ solution and further saturated NaCl solution. The organic layer dried over Na₂SO₄ was concentrated in vacuo and purified by silica gel column chromatography eluting with methanol in DCM to give compound 54 (40 mg, 40% yield) as a yellow solid.

| | |
|---|---|
| 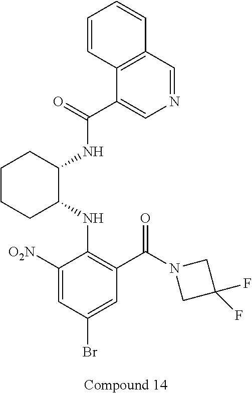 [SMILES: O=C(N[C@H]1CCCC[C@H]1Nc1c(C(=O)N2CC(F)(F)C2)cc(Br)cc1[N+](=O)[O-])c1cncc2ccccc12] | Compound 14: N-((1S, 2R)-2-((4-bromo-2-(3,3-difluoroazetidine-1-carbonyl)-6- nitrophenyl)amino)cyclohexyl)isoquinoline-4-carboxamide ¹H NMR (400 MHz, Methanol-d₄) δ 9.28 (s, 1H), 8.42 (s, 1H), 8.34 (d, J = 2.5 Hz, 1H), 8.15 (d, J = 8.2 Hz, 1H), 8.05 (d, J = 8.5 Hz, 1H), 7.87-7.81 (m, 1H), 7.80-7.70 (m, 2H), 4.57 (t, J = 11.6 Hz, 4H), 4.42 (s, 1H), 4.01 (s, 1H), 1.95-1.48 (m, 8H). LC-MS (ESI) (M + H)⁺ = 588.1. |
| 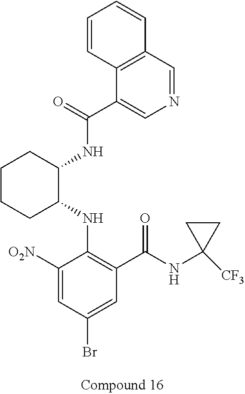 [SMILES: O=C(NC1(C(F)(F)F)CC1)c1cc(Br)cc([N+](=O)[O-])c1N[C@@H]1CCCC[C@@H]1NC(=O)c1cncc2ccccc12] | Compound 16: N-((1S, 2R)-2-((4-bromo-2-nitro-6-((1- (trifluoromethyl)cyclopropyl)carbamoyl)phenyl)amino)cyclohexyl)isoquinoline- 4-carboxamide ¹H NMR (400 MHz, Methanol-d₄) δ 9.29 (s, 1H), 8.43 (s, 1H), 8.35 (t, J = 2.0 Hz, 1H), 8.15 (d, J = 8.0 Hz, 1H), 8.08 (d, J = 8.0 Hz, 1H), 7.84 (t, J = 8.0 Hz, 1H), 7.74 (t, J = 8.0 Hz, 1H), 7.62 (d, J = 2.0 Hz, 1H), 4.43 (s, 1H), 4.01 (s, 1H), 1.85-1.26 (m, 12H). LC-MS (ESI) (M + H)+ = 620.1. |
| 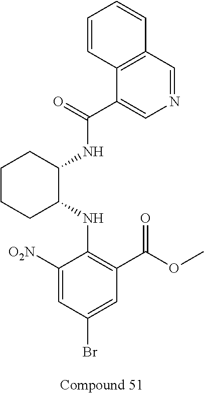 [SMILES: COC(=O)c1cc(Br)cc([N+](=O)[O-])c1N[C@@H]1CCCC[C@@H]1NC(=O)c1cncc2ccccc12] | Compound 51: methyl 5-bromo-2-(((1R, 2S)-2-(isoquinoline-4- carboxamido)cyclohexyl)amino)-3-nitrobenzoate ¹H NMR (500 MHz, CDCl₃): δ 9.25 (s, 1H), 8.63 (s, 1H), 8.47 (d, J = 10.5 Hz, 1H), 8.30 (d, J = 8.5 Hz, 1H), 8.10 (d, J = 2.4 Hz, 1H), 7.98 (m, 2H), 7.78 (t, J = 7.7 Hz, 1H), 7.66 (t, J = 7.5 Hz, 1H), 6.42 (d, J = 8.1 Hz, 1H), 4.37 (m, 1H), 3.82 (s, 3H), 3.71 (m, 1H), 1.81-1.46 (m, 8H). LC-MS (ESI) (M + H)⁺ = 527.29. |
| 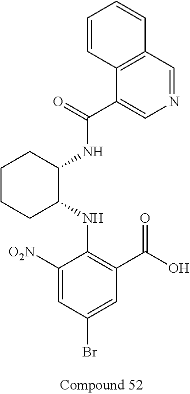 [SMILES: O=C(O)c1cc(Br)cc([N+](=O)[O-])c1N[C@@H]1CCCC[C@@H]1NC(=O)c1cncc2ccccc12] | Compound 52: 5-bromo-2-(((1R, 2S)-2-(isoquinoline-4- carboxamido)cyclohexyl)amino)-3-nitrobenzoic acid 1H NMR (500 MHz, CDCl₃): δ 9.04 (s, 1H), 8.71 (s, 1H), 8.34 (d, J = 8.0 Hz, 1H), 8.08 (s, 1H), 7.87 (m, 3H), 7.66 (t, J = 7.5 Hz, 1H), 7.43 (s, 1H), 4.41 (s, 1H), 3.70 (s, 1H), 1.87-1.52 (m, 8H). LC-MS (ESI) (M + H)⁺ = 518.28. |
| 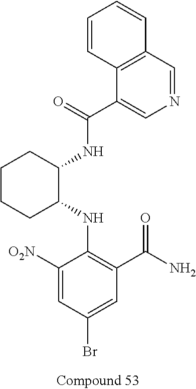 [SMILES: NC(=O)c1cc(Br)cc([N+](=O)[O-])c1N[C@@H]1CCCC[C@@H]1NC(=O)c1cncc2ccccc12] | Compound 53: N-((1S,2R)-2-((4-bromo-2-carbamoyl-6- nitrophenyl)amino)cyclohexyl)isoquinoline-4-carboxamide 1H NMR (500 MHz, CDCl₃): δ 9.16 (s, 1H), 8.48 (s, 1H), 8.21 (d, J = 2.1 Hz, 1H), 8.17 (d, J = 8.5 Hz, 1H), 7.93 (d, J = 8.1 Hz, 1H), 7.89-7.82 (m, 1H), 7.80- 7.72 (m, 2H), 7.65 (t, J = 7.5 Hz, 1H), 6.97 (s, 1H), 6.71 (d, J = 7.6 Hz, 1H), 6.09 (s, 1H), 4.46 (s, 1H), 4.02 (s, 1H), 1.95-1.45 (m, 8H). LC-MS (ESI) (M + H)⁺ = 512.24. |
| 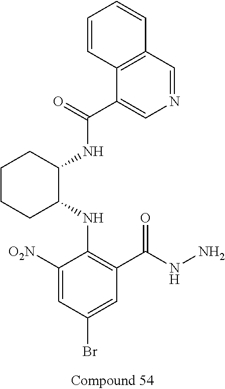 [SMILES: NNC(=O)c1cc(Br)cc([N+](=O)[O-])c1N[C@@H]1CCCC[C@@H]1NC(=O)c1cncc2ccccc12] | Compound 54: N-((1S,2R)-2-((4-bromo-2-(hydrazinecarbonyl)-6- nitrophenyl)amino)cyclohexyl)isoquinoline-4-carboxamide ¹H NMR (500 MHz, CDCl₃) δ 9.11 (s, 1H), 8.47 (s, 1H), 8.22 (d, J = 2.3 Hz, 1H), 8.13 (d, J = 8.5 Hz, 1H), 7.90 (d, J = 8.1 Hz, 2H), 7.80-7.57 (m, 4H), 6.78 (d, J = 6.6 Hz, 1H), 5.47-5.28 (m, 2H), 4.35 (s, 1H), 3.98 (s, 1H), 2.11-1.45 (m, 8H). LC-MS (ESI) (M + H)⁺ = 527.30. |
| 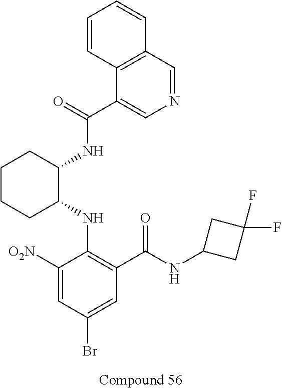 [SMILES: O=C(NC1CC(F)(F)C1)c1cc(Br)cc([N+](=O)[O-])c1N[C@@H]1CCCC[C@@H]1NC(=O)c1cncc2ccccc12] | Compound 56: N-((1S, 2R)-2-((4-bromo-2-((3,3-difluorocyclobutyl)carbamoyl)- 6-nitrophenyl)amino)cyclohexyl)isoquinoline-4-carboxamide ₁H NMR (400 MHz, CDCl₃) δ 9.29 (s, 1H), 8.40 (s, 1H), 8.31 (d, J = 2.0 Hz, 1H), 8.15 (d, J = 8.0 Hz, 1H), 8.10 (d, J = 8.0 Hz, 1H), 7.84 (ddd, J = 8.0, 8.0, 2.0 Hz, 1H), 7.77-7.70 (m, 2H), 4.47-4.26 (m, 2H), 3.99 (s, 1H), 3.00 (q, J = 8.0, 8.0 Hz, 2H), 2.78-2.65 (m, 2H), 1.89-1.47 (m, 8H). LC-MS (ESI) (M + H)⁺ = 602.1. |
| 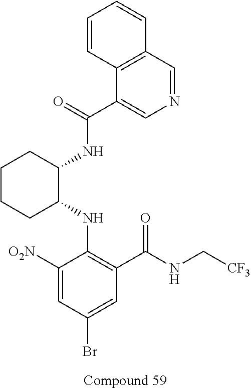 [SMILES: O=C(NCC(F)(F)F)c1cc(Br)cc([N+](=O)[O-])c1N[C@@H]1CCCC[C@@H]1NC(=O)c1cncc2ccccc12] | Compound 59: N-((1S, 2R)-2-((4-bromo-2-nitro-6-((2,2,2- trifluoroethyl)carbamoyl)phenyl)amino)cyclohexyl)isoquinoline-4-carboxamide ¹H NMR (400 MHz, Methanol-d₄) δ 9.29 (s, 1H), 8.46 (s, 1H), 8.33 (d, J = 2.0 Hz, 1H), 8.15 (d, J = 8.0 Hz, 1H), 8.09 (d, J = 8.0 Hz, 1H), 7.85 (ddd, J = 8.0, 8.0, 2.0 Hz, 1H), 7.74 (t, J = 8.0 Hz, 1H), 7.70 (d, J = 2.0 Hz, 1H), 4.80 (s, 2H), 4.43 (s, 1H), 4.10 (tt, J = 16.0, 8.0 Hz, 2H), 3.99 (d, J = 4.0 Hz, 1H), 1.93-1.46 (m, 9H).LC-MS (ESI) (M + H)⁺ = 594.1. |
| 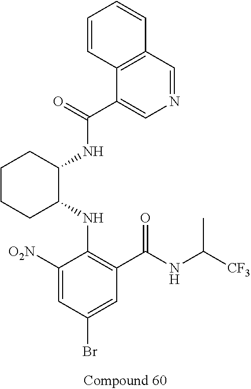 [SMILES: CC(NC(=O)c1cc(Br)cc([N+](=O)[O-])c1N[C@@H]1CCCC[C@@H]1NC(=O)c1cncc2ccccc12)C(F)(F)F] | Compound 60: N-((1S, 2R)-2-((4-bromo-2-nitro-6-((1,1,1-trifluoropropan-2- yl)carbamoyl)phenyl)amino)cyclohexyl)isoquinoline-4-carboxamide ¹H NMR (400 MHz, Methanol-d₄) δ 9.29 (s, 1H), 8.48 (d, J = 12.0 Hz, 1H), 8.35 (dd, J = 8.0, 4.0 Hz, 1H), 8.16 (d, J = 8.0 Hz, 1H), 8.10 (t, J = 8.0 Hz, 1H), 7.84 (t, J = 8.0 Hz, 1H), 7.74 (t, J = 8.0 Hz, 1H), 7.67-7.60 (m, 1H), 4.83-4.79 (m, 1H), 4.45 (s, 1H), 4.01 (s, 1H), 2.10-1.09 (m, 14H). LC-MS (ESI) (M + H)⁺ = 608.1. |
| 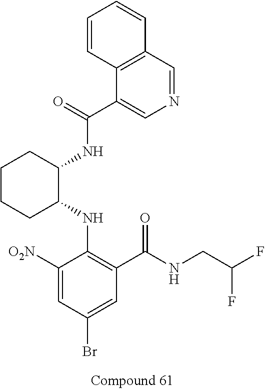 [SMILES: O=C(NCC(F)F)c1cc(Br)cc([N+](=O)[O-])c1N[C@@H]1CCCC[C@@H]1NC(=O)c1cncc2ccccc12] | Compound 61: N-((IS, 2R)-2-((4-bromo-2-((2,2-difluoroethyl)carbamoyl)-6- nitrophenyl)amino)cyclohexyl)isoquinoline-4-carboxamide ¹H NMR (400 MHz, Methanol-d₄) δ 9.29 (s, 1H), 8.46 (s, 1H), 8.31 (s, 1H), 8.15 (d, J = 8.0 Hz, 1H), 8.11 (d, J = 8.0 Hz, 1H), 7.85 (s, 1H), 7.77-7.69 (m, 2H), 6.28-5.92 (m, 1H), 4.45-4.40 (m, 1H), 4.03 (dd, J = 8.0, 2.0 Hz, 1H), 3.82- 3.72 (m, 2H), 1.87-1.53 (m, 8H). LC-MS (ESI) (M + H)⁺ = 576.1. |
| 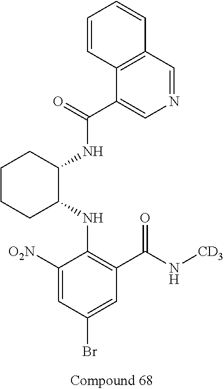 [SMILES: [2H]C([2H])([2H])NC(=O)c1cc(Br)cc([N+](=O)[O-])c1N[C@@H]1CCCC[C@@H]1NC(=O)c1cncc2ccccc12] | Compound 68: N-((1S, 2R)-2-((4-bromo-2-((methyl-d3)carbamoyl)-6- nitrophenyl)amino)cyclohexyl)isoquinoline-4-carboxamide ¹H NMR (400 MHz, Methanol-d₄) δ 9.29 (s, 1H), 8.43 (s, 1H), 8.28 (d, J = 2.0 Hz, 1H), 8.15 (d, J = 8.0 Hz, 1H), 8.09 (d, J = 8.0 Hz, 1H), 7.84 (ddd, J = 8.0, 7.0, 4.0 Hz, 1H), 7.78-7.71 (m, 1H), 7.67 (d, J = 2.0 Hz, 1H), 4.41 (s, 1H), 3.97 (s, 1H), 1.95-1.44 (m, 8H). LC-MS (ESI) (M + H)⁺ = 529.1. |
| 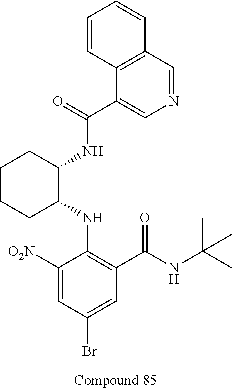 [SMILES: CC(C)(C)NC(=O)c1cc(Br)cc([N+](=O)[O-])c1N[C@@H]1CCCC[C@@H]1NC(=O)c1cncc2ccccc12] | Compound 85: N-((1S, 2R)-2-((4-bromo-2-(tert-butylcarbamoyl)-6- nitrophenyl)amino)cyclohexyl)isoquinoline-4-carboxamide ¹H NMR (400 MHz, Methanol-d₄) δ 9.29 (s, 1H), 8.46 (s, 1H), 8.32 (d, J = 2.0 Hz, 1H), 8.16 (d, J = 8.0 Hz, 1H), 8.09 (d, J = 8.0 Hz, 1H), 7.84 (t, J = 8.0 Hz, 1H), 7.74 (t, J = 8.0 Hz, 1H), 7.58 (d, J = 2.0 Hz, 1H), 4.59 (s, 1H), 4.46 (s, 1H), 4.17 (s, 1H), 1.50 (s, 19H). LC-MS (ESI) (M + H)⁺ = 568.1. |

### Synthesis of Compounds 27, 29 and 78

The synthesis route of compound 27 and 78 is similar to that of compound 14.

| | |
|---|---|
| 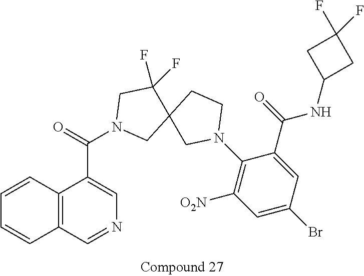 [SMILES: O=C(NC1CC(F)(F)C1)c1cc(Br)cc([N+](=O)[O-])c1N1CCC2(CN(C(=O)c3cncc4ccccc34)CC2(F)F)C1] | Compound 27: 5-bromo-2-(9,9-difluoro-7-(isoquinoline-4- carbonyl)-2,7-diazaspiro[4. 4]nonan-2-yl)-N-(3,3- difluorocyclobutyl)-3-nitrobenzamide ¹H NMR (400 MHz, DMSO-d₆) δ 9.42 (s, 1H), 9.08 (m, 1H), 8.60 (d, J = 16.0 Hz, 1H), 8.23 (d, J = 8.0 Hz, 1H), 8.08 (m, 1H), 7.91-7.84 (m, 2H), 7.81-7.65 (m, 2H), 4.22-3.40 (m, 7H), 3.14 (m, 2H), 3.02-2.67 (m, 4H), 2.20-1.82 (m, 2H). LC-MS (ESI) (M + H)⁺ = 650.1. |
| 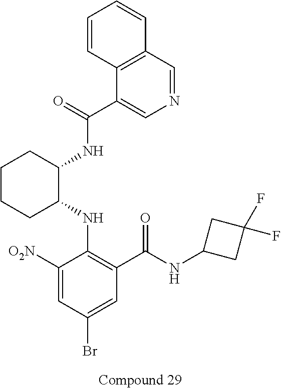 [SMILES: O=C(NC1CC(F)(F)C1)c1cc(Br)cc([N+](=O)[O-])c1N[C@@H]1CCCC[C@@H]1NC(=O)c1cncc2ccccc12] | Compound 29: N-((3S, 4S)-3-((4-chloro-2-((3,3- difluorocyclobutyl)carbamoyl)-6-nitrophenyl)amino)tetrahydro- 2H-pyran-4-yl)isoquinoline-4-carboxamide ¹H NMR (400 MHz, DMSO-d₆) δ 9.34 (s, 1H), 9.08 (d, J = 6.0 Hz, 1H), 8.73 (d, J = 8.0 Hz, 1H), 8.45 (d, J = 10.0 Hz, 1H), 8.20-8.08 (m, 4H), 7.81-7.69 (m, 3H), 4.35 (d, J = 4.0 Hz, 1H), 4.16 (dd, J = 12.0, 6.0 Hz, 1H), 3.94 (d, J = 12.0 Hz, 2H), 3.84 (dd, J = 12.0, 4.0 Hz, 1H), 3.64 (d, J = 10.0 Hz, 1H), 3.53 (t, J = 10.0 Hz, 1H), 2.94 (m, 2H), 2.72 (m, 2H), 1.91 (d, J = 10.0 Hz, 1H), 1.70 (d, J = 10.0 Hz, 1H). LC-MS (ESI) (M + H)⁺ = 560.1. |
| 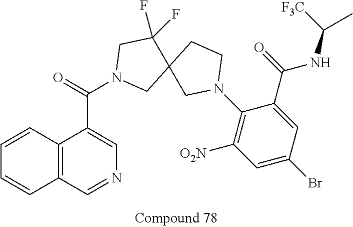 [SMILES: C[C@@H](NC(=O)c1cc(Br)cc([N+](=O)[O-])c1N1CCC2(CN(C(=O)c3cncc4ccccc34)CC2(F)F)C1)C(F)(F)F] | Compound 78: 5-bromo-2-(9,9-difluoro-7-(isoquinoline-4- carbonyl)-2,7-diazaspiro[4. 4]nonan-2-yl)-3-nitro-N-((R)- 1,1,1-trifluoropropan-2-yl)benzamide ¹H NMR (400 MHz, DMSO-d₆) δ 9.42 (s, 1H), 9.24 (ddd, J = 76.0, 8.0, 4.0 Hz, 1H), 8.63-8.55 (m, 1H), 8.23 (d, J = 8.0 Hz, 1H), 8.10 (dt, J = 40.0, 4.0 Hz, 1H), 7.90-7.84 (m, 2H), 7.80-7.74 (m, 1H), 7.70-7.54 (m, 1H), 4.85-4.42 (m, 1H), 4.22-3.39 (m, 5H), 3.31- 2.94 (m, 3H), 2.20-1.81 (m, 2H), 1.39-1.18 (m, 3H). LC-MS (ESI) (M + H)⁺ = 656.0. |

### Synthesis of Compounds 116, 117, 124, 128 and 130

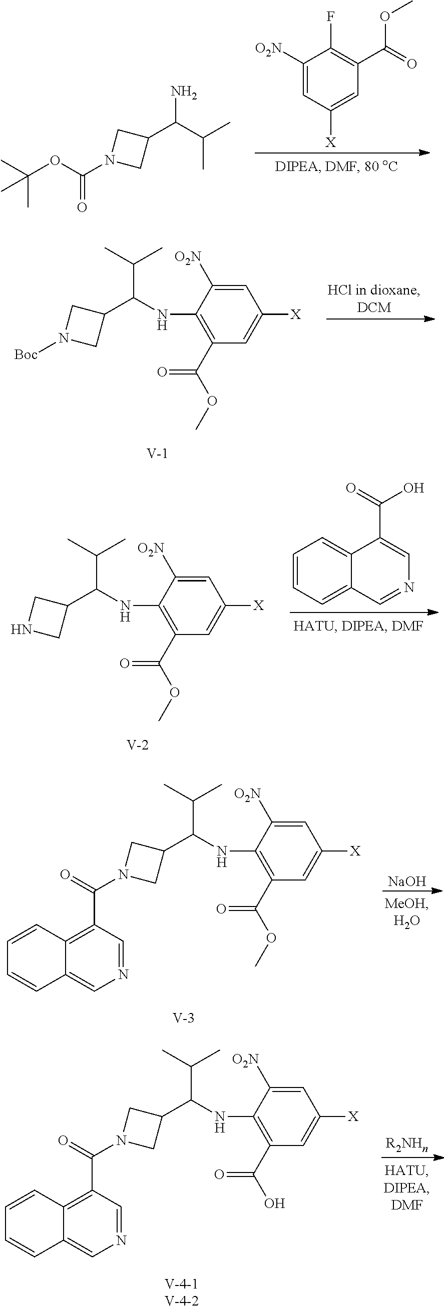

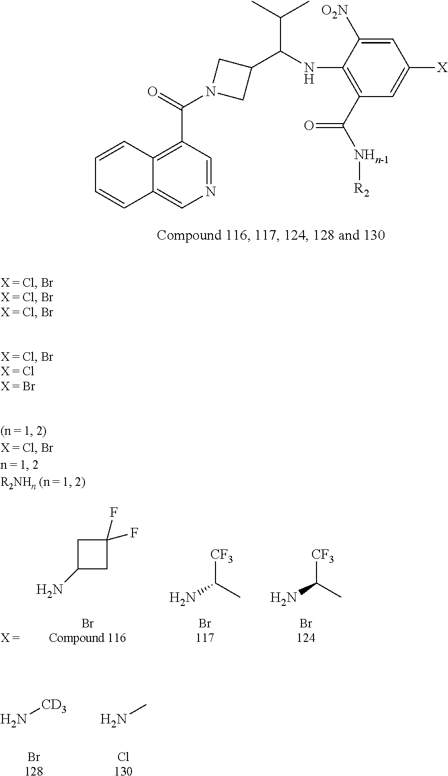

### Intermediates V-1~V-4

The synthetic procedures of intermediates V-1~V-4 are similar to that of intermediates IV-2~IV-5.

### Compounds 116, 117, 124, 128 and 130

The synthetic procedure is similar to the last step of the synthesis route of compound 14.

| | |
|---|---|
| 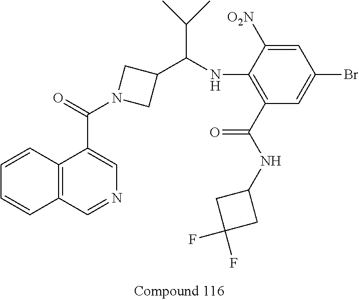 [SMILES: CC(C)C(Nc1c(C(=O)NC2CC(F)(F)C2)cc(Br)cc1[N+](=O)[O-])C1CN(C(=O)c2cncc3ccccc23)C1] | Compound 116: 5-bromo-N-(3,3-difluorocyclobutyl)-2-((1-(1-(isoquinoline-4- carbonyl)azetidin-3-yl)-2-methylpropyl)amino)-3-nitrobenzamide ¹H NMR (400 MHz, DMSO-d₆) δ 9.39 (d, J = 6.0 Hz, 1H), 9.33 (t, J = 6.0 Hz, 1H), 8.60 (s, 1H), 8.21 (dd, J = 12.0, 4.0 Hz, 2H), 8.13-7.93 (m, 2H), 7.89-7.73 (m, 3H), 4.24-3.99 (m, 4H), 3.90-3.67 (m, 1H), 3.52 (s, 2H), 2.97 (d, J = 14.0 Hz, 3H), 2.73 (s, 2H), 1.73 (d, J = 18.0 Hz, 1H), 0.80-0.65 (m, 5H). LC-MS (ESI) (M + H)⁺ = 616.1. |
| 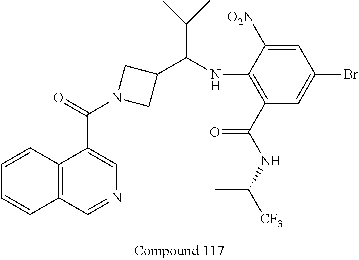 [SMILES: CC(C)C(Nc1c(C(=O)N[C@@H](C)C(F)(F)F)cc(Br)cc1[N+](=O)[O-])C1CN(C(=O)c2cncc3ccccc23)C1] | Compound 117: 5-bromo-2-((1-(1-(isoquinoline-4-carbonyl)azetidin-3-yl)-2- methylpropyl)amino)-3-nitro-N-((S)-1,1,1-trifluoropropan-2-yl)benzamide ¹H NMR (400 MHz, DMSO-d₆) δ 9.44 (dd, J = 12.0, 8.0 Hz, 1H), 9.36 (s, 1H), 8.50 (d, J = 44.0 Hz, 1H), 8.28-8.16 (m, 2H), 8.07 (dd, J = 24.0, 8.0 Hz, 1H), 7.92-7.68 (m, 4H), 4.71 (s, 1H), 4.22-3.53 (m, 4H), 2.94 (dd, J = 16.0, 8.0 Hz, 1H), 1.73-1.60 (m, 1H), 1.43-1.17 (m, 4H), 0.70 (dd, J = 16.0, 8.0 Hz, 6H). LC- MS (ESI) (M + H)⁺ = 622.1. |
| 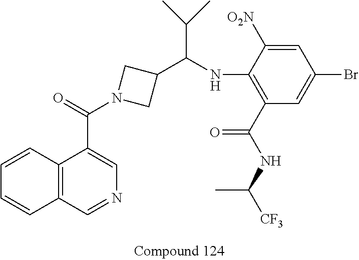 [SMILES: CC(C)C(Nc1c(C(=O)N[C@H](C)C(F)(F)F)cc(Br)cc1[N+](=O)[O-])C1CN(C(=O)c2cncc3ccccc23)C1] | Compound 124: 5-bromo-2-((1-(1-(isoquinoline-4-carbonyl)azetidin-3-yl)-2- methylpropyl)amino)-3-nitro-N-((R)-1,1,1-trifluoropropan-2-yl)benzamide ¹H NMR (400 MHz, DMSO-d₆) δ 9.47 (dd, J = 12.0, 8.0 Hz, 1H), 9.38 (s, 1H), 8.58 (s, 1H), 8.27 (dd, J = 16.0, 2.0 Hz, 1H), 8.20 (d, J = 8.0 Hz, 1H), 8.09 (dd, J = 24.0, 8.0 Hz, 1H), 7.96-7.68 (m, 4H), 4.73 (s, 1H), 4.24-3.70 (m, 5H), 2.96 (dd, J = 16.0, 8.0 Hz, 1H), 1.70 (s, 1H), 1.39 (dd, J = 16.0, 8.0 Hz, 3H), 0.91- 0.53 (m, 6H). LC-MS (ESI) (M + H)⁺ = 622.1. |
| 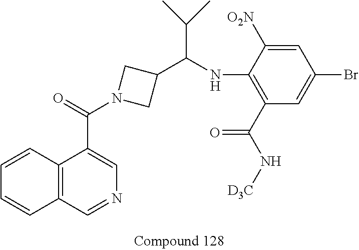 [SMILES: [2H]C([2H])([2H])NC(=O)c1cc(Br)cc([N+](=O)[O-])c1NC(C(C)C)C1CN(C(=O)c2cncc3ccccc23)C1] | Compound 128: N-((1S, 2R)-2-((4-bromo-2-((methyl-d3)carbamoyl)-6- nitrophenyl)amino)cyclohexyl)isoquinoline-4-carboxamide ¹H NMR (400 MHz, Methanol-d₄) δ 9.32 (d, J = 8.0 Hz, 1H), 8.54 (d, J = 64.0 Hz, 1H), 8.25 (dd, J = 16.0, 2.0 Hz, 1H), 8.18 (dd, J = 8.0, 4.0 Hz, 1H), 8.10 (dd, J = 24.0, 8.0 Hz, 1H), 7.89 (dt, J = 24.0, 8.0 Hz, 1H), 7.77 (q, J = 8.0 Hz, 1H), 7.62 (dd, J = 8.0, 2.0 Hz, 1H), 4.37 (t, J = 8.0 Hz, 1H), 4.15-4.03 (m, 2H), 3.79 (t, J = 8.0 Hz, 1H), 3.61 (t, J = 8.0 Hz, 1H), 3.09 (dt, J = 16.0, 8.0 Hz, 1H), 1.79 (dt, J = 12.0, 8.0 Hz, 1H), 0.83 (ddd, J = 20.0, 8.0, 8.0Hz, 6H). LC-MS (ESI) (M + H)⁺ = 543.1. |
| 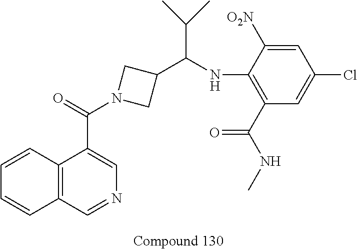 [SMILES: CNC(=O)c1cc(Cl)cc([N+](=O)[O-])c1NC(C(C)C)C1CN(C(=O)c2cncc3ccccc23)C1] | Compound 130: 5-chloro-2-((1-(1-(isoquinoline-4-carbonyl)azetidin-3-yl)-2- methylpropyl)amino)-N-methyl-3-nitrobenzamide acid ¹H NMR (400 MHz, DMSO-d₆) δ 9.37 (d, J = 4.0 Hz, 1H), 8.81 (s, 1H), 8.60- 8.42 (m, 1H), 8.18 (d, J = 8.0 Hz, 1H), 8.12-7.97 (m, 3H), 7.88-7.72 (m, 2H), 7.61 (s, 1H), 4.24-3.93 (m, 3H), 3.92-3.62 (m, 1H), 3.58-3.39 (m, 1H), 3.00-2.90 (s, 1H), 2.74 (t, J = 8.0 Hz, 3H), 1.77-1.64 (m, 1H), 0.75-0.65 (m, 6H). LC-MS (ESI) (M + H)⁺ = 496.2. |

### Synthesis of Compound 17

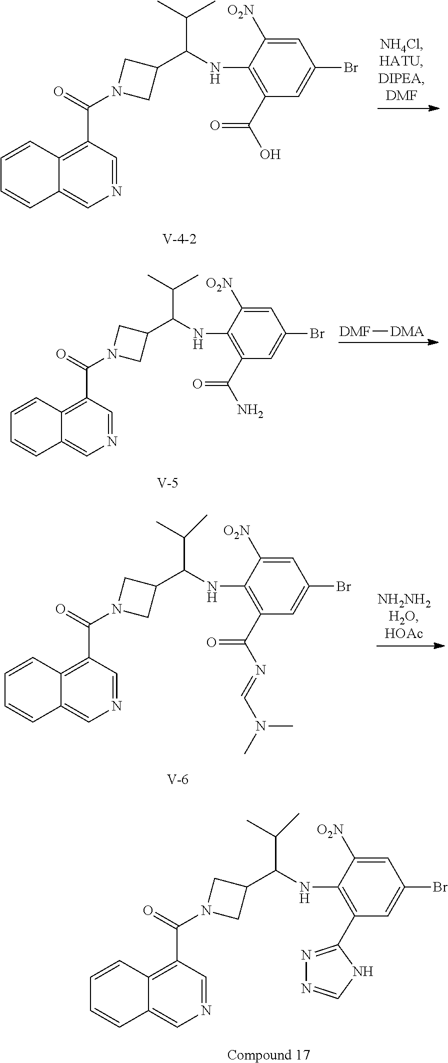

### Intermediate V-5: 5-bromo-2-((1-(1-(isoquinoline-4-carbonyl)azetidin-3-yl)-2-methylpropyl)amino)-3-nitrobenzamide

To a solution of intermediate V-4 (200 mg, 0.38 mmol) and NH₄Cl (41 mg, 0.76 mmol) in DMF (5.0 mL), DIPEA (147 mg, 1.14 mmol) and HATU (173 mg, 0.46 mmol) were added and the reaction was stirred at room temperature for 4 h. The reaction mixture was poured into water (50.0 mL) and then extracted with EtOAc (30.0 mL*3). The combined organic layer was washed with brine (30.0 mL*3), dried over Na₂SO₄ and concentrated to give a crude product, which was purified by Flash Chromatography eluting with methanol in DCM (MeOH:DCM=1: 40) to afford the title compound (190 mg, 95% yield) as a light yellow liquid. LC-MS (ESI) (M+H)⁺=526.1.

### Intermediate V-6: (E)-5-bromo-N-((dimethylamino)methylene)-2-((1-(1-(isoquinoline-4-carbonyl)azetidin-3-yl)-2-methylpropyl)amino)-3-nitrobenzamide

A solution of 5-bromo-2-((1-(1-(isoquinoline-4-carbonyl)azetidin-3-yl)-2-methylpropyl)amino)-3-nitrobenzamide (190 mg, 0.36 mmol) in DMF-DMA (5.0 mL) was stirred at 120° C. for 4 hr. The reaction mixture was poured into water (50.0 mL) and then extracted with EtOAc (30.0 mL*3). The combined organic layer was washed with brine (30.0 mL*3), dried over Na₂SO₄ and concentrated to give a crude product, which was purified by Flash Chromatography eluting with methanol in DCM (MeOH:DCM=1:40) to afford the title compound (200 mg, 95% yield) as a light yellow liquid. LC-MS (ESI) (M+H)⁺=581.2.

### Compound 17: (3-(1-((4-bromo-2-nitro-6-(4H-1,2,4-triazol-3-yl)phenyl)amino)-2-methylpropyl)azetidin-1-yl)(isoquinolin-4-yl)methanone

Hydrazine monohydrate (13 mg, 0.41 mmol) was added to a solution of intermediate V-6 (200 mg, 0.34 mmol) in HOAc (5.0 mL). The solution was stirred at 120° C. for 2 h. The solvent was removed under vacuum. The residue was purified by prep-HPLC to give compound 17 (67.0 mg, 36% yield) as a yellow solid.

| | |
|---|---|
| 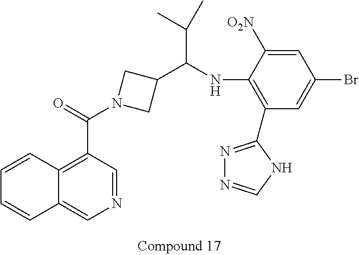 [SMILES: CC(C)C(Nc1c(-c2nnc[nH]2)cc(Br)cc1[N+](=O)[O-])C1CN(C(=O)c2cncc3ccccc23)C1] | Compound 17: (3-(1-((4-bromo-2-nitro-6-(4H-1,2,4-triazol-3-yl)phenyl)amino)-2 -methylpropyl)azetidin-1-yl)(isoquinolin-4-yl)methanone ¹H NMR (400 MHz, DMSO-d₆) δ 14.60 (br, 1H),9.36 (d, J = 13.1 Hz, 1H), 8.83 (s, 1H), 8.58-8.27 (m, 3H), 8.17 (t, J = 8.6 Hz, 1H), 7.99-7.85 (m, 2H), 7.73 (s, 2H), 4.18-3.90 (m, 2H), 3.29-3.01 (m, 3H), 2.96 (d, J = 9.0 Hz, 1H), 1.69 (s, 1H), 0.70 (d, J = 7.3 Hz, 6H). LC-MS (ESI) (M + H)⁺ = 550.1. |

### Synthesis of Compound 65

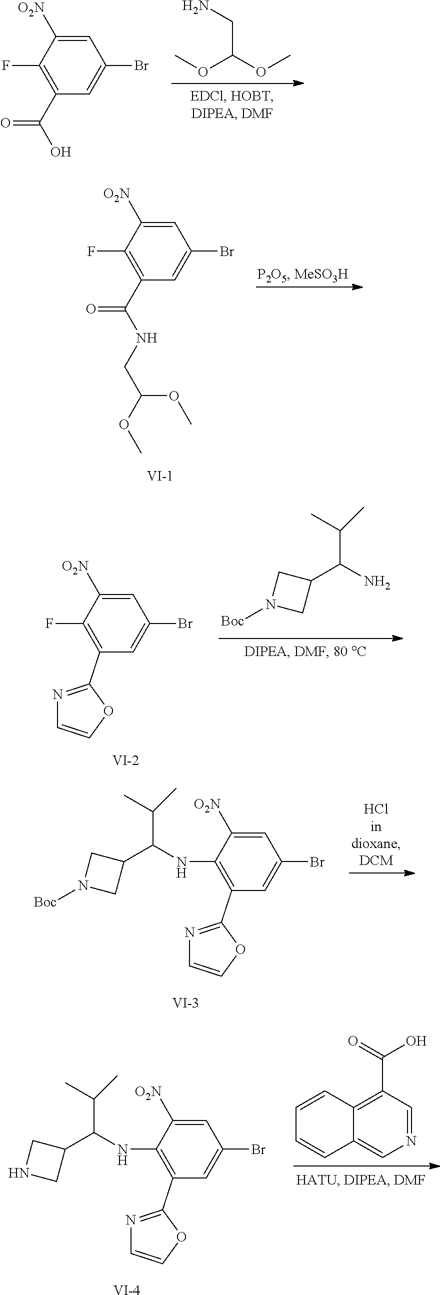

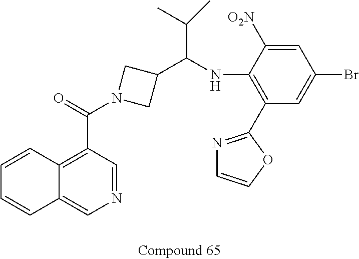 [SMILES: CC(C)C(Nc1c(-c2ncco2)cc(Br)cc1[N+](=O)[O-])C1CN(C(=O)c2cncc3ccccc23)C1]

### Intermediate VI-1: 5-bromo-N-(2, 2-dimethoxyethyl)-2-fluoro-3-nitrobenzamide

EDCI (1.09 g, 5.69 mmol), HOBT (767 mg, 5.69 mmol) and DIPEA (1.47 g, 11.37 mmol) were added to a solution of 5-bromo-2-fluoro-3-nitrobenzoic acid (1.00 g, 3.79 mmol) and 2,2-dimethoxyethan-1-amine (478 mg, 4.55 mmol) in DMF (15 mL). The reaction mixture was stirred for 2 hr. The reaction mixture was poured into water (20 mL), and extracted with EtOAc (20 mL×2). The combined organic layers were washed with brine (20 mL), dried over Na₂SO₄, filtered and concentrated under reduced pressure to give a residue, which was purified by Flash Chromatography eluting with ethyl acetate in petroleum ether (EtOAc:PE=1:5) to afford the title compound (480 mg, 36% yield) as a yellow solid. LC-MS (ESI) (M+H)⁺=351.1.

### Intermediate VI-2: 2-(5-bromo-2-fluoro-3-nitrophenyl)oxazole

To a solution of intermediate VI-1 (480 mg, 1.37 mmol) in MeSO₃H (5 mL), P2O5 (777 mg, 5.48 mmol) was added. The reaction mixture was heated and stirred at 140° C. for 6 hr. After cooling to room temperature, the reaction mixture was poured into ice-water (20 mL) and extracted with EtOAc (20 mL×2). The combined organic layers were washed with brine (20 mL), dried over Na₂SO₄, filtered and concentrated under reduced pressure to give a residue, which was purified by Flash Chromatography eluting with ethyl acetate in petroleum ether (EtOAc:PE=1:3) to afford the title compound (180 mg, 45% yield) as a yellow solid. LC-MS (ESI) (M+H)⁺=287.1.

### Intermediates VI-3-VI-4

The synthetic procedures of intermediate VI-3~VI-4 are similar to that of intermediate I-2~I-3.

### Compound 65: (3-(1-((4-bromo-2-nitro-6-(oxazol-2-yl)phenyl)amino)-2-methylpropyl)azetidin-1-yl)(isoquinolin-4-yl)methanone

The synthesis of compound 65 is similar to that of intermediate IV-4.

| | |
|---|---|
| 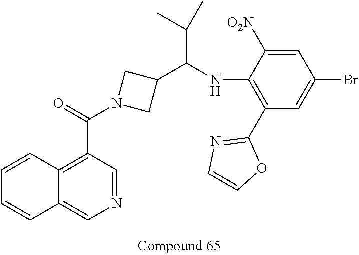 [SMILES: CC(C)C(Nc1c(-c2ncco2)cc(Br)cc1[N+](=O)[O-])C1CN(C(=O)c2cncc3ccccc23)C1] | Compound 65: (3-(1-((4-bromo-2-nitro-6-(oxazol-2-yl)phenyl)amino)-2- methylpropyl)azetidin-1-yl)(isoquinolin-4-yl)methanone ¹H NMR (400 MHz, DMSO-d₆) δ 9.37 (d, J = 12.0 Hz, 1H), 8.72 (dd, J = 60.0, 12.0 Hz, 1H), 8.53-8.12 (m, 4H), 8.10-7.86 (m, 2H), 7.77 (dt, J = 24.0, 8.0 Hz, 2H), 7.39 (d, J = 200.0 Hz, 1H), 4.29-3.99 (m, 3H), 3.99- 3.60 (m, 2H), 3.00 (s, 1H), 1.80-1.63 (m, 1H), 0.77-0.60 (m, 6H). LC-MS (ESI) (M + H)⁺ = 550.1. |

### Synthesis of Compounds 71 and 73

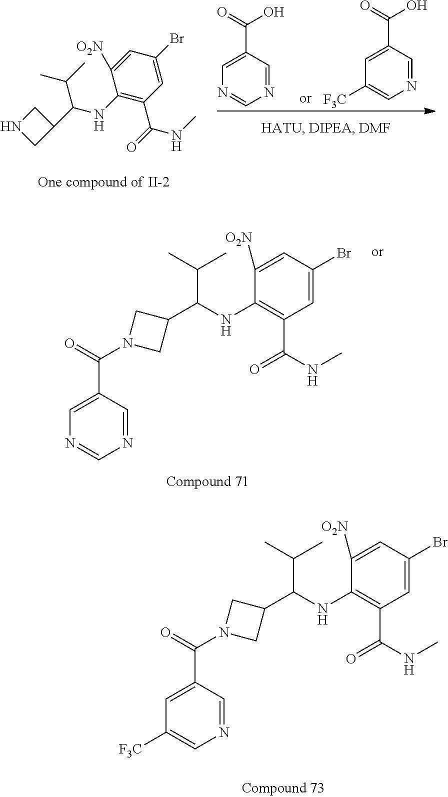

| | |
|---|---|
| 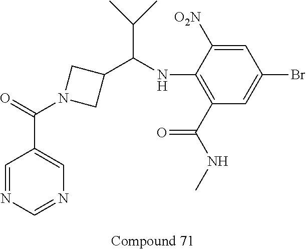 [SMILES: CNC(=O)c1cc(Br)cc([N+](=O)[O-])c1NC(C(C)C)C1CN(C(=O)c2cncnc2)C1] | Compound 71: 5-bromo-N-methyl-2-((2-methyl-1-(1-(pyrimidine-5- carbonyl)azetidin-3-yl)propyl)amino)-3-nitrobenzamide ¹H NMR (400 MHz, DMSO-d₆) δ 9.30 (d, J = 4.0 Hz, 1H), 9.02 (s, 1H), 8.97 (s, 1H), 8.83 (dd, J = 12.0, 4.6 Hz, 1H), 8.16 (dd, J = 4.0, 2.4 Hz, 1H), 7.99 (dd, J = 32.0, 11.0 Hz, 1H), 7.71 (dd, J = 12.0, 2.1 Hz, 1H), 4.44 (dt, J = 16.0, 8.7 Hz, 1H), 4.30-4.06 (m, 2H), 3.98-3.75 (m, 1H), 3.51 (d, J = 8.0 Hz, 1H), 3.01- 2.92 (m, 1H), 2.75 (dd, J = 12.0, 4.5 Hz, 3H), 1.76 (dt, J = 16.0, 7.9 Hz, 1H), 0.75 (dt, J = 16.0, 8.0 Hz, 6H). LC-MS (ESI) (M + H)⁺ = 491.1. |
| 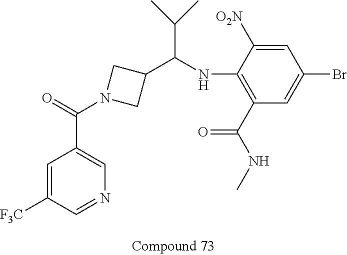 [SMILES: CNC(=O)c1cc(Br)cc([N+](=O)[O-])c1NC(C(C)C)C1CN(C(=O)c2cncc(C(F)(F)F)c2)C1] | Compound 73: 5-bromo-N-methyl-2-((2-methyl-1-(1-(5- (trifluoromethyl)nicotinoyl)azetidin-3-yl)propyl)amino)-3-nitrobenzamide ¹H NMR (400 MHz, DMSO-d₆) δ 9.14-9.01 (m, 2H), 8.90-8.75 (m, 1H), 8.31 (d, J = 24.0 Hz, 1H), 8.16 (d, J = 8.0 Hz, 1H), 7.97 (dd, J = 32.0, 12.0 Hz, 1H), 7.71 (d, J = 4.0 Hz, 1H), 4.49-4.36 (m, 1H), 4.29-4.07 (m, 2H), 3.99-3.73 (m, 1H), 3.51 (s, 1H), 2.96 (s, 1H), 2.75 (dd, J = 12.0, 4.0 Hz, 3H), 1.76 (s, 1H), 0.75 (dt, J = 16.0, 8.0 Hz, 6H). LC-MS (ESI) (M + H)⁺ = 558.1. |

### Synthesis of Compounds 19, 33, 36, 37, 38, 40, 41, 42, 43, 44, 45, 46, 47, 48, 91, 93, 94, 95, 111, 112, 139, 142, 143, 147, 148, 149, 151, 152, 153, 154, 155, 156, 157, 158 and 159

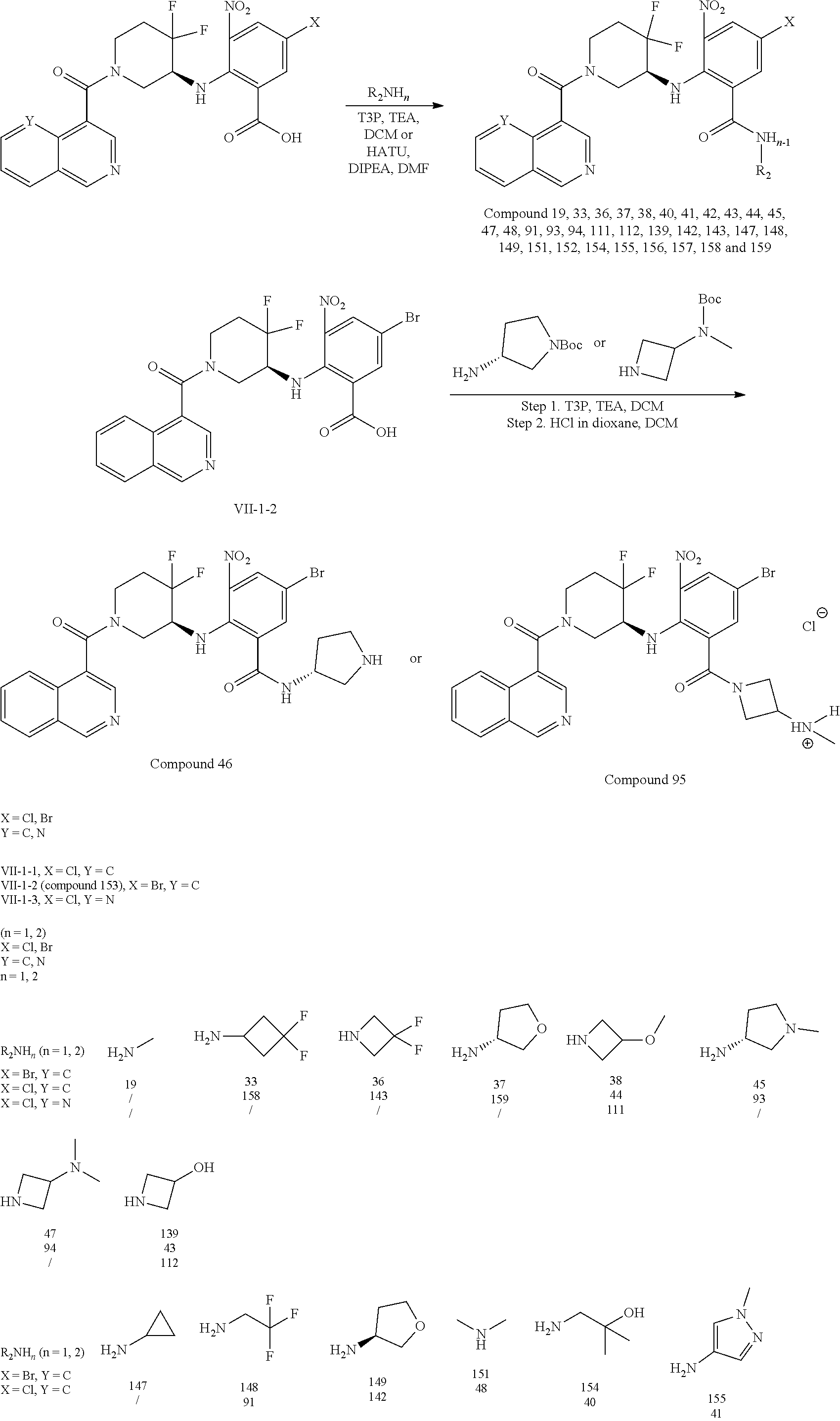

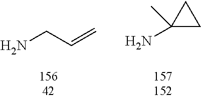

### Intermediates VII-1-1 and VII-1-2 (Compound 153)

The synthetic procedures of intermediates VII-1-1 and VII-1-2 are similar to that of intermediate IV-5.

### Compounds 19, 33, 36, 37, 38, 40, 41, 42, 43, 44, 45, 47, 48, 91, 93, 94, 111, 112, 139, 142, 143, 147, 148, 149, 151, 152, 154, 155, 156, 157, 158 and 159

Method 1: Corresponding amine (1.05 equiv), TEA (5.0 equiv) and T³P (2.0 equiv) were added to a solution of intermediate VII-1-1 or VII-1-2 (1.0 equiv) in DCM, and stirred at room temperature for 2 hr. The reaction mixture was poured into water and then extracted with DCM three times. The combined organic layer was washed with brine, dried over Na₂SO₄ and concentrated to give a crude product which was purified by prep-HPLC to give the target compounds as yellow solids.

Method 2: To a solution of intermediate VII-1-1 or VII-1-2 (1.0 equiv) in DMF (2.5 mL), corresponding amine (1.2 equiv), HATU (1.5 equiv) and DIPEA (3.0 equiv) were added and the reaction was stirred at room temperature overnight. The reaction was diluted with water (50.0 mL) and extracted with EtOAc (3*20.0 mL). The organic layer was separated, washed with water, NH₄Cl solution (20 mL), NaHCO₃ solution (20 mL) and further saturated NaCl solution (20 mL), and it was dried over Na₂SO₄ and concentrated in vacuo. The residue was purified using prep-TLC with ethyl acetate in petroleum ether and Prep-HPLC to give the target compounds as yellow solids.

### Compounds 46 and 95

Compounds 46 and 95 were synthesized from VII-1-2 with two steps (step1: described in method 1 of compound 19, step 2: described in the procedure of I-3).

| | |
|---|---|
| 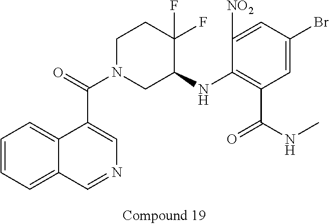 [SMILES: CNC(=O)c1cc(Br)cc([N+](=O)[O-])c1N[C@H]1CN(C(=O)c2cncc3ccccc23)CCC1(F)F] | Compound 19: (S)-5-bromo-2-((4,4-difluoro-1-(isoquinoline-4- carbonyl)piperidin-3-yl)amino)-N-methyl-3-nitrobenzamide ¹H NMR (400 MHz, DMSO-d₆) δ 9.41 (d, J = 4.0 Hz, 1H), 9.01- 8.51 (m, 2H), 8.40-8.01 (m, 3H), 7.97-7.71 (m, 4H), 4.68-3.67 (m, 4H), 2.79 (d, J = 56.0 Hz, 3H), 2.36-1.71 (m, 3H). LC-MS (ESI) (M + H)⁺ = 548.1. |
| 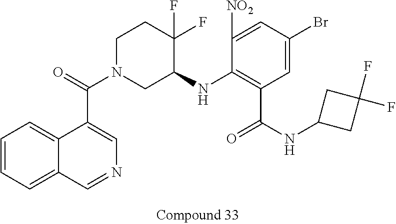 [SMILES: O=C(NC1CC(F)(F)C1)c1cc(Br)cc([N+](=O)[O-])c1N[C@H]1CN(C(=O)c2cncc3ccccc23)CCC1(F)F] | Compound 33: (S)-5-bromo-2-((4,4-difluoro-1-(isoquinoline-4- carbonyl)piperidin-3-yl)amino)-N-(3,3-difluorocyclobutyl)-3- nitrobenzamide ¹H NMR (400 MHz, DMSO-d₆) δ 9.47-9.13 (m, 2H), 8.70-7.43 (m, 8H), 4.04 (m, 4H), 2.88 (m, 4H), 2.24 (s, 3H), 1.23 (s, 1H). LC-MS (ESI) (M + H)⁺ = 624.0. |
| 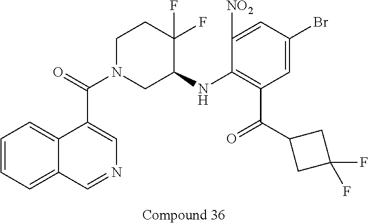 [SMILES: O=C(c1cc(Br)cc([N+](=O)[O-])c1N[C@H]1CN(C(=O)c2cncc3ccccc23)CCC1(F)F)C1CC(F)(F)C1] | Compound 36: (S)-(5-bromo-2-((4,4-difluoro-1-(isoquinoline-4- carbonyl)piperidin-3-yl)amino)-3-nitrophenyl)(3,3-difluoroazetidin- 1-yl)methanone ¹H NMR (400 MHz, DMSO-d₆) δ 9.42 (d, J = 6.0 Hz, 1H), 8.57 (s, 1H), 8.41-8.18 (m, 2H), 8.08-7.74 (m, 4H), 7.16 (m, 1H), 4.94- 3.48 (m, 7H), 3.31 (m, 1H), 2.08 (s, 3H). LC-MS (ESI) (M + H)⁺ = 610.1. |
| 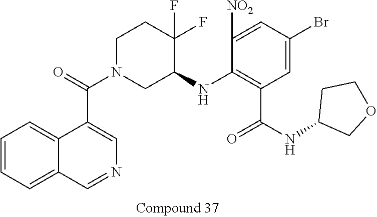 [SMILES: O=C(N[C@@H]1CCOC1)c1cc(Br)cc([N+](=O)[O-])c1N[C@H]1CN(C(=O)c2cncc3ccccc23)CCC1(F)F] | Compound 37: 5-bromo-2-(((S)-4,4-difluoro-1-(isoquinoline-4- carbonyl)piperidin-3-yl)amino)-3-nitro-N-((R)-tetrahydrofuran- 3-yl)benzamide ¹H NMR (400 MHz, DMSO-d₆) δ 9.41 (s, 1H), 9.21-7.32 (m, 9H), 3.77 (d, J = 48 Hz, 7H), 1.22 (s, 7H). LC-MS (ESI) (M + H)⁺ = 604.1. |
| 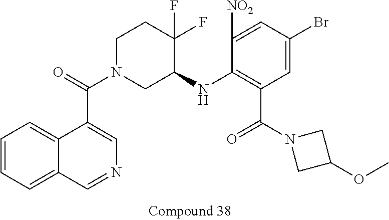 [SMILES: COC1CN(C(=O)c2cc(Br)cc([N+](=O)[O-])c2N[C@H]2CN(C(=O)c3cncc4ccccc34)CCC2(F)F)C1] | Compound 38: (S)-(5-bromo-2-((4,4-difluoro-1-(isoquinoline-4- carbonyl)piperidin-3-yl)amino)-3-nitrophenyl)(3-methoxyazetidin- 1-yl)methanone ¹H NMR (400 MHz, Methanol-d₄) δ 9.35 (d, J = 4.0 Hz, 1H), 8.57 (s, 1H), 8.41-8.20 (m, 2H), 7.98-7.75 (m, 4H), 4.58-4.37 (m, 2H), 4.32-4.04 (m, 2H), 3.88 (s, 2H), 3.77-3.40 (m, 4H), 3.36-3.31 (m, 3H), 2.39-2.05 (m, 2H). LC-MS (ESI) (M + H)⁺ = 604.1. |
| 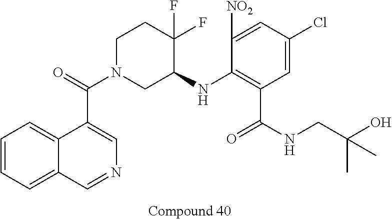 | Compound 40: (S)-5-chloro-2-((4,4-difluoro-1-(isoquinoline-4- carbonyl)piperidin-3-yl)amino)-N-(2-hydroxy-2-methylpropyl)-3- nitrobenzamide ¹H NMR (400 MHz, DMSO-d₆) δ 9.50 (s, 1H), 9.14-8.12 (m, 4H), 7.88 (m, 3H), 6.95 (m, 2H), 5.32 (s, 1H), 4.50 (m, 3H), 3.31 (s, 2H), 2.42-1.59 (m, 3H), 1.18 (m, 7H). LC-MS (ESI) (M + H)⁺ = 526.3. |
| 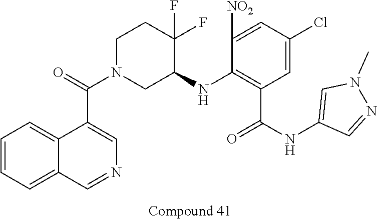 [SMILES: Cn1cc(NC(=O)c2cc(Cl)cc([N+](=O)[O-])c2N[C@H]2CN(C(=O)c3cncc4ccccc34)CCC2(F)F)cn1] | Compound 41: (S)-5-chloro-2-((4,4-difluoro-1-(isoquinoline-4- carbonyl)piperidin-3-yl)amino)-N-(1-methyl-1H-pyrazol-4-yl)-3- nitrobenzamide ¹H NMR (400 MHz, DMSO-d₆) δ 11.01 (d, J = 80.0 Hz, 1H), 9.48 (s, 1H), 8.63 (d, J = 46.8 Hz, 1H), 8.32-8.27 (m, 1H), 8.08 (dd, J = 31.1, 6.0 Hz, 2H), 7.96-7.78 (m, 3H), 7.63 (d, J = 16.0 Hz, 1H), 4.55 (m, 1H), 3.83 (s, 6H), 3.40-3.15 (m, 2H), 2.05 (m, 3H). LC-MS (ESI) (M + H)⁺ = 570.2. |
| 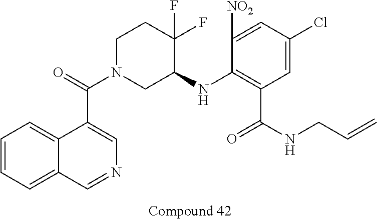 [SMILES: C=CCNC(=O)c1cc(Cl)cc([N+](=O)[O-])c1N[C@H]1CN(C(=O)c2cncc3ccccc23)CCC1(F)F] | Compound 42: (S)-N-allyl-5-chloro-2-((4,4-difluoro-1-(isoquinoline- 4-carbonyl) piperidin-3-yl)amino)-3-nitrobenzamide ¹H NMR (400 MHz, DMSO-d₆) δ 9.43 (s, 1H), 9.07 (d, J = 84.0 Hz, 1H), 8.61 (d, J = 24.0Hz, 1H), 8.29-8.05 (m, 2H), 8.03-7.69 (m, 5H), 5.19 (dd, J = 48.0, 16.0 Hz, 2H), 4.27-3.66 (m, 4H), 3.40-3.01 (m, 3H), 2.37-1.61 (m, 3H). LC-MS (ESI) (M + H)⁺ = 530.4. |
| 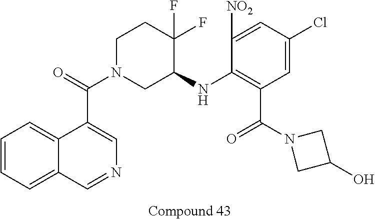 [SMILES: O=C(c1cc(Cl)cc([N+](=O)[O-])c1N[C@H]1CN(C(=O)c2cncc3ccccc23)CCC1(F)F)N1CC(O)C1] | Compound 43: (S)-(5-chloro-2-((4,4-difluoro-1-(isoquinoline-4- carbonyl)piperidin-3-yl)amino)-3-nitrophenyl)(3-hydroxyazetidin-1- yl)methanone ¹H NMR (400 MHz, DMSO-d₆) δ 9.42 (s, 1H), 8.60 (s, 1H), 8.30- 8.03 (m, 2H), 7.97-7.32 (m, 5H), 5.85 (s, 1H), 4.87-4.12 (m, 4H), 4.03-3.48 (m, 5H), 2.38-1.51 (m, 3H). LC-MS (ESI) (M + H)⁺ = 546.2. |
| 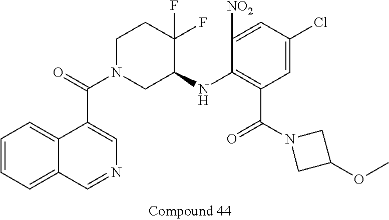 [SMILES: COC1CN(C(=O)c2cc(Cl)cc([N+](=O)[O-])c2N[C@H]2CN(C(=O)c3cncc4ccccc34)CCC2(F)F)C1] | Compound 44: (S)-(5-chloro-2-((4,4-difluoro-1-(isoquinoline-4- carbonyl)piperidin-3-yl)amino)-3-nitrophenyl)(3-methoxyazetidin-1- yl)methanone ¹H NMR (400 MHz, DMSO-d₆) δ 9.42 (s, 1H), 8.58 (d, J = 8.0 Hz, 1H), 8.26-8.06 (m, 2H), 7.99-7.45 (m, 5H), 4.45-3.62 (m, 8H), 3.32-2.62 (m, 5H), 2.36-1.66 (m, 2H). LC-MS (ESI) (M + H)⁺ = 558.2. |
| 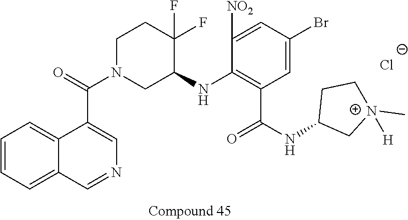 [SMILES: C[NH+]1CC[C@@H](NC(=O)c2cc(Br)cc([N+](=O)[O-])c2N[C@H]2CN(C(=O)c3cncc4ccccc34)CCC2(F)F)C1 ; [Cl-]] | Compound 45: 5-bromo-2-(((S)-4,4-difluoro-1-(isoquinoline-4- carbonyl)piperidin-3-yl)amino)-N-((R)-1-methylpyrrolidin-3-yl)-3- nitrobenzamide hydrochloride ¹H NMR (400 MHz, DMSO-d₆) δ 9.97 (s, 1H), 9.48 (s, 1H), 9.18 (s, 1H), 8.65 (s, 1H), 8.37 - 7.62 (m, 6H), 4.59 (s, 3H), 3.36 (t, J = 97.3 Hz, 7H), 2.90 (d, J = 3.2 Hz, 3H), 2.13 (m, 4H). LC-MS (ESI) (M + H)+ = 617.1. |
| 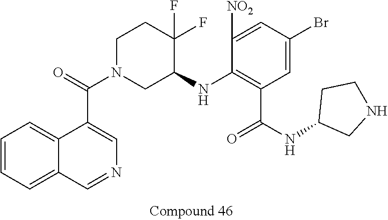 [SMILES: O=C(N[C@@H]1CCNC1)c1cc(Br)cc([N+](=O)[O-])c1N[C@H]1CN(C(=O)c2cncc3ccccc23)CCC1(F)F] | Compound 46: 5-bromo-2-(((S)-4,4-difluoro-1-(isoquinoline-4- carbonyl)piperidin-3-yl)amino)-3-nitro-N-((R)-pyrrolidin-3-yl) benzamide ¹H NMR (400 MHz, DMSO-d₆) δ 9.41 (s, 1H), 9.21-8.43 (m, 3H), 8.38-7.58 (m, 6H), 4.72-3.81 (m, 4H), 2.74 (s, 3H), 2.35-1.48 (m, 8H). LC-MS (ESI) (M + H)⁺ = 603.2. |
| 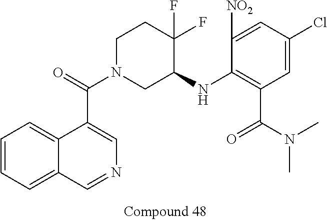 | Compound 48: (S)-5-chloro-2-((4,4-difluoro-1-(isoquinoline-4- carbonyl)piperidin-3-yl)amino)-N-(1-methyl-1H-pyrazol-4-yl)-3- nitrobenzamide ¹H NMR (400 MHz, DMSO-d₆) δ 11.01 (d, J = 80.0 Hz, 1H), 9.48 (s, 1H), 8.63 (d, J = 46.8 Hz, 1H), 8.32-8.27 (m, 1H), 8.08 (dd, J = 31.1, 6.0 Hz, 2H), 7.96-7.78 (m, 3H), 7.63 (d, J = 16.0 Hz, 1H), 4.55 (m, 1H), 3.83 (s, 6H), 3.40-3.15 (m, 2H), 2.05 (m, 3H). LC-MS (ESI) (M + H)⁺ = 570.2 |
| 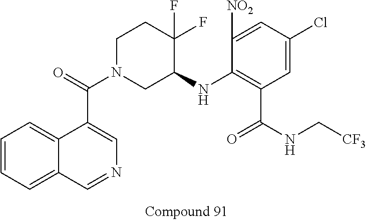 [SMILES: O=C(NCC(F)(F)F)c1cc(Cl)cc([N+](=O)[O-])c1N[C@H]1CN(C(=O)c2cncc3ccccc23)CCC1(F)F] | Compound 91: (S)-5-chloro-2-((4,4-difluoro-1-(isoquinoline-4- carbonyl)piperidin-3-yl)amino)-3-nitro-N-(2,2,2-trifluoroethyl) benzamide ¹H NMR (400 MHz, DMSO-d₆) δ 9.53 (d, J = 104.0 Hz, 2H), 8.71- 6.51 (m, 8H), 5.40-4.37 (m, 2H), 4.18 (s, 2H), 3.81 (d, J = 52.0 Hz, 1H), 2.34-1.88 (m, 2H), 1.22 (s, 2H). LC-MS (ESI) (M + H)⁺ = 572.2. |
| 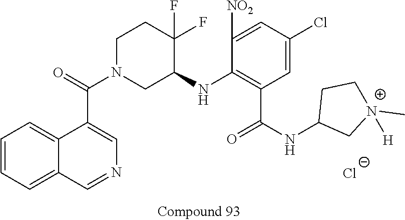 [SMILES: C[NH+]1CCC(NC(=O)c2cc(Cl)cc([N+](=O)[O-])c2N[C@H]2CN(C(=O)c3cncc4ccccc34)CCC2(F)F)C1 ; [Cl-]] | Compound 93: 5-chloro-2-(((S)-4,4-difluoro-1-(isoquinoline-4- carbonyl)piperidin-3-yl)amino)-N-(1-methylpyrrolidin-3-yl)-3- nitrobenzamide hydrochloride 1H NMR (400 MHz, DMSO-d₆) δ 10.08 (s, 1H), 9.48 (s, 1H), 9.19 (s, 1H), 8.64 (s, 1H), 8.34-7.50 (m, 6H), 4.51 (d, J = 62.0 Hz, 3H), 3.61- 2.63 (m, 10H), 2.13 (d, J = 92.0 Hz, 4H). LC-MS (ESI) (M + H)⁺ = 573.3. |
| 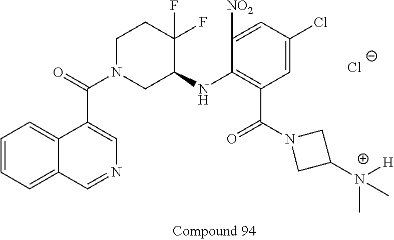 [SMILES: C[NH+](C)C1CN(C(=O)c2cc(Cl)cc([N+](=O)[O-])c2N[C@H]2CN(C(=O)c3cncc4ccccc34)CCC2(F)F)C1 ; [Cl-]] | Compound 94: (S)-(5-chloro-2-((4,4-difluoro-1-(isoquinoline-4- carbonyl)piperidin -3-yl)amino)-3-nitrophenyl)(3-(dimethylamino) azetidin-1-yl)methanone hydrochloride ¹H NMR (400 MHz, DMSO-d₆) δ 10.65 (s, 1H), 9.47 (s, 1H), 8.87- 6.45 (m, 7H), 4.88-4.12 (m, 8H), 3.76-2.57 (m, 8H), 2.32 (s, 2H). LC-MS (ESI) (M + H)⁺ = 573.3. |
| 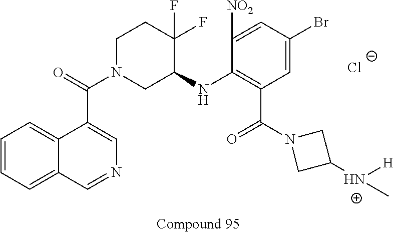 [SMILES: C[NH2+]C1CN(C(=O)c2cc(Br)cc([N+](=O)[O-])c2N[C@H]2CN(C(=O)c3cncc4ccccc34)CCC2(F)F)C1 ; [Cl-]] | Compound 95: (S)-(5-bromo-2-((4,4-difluoro-1-(isoquinoline-4- carbonyl)piperidin- 3-yl)amino)-3-nitrophenyl)(3-(methylamino) azetidin-1-yl)methanone hydrochloride ¹H NMR (400 MHz, DMSO-d₆) δ 9.48 (s, 1H), 9.15 (s, 2H), 8.64 (s, 1H), 8.42-8.15 (m, 2H), 7.98-7.79 (m, 4H), 4.13 (s, 6H), 3.35 (s, 3H), 2.60 (s, 3H), 2.08 (s, 3H). LC-MS (ESI) (M + H)⁺ = 603.2. |
| 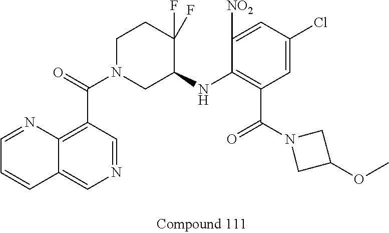 [SMILES: COC1CN(C(=O)c2cc(Cl)cc([N+](=O)[O-])c2N[C@H]2CN(C(=O)c3cncc4cccnc34)CCC2(F)F)C1] | Compound 111: (S)-(5-chloro-2-((4,4-difluoro-1-(1,6-naphthyridine- 8-carbonyl)piperidin-3-yl)amino)-3-nitrophenyl)(3-methoxyazetidin- 1-yl)methanone ¹H NMR (400 MHz, DMSO-d₆) δ 9.53 (m, 1H), 9.23 (m, 1H), 8.64- 8.86 (m, 2H), 8.01-8.25 (m, 1H), 7.59-7.85 (m, 2H), 6.82-7.46 (m, 1H), 4.41-4.68 (m, 1H), 4.28-4.38 (m, 2H), 4.09-4.21 (m, 2H), 3.90- 4.07 (m, 2H), 3.64-3.85 (m, 2H), 3.44-3.59 (m, 1H), 3.22 m, 3H), 1.93-2.29 (m, 2H). LC-MS (ESI) (M + H)⁺ = 561.4. |
| 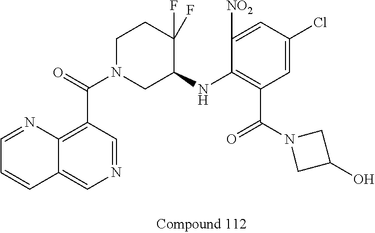 [SMILES: O=C(c1cc(Cl)cc([N+](=O)[O-])c1N[C@H]1CN(C(=O)c2cncc3cccnc23)CCC1(F)F)N1CC(O)C1] | Compound 112: (S)-(5-chloro-2-((4,4-difluoro-1-(1,6-naphthyridine- 8-carbonyl)piperidin-3-yl)amino)-3-nitrophenyl)(3-hydroxyazetidin- 1-yl)methanone ¹H NMR (400 MHz, DMSO-d₆) δ 9.43-9.53 (m, 1H), 9.09-9.27 (m, 1H), 8.74-8.83 (m, 1H), 8.60-8.72 (m, 1H), 7.99-8.25 (m, 1H), 7.70- 7.86 (m, 2H), 7.29-7.60 (m, 0.8H), 6.91-7.09 (m, 0.2H), 4.32-4.66 (m, 3H), 3.90-4.21 (m, 2H), 3.58-3.90 (m, 3H), 3.50-3.57 (m, 1H), 3.08-3.27 (m, 2H), 2.09-2.34 (m, 1H), 1.92-2.08 (m, 1H). LC-MS (ESI) (M + H)⁺ = 547.4. |
| 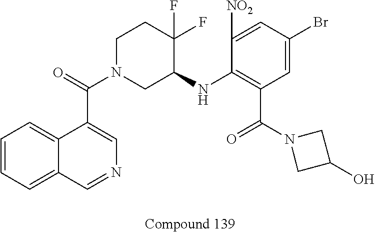 [SMILES: O=C(c1cc(Br)cc([N+](=O)[O-])c1N[C@H]1CN(C(=O)c2cncc3ccccc23)CCC1(F)F)N1CC(O)C1] | Compound 139: (S)-(5-bromo-2-((4,4-difluoro-1-(isoquinoline-4- carbonyl)piperidin-3-yl)amino)-3-nitrophenyl)(3-hydroxyazetidin-1- yl)methanone ¹H NMR (400 MHz, DMSO-d₆) δ 9.40 (s, 1H), 8.58 (s, 1H), 8.26 (d, J = 32.0 Hz, 2H), 7.80 (d, J = 24.0 Hz, 5H), 4.35 (s, 10H), 2.31 (s, 2H). LC-MS (ESI) (M + H)⁺ = 590.1. |
| 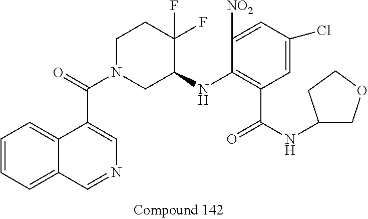 [SMILES: O=C(NC1CCOC1)c1cc(Cl)cc([N+](=O)[O-])c1N[C@H]1CN(C(=O)c2cncc3ccccc23)CCC1(F)F] | Compound 142: 5-chloro-2-(((S)-4,4-difluoro-1-(isoquinoline-4- carbonyl)piperidin-3-yl)amino)-3-nitro-N-((R)-tetrahydrofuran- 3-yl)benzamide ¹H NMR (400 MHz, DMSO-d₆) δ 9.42 (s, 1H), 9.20-8.89 (m, 1H), 8.57 (s, 1H), 8.27-8.00 (m, 2H), 7.95-7.53 (m, 5H), 4.74- 4.23 (m, 2H), 3.93-3.65 (m, 5H), 3.62 (d, J = 12.0 Hz, 2H), 2.36- 1.33 (m, 5H). LC-MS (ESI) (M + H)⁺ = 558.2. |
| 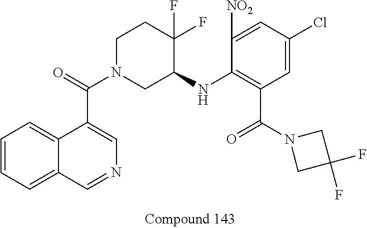 [SMILES: O=C(c1cc(Cl)cc([N+](=O)[O-])c1N[C@H]1CN(C(=O)c2cncc3ccccc23)CCC1(F)F)N1CC(F)(F)C1] | Compound 143: (S)-(5-chloro-2-((4,4-difluoro-1-(isoquinoline-4- carbonyl)piperidin-3-yl)amino)-3-nitrophenyl)(3,3-difluoroazetidin- 1-yl)methanone ¹H NMR (400 MHz, DMSO-d₆) δ 9.41 (d, J = 6.0 Hz, 1H), 8.57 (s, 1H), 8.36-8.05 (m, 2H), 8.00-6.83 (m, 5H), 5.14-3.48 (m, 8H), 2.08 (s, 3H). LC-MS (ESI) (M + H)⁺ = 566.2. |
| 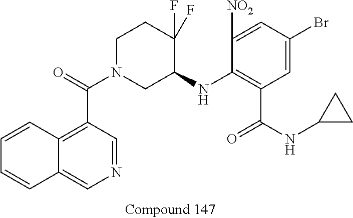 [SMILES: O=C(NC1CC1)c1cc(Br)cc([N+](=O)[O-])c1N[C@H]1CN(C(=O)c2cncc3ccccc23)CCC1(F)F] | Compound 147: (S)-5-bromo-N-cyclopropyl-2-((4,4-difluoro-1- (isoquinoline-4-carbonyl)piperidin-3-yl)amino)-3-nitrobenzamide ¹H NMR (400 MHz, DMSO-d₆) δ 9.41 (s, 1H), 9.07-8.37 (m, 2H), 8.23 (d, J = 12.0 Hz, 2H), 7.82 (d, J = 40.0 Hz, 5H), 3.67 (s, 3H), 2.89 (s, 2H), 2.24-0.36 (m, 7H). LC-MS (ESI) (M + H)⁺ = 574.2. |
| 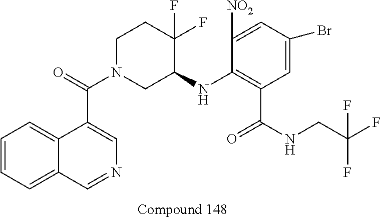 [SMILES: O=C(NCC(F)(F)F)c1cc(Br)cc([N+](=O)[O-])c1N[C@H]1CN(C(=O)c2cncc3ccccc23)CCC1(F)F] | Compound 148: (S)-5-bromo-2-((4,4-difluoro-1-(isoquinoline- 4-carbonyl)piperidin-3-yl)amino)-3-nitro-N-(2,2,2-trifluoroethyl) benzamide ¹H NMR (400 MHz, DMSO-d₆) δ 9.72 (s, 1H), 9.55 (s, 1H), 8.65 (s, 1H), 8.47-8.21 (m, 3H), 7.93 (dd, J = 32.0, 8.0 Hz, 4H), 4.24 (s, 4H), 2.87 (d, J = 64.0 Hz, 1H), 2.44-1.97 (m, 4H). LC-MS (ESI) (M + H)⁺ = 616.1. |
| 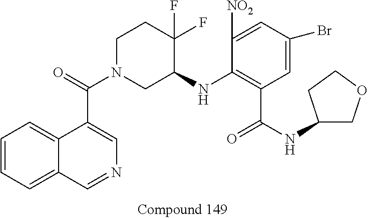 [SMILES: O=C(N[C@H]1CCOC1)c1cc(Br)cc([N+](=O)[O-])c1N[C@H]1CN(C(=O)c2cncc3ccccc23)CCC1(F)F] | Compound 149: 5-bromo-2-(((S)-4,4-difluoro-1-(isoquinoline- 4-carbonyl)piperidin-3-yl)amino)-3-nitro-N-((S)-tetrahydrofuran- 3-yl)benzamide ¹H NMR (400 MHz, DMSO-d₆) δ 9.49 (s, 1H), 9.11 (dd, J = 72.0, 6.0 Hz, 1H), 8.64 (s, 1H), 8.38-7.57 (m, 7H), 4.79-3.59 (m, 9H), 2.42-1.32 (m, 5H). LC-MS (ESI) (M + H)⁺ = 604.1. |
| 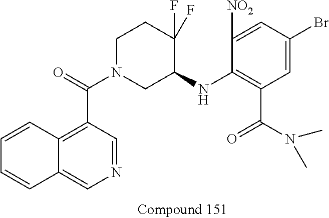 [SMILES: CN(C)C(=O)c1cc(Br)cc([N+](=O)[O-])c1N[C@H]1CN(C(=O)c2cncc3ccccc23)CCC1(F)F] | Compound 151: (S)-5-bromo-2-((4,4-difluoro-1-(isoquinoline-4- carbonyl)piperidin-3-yl)amino)-N, N-dimethyl-3-nitrobenzamide ¹H NMR (400 MHz, DMSO-d₆) δ 9.41 (d, J = 8.0 Hz, 1H), 8.57 (s, 1H), 8.40-8.10 (m, 2H), 7.85 (m, 4H), 7.08 (s, 1H), 4.51 (m, 1H), 3.78 (m, 4H), 3.04 (m, 6H), 2.08 (s, 2H). LC-MS (ESI) (M + H)⁺ = 562.1. |
| 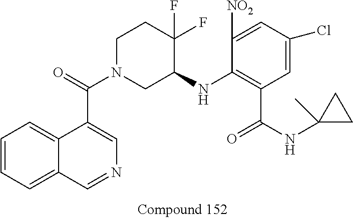 [SMILES: CC1(NC(=O)c2cc(Cl)cc([N+](=O)[O-])c2N[C@H]2CN(C(=O)c3cncc4ccccc34)CCC2(F)F)CC1] | Compound 152: (S)-5-chloro-2-((4,4-difluoro-1-(isoquinoline- 4-carbonyl)piperidin-3-yl)amino)-N-(1-methylcyclopropyl)- 3-nitrobenzamide ¹H NMR (400 MHz, DMSO-d₆) δ 9.42 (d, J = 8.0 Hz, 1H), 9.12 (d, J = 60.0 Hz, 1H), 8.65 (d, J = 88.0 Hz, 1H), 8.22 (dd, J = 16, 10.2 Hz, 2H), 8.03-7.56 (m, 5H), 4.65-3.47 (m, 4H), 2.15 (d, J = 88.0 Hz, 2H), 1.56-0.44 (m, 8H). LC-MS (ESI) (M + H)⁺ = 544.2. |
| 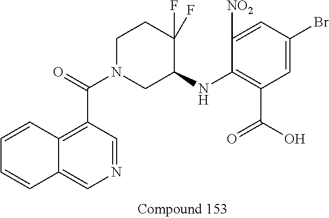 [SMILES: O=C(O)c1cc(Br)cc([N+](=O)[O-])c1N[C@H]1CN(C(=O)c2cncc3ccccc23)CCC1(F)F] | Compound 153: (S)-5-bromo-2-((4,4-difluoro-1-(isoquinoline-4- carbonyl)piperidin-3-yl)amino)-3-nitrobenzoic acid ¹H NMR (400 MHz, DMSO-d₆) δ 9.41 (m, 1H), 8.47-9.01 (m, 2H), 8.04-8.35 (m, 3H), 7.71-7.92 (m, 3H), 4.24-4.72 (m, 1H), 4.04-4.20 (m, 1H), 3.55-3.74 (m, 3H), 2.26 (m, 1H), 1.88-2.12 (m, 1H). LC-MS (ESI) (M + H)⁺ = 535.1. |
| 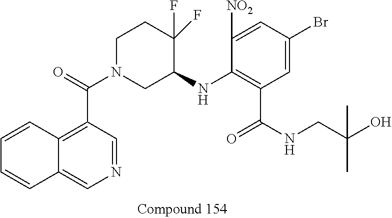 [SMILES: CC(C)(O)CNC(=O)c1cc(Br)cc([N+](=O)[O-])c1N[C@H]1CN(C(=O)c2cncc3ccccc23)CCC1(F)F] | Compound 154: (S)-5-bromo-2-((4,4-difluoro-1-(isoquinoline-4- carbonyl)piperidin-3-yl)amino)-N-(2-hydroxy-2-methylpropyl)-3- nitrobenzamide ¹H NMR (400 MHz, DMSO-d₆) δ 9.39 (s, 1H), 8.71 (d, J = 88.0 Hz, 2H), 8.28-7.72 (m, 7H), 4.60-3.60 (m, 5H), 2.11 (d, J = 92.0 Hz, 2H), 1.51-0.91 (m, 9H). LC-MS (ESI) (M + H)⁺ = 606.1. |
| 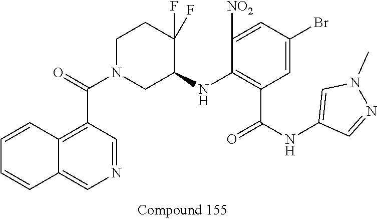 [SMILES: Cn1cc(NC(=O)c2cc(Br)cc([N+](=O)[O-])c2N[C@H]2CN(C(=O)c3cncc4ccccc34)CCC2(F)F)cn1] | Compound 155: (S)-5-bromo-2-((4,4-difluoro-1-(isoquinoline-4- carbonyl)piperidin-3-yl)amino)-N-(1-methyl-1H-pyrazol-4-yl)-3- nitrobenzamide ¹H NMR (400 MHz, DMSO-d₆) δ 10.94 (d, J = 76.0 Hz, 1H), 9.42 (s, 1H), 8.68-8.14 (m, 3H), 8.12-6.91 (m, 7H), 3.74 (d, J = 20.0 Hz, 7H), 2.37-1.24 (m, 3H). LC-MS (ESI) (M + H)⁺ = 614.1. |
| 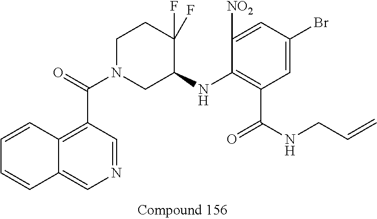 [SMILES: C=CCNC(=O)c1cc(Br)cc([N+](=O)[O-])c1N[C@H]1CN(C(=O)c2cncc3ccccc23)CCC1(F)F] | Compound 156: (S)-N-ally1-5-bromo-2-((4,4-difluoro-1- (isoquinoline-4-carbonyl)piperidin-3-yl)amino)-3-nitrobenzamide ¹H NMR (400 MHz, DMSO-d₆) δ 9.39 (s, 1H), 8.94 (s, -1H), 8.24 (d, J = 20.0 Hz, 2H), 7.93 (s, 1H), 7.86-7.69 (m, 5H), 5.11 (d, J = 12.0 Hz, 1H), 3.78 (d, J = 132.0 Hz, 4H), 2.31 (s, 4H), 1.22 (s, 3H). LC-MS (ESI) (M + H)⁺ = 574.2. |
| 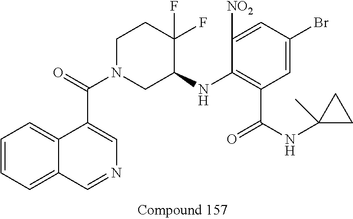 [SMILES: CC1(NC(=O)c2cc(Br)cc([N+](=O)[O-])c2N[C@H]2CN(C(=O)c3cncc4ccccc34)CCC2(F)F)CC1] | Compound 157: (S)-5-bromo-2-((4,4-difluoro-1-(isoquinoline- 4-carbonyl)piperidin-3-yl)amino)-N-(1-methylcyclopropyl)- 3-nitrobenzamide ¹H NMR (400 MHz, DMSO-d₆) δ 9.47 (s, 1H), 9.15 (d, J = 60.0 Hz, 1H), 8.71 (d, J = 80.0 Hz, 1H), 8.43-8.21 (m, 2H), 8.17-7.72 (m, 5H), 4.42-3.55 (m, 4H), 2.44-1.86 (m, 2H), 1.60-1.23 (m, 4H), 0.97-0.57 (m, 4H). LC-MS (ESI) (M + H)⁺ = 588.1. |
| 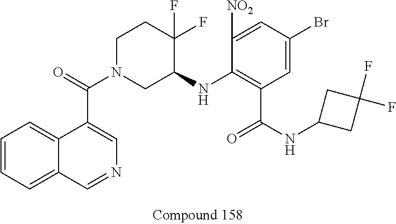 [SMILES: O=C(NC1CC(F)(F)C1)c1cc(Br)cc([N+](=O)[O-])c1N[C@H]1CN(C(=O)c2cncc3ccccc23)CCC1(F)F] | Compound 158: (S)-5-chloro-2-((4,4-difluoro-1-(isoquinoline-4- carbonyl)piperidin-3-yl)amino)-N-(3,3-difluorocyclobutyl)-3- nitrobenzamide ¹H NMR (400 MHz, DMSO-d₆) δ 9.42 (d, J = 6.0 Hz, 1H), 9.27 (dd, J = 74.0, 6.0 Hz, 1H), 8.59 (s, 1H), 8.30-7.63 (m, 7H), 4.46 (d, J = 146 Hz, 5H), 2.78 (s, 4H), 2.25 (s, 3H). LC-MS (ESI) (M + H)⁺ = 580.4. |
| 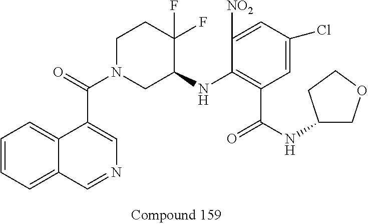 [SMILES: O=C(N[C@@H]1CCOC1)c1cc(Cl)cc([N+](=O)[O-])c1N[C@H]1CN(C(=O)c2cncc3ccccc23)CCC1(F)F] | Compound 159: 5-chloro-2-(((S)-4,4-difluoro-1-(isoquinoline-4- carbonyl)piperidin-3-yl)amino)-3-nitro-N-((S)-tetrahydrofuran-3- yl)benzamide ¹H NMR (400 MHz, DMSO-d₆) δ 9.48 (s, 1H), 9.12 (dd, J = 72.0, 8.0 Hz, 1H), 8.64 (s, 1H), 8.35-7.57 (m, 7H), 4.74-3.47 (m, 10H), 2.42-1.85 (m, 4H). LC-MS (ESI) (M + H)⁺ = 560.2. |

### Synthesis of Compounds 150

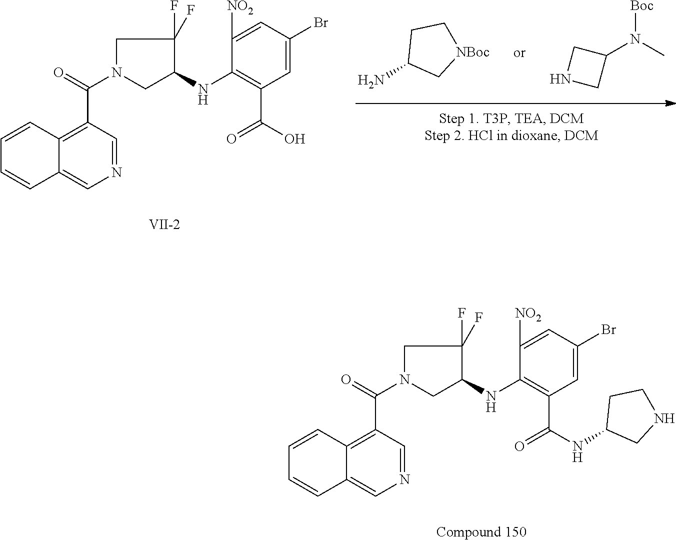 [SMILES: O=C(O)c1cc(Br)cc([N+](=O)[O-])c1N[C@H]1CN(C(=O)c2cncc3ccccc23)CC1(F)F ; CC(C)(C)OC(=O)N1CC[C@@H](N)C1 ; CN(C(=O)OC(C)(C)C)C1CNC1 ; "VII-2" ; O=C(N[C@@H]1CCNC1)c1cc(Br)cc([N+](=O)[O-])c1N[C@H]1CN(C(=O)c2cncc3ccccc23)CC1(F)F ; "Compound 150"]

### Intermediate VII-2: 5-bromo-2-[[(3S)-4,4-difluoro-1-(isoquinoline-4-carbonyl)pyrrolidin-3-yl]amino]-3-nitro-benzoic acid

The synthetic procedure of intermediate VIII-2 is similar to that of intermediate IV-5.

### Compounds 150

Compound 150 was synthesized from VII-1-2 with two steps (step1: described in method 1 of compound 19, step 2: described in the procedure of I-3).

| | |
|---|---|
| 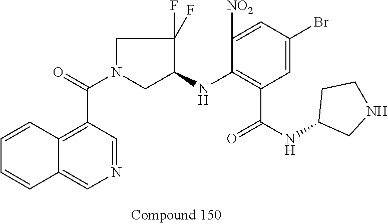 [SMILES: O=C(N[C@@H]1CCNC1)c1cc(Br)cc([N+](=O)[O-])c1N[C@H]1CN(C(=O)c2cncc3ccccc23)CC1(F)F] | Compound 150: 5-bromo-2-[[(3S)-4,4-difluoro-1-(isoquinoline-4- carbonyl)pyrrolidin-3-yl]amino]-3-nitro-N-[(3R)-pyrrolidin-3-yl]benzamide ¹H NMR (400 MHz, methanol-d₄) δ 9.84 (s, 1H), 8.80 (br d, J = 6.0 Hz, 1H), 8.58 (br d, J = 8.0 Hz, 1H), 8.24-8.34 (m, 2H), 8.23 (d, J = 2.8 Hz, 1H), 8.08-8.14 (m, 1H), 7.92-8.01 (m, 1H), 4.53-4.70 (m, 1H), 4.39-4.52 (m, 1H), 4.16-4.33 (m, 2H), 3.73-3.96 (m, 2H), 3.53-3.70 (m, 2H), 3.40-3.48 (m, 2H), 2.27-2.51 (m, 1H), 2.04-2.23 (m, 1H). LC-MS (ESI) (M + 3)+ = 591.3. |

### Synthesis of Compounds 35, 82, 83, 84, 118, 144, 160, 164, 165, 172, 173 and 182

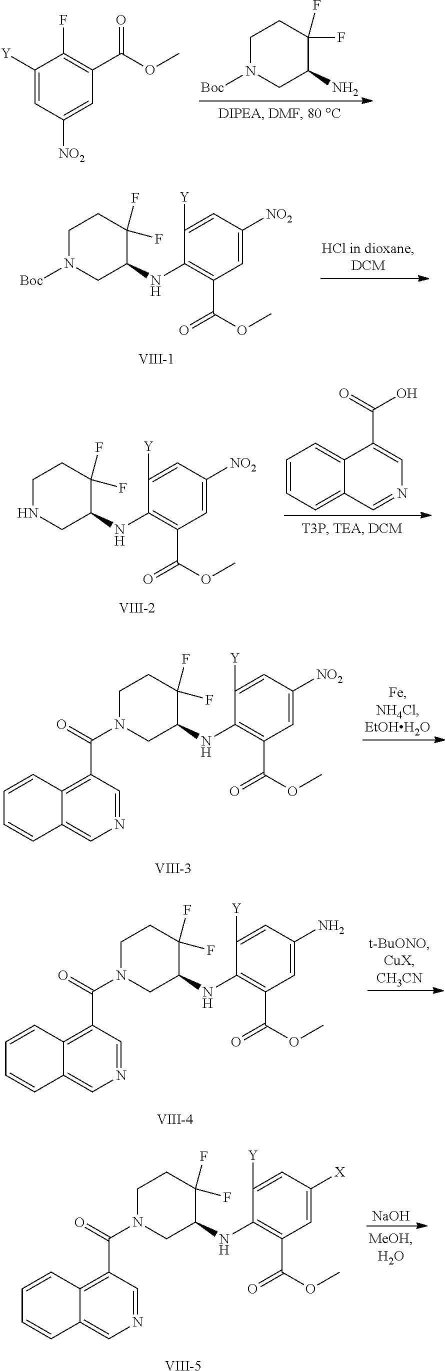

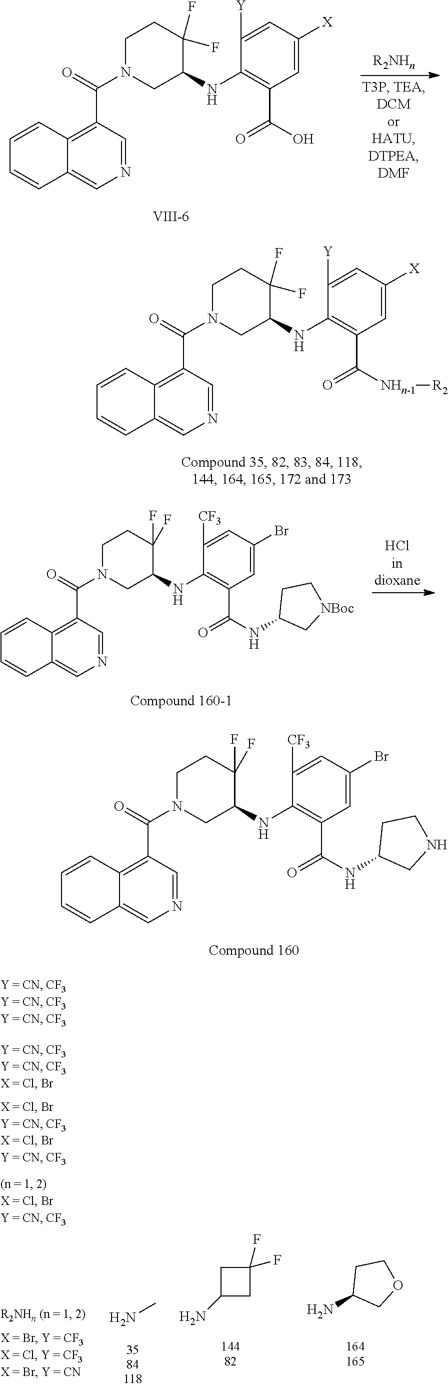

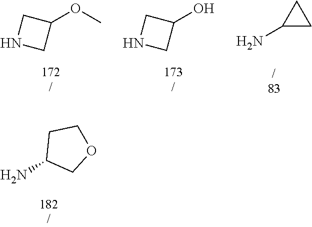

### Intermediates VIII-1~VIII-3

The synthetic procedures of intermediates VIII-1~VIII-3 are similar to that of intermediate IV-2~IV-4.

### Intermediate VIII-4

To a solution of intermediate VIII-3 (1.0 equiv) in EtOH/H₂O=4:1 (10 mL), Fe (8.0 equiv) and NH₄Cl (8.0 equiv) were added and the reaction was stirred at 40° C. for 16 hr. The reaction was filtered through a celite pad, and the filtrate was concentrated to give the crude product. The crude product was resuspended with water and extracted with EtOAc (50 mL*3). The organic layer was separated, and washed with saturated NaCl solution. The combined organic layers dried over Na₂SO₄ was filtered and concentrated under reduced pressure to dryness. The crude product was purified by silica gel chromatography eluted with ethyl acetate in petroleum ether to afford intermediate VIII-4.

### Intermediate VIII-5

t-BuONO (3.0 equiv) was slowly added to a solution of intermediate VIII-4 (1.0 equiv) in CH₃CN (2 mL) for 20 min, then CuX (5.0 equiv) was added and the reaction mixture was stirred at 70° C. for 2 hr. The reaction was concentrated under vacuum then added water (20 mL) and extracted with EtOAc (30 mL*3). The organic layer was separated, and washed with saturated NaCl solution. The combined organic layers dried over Na₂SO₄ was filtered and concentrated under reduced pressure to dryness. The crude product was purified by silica gel chromatography eluted with ethyl acetate in petroleum ether to afford intermediate VIII-5.

### Intermediate VIII-6

The synthetic procedure of intermediate VIII-6 is similar to that of intermediate IV-5.

### Compounds 35, 82, 83, 84, 118, 144, 160-1, 164, 165, 172, 173 and 182

The synthetic procedure is similar to the last step of the synthesis route of compound 14.

### Compound 160: 5-bromo-2-[[(3S)-4,4-difluoro-1-(isoquinoline-4-carbonyl)-3-piperidyl]amino]-N-[(3R)-pyrrolidin-3-yl]-3-(trifluoromethyl)benzamide

Compound 160 was obtained by removing protective group Boc using HCl in dioxane described in the procedure of intermediate I-3.

| | |
|---|---|
| 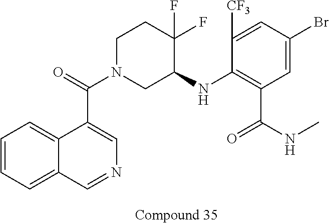 [SMILES: CNC(=O)c1cc(Br)cc(C(F)(F)F)c1N[C@H]1CN(C(=O)c2cncc3ccccc23)CCC1(F)F] | Compound 35: (S)-5-bromo-2-((4,4-difluoro-1-(isoquinoline-4- carbonyl)piperidin-3-yl)amino)-N-methyl-3-(trifluoromethyl)benzamide ¹H NMR (400 MHz, DMSO-d₆) δ 9.42 (s, 1H), 9.07-7.59 (m, 8H), 7.00 (m, 1H), 4.29 (m, 1H), 3.85 (s, 2H), 3.33-2.54 (m, 5H), 2.37-1.91 (m, 2H). LC- MS (ESI) (M + H)⁺ = 571.1. |
| 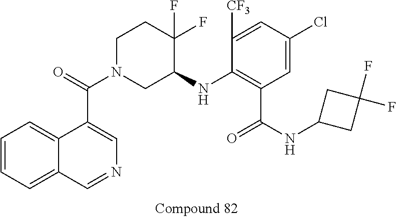 [SMILES: O=C(NC1CC(F)(F)C1)c1cc(Cl)cc(C(F)(F)F)c1N[C@H]1CN(C(=O)c2cncc3ccccc23)CCC1(F)F] | Compound 82: (S)-5-chloro-2-((4,4-difluoro-1 -(isoquinoline-4- carbonyl)piperidin-3-yl)amino)-N-(3,3-difluorocyclobutyl)-3- (trifluoromethyl)benzamide ¹H NMR (400 MHz, DMSO-d₆) δ 9.60-9.16 (m, 2H), 8.57 (s, 1H), 8.24 (d, J = 8.0 Hz, 1H), 7.98-7.60 (m, 5H), 6.96-6.48 (m, 1H), 4.26 (s, 4H), 3.01 (t, J = 104.0 Hz, 6H), 2.04 (d, J = 176.0 Hz, 2H). LC-MS (ESI) (M + H)⁺ = 603.1. |
| 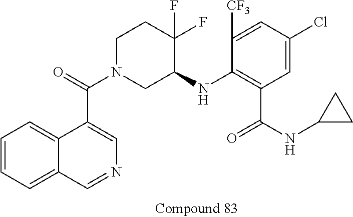 [SMILES: O=C(NC1CC1)c1cc(Cl)cc(C(F)(F)F)c1N[C@H]1CN(C(=O)c2cncc3ccccc23)CCC1(F)F] | Compound 83: (S)-5-chloro-N-cyclopropyl-2-((4,4-difluoro-1- (isoquinoline-4-carbonyl)piperidin-3-yl)amino)-3- (trifluoromethyl)benzamide ¹H NMR (400 MHz, DMSO-d₆) δ 9.41 (s, 1H), 9.05-8.47 (m, 2H), 8.23 (d, J = 10.0 Hz, 1H), 7.96-7.56 (m, 5H), 7.20-6.53 (m, 1H), 4.26 (d, J = 52.0 Hz, 1H), 3.69 (d, J = 154.0 Hz, 2H), 3.31-2.58 (m, 3H), 2.09 (dd, J = 122.0, 64.0 Hz, 2H), 1.02-0.42 (m, 4H). LC-MS (ESI) (M + H)⁺ = 553.2. |
| 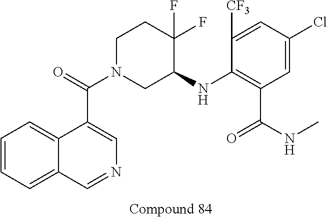 [SMILES: CNC(=O)c1cc(Cl)cc(C(F)(F)F)c1N[C@H]1CN(C(=O)c2cncc3ccccc23)CCC1(F)F] | Compound 84: (S)-5-chloro-2-((4,4-difluoro-1-(isoquinoline-4- carbonyl)piperidin -3-yl)amino)-N-methyl-3-(trifluoromethyl)benzamide ¹H NMR (400 MHz, DMSO-d₆) δ 9.42 (s, 1H), 8.74 (t, J = 80.0 Hz, 2H), 8.24 (d, J = 8.0 Hz, 1H), 7.96-7.57 (m, 4H), 6.97 (d, J = 80.0 Hz, 2H), 4.65- 3.50 (m, 4H), 3.27-2.60 (m, 4H), 2.37-1.97 (m, 2H). LC-MS (ESI) (M + H)⁺ = 527.2. |
| 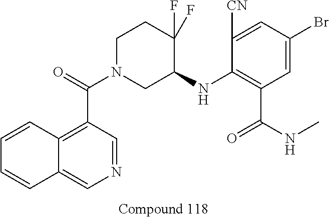 [SMILES: CNC(=O)c1cc(Br)cc(C#N)c1N[C@H]1CN(C(=O)c2cncc3ccccc23)CCC1(F)F] | Compound 118: (S)-5-bromo-3-cyano-2-((4,4-difluoro-1-(isoquinoline-4- carbonyl)piperidin-3-yl)amino)-N-methylbenzamide ¹H NMR (400 MHz, DMSO-d₆) δ 9.37 (d, J = 30.0 Hz, 1H), 9.02-8.42 (m, 4H), 8.26-8.01 (m, 2H), 7.93-7.61 (m, 3H), 4.92 - 3.85 (m, 5H), 2.89-2.64 (m, 3H), 2.27 (s, 2H). LC-MS (ESI) (M + H)⁺ = 528.2. |
| 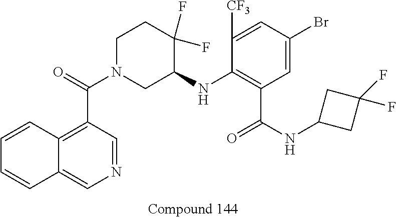 [SMILES: O=C(NC1CC(F)(F)C1)c1cc(Br)cc(C(F)(F)F)c1N[C@H]1CN(C(=O)c2cncc3ccccc23)CCC1(F)F] | Compound 144: (S)-5-bromo-2-((4,4-difluoro-1-(isoquinoline-4- carbonyl)piperidin-3-yl)amino)-N-(3,3-difluorocyclobutyl)-3- (trifluoromethyl)benzamide ¹H NMR (400 MHz, DMSO-d₆) δ 9.43 (s, 1H), 9.38-9.13 (m, 1H), 8.62 (d, J = 54.0 Hz, 1H), 8.24 (d, J = 10.0 Hz, 1H), 8.05-7.66 (m, 5H), 7.03-6.36 (m, 1H), 4.83-3.42 (m, 5H), 2.87 (d, J = 74.0 Hz, 4H), 2.28 (d, J = 10.0 Hz, 3H). LC-MS (ESI) (M + H)⁺ = 647.1. |
| 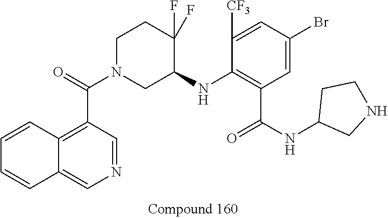 [SMILES: O=C(NC1CCNC1)c1cc(Br)cc(C(F)(F)F)c1N[C@H]1CN(C(=O)c2cncc3ccccc23)CCC1(F)F] | Compound 160: 5-bromo-2-[(3S)-4,4-difluoro-1-(isoquinoline-4- carbonyl)-3-piperidyl]amino]-N-[(3R)-pyrrolidin-3-y1]-3- (trifluoromethyl)benzamide ¹H NMR (400 MHz, DMSO-d₆) δ 9.36-9.48 (m, 1H), 8.93-9.15 (m, 1H), 8.49-8.74 (m, 1H), 8.23 (m, 1H), 7.70-7.95 (m, 5H), 6.39-6.89 (m, 1H), 4.22-4.68 (m, 2H), 3.41-3.95 (m, 7H), 1.69-2.30 (m, 6H). LC-MS (ESI) (M + H)⁺ = 626.4. |
| 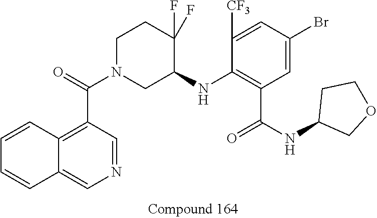 [SMILES: O=C(N[C@H]1CCOC1)c1cc(Br)cc(C(F)(F)F)c1N[C@H]1CN(C(=O)c2cncc3ccccc23)CCC1(F)F] | Compound 164: 5-bromo-2-(((S)-4,4-difluoro-1-(isoquinoline-4- carbonyl)piperidin-3-yl)amino)-N-((S)-tetrahydrofuran-3-yl)-3- (trifluoromethyl)benzamide ¹H NMR (400 MHz, DMSO-d₆) δ 9.42 (s, 1H), 9.19-8.96 (m, 1H), 8.75- 8.47 (m, 1H), 8.24 (d, J = 8.0 Hz, 1H), 7.99-7.64 (m, 5H), 7.03-6.55 (m, 1H), 4.41 (d, J = 40.0 Hz, 2H), 3.93-3.45 (m, 6H), 3.30 (s, 1H), 2.13 (d, J = 40.0 Hz, 5H). LC-MS (ESI) (M + H)⁺ = 627.1. |
| 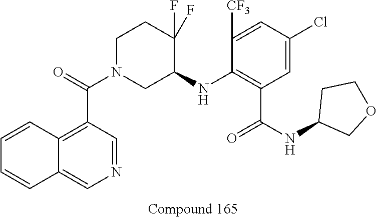 [SMILES: O=C(N[C@H]1CCOC1)c1cc(Cl)cc(C(F)(F)F)c1N[C@H]1CN(C(=O)c2cncc3ccccc23)CCC1(F)F] | Compound 165: 5-chloro-2-(((S)-4,4-difluoro-1-(isoquinoline-4-carbonyl) piperidin-3-yl)amino)-N-((S)-tetrahydrofuran-3-yl)-3- (trifluoromethyl)benzamide ¹H NMR (400 MHz, DMSO-d₆) δ 9.40 (s, 1H), 9.17-8.91 (m, 1H), 8.53 (s, 1H), 8.22 (d, J = 8.4 Hz, 1H), 7.94-7.56 (m, 4H), 6.59 (s, 2H), 4.68-3.45 (m, 8H), 3.29 (s, 2H), 2.35-1.84 (m, 4H). LC-MS (ESI) (M + H)⁺ = 583.2. |
| 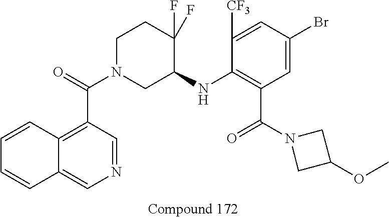 [SMILES: COC1CN(C(=O)c2cc(Br)cc(C(F)(F)F)c2N[C@H]2CN(C(=O)c3cncc4ccccc34)CCC2(F)F)C1] | Compound 172: (S)-(5-bromo-2-((4,4-difluoro-1-(isoquinoline-4- carbonyl)piperidin-3-yl)amino)-3-(trifluoromethyl)phenyl)(3- methoxyazetidin-1-yl)methanone ¹H NMR (400 MHz, DMSO-d₆) δ 9.39 (s, 1H), 8.66-7.59 (m, 7H), 5.47 (s, 1H), 4.08 (d, J = 120.0 Hz, 4H), 3.24 (d, J = 40.0 Hz, 8H), 2.53-2.46 (m, 1H), 2.15 (d, J = 80.0 Hz, 2H). LC-MS (ESI) (M + H)⁺ = 627.2. |
| 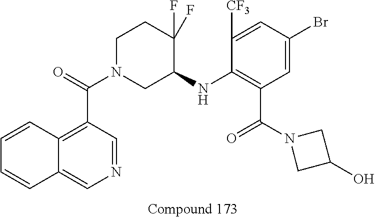 [SMILES: O=C(c1cc(Br)cc(C(F)(F)F)c1N[C@H]1CN(C(=O)c2cncc3ccccc23)CCC1(F)F)N1CC(O)C1] | Compound 173: (S)-(5-bromo-2-((4,4-difluoro-1-(isoquinoline-4- carbonyl)piperidin-3-yl)amino)-3-(trifluoromethyl)phenyl)(3- hydroxyazetidin-1-yl)methanone 1H NMR (400 MHz, DMSO-d₆) δ 9.49 (s, 1H), 8.74-8.22 (m, 2H), 8.02- 7.46 (m, 5H), 5.43 (d, J = 40.0 Hz, 1H), 4.82-4.18 (m, 3H), 3.44 (d, J = 100.0 Hz, 8H), 2.34-1.96 (m, 2H). LC-MS (ESI) (M + H)⁺ = 613.2. |
| 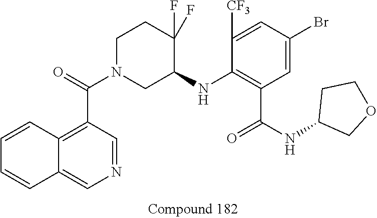 [SMILES: O=C(N[C@@H]1CCOC1)c1cc(Br)cc(C(F)(F)F)c1N[C@H]1CN(C(=O)c2cncc3ccccc23)CCC1(F)F] | Compound 182: 5-bromo-2-(((S)-4,4-difluoro-1-(isoquinoline-4- carbonyl)piperidin-3-yl)amino)-N-((R)-tetrahydrofuran-3-yl)-3- (trifluoromethyl)benzamide 1H NMR (400 MHz, DMSO-d₆) δ 9.41 (s, 1H), 8.99-9.20 (m, 1H), 8.46- 8.73 (m, 1H), 8.19-8.35 (m, 2H), 7.71-7.96 (m, 5H), 6.48-6.97 (m, 1H), 4.24-4.72 (m, 3H), 3.76-3.90 (m, 2H), 2.98-3.14 (m, 4H), 1.93-2.31 (m, 3H), 1.74-1.91 (m, 1H). LC-MS (ESI) (M + 3)+ = 629.3. |

### Synthesis of Compounds 109, 110, 145, 146, 167 and 183

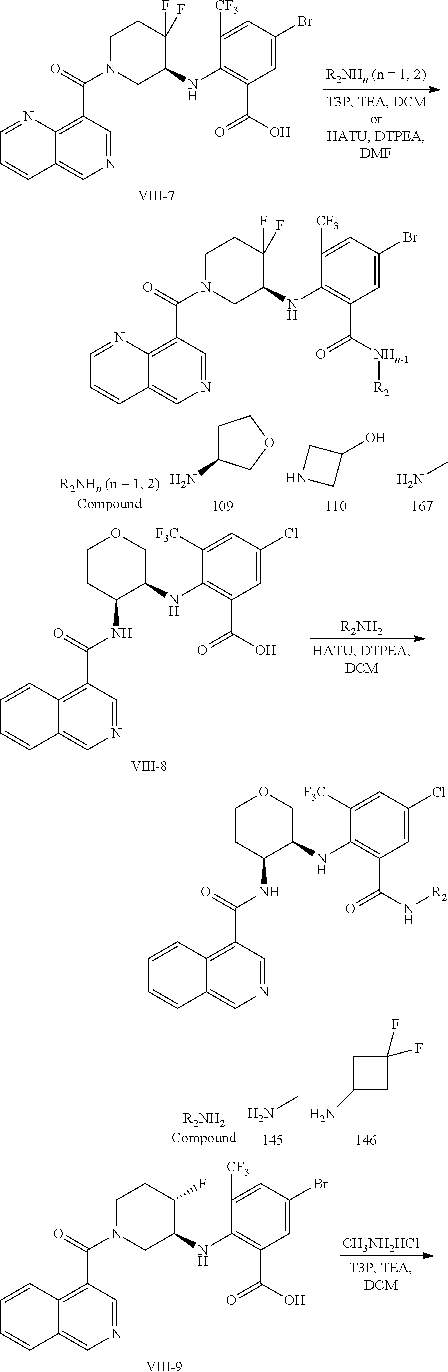

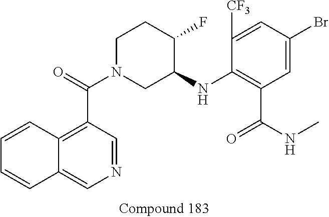 [SMILES: CNC(=O)c1cc(Br)cc(C(F)(F)F)c1N[C@H]1CN(C(=O)c2cncc3ccccc23)CC[C@@H]1F]

### Intermediates VIII-7, VIII-8 and VIII-9

The synthetic procedures of intermediate VIII-7, VIII-8 and VIII-9 are similar to that of intermediate VIII-6.

### Compounds 109, 110, 145, 146, 167 and 183

The synthetic procedure is similar to the last step of the synthesis route for compound 14.

| | |
|---|---|
| 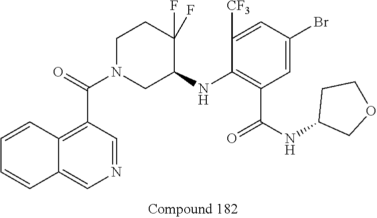 [SMILES: O=C(N[C@@H]1CCOC1)c1cc(Br)cc(C(F)(F)F)c1N[C@H]1CN(C(=O)c2cncc3ccccc23)CCC1(F)F] | Compound 109: 5-bromo-2-(((S)-4,4-difluoro-1-(1,6-naphthyridine-8- carbonyl)piperidin-3-yl)amino)-N-((S)-tetrahydrofuran-3-y1)-3- (trifluoromethyl)benzamide ¹H NMR (400 MHz, Methanol-d₄) δ 9.43-9.52 (m, 1H), 9.13-9.26 (m, 1H), 8.73-8.90 (m, 1H), 8.62-8.71 (m, 1H), 7.96-8.21 (m, 1H), 7.64- 7.85 (m, 3H), 4.47-4.83 (m, 2H), 4.11-4.46 (m, 1H), 3.86-3.99 (m, 2H), 3.70-3.85 (m, 2H), 3.66 (br dd, J = 9.2, 3.7 Hz, 1H), 3.40-3.60 (m, 2H), 2.17-2.42 (m, 2H), 1.87-2.15 (m, 2H). LC-MS (ESI) (M + H)⁺ = 630.3. |
| 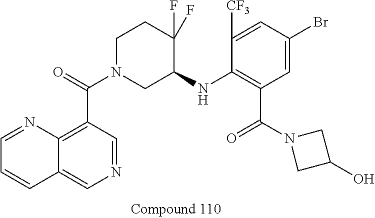 [SMILES: O=C(c1cc(Br)cc(C(F)(F)F)c1N[C@H]1CN(C(=O)c2cncc3cccnc23)CCC1(F)F)N1CC(O)C1] | Compound 110: (S)-(5-bromo-2-((4,4-difluoro-1-(1,6-naphthyridine-8- carbonyl)piperidin-3-yl)amino)-3-(trifluoromethyl)phenyl)(3- hydroxyazetidin-1-yl)methanone ¹H NMR (400 MHz, DMSO-d₆) δ 9.41-9.58 (m, 1H), 9.01-9.26 (m, 1H), 8.61-8.83 (m, 2H), 7.59-7.86 (m, 3H), 5.73-6.00 (m, 1H), 5.16-5.54 (m, 1H), 4.25-4.72 (m, 3H), 3.98-4.22 (m, 1H), 3.58-3.96 (m, 3H), 3.03-3.25 (m, 3H), 1.96-2.31 (m, 2H). LC-MS (ESI) (M + H)⁺ = 616.3. |
| 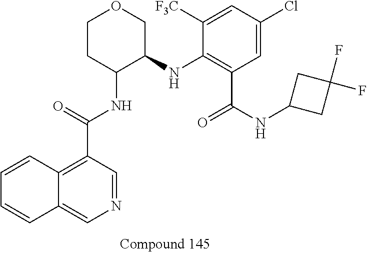 [SMILES: O=C(NC1CC(F)(F)C1)c1cc(Cl)cc(C(F)(F)F)c1N[C@@H]1COCCC1NC(=O)c1cncc2ccccc12] | Compound 145: N-((3S, 4S)-3-((4-chloro-2-((3,3- difluorocyclobutyl)carbamoyl)-6- (trifluoromethyl)phenyl)amino) tetrahydro-2H-pyran-4-yl)isoquinoline-4-carboxamide ¹H NMR (400 MHz, DMSO-d₆) δ 9.39 (s, 1H), 9.09 (d, J = 8.0 Hz, 1H), 8.73 (d, J = 8.0 Hz, 1H), 8.51 (s, 1H), 8.19 (t, J = 8.0 Hz, 2H), 7.82 (t, J = 8.0 Hz, 1H), 7.73 (t, J = 8.0 Hz, 1H), 7.69 (s, 2H), 6.31 (s, 1H), 4.43 (s, 1H), 4.13 (s, 1H), 3.84 (d, J = 12.0 Hz, 2H), 3.62 (d, J = 14.0 Hz, 1H), 3.50 (d, J = 12.0 Hz, 2H), 2.90 (s, 2H), 2.73 (s, 2H), 2.03-1.67 (m, 2H). LC-MS (ESI) (M + H)⁺ = 583.1. |
| 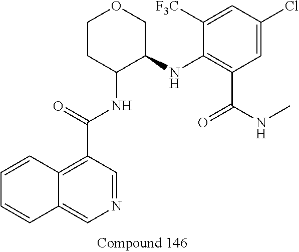 [SMILES: CNC(=O)c1cc(Cl)cc(C(F)(F)F)c1N[C@@H]1COCCC1NC(=O)c1cncc2ccccc12] | Compound 146: N-((3S, 4S)-3-((4-chloro-2-(methylcarbamoyl)-6- (trifluoromethyl)phenyl)amino)tetrahydro-2H-pyran-4-yl)isoquinoline- 4-carboxamide ¹H NMR (400 MHz, DMSO-d₆) δ 9.37 (s, 1H), 8.71 (d, J = 8.0 Hz, 1H), 8.62 (d, J = 6.0 Hz, 1H), 8.50 (s, 1H), 8.22-8.12 (m, 2H), 7.81 (t, J = 8.0 Hz, 1H), 7.73 (t, J = 7.6 Hz, 1H), 7.64 (s, 1H), 7.59 (s, 1H), 6.36 (s, 1H), 4.42 (s, 1H), 3.84 (d, J = 12.0 Hz, 2H), 3.65 (dd, J = 12.0, 6.0 Hz, 1H), 3.51 (d, J = 12.0 Hz, 2H), 2.71 (d, J = 6.0 Hz, 3H), 1.96 (d, J = 10.0 Hz, 1H), 1.73 (d, J = 14.0 Hz, 1H). LC-MS (ESI) (M + H)⁺ = 507.1. |
| 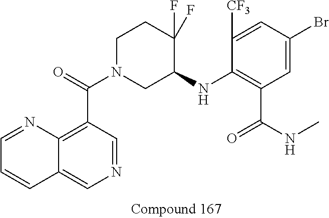 [SMILES: CNC(=O)c1cc(Br)cc(C(F)(F)F)c1N[C@H]1CN(C(=O)c2cncc3cccnc23)CCC1(F)F] | Compound 167: (S)-5-bromo-2-((4,4-difluoro-1-(1,6-naphthyridine-8- carbonyl)piperidin-3-yl)amino)-N-methyl-3-(trifluoromethyl)benzamide ¹H NMR (400 MHz, DMSO-d₆) δ 9.53-9.43 (m, 1H), 9.12 (d, J = 12.0 Hz, 1H), 8.95-8.59 (m, 3H), 7.95-7.43 (m, 4H), 3.57 (s, 2H), 3.23- 2.56 (m, 6H), 1.83 (s, 2H). LC-MS (ESI) (M + H)⁺ = 572.1. |
| 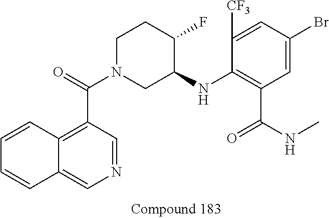 [SMILES: CNC(=O)c1cc(Br)cc(C(F)(F)F)c1N[C@H]1CN(C(=O)c2cncc3ccccc23)CC[C@@H]1F] | Compound 183: 5-bromo-2-(((3S, 4S)-4-fluoro-1-(isoquinoline-4- carbonyl)piperidin -3-yl)amino)-N-methyl-3-(trifluoromethyl)benzamide 1H NMR (400 MHz, DMSO-d6) δ 9.47 (s, 1H), 8.88-8.53 (m, 2H), 8.30 (d, J = 8.0 Hz, 1H), 8.00-7.67 (m, 4H), 6.55 (d, J = 68.0 Hz, 2H), 4.88-4.05 (m, 3H), 3.31-2.72 (m, 5H), 2.25-1.55 (m, 3H). LC-MS (ESI) (M + H)⁺ = 553.1. |

### Synthesis of Compounds 22, 23, 24, 26, 76 and 115

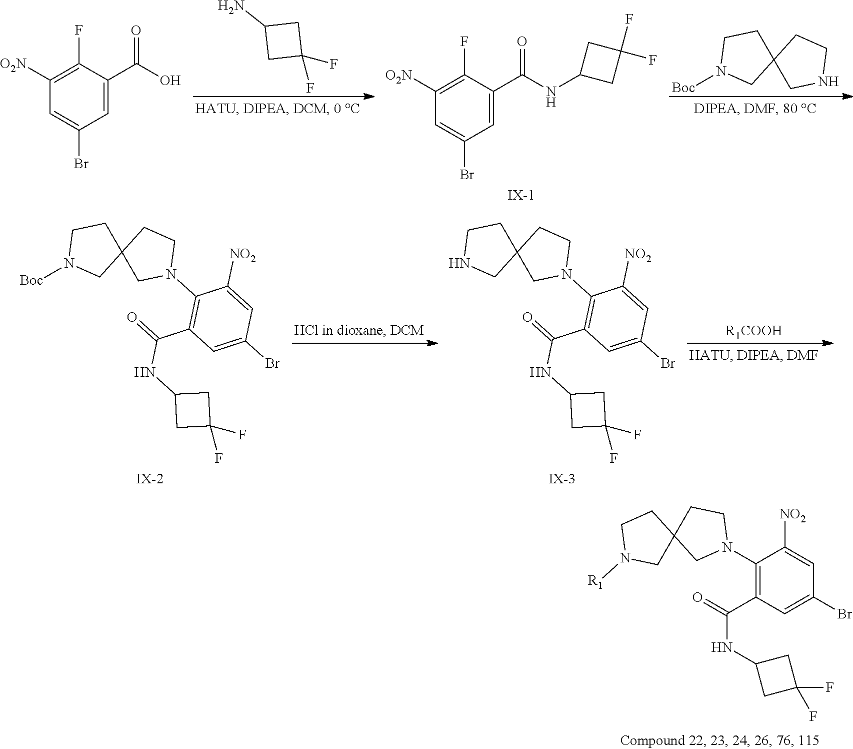

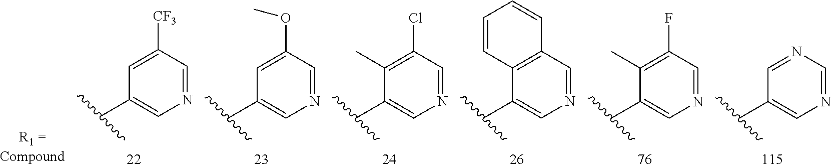

The synthetic procedures are similar to compound 1.

| | |
|---|---|
| 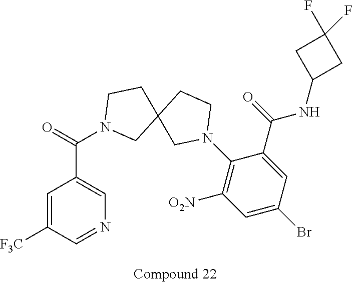 [SMILES: O=C(NC1CC(F)(F)C1)c1cc(Br)cc([N+](=O)[O-])c1N1CCC2(CCN(C(=O)c3cncc(C(F)(F)F)c3)C2)C1] | Compound 22: 5-bromo-N-(3,3-difluorocyclobutyl)-3-nitro-2-(7-(5- (trifluoromethyl)nicotinoyl)-2,7-diazaspiro[4.4]nonan-2-yl)benzamide ¹H NMR (400 MHz, Methanol-d₄) δ 9.02 (d, J = 2.0 Hz, 2H), 8.35 (dt, J = 4.0, 2.0 Hz, 1H), 7.92 (dd, J = 20.0, 2.0 Hz, 1H), 7.64 (dd, J = 12.0, 2.0 Hz, 1H), 3.82-3.40 (m, 6H), 3.29-3.18 (m, 2H), 3.07-2.89 (m, 2H), 2.73-2.57 (m, 2H), 2.22-1.87 (m, 5H). LC-MS (ESI) (M + H)⁺ = 632.1. |
| 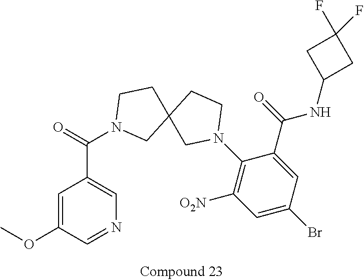 [SMILES: COc1cncc(C(=O)N2CCC3(CCN(c4c(C(=O)NC5CC(F)(F)C5)cc(Br)cc4[N+](=O)[O-])C3)C2)c1] | Compound 23: 5-bromo-N-(3,3-difluorocyclobutyl)-2-(7-(5- methoxynicotinoyl)-2,7-diazaspiro[4.4]nonan-2-yl)-3-nitrobenzamide ¹H NMR (400 MHz, Methanol-d₄) δ 8.34 (d, J = 4.0 Hz, 1H), 8.29 (s, 1H), 7.90 (dd, J = 16.0, 2.0 Hz, 1H), 7.62 (dd, J = 10.0, 2.0 Hz, 1H), 7.54 (s, 1H), 3.92 (d, J = 2.0 Hz, 3H), 3.74-3.38 (m, 6H), 3.23-3.09 (m, 2H), 3.05-2.87 (m, 2H), 2.76-2.52 (m, 2H), 2.09-1.93 (m, 5H). LC-MS (ESI) (M + H)⁺ = 594.1. |
| 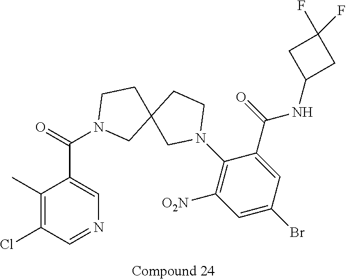 [SMILES: Cc1c(Cl)cncc1C(=O)N1CCC2(CCN(c3c(C(=O)NC4CC(F)(F)C4)cc(Br)cc3[N+](=O)[O-])C2)C1] | Compound 24: 5-bromo-2-(7-(5-chloro-4-methylnicotinoyl)-2,7-diazaspiro[4.4] nonan-2-yl)-N-(3,3-difluorocyclobutyl)-3-nitrobenzamide ¹H NMR (400 MHz, Methanol-d₄) δ 8.47 (s, 1H), 8.27 (d, J = 4.0 Hz, 1H), 7.81 (dd, J = 10.0, 2.0 Hz, 1H), 7.53 (dd, J = 6.0, 2.0 Hz, 1H), 4.14 (m, 2H), 3.71- 3.46 (m, 3H), 3.14-3.09 (m, 2H), 3.01-2.80 (m, 3H), 2.66-2.40 (m, 3H), 2.27 (d, J = 10.0 Hz, 3H), 1.98-1.81 (m, 4H). LC-MS (ESI) (M + H)⁺ = 612.1. |
| 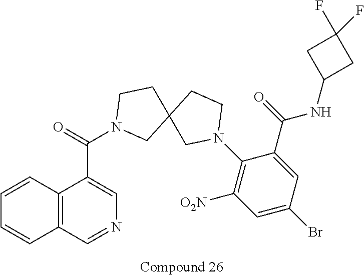 [SMILES: O=C(NC1CC(F)(F)C1)c1cc(Br)cc([N+](=O)[O-])c1N1CCC2(CCN(C(=O)c3cncc4ccccc34)C2)C1] | Compound 26: 5-bromo-N-(3,3-difluorocyclobutyl)-2-(7-(isoquinoline-4- carbonyl)-2,7-diazaspiro[4.4]nonan-2-yl)-3-nitrobenzamide ¹H NMR (400 MHz, DMSO-d₆) δ 9.38 (d, J = 4.1 Hz, 1H), 9.04 (dd, J = 68.9, 6.2 Hz, 1H), 8.50 (d, J = 15.2 Hz, 1H), 8.21 (dd, J = 8.1, 5.5 Hz, 1H), 7.96 (d, J = 2.5 Hz, 1H), 7.91-7.70 (m, 3H), 7.61 (dd, J = 38.6, 2.5 Hz, 1H), 4.07 (dd, J = 82.1, 8.7 Hz, 2H), 3.84-3.43 (m, 3H), 3.25-2.64 (m, 8H), 2.06-1.72 (m, 4H). LC-MS (ESI) (M + H)⁺ = 614.1. |
| 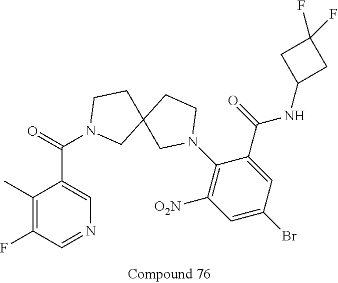 [SMILES: Cc1c(F)cncc1C(=O)N1CCC2(CCN(c3c(C(=O)NC4CC(F)(F)C4)cc(Br)cc3[N+](=O)[O-])C2)C1] | Compound 76: 5-bromo-N-(3,3-difluorocyclobutyl)-2-(7-(5-fluoro-4- methylnicotinoyl)-2,7-diazaspiro[4.4]nonan-2-yl)-3-nitrobenzamide ¹H NMR (400 MHz, Methanol-d₄) δ 8.34 (d, J = 2.0 Hz, 1H), 8.20 (d, J = 4.0 Hz, 1H), 7.82-7.79 (m, 1H), 7.54-7.50 (m, 1H), 4.14 (dd, J = 56.0, 8.0 Hz, 2H), 3.68-3.46 (m, 3H), 3.14-3.09 (m, 2H), 2.97-2.75 (m, 3H), 2.65-2.41 (m, 3H), 2.19 (dd, J = 6.0, 2.0 Hz, 3H), 1.95-1.81 (m, 4H). LC-MS (ESI) (M + H)⁺ = 596.0. |
| 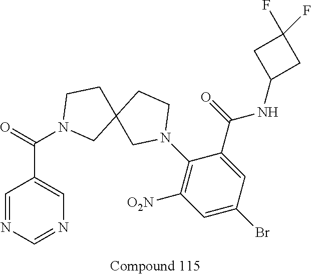 [SMILES: O=C(NC1CC(F)(F)C1)c1cc(Br)cc([N+](=O)[O-])c1N1CCC2(CCN(C(=O)c3cncnc3)C2)C1] | Compound 115: 5-bromo-N-(3,3-difluorocyclobutyl)-3-nitro-2-(7-(pyrimidine- 5-carbonyl)-2,7-diazaspiro[4.4]nonan-2-yl)benzamide ¹H NMR (400 MHz, Methanol-d₄) δ 10.08 (s, 1H), 9.79 (d, J = 10.0 Hz, 2H), 8.86-8.80 (m, 1H), 8.47-8.42 (m, 1H), 4.98 (d, J = 8.0 Hz, 1H), 4.46-4.20 (m, 5H), 3.91 (d, J = 28.0 Hz, 2H), 3.82-3.69 (m, 2H), 3.55-3.42 (m, 2H), 2.85-2.62 (m, 5H). LC-MS (ESI) (M + H)⁺ = 565.1. |

### Synthesis of Compounds 79 and 80

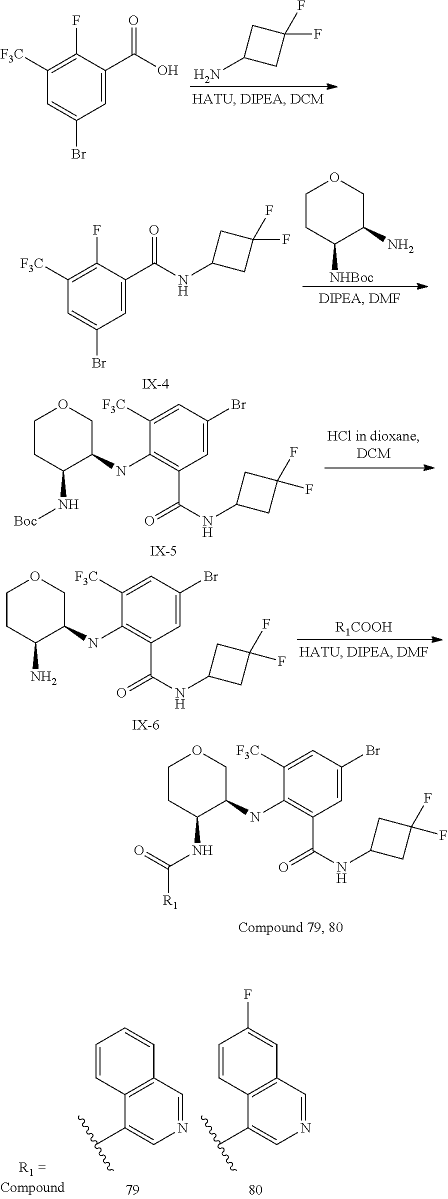

Compounds 79 and 80 were synthesized using a similar synthetic route for compound 22.

| | |
|---|---|
| 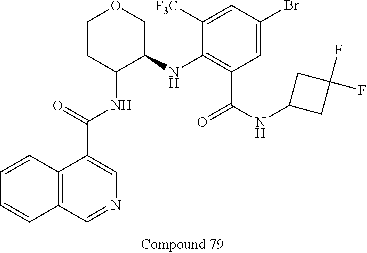 [SMILES: O=C(NC1CC(F)(F)C1)c1cc(Br)cc(C(F)(F)F)c1N[C@@H]1COCCC1NC(=O)c1cncc2ccccc12] | Compound 79: N-((3S, 4S)-3-((4-bromo-2-((3,3- difluorocyclobutyl)carbamoyl)-6-(trifluoromethyl)phenyl)amino)tetrahydro- 2H-pyran-4-yl)isoquinoline-4-carboxamide ¹H NMR (400 MHz, Methanol-d₄) δ 9.29 (s, 1H), 8.43 (s, 1H), 8.21 (d, J = 8.0 Hz, 1H), 8.15 (d, J = 8.0 Hz, 1H), 7.84 (ddd, J = 8.0, 8.0, 4.0 Hz, 1H), 7.78-7.71 (m, 2H), 7.65 (d, J = 4.0 Hz, 1H), 4.60 (s, 1H), 4.44 (dt, J = 12.0, 4.0 Hz, 1H), 4.22 (tdd, J = 8.0, 4.0, 4.0 Hz, 1H), 4.03-3.93 (m, 2H), 3.79 (dd, J = 12.0, 4.0 Hz, 1H), 3.63-3.54 (m, 2H), 3.31 (d, J = 4.0 Hz, 1H), 3.29 (d, J = 4.0 Hz, 1H), 2.93 (qd, J = 12.0, 8.0, 4.0 Hz, 2H), 2.75-2.63 (m, 2H), 2.08-1.98 (m, 1H), 1.87 (dd, J = 16.0, 4.0 Hz, 1H). LC-MS (ESI) (M + H)⁺ = 627.1. |
| 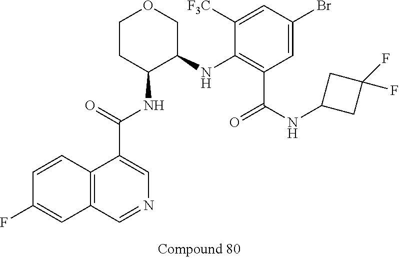 [SMILES: O=C(NC1CC(F)(F)C1)c1cc(Br)cc(C(F)(F)F)c1N[C@@H]1COCC[C@@H]1NC(=O)c1cncc2cc(F)ccc12] | Compound 80: N-((3S, 4S)-3-((4-bromo-2-((3,3- difluorocyclobutyl)carbamoyl)-6-(trifluoromethyl)phenyl)amino)tetrahydro- 2H-pyran-4-yl)-7-fluoroisoquinoline-4-carboxamide ¹H NMR (400 MHz, Methanol-d₄) δ 9.31 (s, 1H), 8.47 (s, 1H), 8.34 (dd, J = 9.4, 6.0 Hz, 1H), 7.88 (dd, J = 8.8, 4.0 Hz, 1H), 7.80-7.63 (m, 3H), 4.46 (dt, J = 11.8, 6.0 Hz, 1H), 4.24 (s, 1H), 4.09-3.94 (m, 2H), 3.86-3.77 (m, 1H), 3.60 (ddd, J = 12.2, 9.4, 6.0 Hz, 2H), 2.95 (dq, J = 14.1, 8.0 Hz, 2H), 2.81-2.62 (m, 2H), 2.05 (td, J = 12.5, 11.8, 6.0 Hz, 1H), 1.89 (d, J = 14.0 Hz, 1H). LC-MS (ESI) (M + H)⁺ = 645.1. |

### Synthesis of Compounds 134 and 137

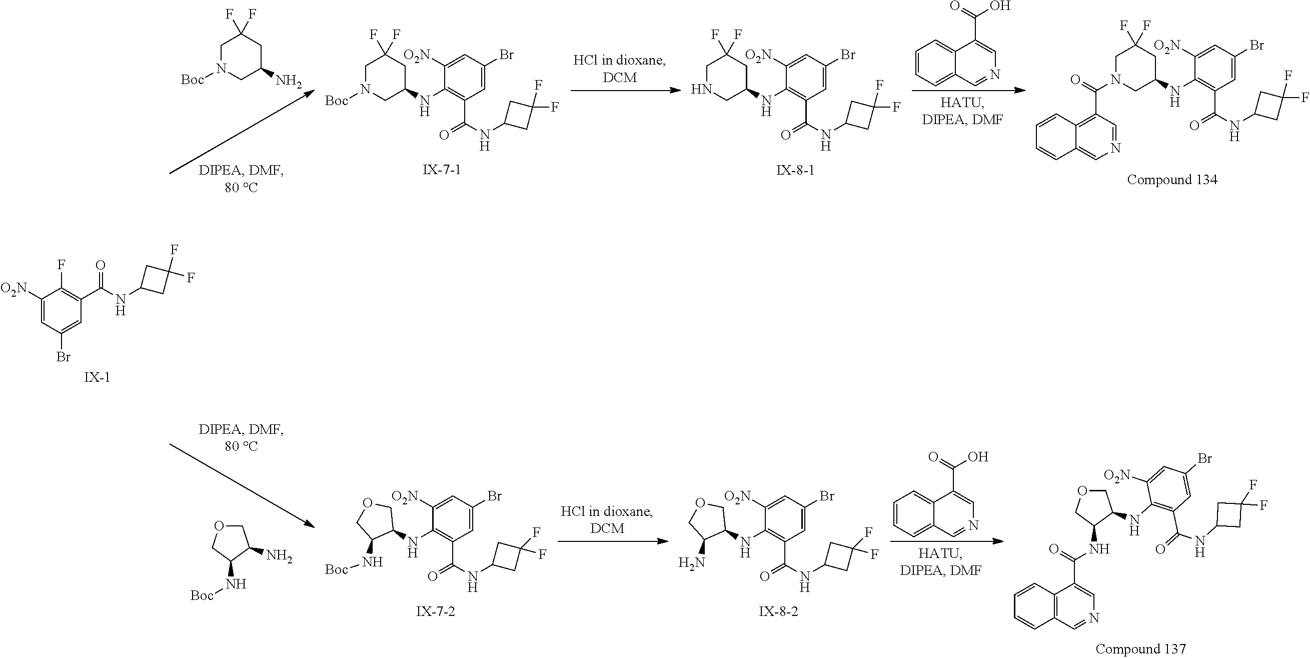

Compounds 134 and 137 were synthesized using a similar synthesis route of compound 22.

| | |
|---|---|
| 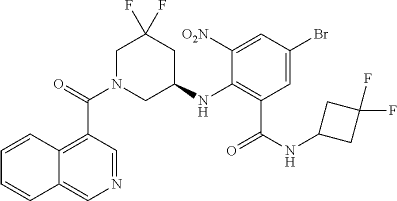 [SMILES: O=C(NC1CC(F)(F)C1)c1cc(Br)cc([N+](=O)[O-])c1N[C@H]1CN(C(=O)c2cncc3ccccc23)CC(F)(F)C1] | Compound 134: (R)-5-bromo-2-((5,5-difluoro-1-(isoquinoline-4- carbonyl)piperidin-3-yl)amino)-N-(3,3-difluorocyclobutyl)-3- nitrobenzamide ¹H NMR (400 MHz, DMSO-d₆) δ 9.46-8.98 (m, 2H), 8.45 (d, J = 40.0 Hz, 1H), 8.33-7.61 (m, 7H), 4.29-3.67 (m, 4H), 3.56 (d, J = 12.0 Hz, 2H), 2.84 (d, J = 76.0 Hz, 5H), 2.33 (d, J = 16.0 Hz, 1H). LC-MS (ESI) (M + H)⁺ = 624.1. |
| Compound 134 | |
| 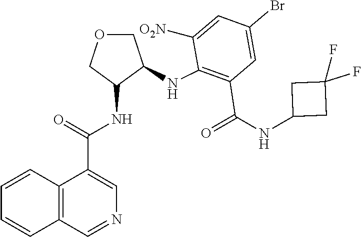 | Compound 137: N-((3R,4S)-4-((4-bromo-2-((3,3- difluorocyclobutyl)carbamoyl)-6-nitrophenyl)amino)tetrahydrofuran-3- yl)isoquinoline-4-carboxamide ¹H NMR (400 MHz, DMSO-d₆) δ 9 9.42 (s, 1H), 9.16 (d, J = 8.0 Hz, 1H), 9.06 (d, J = 8.0 Hz, 1H), 8.68 (s, 1H), 8.29 (d, J = 8.0 Hz, 1H), 8.23-8.15 (m, 2H), 8.05 (d, J = 8.0 Hz, 1H), 7.86 (d, J = 4.0 Hz, 1H), 7.80-7.71 ( m,2H), 4.70-4.61 (m, 1H), 4.22 (dd, J = 8.0, 4.0Hz, 1H), 4.08 (dd, J = 12.0, 8.0 Hz, 2H), 3.99 (dd, J = 12.0, 4.0 Hz, 1H), 3.84 (dd, J = 8.0, 4.0 Hz, 1H), 3.65 (dd, J = 8.0, 4.0 Hz, 1H), 2.97-2.78 (m, 2H), 2.75-2.64 (m, 2H). LC-MS (ESI) (M + H)⁺ = 590.1. |
| Compound 137 | |

### Synthesis of Compound 30

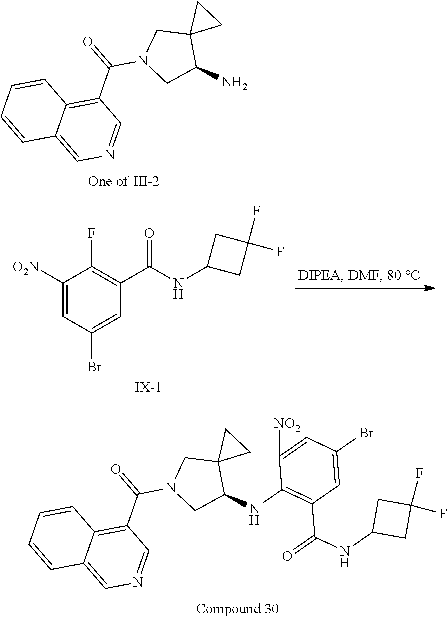

### Compound 30: (R)-5-bromo-N-(3,3-difluorocyclobutyl)-2-((5-(isoquinoline-4-carbonyl)-5-azaspiro[2.4]heptan-7-yl)amino)-3-nitrobenzamide

To a solution of intermediate IX-1 (95 mg, 0.27 mmol) and (R)-(7-amino-5-azaspiro[2.4]heptan-5-yl)(isoquinolin-4-yl) methanone (60 mg, 0.22 mmol) in DMF (2.0 mL), DIPEA (0.1 mL, 0.67 mmol) was added and the reaction was stirred at 80° C. at N₂ atmosphere for 16 hr. The reaction mixture was poured into water and then extracted with EtOAc (20.0 mL*3). The combined organic layer was washed with brine (20.0 mL*3), dried over Na₂SO₄ and concentrated to give a crude product, which was purified by Flash Chromatography eluting with EtOAc in PE (PE:EtOAc=2:1) and prep-HPLC to give compound 30 (2.0 mg, 2% yield) as a yellow solid.

| | |
|---|---|
| 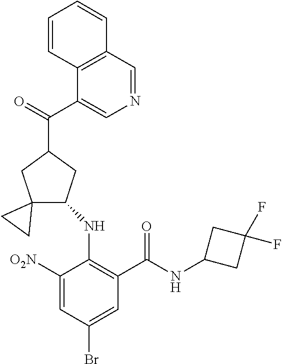 [SMILES: O=C(NC1CC(F)(F)C1)c1cc(Br)cc([N+](=O)[O-])c1N[C@H]1CC(C(=O)c2cncc3ccccc23)CC12CC2] | Compound 30: (R)-5-bromo-N-(3,3-difluorocyclobutyl)-2-((5-(isoquinoline-4- carbonyl)-5-azaspiro[2.4]heptan-7-yl)amino)-3-nitrobenzamide ¹H NMR (400 MHz, DMSO-d₆) δ 9.36 (d, J = 7.7 Hz, 1H), 9.20 (m, 1H), 8.42 (d, J = 36.0 Hz, 1H), 8.29-8.16 (m, 2H), 7.97 (d, J = 3.0 Hz, 1H), 7.86-7.48 (m, 4H), 4.19-3.46 (m, 4H), 3.08-2.62 (m, 6H), 0.86-0.56 (m, 4H). LC-MS (ESI) (M + H)⁺ = 600.1. |
| Compound 30 | |

### Synthesis of Compound 32

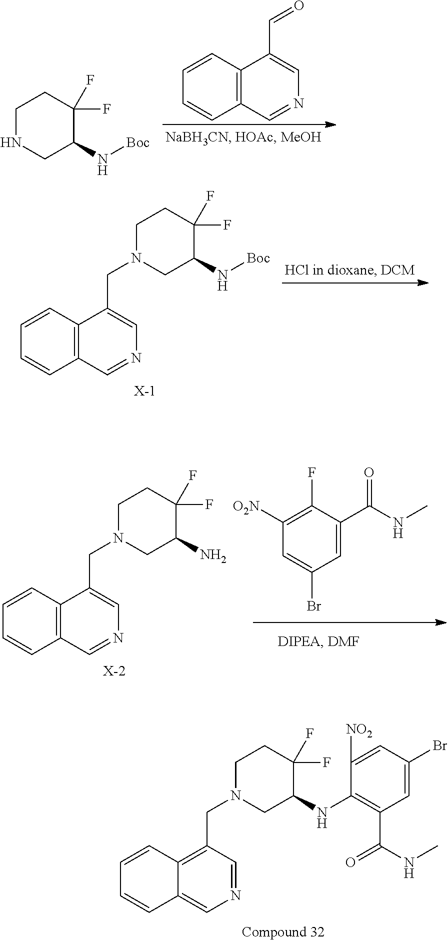

### Intermediate X-1: tert-butyl (S)-(4,4-difluoro-1-(isoquinolin-4-ylmethyl)piperidin-3-yl)carbamate

To a solution of tert-butyl (S)-(4,4-difluoropiperidin-3-yl)carbamate (2.0 g, 8.47 mmol) and isoquinoline-4-carbaldehyde (1.30 g, 8.47 mmol) in MeOH (30.0 mL), HOAc (60 mg, 1 mmol) and NaBH₃CN (1.06 g, 16.94 mmol) were added and the reaction mixture was stirred at room temperature for 2 hr. The reaction mixture was poured into water and then extracted with EtOAc (50.0 mL*3). The combined organic layer was washed with brine (50.0 mL*3), dried over Na2_{S}O₄ and concentrated to give a crude product which was purified by Flash Chromatography eluting with ethyl acetate in petroleum ether (EtOAc:PE=1:10) to afford the title compound (3.0 g, 97% yield) as a light yellow liquid. LC-MS (ESI) (M+H)⁺=378.1.

### Intermediate X-2: (S)-4,4-difluoro-1-(isoquinolin-4-ylmethyl)piperidin-3-amine

The synthetic procedure is similar to that of intermediate I-3.

### Compound 32: (S)-5-bromo-2-((4,4-difluoro-1-(isoquinolin-4-ylmethyl)piperidin-3-yl)amino)-N-methyl-3-nitrobenzamide

DIPEA (136 mg, 1.05 mmol) was added to a solution of intermediate X-2 (60 mg, 0.21 mmol) and 5-bromo-2-fluoro-N-methyl-3-nitrobenzamide (59 mg, 0.21 mmol) in DMF (2.0 mL). The solution was stirred at 80° C. for 16 hr. The reaction mixture was poured into water and then extracted with EtOAc (30.0 mL*3). The combined organic layer was washed with brine (30.0 mL*3), dried over Na₂SO₄ and concentrated to give a crude product which was purified by prep-HPLC to give compound 32 (39.4 mg, 35% yield) as a yellow solid.

| | |
|---|---|
| 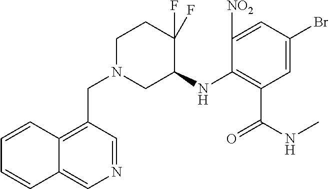 [SMILES: CNC(=O)c1cc(Br)cc([N+](=O)[O-])c1N[C@H]1CN(Cc2cncc3ccccc23)CCC1(F)F] | Compound 32: (S)-5-bromo-2-((4,4-difluoro-1-(isoquinolin-4- ylmethyl)piperidin-3-yl)amino)-N-methyl-3-nitrobenzamide ¹H NMR (400 MHz, DMSO-d₆) δ 9.27 (s, 1H), 8.70 (d, J = 4.0 Hz, 1H), 8.45 (s, 1H), 8.33 (d, J = 8.0 Hz, 1H), 8.20-8.13 (m, 2H), 8.07 (s, 1H), 7.82 (t, J = 8.0 Hz, 1H), 7.74-7.68 (m, 2H), 3.98 (s, 2H), 3.61 (s, 1H), 2.84 (s, 1H), 2.57 (s, 6H), 2.14-1.87 (m, 2H). LC-MS (ESI) (M + H)⁺ = 534.1. |
| Compound 32 | |

### Synthesis of Compounds 34, 81 and 125

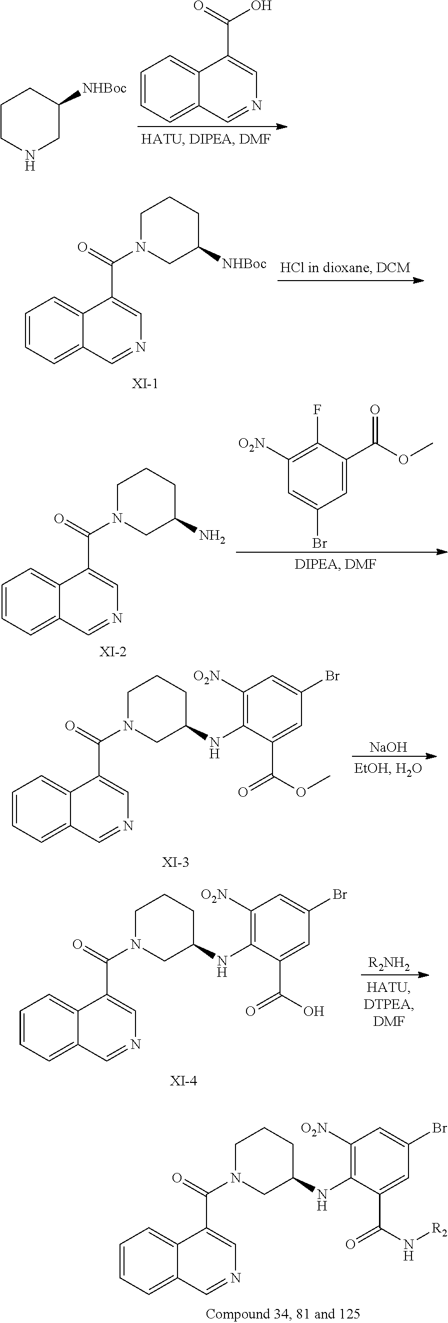

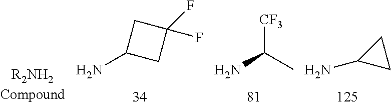

### Intermediate XI-1: tert-butyl (R)-(1-(isoquinoline-4-carbonyl)piperidin-3-yl)carbamate

The synthetic procedure is similar to that of intermediate III-1.

### Intermediate XI-2: (R)-(3-aminopiperidin-1-yl)(isoquinolin-4-yl)methanone

The synthetic procedure is similar to that of intermediate I-3.

### Intermediate XI-3: methyl (R)-5-bromo-2-((1-(isoquinoline-4-carbonyl)piperidin-3-yl)amino)-3-nitrobenzoate

The synthetic procedure is similar to that of intermediate I-2.

### Intermediate XI-4: (R)-5-bromo-2-((1-(isoquinoline-4-carbonyl)piperidin-3-yl)amino)-3-nitrobenzoic acid

The synthetic procedure is similar to that of intermediate IV-5.

### Compounds 34, 81 and 125

The synthetic procedure is similar to the last step of the synthetic route of compound 14.

| | |
|---|---|
| 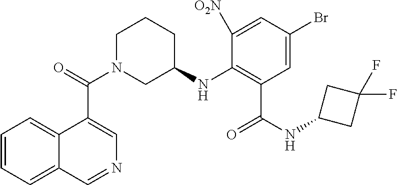 | Compound 34: (R)-5-bromo-N-(3,3-difluorocyclobutyl)-2-((1-(isoquinoline- 4-carbonyl)piperidin-3-yl)amino)-3-nitrobenzamide ¹H NMR (400 MHz, DMSO-d₆) δ 9.42-9.23 (m, 2H), 8.58-7.65 (m, 9H), 4.36-3.49 (m, 4H), 3.17-2.69 (m, 7H), 1.95-1.31 (m, 5H). LC-MS (ESI) (M + H)⁺ = 588.1. |
| Compound 34 | |
| 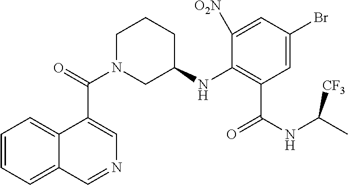 [SMILES: C[C@@H](NC(=O)c1cc(Br)cc([N+](=O)[O-])c1N[C@@H]1CCCN(C(=O)c2cncc3ccccc23)C1)C(F)(F)F] | Compound 81: 5-bromo-2-(((R)-1-(isoquinoline-4-carbonyl)piperidin-3- yl)amino)-3-nitro-N-((R)-1,1,1-trifluoropropan-2-yl)benzamide ¹H NMR (400 MHz, DMSO-d₆) δ 9.54-9.09 (m, 2H), 8.52-7.91 (m, 4H), 7.87-7.49 (m, 4H), 4.91-4.47 (m, 1H), 4.21-3.37 (m, 4H), 3.13 (s, 1H), 1.85- 1.23 (m, 7H). LC-MS (ESI) (M + H)⁺ = 594.1. |
| Compound 81 | |
| 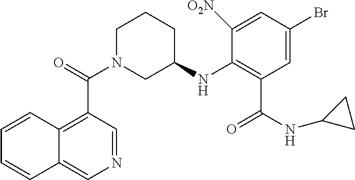 [SMILES: O=C(NC1CC1)c1cc(Br)cc([N+](=O)[O-])c1N[C@@H]1CCCN(C(=O)c2cncc3ccccc23)C1] | Compound 125: (R)-5-bromo-N-cyclopropyl-2-((1-(isoquinoline-4- carbonyl)piperidin-3-yl)amino)-3-nitrobenzamide ¹H NMR (400 MHz, DMSO-d₆) δ 9.44-8.86 (m, 1H), 8.64-8.12 (m, 4H), 7.87-7.67 (m, 5H), 4.18-3.40 (m, 2H), 3.01 (d, J = 104.0 Hz, 4H), 1.93-1.32 (m, 4H), 0.75-0.53 (m, 4H). LC-MS (ESI) (M + H)⁺ = 538.1. |
| Compound 125 | |

### Synthesis of Compounds 39, 49, 86, 92, 97, 98, 100, 101, 103, 104, 105, 106, 107, 113, 114, 119, 120, 123, 140, 168, 169, 174, 175 and 176

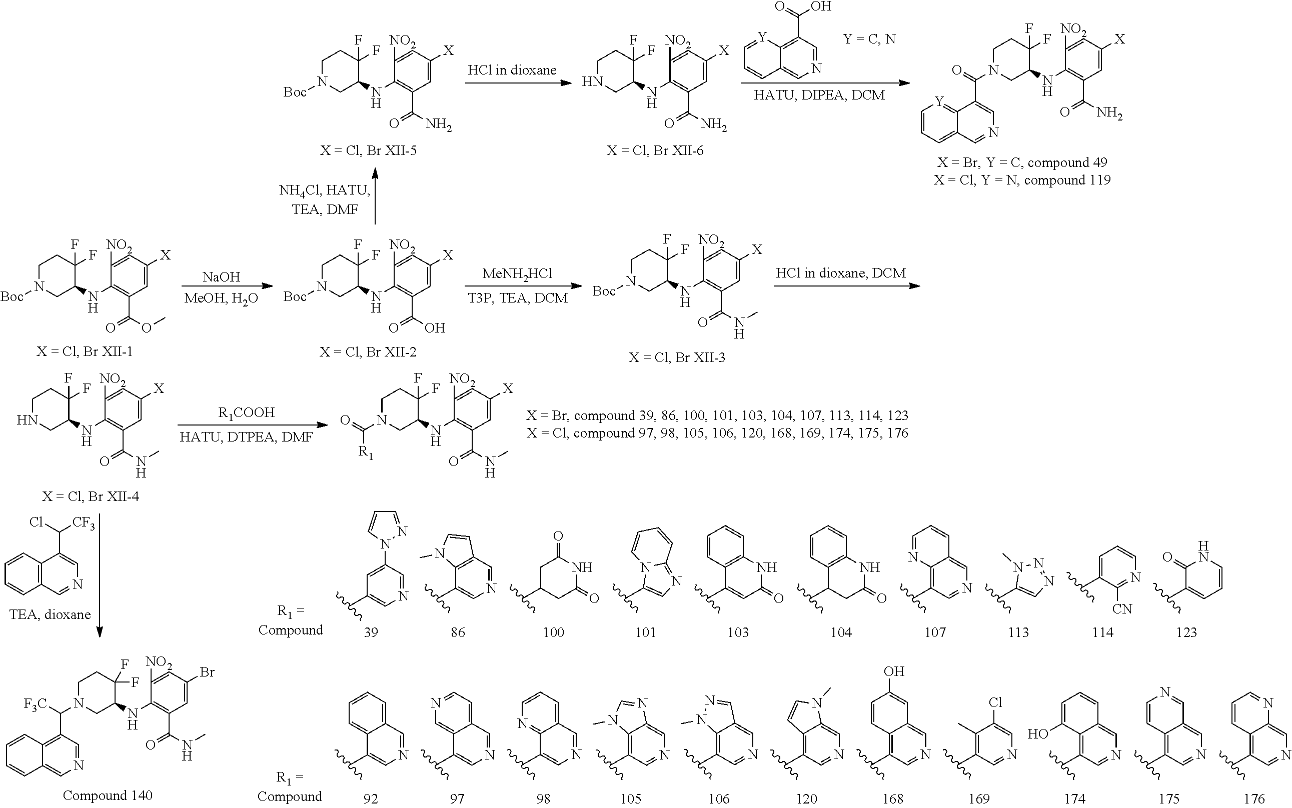

### Intermediate XII-1

The synthetic procedure is similar to that of intermediate IV-2.

### Intermediate XII-2

The synthetic procedure is similar to that of intermediate IV-5.

### Intermediate XII-3

The synthetic procedure is similar to that of intermediate I-1.

### Intermediate XII-4 and XII-6

The synthetic procedure is similar to that of intermediate 1-3.

### Intermediate XII-5

A mixture of 5-bromo-2-[[(3S)-1-tert-butoxycarbonyl-4,4-difluoro-3-piperidyl]amino]-3-nitro-benzoic acid (2.00 g, 4.16 mmol), TEA (2.53 g, 24.9 mmol, 3.48 mL), HATU (3.17 g, 8.33 mmol) and NH₄Cl (1.34 g, 24.9 mmol) in DMF (20 mL) was degassed and purged with N₂ for 3 times, and then the mixture was stirred at 25° C. for 2 hrs under N₂ atmosphere. The reaction mixture was washed with water and filtered to give a residue. The residue was purified by reversed-phase HPLC (column: C18-6 100*30 mm*5 μm; mobile phase: [water (FA)-ACN]; B %: 10%-70%, 15 min). Intermediate XII-5 (620 mg, 1.29 mmol, 31.1% yield) was obtained as a white solid.

### Compounds 39, 49, 86, 92, 97, 98, 100, 101, 103, 104, 105, 106, 107, 113, 114, 120, 123, 168, 169, 174, 175 and 176

The synthetic procedure is similar to the last step of the synthetic route for compound 1.

### Compounds 140: 5-bromo-2-(((3S)-4,4-difluoro-1-(2,2,2-trifluoro-1-(isoquinolin-4-yl)ethyl)piperidin-3-yl)amino)-N-methyl-3-nitrobenzamide

To a solution of 4-(1-chloro-2,2,2-trifluoroethyl)isoquinoline (110 mg, 0.45 mmol) in dioxane (2 mL), (S)-5-bromo-2-((4,4-difluoropiperidin-3-yl)amino)-N-methyl-3-nitrobenzamide (350 mg, 0.89 mmol) and TEA (135 mg, 1.34 mmol) were added, and the reaction was stirred at 80° C. for 12 hr. The mixture was filtered and the filtrate was evaporated under reduced pressure. The residue was purified using prep-TLC with ethyl acetate in petroleum ether (EtOAc:PE=1:2) and Prep-HPLC to give compound 140 (48 mg, 18% yield) as a yellow solid.

| | |
|---|---|
| 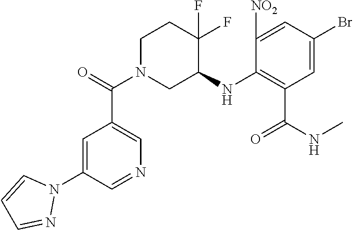 [SMILES: CNC(=O)c1cc(Br)cc([N+](=O)[O-])c1N[C@H]1CN(C(=O)c2cncc(-n3cccn3)c2)CCC1(F)F] | Compound 39: (S)-2-((1-(5-(1H-pyrazol-1-yl)nicotinoyl)-4,4- difluoropiperidin-3-yl)amino)-5-bromo-N-methyl-3-nitrobenzamide ¹H NMR (400 MHz, DMSO-d₆) δ 9.28 (d, J = 2.0 Hz, 1H), 8.90 (s, 1H), 8.73 (d, J = 2.0 Hz, 1H), 8.68 (s, 1H), 8.43 (t, J = 2.0 Hz, 1H), 8.19 (d, J = 60.0 Hz, 2H), 7.91 (d, J = 2.0 Hz, 2H), 6.70 (t, J = 2.0 Hz, 1H), 4.11 (d, J = 110.0 Hz, 2H), 2.96-2.70 (m, 4H), 2.35-1.94 (m, 4H). LC-MS (ESI) (M + H)⁺ = 564.1. |
| Compound 39 | |
| 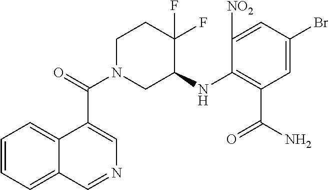 [SMILES: NC(=O)c1cc(Br)cc([N+](=O)[O-])c1N[C@H]1CN(C(=O)c2cncc3ccccc23)CCC1(F)F] | Compound 49: (S)-5-bromo-2-((4,4-difluoro-1-(isoquinoline-4- carbonyl)piperidin-3-yl)amino)-3-nitrobenzamide ¹H NMR (400 MHz, DMSO-d₆) δ 9.40 (br d, J = 6.4 Hz, 1H), 8.18-8.73 (m, 5H), 8.04 (br s, 1H), 7.72-7.94 (m, 4H), 3.79-4.75 (m, 2H), 3.42-3.77 (m, 2H), 1.60-2.40 (m, 3H). LC-MS (ESI) (M + H)⁺ = 534.3. |
| Compound 49 | |
| 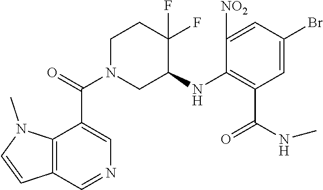 | Compound 86: (S)-5-chloro-2-((4,4-difluoro-1-(1-methyl-1H-pyrrolo [3,2- c]pyridine-7-carbonyl)piperidin-3-yl)amino)-N-methyl-3-nitrobenzamide ¹H NMR (400 MHz, DMSO-d₆) δ 8.75 (d, J = 120.0 Hz, 2H), 8.34-7.99 (m, 2H), 7.92-7.41 (m, 3H), 6.69 (d, J = 4.0 Hz, 1H), 4.80-4.14 (m, 1H), 3.92- 3.45 (m, 7H), 3.22 (dt, J = 24.0, 8.0 Hz, 1H), 2.92-2.64 (m, 2H), 2.28 (d, J = 4.0 Hz, 2H). LC-MS (ESI) (M + H)⁺ = 507.3. |
| Compound 86 | |
| 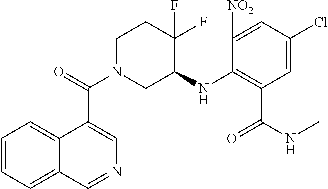 [SMILES: CNC(=O)c1cc(Cl)cc([N+](=O)[O-])c1N[C@H]1CN(C(=O)c2cncc3ccccc23)CCC1(F)F] | Compound 92: (S)-5-chloro-2-((4,4-difluoro-1-(isoquinoline-4-carbonyl) piperidin-3-yl)amino)-N-methyl-3-nitrobenzamide ¹H NMR (400 MHz, DMSO-d₆) δ 9.40 (s, 1H), 8.87-8.52 (m, 2H), 8.27-7.61 (m, 7H), 4.80-3.39 (m, 5H), 2.77 (d, J = 48.0 Hz, 3H), 2.30-1.93 (m, 2H). LC-MS (ESI) (M + H)⁺ = 504.3-2. |
| Compound 92 | |
| 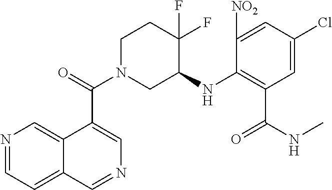 [SMILES: CNC(=O)c1cc(Cl)cc([N+](=O)[O-])c1N[C@H]1CN(C(=O)c2cncc3ccncc23)CCC1(F)F] | Compound 97: (S)-5-chloro-2-((4,4-difluoro-1-(2,6-naphthyridine-4- carbonyl) piperidin-3-yl)amino)-N-methyl-3-nitrobenzamide ¹H NMR (400 MHz, DMSO-d₆) δ 9.56 (s, 1H), 9.39 (s, 1H), 8.98-8.68 (m, 3H), 8.21-7.69 (m, 4H), 4.36 (s, 1H), 3.82 (s, 2H), 2.85 (s, 3H), 2.28 (s, 4H). LC-MS (ESI) (M + H)⁺ = 505.1. |
| Compound 97 | |
| 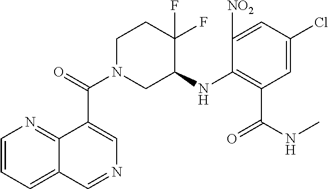 [SMILES: CNC(=O)c1cc(Cl)cc([N+](=O)[O-])c1N[C@H]1CN(C(=O)c2cncc3cccnc23)CCC1(F)F] | Compound 98: (S)-5-chloro-2-((4,4-difluoro-1-(1,6-naphthyridine-8- carbonyl) piperidin-3-yl)amino)-N-methyl-3-nitrobenzamide ¹H NMR (400 MHz, DMSO-d₆) δ 9.54 (d, J = 19.9 Hz, 1H), 9.17 (s, 1H), 9.03-8.51 (m, 3H), 8.22-7.51 (m, 4H), 4.55-3.05 (m, 5H), 2.95-2.61 (m, 3H), 2.26 (s, 2H). LC-MS (ESI) (M + H)⁺ = 505.12. |
| Compound 98 | |
| 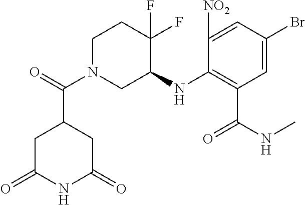 [SMILES: CNC(=O)c1cc(Br)cc([N+](=O)[O-])c1N[C@H]1CN(C(=O)C2CC(=O)NC(=O)C2)CCC1(F)F] | Compound 100: (S)-5-bromo-2-((1-(2,6-dioxopiperidine-4-carbonyl)-4,4- difluoropiperidin-3-yl)amino)-N-methyl-3-nitrobenzamide ¹H NMR (400 MHz, DMSO-d₆) δ 10.71 (s, 1H), 8.75-8.95 (m, 1H), 8.23 (dd, J = 19.2, 2.0 Hz, 1H), 7.81-8.04 (m, 2H), 4.01 (br d, J = 13.2 Hz, 1H), 3.58-3.73 (m, 2H), 3.43-3.57 (m, 2H), 3.34 (s, 2H), 2.75 (br t, J = 4.4 Hz, 3H), 2.64-2.70 (m, 1H), 2.59 (br dd, J = 16.4, 6.0 Hz, 2H), 1.96-2.16 (m, 2H). LC-MS (ESI) (M + 3)⁺ = 534.3. |
| Compound 100 | |
| 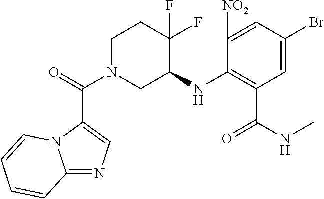 [SMILES: CNC(=O)c1cc(Br)cc([N+](=O)[O-])c1N[C@H]1CN(C(=O)c2cnc3ccccn23)CCC1(F)F] | Compound 101: (S)-5-bromo-2-((4,4-difluoro-1-(imidazo[1,2-a]pyridine-3- carbonyl)piperidin-3-yl)amino)-N-methyl-3-nitrobenzamide ¹H NMR (400 MHz, DMSO-d₆) δ 8.89 (d, J = 7.2 Hz, 1H), 8.81 (q, J = 4.0 Hz, 1H), 8.16-8.23 (m, 2H), 8.01 (br d, J = 9.6 Hz, 1H), 7.83 (d, J = 2.4 Hz, 1H), 7.75 (d, J = 9.2 Hz, 1H), 7.49-7.56 (m, 1H), 7.17 (td, J = 6.8, 0.8 Hz, 1H), 4.21-4.30 (m, 1H), 3.94-4.04 (m, 1H), 3.67 (br d, J = 7.2 Hz, 3H), 2.66 (d, J = 4.4 Hz, 3H), 2.14-2.28 (m, 2H). LC-MS (ESI) (M + 3)⁺ = 539.3. |
| Compound 101 | |
| 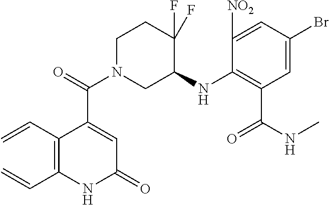 [SMILES: C=Cc1[nH]c(=O)cc(C(=O)N2CCC(F)(F)[C@@H](Nc3c(C(=O)NC)cc(Br)cc3[N+](=O)[O-])C2)c1C=C] | Compound 103: (S)-5-bromo-2-((4,4-difluoro-1-(2-oxo-1,2- dihydroquinoline-4-carbonyl)piperidin-3-yl)amino)-N-methyl-3- nitrobenzamide ¹H NMR (400 MHz, DMSO-d₆) δ 11.79-12.01 (m, 1H), 8.68-8.99 (m, 1H), 8.11-8.28 (m, 1H), 7.70-8.03 (m, 2H), 7.30-7.64 (m, 3H), 7.10-7.26 (m, 1H), 6.49-6.81 (m, 1H), 3.51-4.60 (m, 3H), 3.04-3.39 (m, 2H), 2.54-2.93 (m, 3H), 1.79-2.29 (m, 2H). LC-MS (ESI) (M + 3)⁺ = 566.3. |
| Compound 103 | |
| 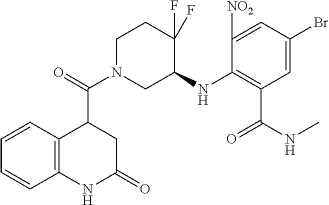 [SMILES: CNC(=O)c1cc(Br)cc([N+](=O)[O-])c1N[C@H]1CN(C(=O)C2CC(=O)Nc3ccccc32)CCC1(F)F] | Compound 104: 5-bromo-2-[(3S)-4,4-difluoro-1-(2-oxo-3,4-dihydro-1H- quinoline-4-carbonyl)-3-piperidyl]amino]-N-methyl-3-nitro-benzamide ¹H NMR (400 MHz, DMSO-d₆) δ 10.08 (s, 1H), 8.77-8.98 (m, 1H), 7.75- 8.56 (m, 3H), 6.83-7.22 (m, 4H), 4.27-4.67 (m, 2H), 3.83-4.11 (m, 1H), 3.50-3.82 (m, 2H), 3.09-3.29 (m, 1H), 2.67-2.86 (m, 3H), 2.54-2.64 (m, 2H), 1.94-2.22 (m, 2H). LC-MS (ESI) (M + 3)⁺ = 568.3. |
| Compound 104 | |
| 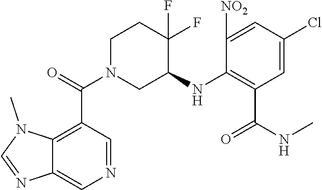 [SMILES: CNC(=O)c1cc(Cl)cc([N+](=O)[O-])c1N[C@H]1CN(C(=O)c2cncc3ncn(C)c23)CCC1(F)F] | Compound 105: (S)-5-chloro-2-((4,4-difluoro-1-(1-methyl-1H-imidazo[4,5- c]pyridine-7-carbonyl)piperidin-3-yl)amino)-N-methyl-3-nitrobenzamide ¹H NMR (400 MHz, DMSO-d₆) δ 9.02 (s, 1H), 8.57-8.90 (m, 1H), 8.35-8.49 (m, 2H), 7.67-8.20 (m, 3H), 4.21-4.74 (m, 1H), 3.78 (br s, 5H), 3.39-3.56 (m, 2H), 2.83 (br s, 2H), 2.01-2.36 (m, 3H). LC-MS (ESI) (M + H)⁺ = 508.4. |
| Compound 105 | |
| 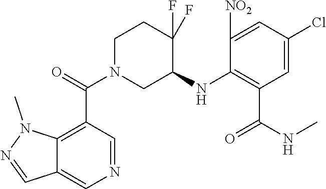 [SMILES: CNC(=O)c1cc(Cl)cc([N+](=O)[O-])c1N[C@H]1CN(C(=O)c2cncc3cnn(C)c23)CCC1(F)F] | Compound 106: (S)-5-chloro-2-((4,4-difluoro-1-(1-methyl-1H-pyrazolo[4,3- c]pyridine-7-carbonyl)piperidin-3-yl)amino)-N-methyl-3-nitrobenzamide ¹H NMR (400 MHz, DMSO-d₆) δ 9.18 (s, 1H), 8.94-8.39 (m, 3H), 8.21-7.65 (m, 3H), 3.97 (s, 8H), 2.84 (s, 3H), 2.31 (s, 2H). LC-MS (ESI) (M + H)⁺ = 508.3. |
| Compound 106 | |
| 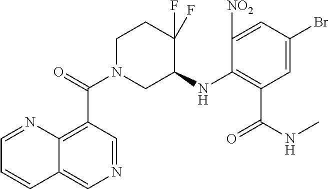 [SMILES: CNC(=O)c1cc(Br)cc([N+](=O)[O-])c1N[C@H]1CN(C(=O)c2cncc3cccnc23)CCC1(F)F] | Compound 107: (S)-5-bromo-2-((4,4-difluoro-1-(1,6-naphthyridine -8- carbonyl)piperidin-3-yl)amino)-N-methyl-3-nitrobenzamide ¹H NMR (400 MHz, DMSO-d₆) δ 9.60-9.50 (m, 1H), 9.26 (d, J = 24.0 Hz, 1H), 9.04-8.64 (m, 3H), 8.36-7.57 (m, 4H), 4.74-3.51 (m, 3H), 3.24 (s, 2H), 2.80 (dd, J = 80.0, 4.0 Hz, 3H), 2.37-1.93 (m, 2H). LC-MS (ESI) (M + 3)⁺ = 551.1. |
| Compound 107 | |
| 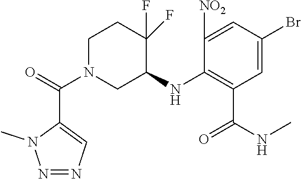 | Compound 113: (S)-5-bromo-2-((4,4-difluoro-1-(1-methyl-1H-1,2,3- triazole-5-carbonyl)piperidin-3-yl)amino)-N-methyl-3-nitrobenzamide ¹H NMR (400 MHz, DMSO-d₆) δ 8.84 (m, 1H), 8.22 (m, 1H), 8.09 (m, 1H), 7.80-8.02 (m, 2H), 4.11-4.45 (m, 1H), 4.09 (s, 3H), 3.85-4.06 (m, 1H), 3.39- 3.70 (m, 3H), 2.64-2.81 (m, 3H), 2.19 (m, 2H). LC-MS (ESI) (M + H)⁺ = 502.3. |
| Compound 113 | |
| 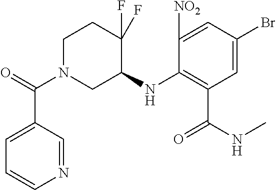 | Compound 114: (S)-5-bromo-2-((1-(2-cyanonicotinoyl)-4,4- difluoropiperidin-3-yl)amino)-N-methyl-3-nitrobenzamide ¹H NMR (400 MHz, DMSO-d₆) δ 8.69-8.95 (m, 2H), 8.13-8.33 (m, 2H), 7.76-7.97 (m, 3H), 4.20-4.60 (m, 1H), 3.63-3.77 (m, 1H), 3.43 (m, 2H), 3.17-3.30 (m, 1H), 2.65-2.83 (m, 3H), 2.06-2.36 (m, 2H). LC-MS (ESI) (M + H)⁺ = 523.3. |
| Compound 114 | |
| 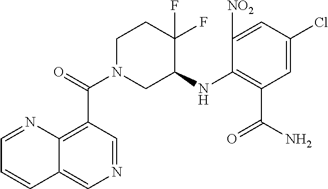 [SMILES: NC(=O)c1cc(Cl)cc([N+](=O)[O-])c1N[C@H]1CN(C(=O)c2cncc3cccnc23)CCC1(F)F] | Compound 119: (S)-5-chloro-2-((4,4-difluoro-1-(1,6-naphthyridine-8- carbonyl) piperidin-3-yl)amino)-3-nitrobenzamide ¹H NMR (400 MHz, DMSO-d₆) δ 9.53-9.45 (m, 1H), 9.30-9.11 (m, 1H), 8.91-8.60 (m, 2H), 8.48-7.62 (m, 6H), 4.17-3.42 (m, 3H), 3.19 (d, J = 32.0 Hz, 2H), 2.31-1.92 (m, 2H). LC-MS (ESI) (M + H)⁺ = 491.2. |
| Compound 119 | |
| 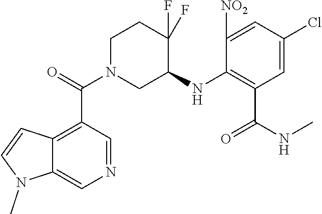 [SMILES: CNC(=O)c1cc(Cl)cc([N+](=O)[O-])c1N[C@H]1CN(C(=O)c2cncc3c2ccn3C)CCC1(F)F] | Compound 120: (S)-5-chloro-2-((4,4-difluoro-1-(1-methyl-1H-pyrrolo[2,3- c]pyridine-4-carbonyl)piperidin-3-yl)amino)-N-methyl-3-nitrobenzamide ¹H NMR (400 MHz, DMSO-d₆) δ 8.93 (s, 1H), 8.85-7.58 (m, 6H), 6.52 (d, J = 4.0 Hz, 1H), 3.96 (s, 4H), 3.87-3.38 (m, 3H), 2.74 (s, 4H), 2.09 (s, 2H). LC-MS (ESI) (M + H)⁺ = 507.2. |
| Compound 120 | |
| 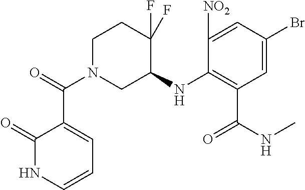 [SMILES: CNC(=O)c1cc(Br)cc([N+](=O)[O-])c1N[C@H]1CN(C(=O)c2ccc[nH]c2=O)CCC1(F)F] | Compound 123: (S)-5-bromo-2-((4,4-difluoro-1-(2-oxo-1,2- dihydropyridine-3-carbonyl)piperidin-3-yl)amino)-N-methyl-3- nitrobenzamide ¹H NMR (400 MHz, DMSO-d₆) δ 11.89-12.07 (m, 1H), 8.72-8.86 (m, 1H), 7.75-7.91 (m, 2H), 7.46-7.66 (m, 2H), 6.23-6.33 (m, 1H), 4.29 (m, 1H), 3.40-3.76 (m, 3H), 3.17-3.29 (m, 2H), 2.66-2.79 (m, 3H), 2.01-2.19 (m, 2H). LC-MS (ESI) (M + H)⁺ = 514.3. |
| Compound 123 | |
| 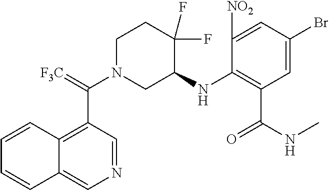 | Compound 140: 5-bromo-2-(((35)-4,4-difluoro-1-(2,2,2-trifluoro-1- (isoquinolin-4-yl)ethyl)piperidin-3-yl)amino)-N-methyl-3-nitrobenzamide ¹H NMR (400 MHz, DMSO-d₆) δ 8.78 (q, J = 4.0 Hz, 1H), 8.30 (d, J = 8.0 Hz, 1H), 8.22 (d, J = 4.0 Hz, 1H), 8.17 (d, J = 12.0 Hz, 2H), 7.89-7.78 (m, 2H), 7.71 (dd, J = 8.0, 4.0 Hz, 1H), 4.07 (qd, J = 12.0, 4.0 Hz, 2H), 3.95- 3.52 (m, 5H), 3.12 (s, 1H), 2.62 (d, J = 4.0 Hz, 3H), 2.37 (s, 2H). LC-MS (ESI) (M + H)⁺ = 602.1. |
| Compound 140 | |
| 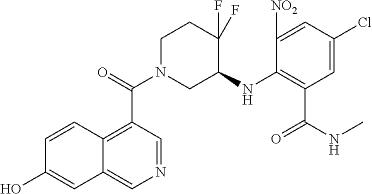 [SMILES: CNC(=O)c1cc(Cl)cc([N+](=O)[O-])c1N[C@H]1CN(C(=O)c2cncc3cc(O)ccc23)CCC1(F)F] | Compound 168: (S)-5-chloro-2-((4,4-difluoro-1-(7-hydroxyisoquinoline-4- carbonyl)piperidin-3-yl)amino)-N-methyl-3-nitrobenzamide ¹H NMR (400 MHz, DMSO-d₆) δ 10.40 (s, 1H), 9.18 (s, 1H), 8.97-8.16 (m, 3H), 8.11-7.55 (m, 3H), 7.41 (d, J = 20.0 Hz, 2H), 4.57-3.66 (m, 5H), 3.29- 2.61 (m, 3H), 2.35-1.76 (m, 2H). LC-MS (ESI) (M + H)⁺ = 520.2. |
| Compound 168 | |
| 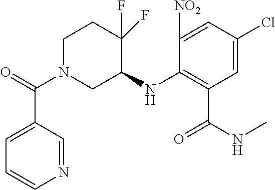 | Compound 169: (S)-5-chloro-2-((1-(5-chloro-4-methylnicotinoyl)-4,4- difluoropiperidin-3-yl)amino)-N-methyl-3-nitrobenzamide ¹H NMR (400 MHz, DMSO-d₆) δ 8.77 (s, 1H), 8.64 (s, 1H), 8.58-8.37 (m, 1H), 8.11 (d, J = 36.0 Hz, 1H), 7.91-7.69 (m, 2H), 4.56- 4.02 (m, 2H), 3.80- 3.55 (m, 1H), 3.31 (s, 2H), 2.83-2.62 (m, 3H), 2.37-2.04 (m, 5H). LC-MS (ESI) (M + H)⁺ = 502.2. |
| Compound 169 | |
| 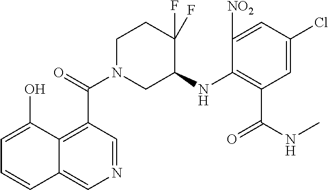 [SMILES: CNC(=O)c1cc(Cl)cc([N+](=O)[O-])c1N[C@H]1CN(C(=O)c2cncc3cccc(O)c23)CCC1(F)F] | Compound 174: (S)-5-chloro-2-((4,4-difluoro-1-(5-hydroxyisoquinoline-4- carbonyl) piperidin-3-yl)amino)-N-methyl-3-nitrobenzamide ¹H NMR (400 MHz, DMSO-d₆) δ 10.69 (d, J = 10.0 Hz, 1H), 9.25 (s, 1H), 9.03-8.32 (m, 2H), 8.27-7.72 (m, 3H), 7.69-7.51 (m, 2H), 7.21-7.06 (m, 1H), 5.24-4.54 (m, 1H), 4.08-3.40 (m, 2H), 3.16 (d, J = 44.0 Hz, 2H), 2.73 (ddd, J = 80.0, 8.0, 4.0 Hz, 3H), 2.10 (d, J = 80.0 Hz, 2H). LC-MS (ESI) (M + H)⁺ = 520.2. |
| Compound 174 | |
| 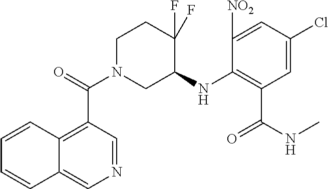 [SMILES: CNC(=O)c1cc(Cl)cc([N+](=O)[O-])c1N[C@H]1CN(C(=O)c2cncc3ccccc23)CCC1(F)F] | Compound 175: (S)-5-chloro-2-((4,4-difluoro-1-(2,7-naphthyridine-4- carbonyl) piperidin-3-yl)amino)-N-methyl-3-nitrobenzamide ¹H NMR (400 MHz, DMSO-d₆) δ 9.64 (s, 2H), 8.81 (t, J = 40.0 Hz, 3H), 8.24-7.60 (m, 4H), 4.47-3.64 (m, 5H), 2.85 (s, 3H), 2.27 (s, 2H). LC-MS (ESI) (M + H)⁺ = 505.1. |
| Compound 175 | |
| 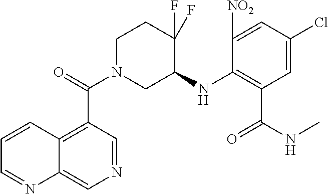 [SMILES: CNC(=O)c1cc(Cl)cc([N+](=O)[O-])c1N[C@H]1CN(C(=O)c2cncc3ncccc23)CCC1(F)F] | Compound 176: (S)-5-chloro-2-((4,4-difluoro-1-(1,7-naphthyridine-5- carbonyl) piperidin-3-yl)amino)-N-methyl-3-nitrobenzamide ¹H NMR (400 MHz, DMSO-d₆) δ 9.47 (s, 1H), 9.13 (s, 1H), 8.94-8.64 (m, 2H), 8.34 (s, 1H), 8.06 (d, J = 80.0 Hz, 2H), 7.76 (d, J = 60.8 Hz, 2H), 3.76 (s, 3H), 3.57 (s, 2H), 2.83 (s,3H),2.09 (s,2H). LC-MS (ESI) (M + H)⁺ = 505.2. |
| Compound 176 | |

### Synthesis of Compounds 50 and 166

The synthesis routes of compounds 50 and 166 are similar to last step synthesis route of compound 39.

| | |
|---|---|
| 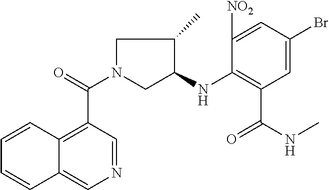 [SMILES: CNC(=O)c1cc(Br)cc([N+](=O)[O-])c1N[C@H]1CN(C(=O)c2cncc3ccccc23)C[C@@H]1C] | Compound 50: 5-bromo-2-[[(3R, 4S)-1-(isoquinoline-4-carbonyl)-4-methyl- pyrrolidin-3-yl]amino]-N-methyl-3-nitro-benzamide ¹H NMR (400 MHz, DMSO-d₆) δ 9.38 (s, 1H), 8.60-8.86 (m, 1H), 8.50 (d, J = 3.6 Hz, 1H), 8.20-8.23 (m, 1H), 8.04-8.11 (m, 1H), 7.62-7.95 (m, 5H), 3.84- 3.99 (m, 1H), 3.57-3.66 (m, 1H), 3.41-3.51 (m, 1H), 3.07-3.31 (m, 2H), 2.59- 2.83 (m, 3H), 2.15-2.36 (m, 1H), 0.81-1.01 (m, 3H). LC-MS (ESI) (M + 3)⁺ = 514.3. |
| Compound 50 | |
| 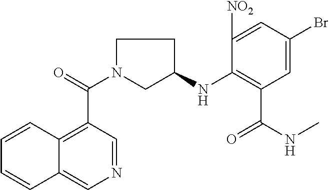 | Compound 166: 5-bromo-2-[[(3S,4R)-4-fluoro-1-(isoquinoline-4- carbonyl)pyrrolidin-3-yl]amino]-N-methyl-3-nitro-benzamide ¹H NMR (400 MHz, DMSO-d₆) δ 9.40 (d, J = 2.8 Hz, 1H), 8.56-8.83 (m, 1H), 8.54 (s, 1H), 8.19-8.28 (m, 2H), 8.08-8.16 (m, 1H), 7.78-7.91 (m, 3H), 7.69- 7.78 (m, 1H), 4.99-5.41 (m, 1H), 3.89-4.25 (m, 3H), 3.52-3.62 (m, 1H), 3.42- 3.50 (m, 1H), 3.16-3.29 (m, 1H), 2.51-2.84 (m, 2H). LC-MS (ESI) (M + H)⁺ = 516.3. |
| Compound 166 | |

### Synthesis of Compounds 170, 171 and 178

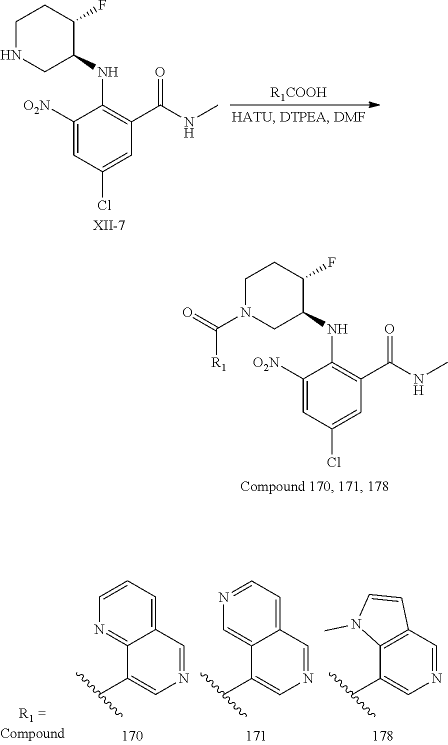

### Intermediate XI-7

The synthesis of intermediate XII-7 is similar to that of intermediate XII-4.

### Compounds 170, 171 and 178

The synthetic procedure is similar to the last step of the synthetic procedure of compound 1.

| | |
|---|---|
| 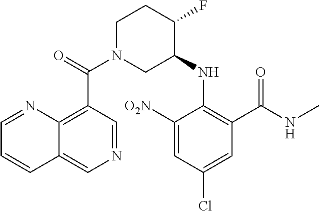 [SMILES: CNC(=O)c1cc(Cl)cc([N+](=O)[O-])c1N[C@H]1CN(C(=O)c2cncc3cccnc23)CC[C@@H]1F] | Compound 170: 5-chloro-2-(((3S, 4S)-4-fluoro-1-(1,6-naphthyridine-8- carbonyl) piperidin-3-yl)amino)-N-methyl-3-nitrobenzamide ¹H NMR (400 MHz, DMSO-d₆) δ 9.46 (d, J = 20.0 Hz, 1H), 9.15 (s, 1H), 8.88- 8.54 (m, 3H), 8.19-8.01 (m, 1H), 7.98-7.52 (m, 3H), 4.89-4.44 (m, 2H), 3.99- 3.77 (m, 1H), 3.09 (d, J = 20.0 Hz, 2H), 2.88-2.58 (m, 3H), 2.15-1.55 (m, 3H). LC-MS (ESI) (M + H)⁺ = 487.3. |
| Compound 170 | |
| 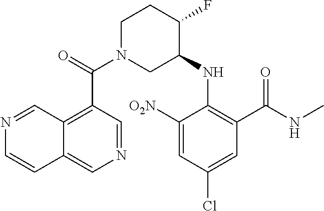 [SMILES: CNC(=O)c1cc(Cl)cc([N+](=O)[O-])c1N[C@H]1CN(C(=O)c2cncc3ccncc23)CC[C@@H]1F] | Compound 171: 5-chloro-2-(((3S, 4S)-4-fluoro-1-(2,6-naphthyridine-4 - carbonyl)piperidin-3-yl)amino)-N-methyl-3-nitrobenzamide ¹H NMR (400 MHz, DMSO-d₆) δ 9.59-9.26 (m, 2H), 8.92-8.75 (m, 2H), 8.66 (d, J = 36.0 Hz, 1H), 8.17-8.01 (m, 2H), 7.94-7.51 (m, 2H), 4.80 (d, J = 48.0 Hz, 1H), 3.62 (s, 3H), 3.27-2.55 (m, 4H), 2.15 (s, 1H), 1.74 (d, J = 80.0 Hz, 2H). LC-MS (ESI) (M + H)⁺ = 487.2. |
| Compound 171 | |
| 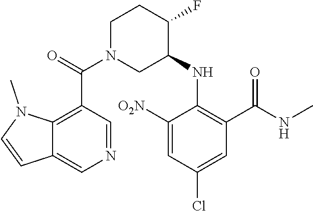 [SMILES: CNC(=O)c1cc(Cl)cc([N+](=O)[O-])c1N[C@H]1CN(C(=O)c2cncc3ccn(C)c23)CC[C@@H]1F] | Compound 178: 5-chloro-2-(((3S, 4S)-4-fluoro-1-(1-methyl-1H-pyrrolo[3,2-c] pyridine-7-carbonyl)piperidin-3-yl)amino)-N-methyl-3-nitrobenzamide ¹H NMR (400 MHz, DMSO-d₆) δ 8.92-8.73 (m, 2H), 8.27 (s, 1H), 8.15-7.95 (m, 2H), 7.72 (d, J = 10.0 Hz, 1H), 7.52 (d, J = 14.0 Hz, 1H), 6.71 (d, J = 4.0 Hz, 1H), 4.78 (s, 5H), 3.72 (d, J = 16.0 Hz, 3H), 2.80 (d, J = 4.0 Hz, 3H), 2.23 (s, 1H), 1.63 (s, 2H). LC-MS (ESI) (M + H)⁺ = 489.2. |
| Compound 178 | |

### Synthesis of Compound 69

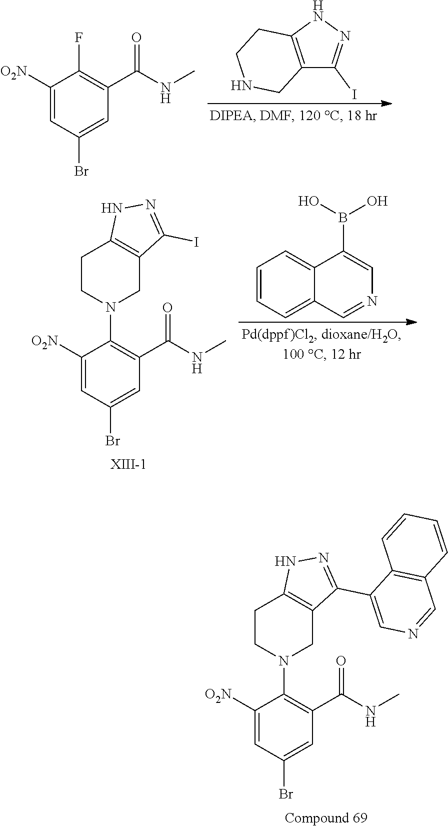

### Intermediate XIII-1: 5-bromo-2-(3-iodo-1,4,6,7-tetrahydro-5H-pyrazolo[4, 3-c]pyridin-5-yl)-N-methyl-3-nitrobenzamide

To a solution of 3-iodo-4,5,6,7-tetrahydro-1H-pyrazolo[4,3-c]pyridine (100 mg, 0.40 mmol) in DMF (5 mL), 5-bromo-2-fluoro-N-methyl-3-nitrobenzamide (133 mg, 0.48 mmol) and DIPEA (156 mg, 1.20 mmol) were added, and the reaction mixture was heated and stirred at 120° C. for 18 hr. After cooling to room temperature, the reaction mixture was poured into water (10 mL) and extracted with EtOAc (30 mL*3). The combined organic layers were washed with brine (20 mL), dried over Na₂SO₄, filtered and concentrated under reduced pressure to get a residue, which was purified by column chromatography on silica gel eluting with ethyl acetate (from 0% to 10%) in petroleum ether to afford the title product (45 mg, 22% yield) as a white solid.LC-MS (ESI) (M+H)⁺=506.0

### Compound 69: 5-bromo-2-(3-(isoquinolin-4-yl)-1,4,6,7-tetrahydro-5H-pyrazolo[4,3-c]pyridin-5-yl)-N-methyl-3-nitrobenzamide

Isoquinolin-4-ylboronic acid (19 mg, 0.11 mmol), K₂CO₃ (37 mg, 0.27 mmol) and Pd(dppf)Cl₂ (7 mg, 0.01 mmol) were added to a solution of intermediate XIII-1 (45 mg, 0.09 mmol) in dioxane (3 mL) and H₂O (1 mL), and the reaction mixture was heated and stirred at 100° C. for 12 hr under a N₂ atmosphere. After cooling to room temperature, the reaction mixture was poured into water (10 mL) and extracted with EtOAc (30 mL*3). The organic layer was separated, washed with water, NH₄C₁ solution (20 mL), NaHCO₃ solution (20 mL) and further saturated NaCl solution (20 mL), and it was dried over Na₂SO₄, and concentrated in vacuo. The residue was purified using prep-TLC with ethyl acetate in petroleum ether (EtOAc:PE=1:2) and Prep-HPLC to give compound 69 (1 mg, 1% yield) as a yellow solid.

| | |
|---|---|
| 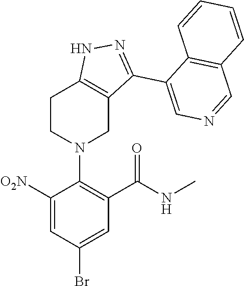 [SMILES: CNC(=O)c1cc(Br)cc([N+](=O)[O-])c1N1CCc2[nH]nc(-c3cncc4ccccc34)c2C1] | Compound 69: 5-bromo-2-(3-(isoquinolin-4-yl)-1,4,6,7-tetrahydro-5H- pyrazolo[4,3-c]pyridin-5-yl)-N-methyl-3-nitrobenzamide ¹H NMR (400 MHz, Methanol-d₄) δ 9.26 (s, 1H), 8.43 (s, 1H), 8.18 (s, 1H), 8.01-7.74 (m, 4H), 7.70 (t, J = 2.4 Hz, 1H), 5.49 (d, J = 2.4 Hz, 2H), 4.12-4.00 (m, 2H), 2.92 (s, 2H), 2.73 (d, J = 2.4 Hz, 3H). LC-MS (ESI) (M + H)⁺ = 507.1. |
| Compound 69 | |

### Synthesis of compounds 89 and 90

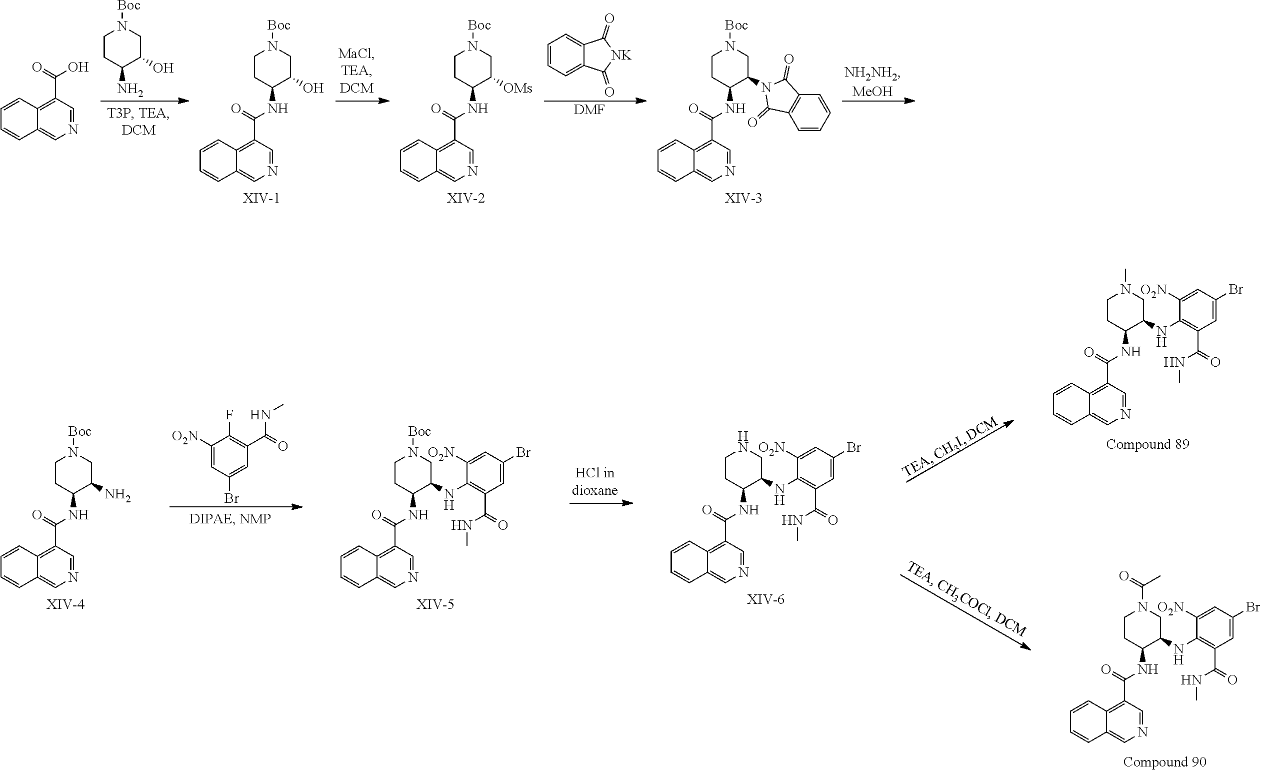

### Intermediate XIV-1: tert-butyl (3S,4S)-3-hydroxy-4-(isoquinoline-4-carboxamido)piperidine-1-carboxylate

Isoquinoline-4-carboxylic acid (841 mg, 4.86 mmol), TEA (2.34 g, 23.12 mmol) and T3P (5.88 g, 9.25 mmol, 50% wt in EtOAc) were added to a solution of tert-butyl (3S,4S)-4-amino-3-hydroxypiperidine-1-carboxylate (1.0 g, 4.62 mmol) in DCM (30 mL), and the reaction was stirred at 25° C. for 2 hr. The reaction was added water (40 mL) and extracted with DCM (40 mL*3). The organic layer was separated, and washed with saturated NaCl solution. The combined organic layers dried over Na₂SO₄ was filtered and concentrated under reduced pressure to dryness. The crude product was purified by silica gel chromatography eluted with methanol in DCM (DCM/Methanol=10/1) to afford intermediate XIV-1 (1.0 g, 58% yield) as a colorless oil. LC-MS (ESI) (M+H)⁺=372.2.

### Intermediate XIV-2: tert-butyl (3S,4S)-4-(isoquinoline-4-carboxamido)-3-((methylsulfonyl)oxy)piperidine-1-carboxylate

To a solution of intermediate XIV-1 (1.0 g, 2.69 mmol) in DCM (20 mL), TEA (818 mg, 8.08 mmol) and MsCl (617 mg, 5.38 mmol) were added, and the reaction was stirred at 25° C. for 16 hr. The reaction was added water (40 mL) and extracted with DCM (40 mL*3). The organic layer was separated, and washed with saturated NaCl solution. The combined organic layers dried over Na₂SO₄ was filtered and concentrated under reduced pressure to dryness. The crude product was purified by silica gel chromatography eluted with methanol in DCM (DCM/Methanol=20/1) to afford intermediate XIV-2 (1000 mg, 83% yield) as a red solid. LC-MS (ESI) (M+H)⁺=450.4.

### Intermediate XIV-3: tert-butyl (3S,4S)-3-(1,3-dioxoisoindolin-2-yl)-4-(isoquinoline-4-carboxamido)piperidine-1-carboxylate

To a solution of intermediate XIV-2 (1000 mg, 2.22 mmol) in DMF (20 mL), potassium 1,3-dioxoisoindolin-2-ide was added (824 mg, 4.44 mmol) and the reaction was stirred at 100° C. for 16 hr. The reaction was added water (40 mL) and extracted with EtOAc (40 mL*3). The organic layer was separated, washed with saturated NaCl solution and concentrated in vacuo. The crude product was purified by silica gel chromatography eluted with methanol in DCM (MeOH:DCM=1/20) to afford intermediate XIV-3 (700 mg, 62% yield) as a colorless solid.

LC-MS (ESI) (M+H)⁺=501.3.

### Intermediate XIV-4: tert-butyl (3R,4S)-3-amino-4-(isoquinoline-4-carboxamido)piperidine-1-carboxylate

Hydrazine hydrate (350 mg, 7.0 mmol) was added to a solution of intermediate XIV-3 (700 mg, 1.40 mmol) in MeOH (10 mL), and the reaction was stirred at 70° C. for 16 hr. The reaction was concentrated under vacuum, then added water (30 mL) and extracted with EtOAc (30 mL*3). The organic layer was separated, and washed with saturated NaCl solution. The combined organic layers dried over Na₂SO₄ was filtered and concentrated under reduced pressure to dryness. The crude product was purified by silica gel chromatography eluted with methanol in DCM (MeOH:DCM=1/20) to afford intermediate XIV-4 (400 mg, 77% yield) as a colorless oil.

LC-MS (ESI) (M+H)⁺=371.3.

### Intermediate XIV-5: tert-butyl (3R,4S)-3-((4-bromo-2-(methylcarbamoyl)-6-nitrophenyl)amino)-4-(isoquinoline-4-carboxamido)piperidine-1-carboxylate

5-bromo-2-fluoro-N-methyl-3-nitrobenzamide (359 mg, 1.30 mmol) and DIPEA (698 mg, 5.40 mmol) were added to a solution of intermediate XIV-4 (400 mg, 1.08 mmol) in NMP (10 mL), and the reaction was stirred at 80° C. for 16 hr. The reaction was added water (40 mL) and extracted with EtOAc (40 mL*3). The organic layer was separated, and washed with saturated NaCl solution. The combined organic layers dried over Na₂SO₄ was filtered and concentrated under reduced pressure to dryness. The crude product was purified by silica gel chromatography eluted with ethyl acetate in petroleum ether (PE:EtOAc=1:1) to afford intermediate XIV-5 (550 mg, 81%) as a black solid. LC-MS (ESI) (M+H)⁺=627.2.

### Intermediate XIV-6: N-((3R,4S)-3-((4-bromo-2-(methylcarbamoyl)-6-nitrophenyl)amino)piperidin-4-yl)isoquinoline-4-carboxamide

The synthetic procedure of intermediate XIV-6 is similar to that of intermediate I-3.

### Compound 89: N-((3R,4S)-3-((4-bromo-2-(methylcarbamoyl)-6-nitrophenyl)amino)-1-methylpiperidin-4-yl)isoquinoline-4-carboxamide

TEA (57.6 mg, 0.57 mmol) and CH₃I (26.9 mg, 0.19 mmol) were added to a solution of intermediate XIV-6 (100 mg, 0.19 mmol) in DCM (5 mL), and the reaction was stirred at room temperature for 2 hr. The reaction was concentrated under vacuum, then added water (20 mL) and extracted with EtOAc (20 mL*3). The organic layer was separated, washed with saturated NaCl solution and concentrated in vacuo. The combined organic layers dried over Na₂SO₄ was filtered and concentrated under reduced pressure to dryness. The residue was purified by prep-HPLC to afford compound 89 (16.4 mg, 16% yield) as a yellow solid.

### Compound 90

CH₃COCl (18 mg, 0.23 mmol) and TEA (47 mg, 0.46 mmol) were added to a solution of intermediate XIV-6 (80 mg, 0.15 mmol) in DCM (2 mL), and the reaction was stirred at 25° C. for 2 hr. The reaction was added water (20 mL) and extracted with EtOAc (20 mL*3). The organic layer was separated, and washed with saturated NaCl solution. The combined organic layers dried over Na₂SO₄ was filtered and concentrated under reduced pressure to dryness. The residue was purified by prep-HPLC to afford compound 90 (30 mg, 34%) as a yellow solid.

| | |
|---|---|
| 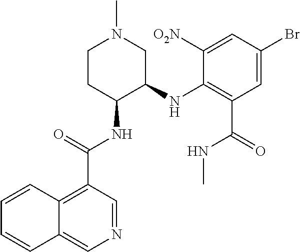 [SMILES: CNC(=O)c1cc(Br)cc([N+](=O)[O-])c1N[C@@H]1CN(C)CC[C@@H]1NC(=O)c1cncc2ccccc12] | Compound 89: N-((3R, 4S)-3-((4-bromo-2-(methylcarbamoyl)-6- nitrophenyl)amino)-1-methylpiperidin-4-yl)isoquinoline-4-carboxamide ¹H NMR (400 MHz, Methanol-d₄) δ 10.20 (s, 1H), 9.39 (d, J = 8.0 Hz, 1H), 9.01-8.79 (m, 5H), 8.65-8.47 (m, 3H), 6.61 (s, 1H), 4.15-3.57 (m, 6H), 3.1- 2.40 (m, 8H). LC-MS (ESI) (M + H)⁺ = 541.2. |
| Compound 89 | |
| 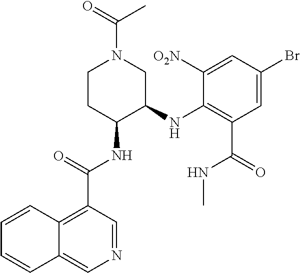 [SMILES: CNC(=O)c1cc(Br)cc([N+](=O)[O-])c1N[C@@H]1CN(C(C)=O)CC[C@@H]1NC(=O)c1cncc2ccccc12] | Compound 90: N-((3R, 4S)-1-acetyl-3-((4-bromo-2-(methylcarbamoyl)-6- nitrophenyl)amino)piperidin-4-yl)isoquinoline-4-carboxamide ¹H NMR (400 MHz, DMSO-d₆) δ 9.36 (s, 1H), 8.70-8.51 (m, 2H), 8.31-7.94 (m, 5H), 7.84-7.65 (m, 3H), 4.50-4.19 (m, 4H), 3.14-3.03 (m, 2H), 2.76 (d, J = 4.0 Hz, 3H), 2.07 (d, J = 6.0 Hz, 3H), 1.78-1.48 (m, 2H). LC-MS (ESI) (M + H)⁺ = 571.2. |
| Compound 90 | |

### Synthesis of Compounds 96, 126, 133, 135, 136 and 177

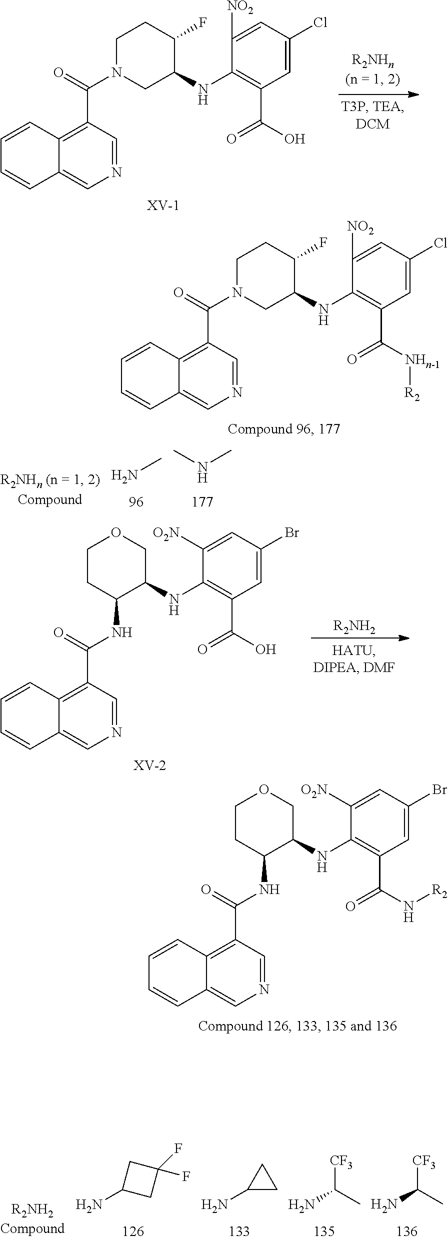

### Intermediates XV-1 and XV-2: 5-bromo-2-(((3S,4S)-4-(isoquinoline-4-carboxamido)tetrahydro-2H-pyran-3-yl)amino)-3-nitrobenzoic

The synthesis routes of intermediate XV-1 and XV-2 are similar to that of intermediate IV-5.

### Compounds 96, 126, 133, 135, 136 and 177

The synthetic routes of compounds 96, 126, 133, 135, 136 and 177 are similar to the last step of the synthesis route of compound 14.

| | |
|---|---|
| 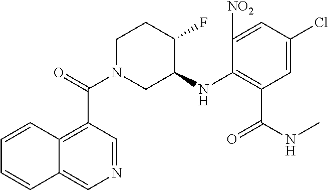 [SMILES: CNC(=O)c1cc(Cl)cc([N+](=O)[O-])c1N[C@H]1CN(C(=O)c2cncc3ccccc23)CC[C@@H]1F] | Compound 96: 5-chloro-2-(((3S, 4S)-4-fluoro-1-(isoquinoline-4- carbonyl)piperidin -3-yl)amino)-N-methyl-3-nitrobenzamide ¹H NMR (400 MHz, DMSO-d₆) δ 10.24 (s, 1H), 9.92-8.23 (m, 9H), 5.58 (s, 1H), 4.75 (t, J = 120.0 Hz, 3H), 3.64 (d, J = 4.0 Hz, 4H), 2.52 (d, J = 100.0 Hz, 3H). LC-MS (ESI) (M + H)⁺ = 486.3. |
| Compound 96 | |
| 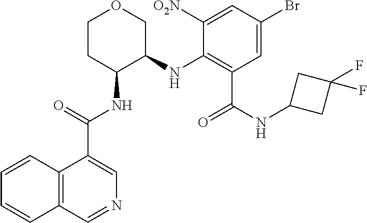 [SMILES: O=C(NC1CC(F)(F)C1)c1cc(Br)cc([N+](=O)[O-])c1N[C@@H]1COCC[C@@H]1NC(=O)c1cncc2ccccc12] | Compound 126: N-((3S, 4S)-3-((4-bromo-2-((3,3- difluorocyclobutyl)carbamoyl)-6-nitrophenyl)amino)tetrahydro-2H-pyran-4- yl)isoquinoline-4-carboxamide ¹H NMR (400 MHz, DMSO-d₆) δ 9.35 (s, 1H), 9.07 (d, J = 8.0 Hz, 1H), 8.73 (d, J = 8.0 Hz, 1H), 8.47 (d, J = 12.0 Hz, 1H), 8.22-8.09 (m, 4H), 7.81-7.70 (m, 3H), 4.38-4.13 (m, 2H), 3.97-3.82 (m, 3H), 3.66-3.50 (m, 2H), 2.98-2.68 (m, 4H), 1.95-1.66 (m, 2H). LC-MS (ESI) (M + H)⁺ = 604.0. |
| Compound 126 | |
| 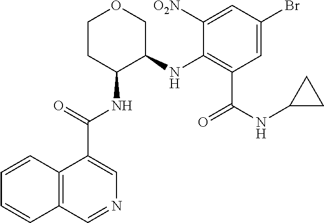 [SMILES: O=C(NC1CC1)c1cc(Br)cc([N+](=O)[O-])c1N[C@@H]1COCC[C@@H]1NC(=O)c1cncc2ccccc12] | Compound 133: N-((3S, 4S)-3-((4-bromo-2-(cyclopropylcarbamoyl)-6- nitrophenyl)amino)tetrahydro-2H-pyran-4-yl)isoquinoline-4-carboxamide ¹H NMR (400 MHz, DMSO-d₆) δ 9.33 (s, 1H), 8.68 (dd, J = 20.0, 4.0 Hz, 2H), 8.45 (d, J = 12.0 Hz, 1H), 8.23-8.05 (m, 4H), 7.79-7.66 (m, 3H), 4.35 (s, 1H), 3.94 (s, 2H), 3.85 (d, J = 12.0 Hz, 1H), 3.63 (d, J = 12.0 Hz, 1H), 3.54 (s, 1H), 2.76 (d, J = 4.0 Hz, 1H), 1.89 (s, 1H), 1.70 (d, J = 8.0 Hz, 1H), 0.67 (d, J = 8.0 Hz, 2H), 0.56 (s, 2H). LC-MS (ESI) (M + H)⁺ = 554.0. |
| Compound 133 | |
| 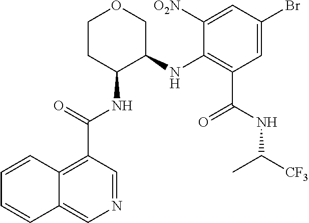 | Compound 135: N-((3S, 4S)-3-((4-bromo-2-nitro-6-(((S)-1,1,1-trifluoropropan- 2-yl)carbamoyl)phenyl)amino)tetrahydro-2H-pyran-4-yl)isoquinoline-4- carboxamide ¹H NMR (400 MHz, DMSO-d₆) δ 9.34 (s, 1H), 9.27 (d, J = 8.0 Hz, 1H), 8.72 (d, J = 8.0 Hz, 1H), 8.40 (d, J = 12.0 Hz, 1H), 8.26 (d, J = 3.0 Hz, 1H), 8.20- 8.13 (m, 2H), 8.05 (d, J = 10.0 Hz, 1H), 7.81-7.76 (m, 1H), 7.74-7.69 (m, 2H), 4.82-4.69 (m, 1H), 4.36 (d, J = 4.0 Hz, 1H), 3.98-3.86 (m, 3H), 3.56 (dd, J = 24.0, 12.0 Hz, 2H), 1.86 (t, J = 12.0 Hz, 1H), 1.72 (d, J = 12.0 Hz, 1H), 1.34 (d, J = 8.0 Hz, 3H). LC-MS (ESI) (M + H)⁺ = 610.0. |
| Compound 135 | |
| 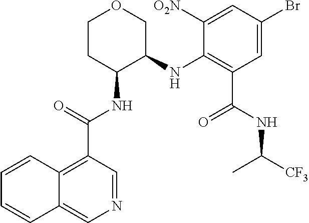 [SMILES: C[C@@H](NC(=O)c1cc(Br)cc([N+](=O)[O-])c1N[C@@H]1COCC[C@@H]1NC(=O)c1cncc2ccccc12)C(F)(F)F] | Compound 136: N-((3S, 4S)-3-((4-bromo-2-nitro-6-(((R)-1,1,1-trifluoropropan- 2-yl)carbamoyl)phenyl)amino)tetrahydro-2H-pyran-4-yl)isoquinoline-4- carboxamide ¹H NMR (400 MHz, DMSO-d₆) δ 9.34 (s, 1H), 9.22 (d, J = 8.0 Hz, 1H), 8.70 (d, J = 8.0 Hz, 1H), 8.42 (d, J = 12.0 Hz, 1H), 8.30-8.19 (m, 2H), 8.15 (d, J = 8.0 Hz, 2H), 7.78 (ddd, J = 8.0, 6.4, 2.0 Hz, 1H), 7.74-7.69 (m, 1H), 7.65 (d, J = 4.0 Hz, 1H), 4.83-4.69 (m, 1H), 4.44-4.32 (m, 1H), 3.97 (dd, J = 22.0, 12.0 Hz, 2H), 3.85 (dd, J = 12.0, 4.0 Hz, 1H), 3.63-3.51 (m, 2H), 1.97-1.85 (m, 1H), 1.70 (d, J = 8.0 Hz, 1H), 1.34 (d, J = 8.0 Hz, 3H). LC-MS (ESI) (M − H)⁻ = 608.1. |
| Compound 136 | |
| 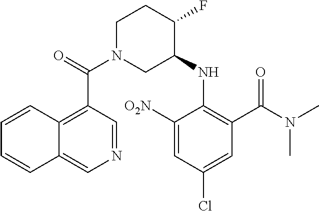 [SMILES: CN(C)C(=O)c1cc(Cl)cc([N+](=O)[O-])c1N[C@H]1CN(C(=O)c2cncc3ccccc23)CC[C@@H]1F] | Compound 177: 5-chloro-2-[(3S,4S)-4-fluoro-1-(isoquinoline-4-carbonyl)-3- piperidyl]amino]-N, N-dimethyl-3-nitro-benzamide ¹H NMR (400 MHz, DMSO-d₆) δ 9.35-9.43 (m, 1H), 8.53 (br s, 1H), 8.08-8.32 (m, 2H), 7.74-7.92 (m, 3H), 7.64 (br d, J = 2.0 Hz, 1H), 7.17-7.54 (m, 1H), 4.73-4.90 (m, 1H), 4.08-4.40 (m, 1H), 3.44-3.83 (m, 3H), 3.09 (br s, 3H), 2.76- 3.01 (m, 3H), 1.71-2.22 (m, 2H), 1.35-1.70 (m, 1H) LC-MS (ESI) (M + H)⁺ = 500.4. |
| Compound 177 | |

### Synthesis of compound 102

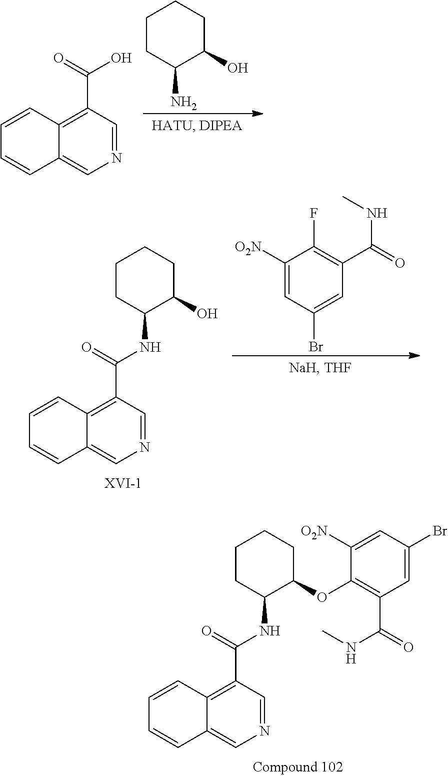

### Intermediate XVI-1: N-[(1,2R)-2-hydroxycyclohexyl]isoquinoline-4-carboxamide

To a solution of isoquinoline-4-carboxylic acid (100 mg, 0.58 mmol) in DCM (5.00 mL) was added HATU (329 mg, 0.87 mmol) and DIPEA (373 mg, 2.89 mmol). After addition, the mixture was stirred for 30 min, and then (1R,2S)-2-aminocyclohexanol (87.6 mg, 0.58 mmol) was added. The mixture was stirred at 25° C. for 12 hrs. The reaction mixture was added to the water and stirred for 30 minutes, and extracted with DCM. The combined organic layer was concentrated under reduced pressure to give intermediate XVI-1 (125 mg, 80.10% yield) as a yellow solid. LC-MS (ESI) (M+H)⁺=271.1.

### Compounds 102: N-[(1S,2R)-2-[4-bromo-2-(methylcarbamoyl)-6-nitro-phenoxy]cyclohexyl]isoquinoline-4-carboxamide

To a solution of intermediate XVI-1 (50.0 mg, 0.18 mmol) in THF (2.00 mL) was added one portion NaH (18.5 mg, 0.46 mmol, 60.0% purity) at 0° C. After addition, the mixture was stirred at this temperature for 30 min, and then 5-bromo-2-fluoro-N-methyl-3-nitro-benzamide (51.2 mg, 0.18 mmol) was added at 25° C. The resulting mixture was stirred at 25° C. for 2 hrs. The reaction mixture was added to water and stirred for 30 minutes, and extracted with DCM. The combined organic layer was concentrated under reduced pressure to give a residue. The residue was purified by prep-HPLC (column: Xtimate C18 100*30 mm*10 um; mobile phase: [water(FA)-ACN]; B %: 40%-70%, 10 min). Compound 102 (20.0 mg, 20.3% yield) was obtained as a yellow solid.

| | |
|---|---|
| 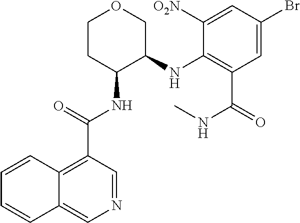 [SMILES: CNC(=O)c1cc(Br)cc([N+](=O)[O-])c1N[C@@H]1COCC[C@@H]1NC(=O)c1cncc2ccccc12] | Compound 102: N-[(1S, 2R)-2-[4-bromo-2-(methylcarbamoyl)-6-nitro- phenoxy]cyclohexyllisoquinoline-4-carboxamide ¹H NMR (400 MHz, DMSO-d₆) δ 9.41 (s, 1H), 8.67 (br d, J = 4.4 Hz, 1H), 8.51-8.62 (m, 2H), 8.07-8.23 (m, 3H), 7.81-7.89 (m, 1H), 7.71-7.78 (m, 1H), 7.69 (d, J = 2.4 Hz, 1H), 4.52 (br s, 1H), 4.14-4.26 (m, 1H), 2.71 (d, J = 4.4 Hz, 3H), 1.74-1.91 (m, 2H), 1.69 (br s, 2H), 1.52-1.63 (m, 1H), 1.31-1.45 (m, 3H). LC-MS (ESI) (M + H)⁺ = 527.3. |
| Compound 102 | |

### Synthesis of compounds 161, 162 and 163

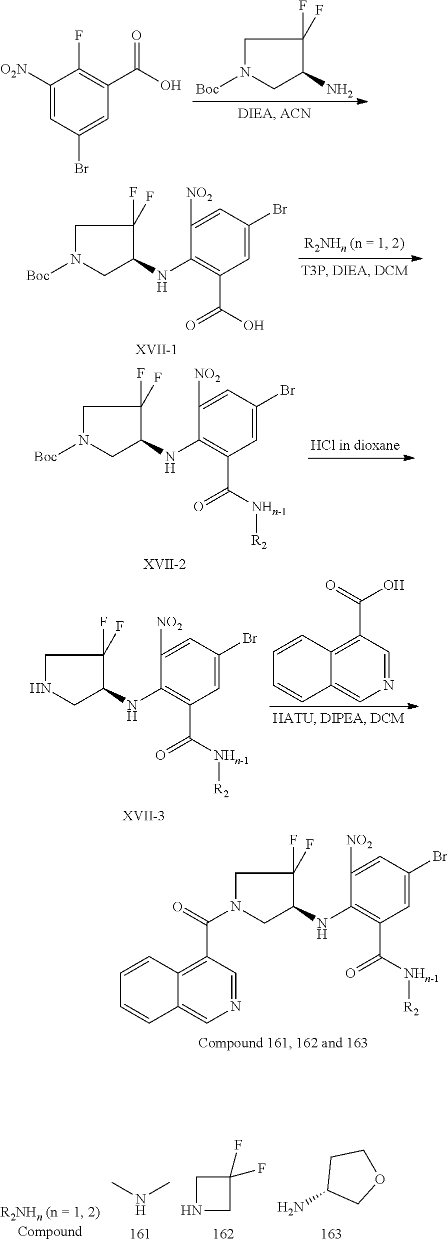

### Intermediate XVI-1: 5-bromo-2-[[(3S)-1-tert-butoxycarbonyl-4,4-difluoro-pyrrolidin-3-yl]amino]-3-nitro-benzoic acid

A mixture of 5-bromo-2-fluoro-3-nitro-benzoic acid (300 mg, 1.14 mmol), tert-butyl (4S)-4-amino-3,3-difluoro-pyrrolidine-1-carboxylate (252 mg, 1.14 mmol) and DIEA (440 mg, 3.41 mmol, 593 uL) in ACN (10.0 mL) was stirred at 85° C. for 8 hrs. The reaction mixture was concentrated under reduced pressure to remove ACN. The residue was washed with water and extracted with DCM (30.0 Ml*3). The combined organic layers were concentrated under reduced pressure to give intermediate XVI-1 (500 mg, crude) as a yellow solid.

### Intermediate XVI-2

The synthetic procedure of intermediate XVI-2 is similar to the last step of the synthetic procedure of compound 14.

### Intermediate XVI-3

The synthetic procedure of intermediate XVI-3 is similar to that of intermediate I-3.

### Compounds 161, 162 and 163

The synthetic procedure of compounds 161, 162 and 163 is similar to the last step of the synthetic procedure of compound 1.

| | |
|---|---|
| 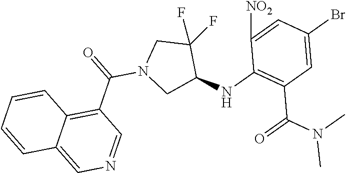 [SMILES: CN(C)C(=O)c1cc(Br)cc([N+](=O)[O-])c1N[C@H]1CN(C(=O)c2cncc3ccccc23)CC1(F)F] | Compound 161: (S)-5-bromo-2-((4,4-difluoro-1-(isoquinoline-4- carbonyl)pyrrolidin-3-yl)amino)-N, N-dimethyl-3-nitrobenzamide ¹H NMR (400 MHz, DMSO-d₆) δ 9.43 (s, 1H), 8.27-8.20 (m, 2H), 7.90-7.89 (m, 2H), 7.78-7.77 (m, 1 H), 7.64-7.63 (m, 1H), 7.38-7.35 (m, 1H), 4.36- 4.17 (m, 3H), 3.75-3.73 (m, 2H), 3.57-3.43 (m, 1H), 3.89 (s, 1H), 3.09 (s, 1H), 2.69-2.64 (m, 4H). LC-MS (ESI) (M + H)⁺ = 548.3. |
| Compound 161 | |
| 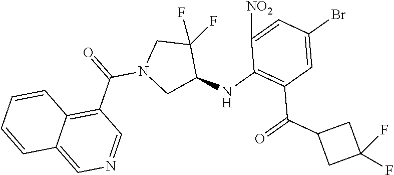 [SMILES: O=C(c1cc(Br)cc([N+](=O)[O-])c1N[C@H]1CN(C(=O)c2cncc3ccccc23)CC1(F)F)C1CC(F)(F)C1] | Compound 162: (S)-(5-bromo-2-((4,4-difluoro-1-(isoquinoline-4- carbonyl)pyrrolidin-3-yl)amino)-3-nitrophenyl)(3,3-difluoroazetidin-1- yl)methanone ¹H NMR (400 MHz, DMSO-d₆) δ 9.42 (s, 1H), 8.58-8.56 (m, 1H), 8.25-8.23 (m, 2H), 7.93-7.91 (m, 2H), 7.89-7.83 (m, 1H), 7.82-7.77 (m, 1H), 7.29-7.20 (m, 1H), 4.61-4.38 (m, 1H), 4.36-4.29 (m, 4H), 4.21-4.14 (m, 2H), 3.66- 3.64 (m, 2H). LC-MS (ESI) (M + H)⁺ = 596.3. |
| Compound 162 | |
| 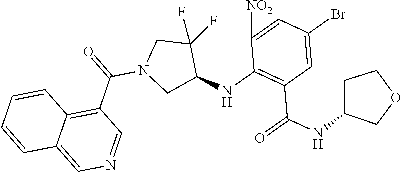 [SMILES: O=C(N[C@@H]1CCOC1)c1cc(Br)cc([N+](=O)[O-])c1N[C@H]1CN(C(=O)c2cncc3ccccc23)CC1(F)F] | Compound 163: 5-bromo-2-(((S)-4,4-difluoro-1-(isoquinoline-4- carbonyl)pyrrolidin-3-yl)amino)-3-nitro-N-((R)-tetrahydrofuran-3- yl)benzamide ¹H NMR (400 MHz, DMSO-d₆) δ 9.43-9.41 (m, 1H), 9.09-8.92 (m, 1H), 8.20-8.19 (m, 2H), 7.92-7.89 (m, 3H), 7.81-7.78 (m, 2H), 4.46-4.16 (m, 2H), 4.16-4.13 (m, 2H), 3.72-3.68 (m, 1H), 3.67-3.65 (m, 3H), 3.65-3.63 (m, 2H), 3.63-3.51 (m, 1H), 2.18-1.86 (m, 1H), 1.85-1.61 (m, 1H). LC-MS (ESI) (M + 3)⁺ = 592.3. |
| Compound 163 | |

### Synthesis of compound 181

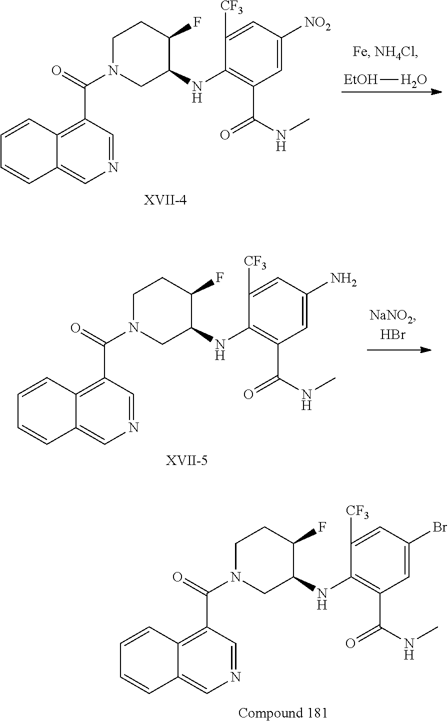

### Intermediate XVI-4

The synthetic procedure of intermediate XVI-4 is similar to that of compound 161.

### Intermediate XVI-5

The synthetic procedure of intermediate XVI-5 is similar to that of compound VIII-4.

### Compounds 181: 5-bromo-2-(((3S,4R)-4-fluoro-1-(isoquinoline-4-carbonyl)piperidin-3-yl)amino)-N-methyl-3-(trifluoromethyl)benzamide

NaNO₂ (29.60 mg, 0.43 mmol) was slowly added to a solution of intermediate XVI-5 (140 mg, 0.29 mmol) in HBr (3.00 mL) at 0° C. The resulting mixture was stirred at 50° C. for 12 hr. The reaction mixture was poured into ice-water, and the pH was adjusted to 8 with Na₂CO₃ solution, and reaction mixture was then extracted with DCM (10 mL*3), dried over Na₂SO₄, and concentrated under reduced pressure. The residue was purified by prep-HPLC (column: C18-6 100*30 mm*5 um; mobile phase: [water (FA)-ACN]; B %: 10%-70%, 15 min) to give compound 181 (10.0 mg, 6.28% yield) as a white solid.

| | |
|---|---|
| 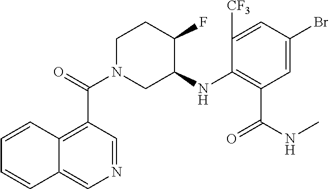 [SMILES: CNC(=O)c1cc(Br)cc(C(F)(F)F)c1N[C@H]1CN(C(=O)c2cncc3ccccc23)CC[C@H]1F] | Compound 181: 5-bromo-2-(((3S, 4R)-4-fluoro-1-(isoquinoline-4- carbonyl)piperidin-3-yl)amino)-N-methyl-3-(trifluoromethyl)benzamide ¹H NMR (400 MHz, DMSO-d₆) δ 9.30-9.44 (m, 1H), 8.42-8.91 (m, 2H), 8.14- 8.25 (m, 1H), 7.61-7.92 (m, 5H), 6.10-6.48 (m, 1H), 4.74-5.04 (m, 1H), 4.14- 4.46 (m, 1H), 3.40-3.78 (m, 2H), 3.02-3.24 (m, 2H), 2.82 (m, 2H), 2.57-2.67 (m, 1H), 1.52-2.16 (m, 2H). LC-MS (ESI) (M + H)⁺ = 553.4. |
| Compound 181 | |

### Example 3. Purification of 3CLpro Enzymes

The 3CLpro enzymes of SARS-CoV-2, SARS-CoV and MERS-CoV were over-expressed in E. coli and purified to homogeneity following protocols described previously (6).

### Example 4. Structural Biology Studies

To understand how the compounds as disclosed herein bind to and inhibit the enzymatic activity of 3CLpro, certain inhibitors were co-crystalized with SARS-CoV-2 3CLpro. Before crystallization, a 10 mg/mL stock of the purified SARS-CoV-2 3CLpro in a buffer containing 20 mM HEPES pH 7.4 and 150 mM NaCl was incubated with 1.5 mM of the inhibitor at room temperature for 2 hours then the precipitate was removed by centrifugation. For crystallization, 0.2 μL of the 3CLpro/inhibitor complex was mixed with 0.2 μL of well buffers from commercially crystallization kits. Crystals were grown at 20° C. in a 96-well plate using the sitting-drop vapor-diffusion method. The diffraction data was collected at 100 K on a Rigaku XtaLAB Synergy Custom diffractometer and solved by molecular replacement using Phaser-MR (7) in the Phenix software suite (8). The crystal structure of the SARS-CoV-2 3CLpro (PDB code: 7EN8) was used as the initial model. The 3CLpro/inhibitor complex structures were manually refined with Coot (9) and Phenix (8).

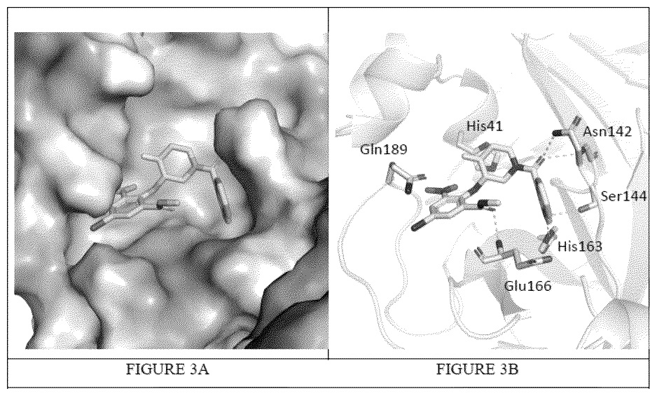

The crystal structure of 3CLpro/Compound 19 complex was determined by molecular replacement and refined to 2.41 Å. Compound 19 formed a 1:1 complex with 3CLpro. In the crystal structure, one molecule of Compound 19 bound into the catalytic pocket of 3CLpro thus competitively inhibiting the binding of 3CLpro substrates (FIG. 3A). Five hydrogen bonds formed between Compound 19 and 3CLpro: two were between the nitrogen atom in the isoquinoline ring of Compound 19 and the side chains of His163 and Ser144; two were between the carbonyl group attached to the isoquinoline ring of Compound 19 and the side chain of Asn142 and the main chain of Gly143; one was between the carbonyl oxygen in the methylcarbamoyl group of Compound 2 and the main chain amide of Glul66 (FIG. 3B).

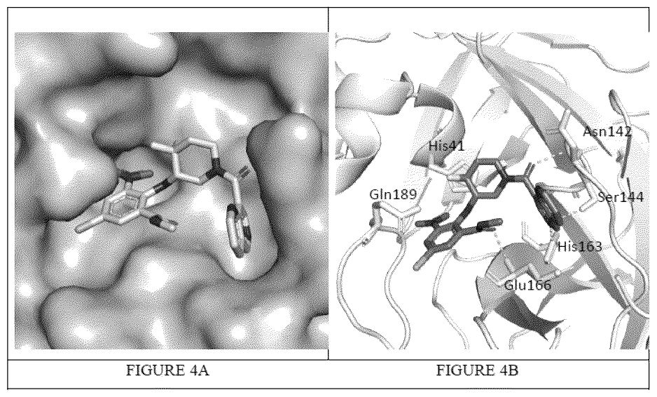

The crystal structure of 3CLpro/Compound 98 complex was refined to 1.93 Å (FIG. 4A). Compound 98 was found to bind to 3CLpro similar to that of Compound 19 (FIG. 4B).

### Example 5. In Vitro Enzymatic Assay Results

The ability of the compounds in Table 1 to inhibit 3CLpro was tested using a fluorescence resonance energy transfer (FRET)-based continuous kinetic assay (6). A fluorescent peptide Dabcyl-KTSAVLQ,SGFRKM-E(Edans) was used as the substrate of purified 3CLpro. After cleavage by 3CLpro, the fragment SGFRKM-E(Edans) was released and its fluorescence could be monitored with the excitation and emission wavelengths of 355 nm and 538 nm, respectively.

For SARS-CoV-2 3CLpro and SARS-CoV 3CLpro, the final concentration of 3CLpro and the fluorogenic substrate (Dabcyl-KTSAVLQ, SGFRKM-E(Edans) were 15 nM and 25 μM, respectively. For MERS-CoV 3CLpro, the final concentration of 3CLpro and the fluorogenic substrate (Dabcyl-KTSAVLQISGFRKM-E(Edans) were 500 nM and 100 μM, respectively. Briefly, 15 μL of the recombinant 3CLpro in the assay buffer (20 mm HEPES 7.4, 150 mM NaCl, 0.01% Triton X-100, 1 mM DTT) was incubated with 1.5 μL of a serial dilution of each compound at room temperature for 1 hour. A 384-well plate (Corning, CLS3575) was pre-warmed for 5 min at 37° C. Then 11 μL of the 3CLpro/inhibitor mixture was transferred to the 384-well plate. The reaction was initiated by adding 29 μL of the fluorogenic substrate in the assay buffer. After that, the fluorescence signal was immediately measured every 1 min at 37° C. using a Thermol Varioskan LUX plate reader with 355 nm excitation and 538 nm emission.

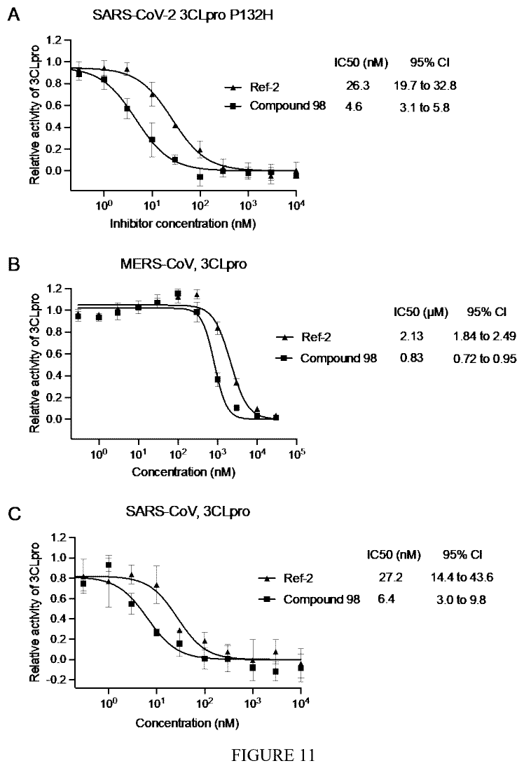

The inhibitory activity data of some of the representative compounds was shown in Table 2 and FIG. 11.

### Example 6. Isothermal Titration Calorimetry (ITC)

ITC experiments were done with an isothermal titration calorimeter MicroCal PEAQ-ITC (Malvern Panalytical). The inhibitor with a concentration of 20 μM in the ITC buffer (20 mM HEPES, pH 7.4, 150 mM NaCl, 0.5% DMSO) was titrated by 200 M 3CLpro in the ITC buffer at 25° C. Control experiments in which the inhibitors were titrated by the ITC buffer were performed and the data was used to correct the 3CLpro titration data. The data was processed using the MicroCal PEAQ-ITC analysis software. Each measurement was repeated three times.

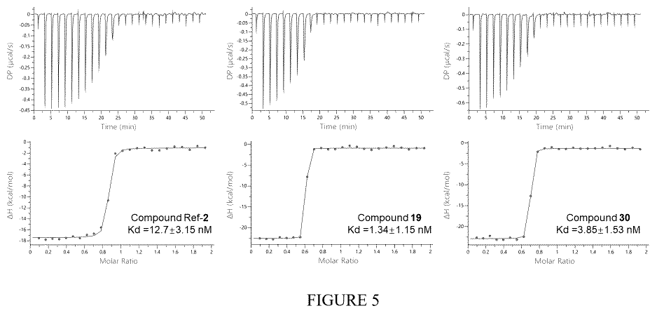

The ITC data of some of the representative compounds was shown in FIG. 5. From the ITC data, compounds 19 and 30 showed a higher binding affinity (dissociation constant, Kd) than compound Ref-2 (compound P2) in previously filed patent (WO2022150962).

### Example 7. Anti-SARS-CoV-2 Activity of 3CLpro Inhibitors in Cellular Assays

The anti-SARS-CoV-2 activity was measured in A549 cells expressing the human ACE2 receptor following protocols described previously (6). Briefly, the cells were first treated with DMSO or various concentrations of the compounds (Table 1) for 1 hour, then infected by severe respiratory syndrome coronavirus 2 (SARS-CoV-2) for 24 hours. After paraformaldehyde fixation, the cells were stained with SARS-CoV-2 nucleocapsid (N) protein antibody and DAPI. The percentage of the cells infected by SARS-CoV-2 was measured by imaging using a high-content imaging system.

The anti-Omicron SARS-CoV-2 activity was measured in Caco-2 cells using similar protocol.

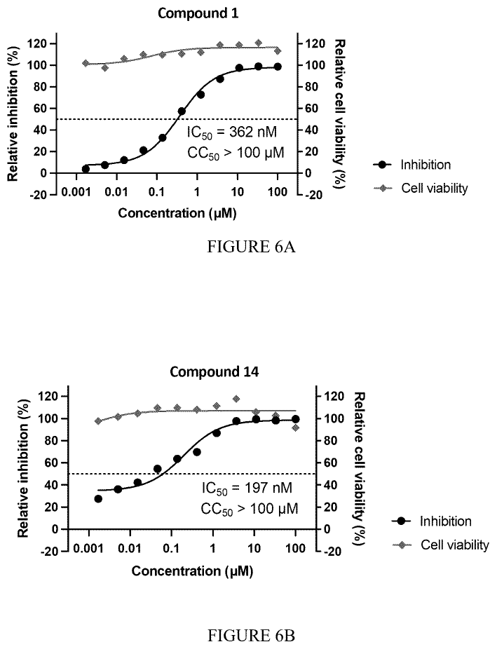

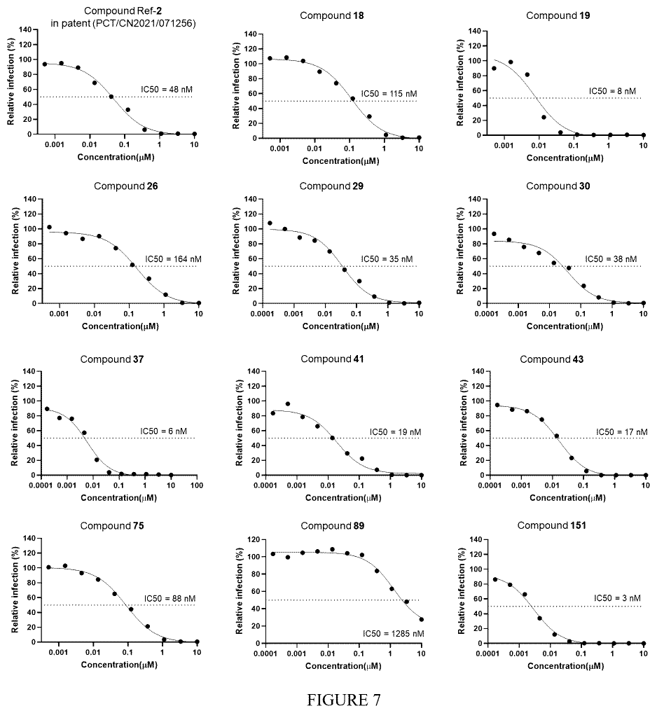

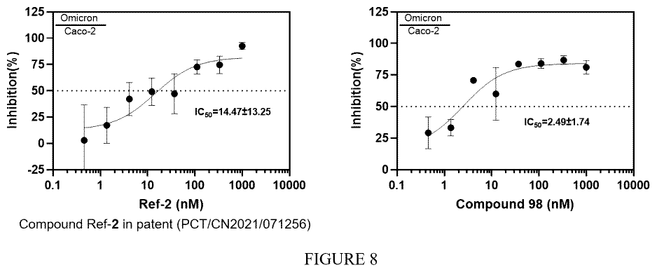

The above testing results showed that the non-covalent small molecule inhibitors of SARS-CoV-2 3CLpro effectively blocked coronavirus replication in host cells (FIG. 6, FIG. 7, FIG. 8, and Table 2). These inhibitors can be used for the treatment of a disease, or a symptom of a disease caused by a coronavirus, such as COVID-19.

Comparing to compound Ref-2 in previously filed patent application (WO2022150962), compounds 19, 29, 30, 37, 41, 43 and 151 showed a better antiviral potency in A549 cells (FIG. 7). Compound 98 demonstrated a stronger activity against Omicron SARS-CoV-2 in Caco-2 cells (FIG. 8).

### Example 8. CYP Inhibition Evaluation of Representative Compounds in Human Liver Microsomes

20 μL of the substrate solutions of CYP1A2, CYP2C9, CYP2C19, CYP2D6 and CYP3A4 were each mixed with 2 L of the test compounds (final concentrations: 50 μM, 15 μM, 5 μM, 1.5 μM, 0.5 μM, 0.15 μM, 0.05 μM, 0 μM), followed by the addition of 158 μL of the HLM (Human liver microsome) working solution. Each of the mixtures was incubated at 37° C. for 10 minutes, and then mixed with 20 μL of NADPH cofactor, followed by incubation at 37° C. for additional 10 minutes. The reactions were terminated by adding 400 μL cold stop solution (200 ng/mL Tolbutamide and Labetalol in ACN). The samples are centrifuged at 4000 rpm for 20 minutes to precipitate protein. Next, 200 μL of the supernatant of each sample was transferred to 100 μL HPLC water and shaken for 10 minutes, and then analyzed by LC/MS/MS.

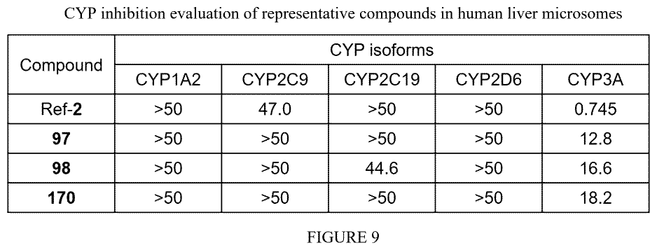

The above testing results showed that compounds 97, 98 and 170 have little effect on different CYP isoforms (FIG. 9).

### Example 9. Plasma Protein Binding (PPB)

The binding of compounds Ref-2, 86, 97, 98 and 170 to human and mouse plasma were measured using equilibrium dialysis. A loading matrix was prepared by diluting of a 400 μM stock of each of the compounds in DMSO by the plasma matrix. To prepare the time zero (To) samples to be used for recovery determination, 50 μL of the loading matrix were transferred in triplicate to the sample collection plate, immediately matched with opposite blank buffer to obtain a final volume of 100 μL of 1:1 matrix/dialysis buffer (v/v) in each well, followed by the addition of 500 μL of stop solution (200 ng/mL of tolbutamide, 200 ng/mL of labetalol, and 50 ng/mL of metformin in acetonitrile) and then stored at 2-8° C. pending further processes along with other post-dialysis samples. To load the dialysis device, 150 μL of the loading matrix and 150 μL of the dialysis buffer were transferred to the donor side and the receiver side of each well, respectively. The dialysis plate was placed in a humidified incubator at 37° C. with 5% CO₂ on a shaking platform that rotated slowly (about 100 rpm) for 4 hours, then 50 μL of samples were taken from both the buffer side and the matrix side, and transferred into new 96-well plates. Each sample was mixed with an equal volume of opposite blank buffer (or matrix) to reach a final volume of 100 μL, followed by the addition of 500 L of stop solution. The mixture was vortexed and centrifuged at 4000 rpm for about 20 minutes, then 100 L of the supernatant of each sample was removed for LC-MS/MS analysis. The single blank samples were prepared by transferring 50 μL of blank matrix to a 96 well plate and adding 50 μL of blank PBS buffer to each well. The blank plasma must match the species of plasma used in the plasma side of the well. Then the matrix-matched samples were further processed by adding 500 μL of stop solution, following the same sample processing method as the dialysis samples. The % Unbound, % Bound and % Recovery were calculated using the following equations:

$$\% Unbound = 100 \times F / T$$

$$\% Bound = 100 - \% Unbound$$

$$\% Recovery = 100 \times ( F + T ) / {T}_{0}$$

in which F is the analyte concentration or peak area ratio of analyte/internal standard on the buffer (receiver) side of the membrane, T is the analyte concentration or peak area ratio of analyte/internal standard on the matrix (donor) side of the membrane, and T₀ is the analyte concentration or the peak area ratio of analyte/internal standard in the loading matrix sample at time zero.

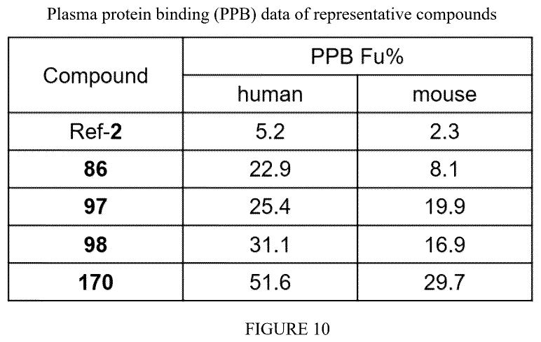

The above testing results showed that compounds 86, 97, 98 and 170 have better free PPB ratios in comparison with compound Ref-2 (FIG. 10).

### REFERENCES

1. A. R. Fehr, S. Perlman, Coronaviruses: an overview of their replication and pathogenesis. Methods Mol Biol 1282, 1-23 (2015).
2. P. Zhou et al., A pneumonia outbreak associated with a new coronavirus of probable bat origin. Nature 579, 270-273 (2020).
3. J. Lei, Y. Kusov, R. Hilgenfeld, Nsp3 of coronaviruses: Structures and functions of a large multi-domain protein. Antiviral Res 149, 58-74 (2018).
4. D. R. Owen et al., An oral SARS-CoV-2 M(pro) inhibitor clinical candidate for the treatment of COVID-19. Science 374, 1586-1593 (2021).
5. Y. Unoh et al., Discovery of S-217622, a Noncovalent Oral SARS-CoV-2 3CL Protease Inhibitor Clinical Candidate for Treating COVID-19. J Med Chem 65, 6499-6512 (2022).
6. N. Hou et al., Development of Highly Potent Noncovalent Inhibitors of SARS-CoV-2 3CLpro. ACS Cent Sci 9, 217-227 (2023).
7. A. J. McCoy et al., Phaser crystallographic software. J Appl Crystallogr 40, 658-674 (2007).
8. D. Liebschner et al., Macromolecular structure determination using X-rays, neutrons and electrons: recent developments in Phenix. Acta Crystallogr D Struct Biol 75, 861-877 (2019).
9. P. Emsley, B. Lohkamp, W. G. Scott, K. Cowtan, Features and development of Coot. Acta Crystallogr D Biol Crystallogr 66, 486-501 (2010).

One skilled in the art will readily recognize from the disclosure and claims that various changes, modifications, and variations can be made therein without departing from the spirit and scope of the disclosure as defined in the following claims.

1. A compound represented by Formula I: 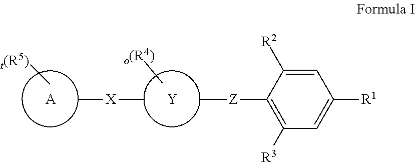
a solvate thereof, a tautomer thereof, a stereoisomer thereof, a deuterated derivative of the compound, or a pharmaceutically acceptable salt of the foregoing, wherein:
R1 is hydrogen, halogen, or C1-C2 alkyl that is optionally substituted with 1 to 3 groups of halogen;
R2 is —NO2, —SO3H, —SO2CH3, —CN or —CF3;
R3 is —C(═O)NRbRc or 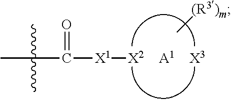
wherein:
Rb is hydrogen, C1-C4 alkyl, C2-C4 alkenyl, or C2-C4 alkynyl, wherein the C1-C4 alkyl, C2-C4 alkenyl, or C2-C4 alkynyl of Rb is optionally substituted with 1-3 groups selected from halogen and deuterium;
Rc is hydrogen, NH2, —OH, C1-C4 alkyl, C2-C4 alkenyl, or C2-C4 alkynyl, wherein the C1-C4 alkyl, C2-C4 alkenyl, or C2-C4 alkynyl of Rc is optionally substituted with 1-3 groups selected from halogen, deuterium, and —OH;
Ring A1 is 3-, 4-, 5- or 6-membered cycloalkyl, or 4-, 5- or 6-membered heterocycloalkyl or 5- or 6-membered heteroaryl, wherein the heterocycloalkyl or heteroaryl of Ring A¹ contains one or more heteroatoms selected from N and 0;
X1 is —CH2—, —NH— or —O—;
X2 is CH or N;
X3 is —O—, —CH2, or —NH—;
R3′, for each occurrence, is independently selected from halogen, —ORd, —NReRf, and C1-C4 alkyl that is optionally substituted with 1-3 groups of halogen; wherein:
Rd is hydrogen, C1-C4 alkyl, C2-C4 alkenyl, or C2-C4 alkynyl, wherein the C1-C4 alkyl, C2-C4 alkenyl, or C2-C4 alkynyl of Rd is optionally substituted with 1-3 groups of halogen;
Re is hydrogen, C1-C4 alkyl, C2-C4 alkenyl, or C2-C4 alkynyl, wherein the C1-C4 alkyl, C2-C4 alkenyl, or C2-C4 alkynyl of Re is optionally substituted with 1-3 groups of halogen;
Rf is hydrogen, C1-C4 alkyl, C2-C4 alkenyl, or C2-C4 alkynyl, wherein the C1-C4 alkyl, C2-C4 alkenyl, or C2-C4 alkynyl of Rf is optionally substituted with 1-3 groups of halogen;
R3′, for each occurrence, may be attached to any of the ring atoms of the Ring A1, as long as valence permits; and
m is an integer selected from 0, 1, 2 and 3;
Z is —NRg-, —C(RhRi)-NRg-, —NRg-C(RhRi)-, or —O—, wherein:
Rg is hydrogen or C1-C6 alkyl;
Ring Y is 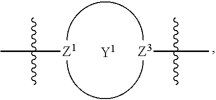
wherein:
Ring Y1 is 4-, 5-, 6-, or 7-membered heterocycloalkyl containing one or more heteroatoms selected from N and 0;
Z1, and Z3 for each occurrence are each independently CH or N;
R4, for each occurrence, is independently halogen, —OH, —C(═O)Rj, C1-C4 alkyl, C2-C4 alkenyl, or C2-C4 alkynyl wherein the C1-C4 alkyl, C2-C4 alkenyl, or C2-C4 alkynyl of R4 is optionally substituted with 1-3 groups of halogen; wherein:
Rj is C1-C4 alkyl, C2-C4 alkenyl, or C2-C4 alkynyl wherein the C1-C4 alkyl, C2-C4 alkenyl, or C2-C4 alkynyl of Rj is optionally substituted with 1-3 groups of halogen;
R4, for each occurrence, may be attached to any of the ring atoms of Ring Y, as long as valence permits;
is an integer selected from 0, 1 or 2;
X is —C(═O)—; wherein:
Ring A is 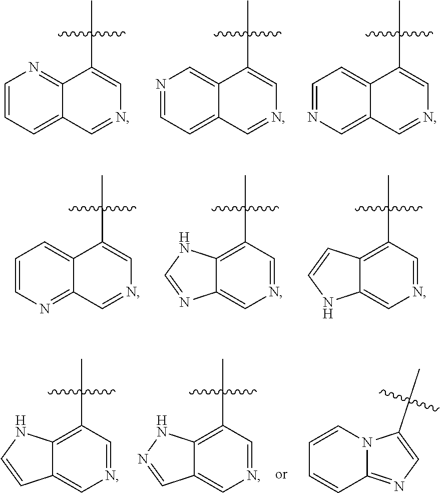
R5, for each occurrence, is independently halogen, —CN, —ORp, or C1-C4 alkyl; wherein:
Rp, for each occurrence, is independently selected from hydrogen and C1-C4 alkyl; and
t is 0, 1, 2, 3, 4, or 5.

2. The compound, solvate, tautomer, stereoisomer, deuterated derivative, or pharmaceutically acceptable salt according to claim 1, wherein R3 is 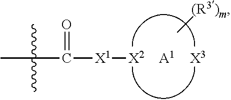
wherein Ring A1, X1, X2, X3, R3′ and m are as defined in claim 1; or
wherein X1 is —CH2—, —NH— or —O—, and Ring A1, X2, X3, R3′ and m are as defined in claim 1; or
wherein X1 is —NH—, and Ring A1, X2, X3, R3′ and m are as defined in claim 1; or
wherein X1 is —NH—, and Ring A¹ is 3-, 4-, 5- or 6-membered cycloalkyl, and X2, X3, R3′ and m are as defined in claim 1; or
wherein X1 is —NH—, and Ring A¹ is 4-, 5- or 6-membered heterocycloalkyl, wherein the heterocycloalkyl of Ring A¹ contains one or more heteroatoms selected from N and O, and
X2, X3, R3′ and m are as defined in claim 1.

3-6. (canceled)

7. The compound, solvate, tautomer, stereoisomer, deuterated derivative, or pharmaceutically acceptable salt according to claim 1, wherein:
Re is H, Rf is H, NH2, —OH, C1-C4 alkyl, C2-C4 alkenyl, or C2-C4 alkynyl, wherein the C1-C4 alkyl, C2-C4 alkenyl, or C2-C4 alkynyl of Rf is optionally substituted with 1-3 groups of halogen.

8. The compound, solvate, tautomer, stereoisomer, deuterated derivative, or pharmaceutically acceptable salt according to claim 1, wherein:
when Rb is H, Rc is H, NH2, —OH, C1-C4 alkyl, C2-C4 alkenyl, or C2-C4 alkynyl, wherein the C1-C4 alkyl, C2-C4 alkenyl, or C2-C4 alkynyl of Rc is optionally substituted with 1-3 groups selected from halogen, deuterium, and —OH.

9. The compound, solvate, tautomer, stereoisomer, deuterated derivative, or pharmaceutically acceptable salt according to claim 1, wherein:
R3 is
R3 is —C(═O)NHCH2CH═CH2, —C(═O)NHINH2, —C(═O)NHCH(CH3)CF3, —C(═O)NHCH2CHF2, —C(═O)NHCH3, —C(═O)NHCD3, —C(═O)NHCH(CH3)CF3, —C(═O)NHCH2(CH3)2CH2OH,

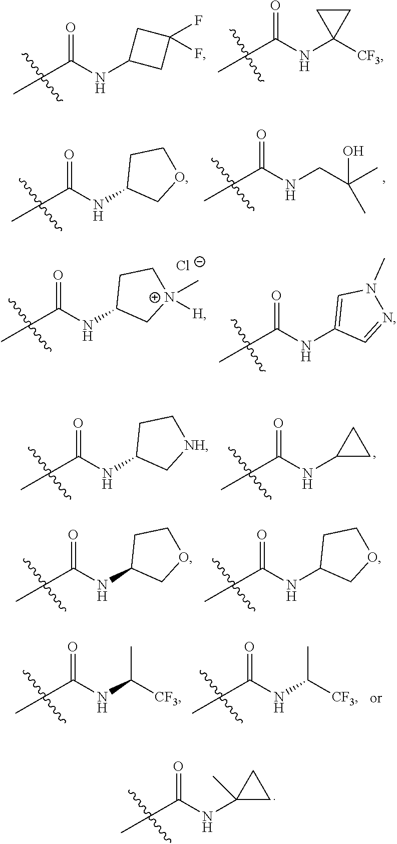

10. The compound, solvate, tautomer, stereoisomer, deuterated derivative, or pharmaceutically acceptable salt according to claim 1, wherein:
the compound is of the following structural Formula II-2, II-3, II-4, or II-5:

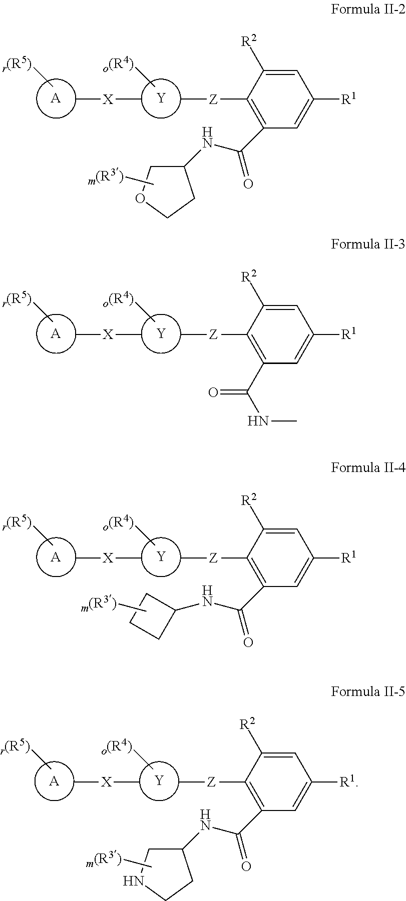

11. The compound, solvate, tautomer, stereoisomer, deuterated derivative, or pharmaceutically acceptable salt according to claim 1, wherein R1 is Br, F or Cl.

12. The compound, solvate, tautomer, stereoisomer, deuterated derivative, or pharmaceutically acceptable salt according to claim 1, wherein:
the compound is of the following structural Formula III or Formula IV: 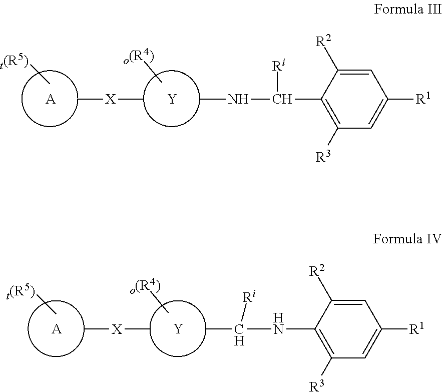
wherein:
Ri is C1-C6 branched alkyl that is optionally substituted with 1-3 groups of halogen, or C4-C8 alkyl with a C3-C6 carbocyclyl side chain and optionally substituted with 1-3 groups of halogen.

13. The compound, solvate, tautomer, stereoisomer, deuterated derivative, or pharmaceutically acceptable salt of the foregoing according to claim 1, wherein:
the compound is of the following structural Formula V: 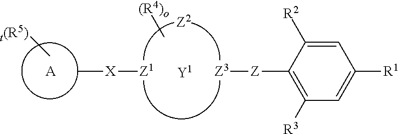
Formula V
wherein:
when Z1 is CH, X is —C(═O)NRn- or -Rm-C(═O)NRnRo-;
when Z3 is CH, Z is —NRg-, —C(RhRi)—NRg-, —NRg-C(RhRi)-, or —O—.

14. The compound, solvate, tautomer, stereoisomer, deuterated derivative, or pharmaceutically acceptable salt according to claim 1, wherein:
Z1 is next to Z3, or Z1 is next but one to Z3.

15-21. (canceled)

22. The compound, solvate, tautomer, stereoisomer, deuterated derivative, or pharmaceutically acceptable salt according to claim 1, wherein:
the moiety

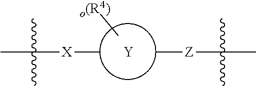

[chemical structure]

23-26. (canceled)

27. The compound, solvate, tautomer, stereoisomer, deuterated derivative, or pharmaceutically acceptable salt of claim 1, wherein the compound is selected from the group consisting of:

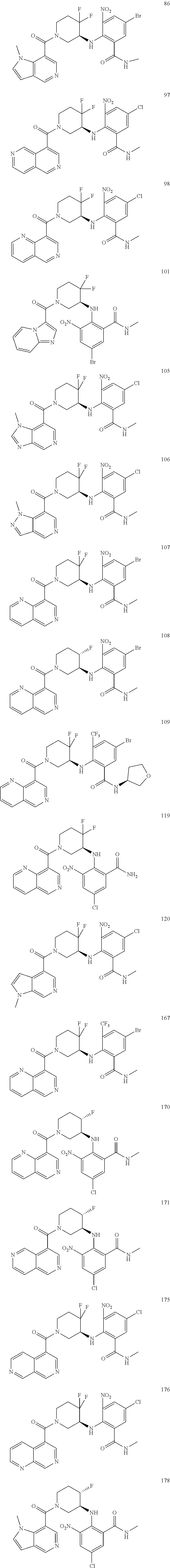

28. A pharmaceutical composition comprising the compound, solvate, tautomer, stereoisomer, deuterated derivative, or pharmaceutically acceptable salt according to claim 1 and at least one pharmaceutically acceptable carrier.

29. The pharmaceutical composition according to claim 28, further comprising one or more additional active pharmaceutical agents useful for treating a coronavirus infection.

30. A method of treating a disease or a symptom of a disease caused by a coronavirus in a subject, comprising administering to the subject, a therapeutically effective amount of the compound, solvate, tautomer, stereoisomer, deuterated derivative, or pharmaceutically acceptable salt according to claim 1.

31. The method according to claim 30, wherein the disease is a respiratory tract infectious disease, preferably a severe acute respiratory syndrome.

32-40. (canceled)
